SEQUENCE LISTING (173 sequences)

SEQ ID NO: 1 - protein, 30 aa, Homo sapiens
HAEGTFTSDVSSYLEGQAAKEFIAWLVKGR

SEQ ID NO: 2 - protein, 39 aa, Heloderma suspectum
HGEGTFTSDLSKQMEEEAVRLFIEWLKNGGPSSGAPPPS

SEQ ID NO: 3 - protein, 40 aa, Artificial sequence
  Exendin-4 peptide with C-terminus lysine addition
HGEGTFTSDLSKQMEEEAVRLFIEWLKNGGPSSGAPPPSK

SEQ ID NO: 4 - protein, 40 aa, Artificial sequence
  Exendin-4 peptide with Gly to Aib2 substitution at position 2 and C-terminal lysine addition
HXEGTFTSDLSKQMEEEAVRLFIEWLKNGGPSSGAPPPSK

SEQ ID NO: 5 - protein, 39 aa, Artificial sequence
  Exendin-4 peptide with Gly to Aib2 substitution at position 2
HXEGTFTSDLSKQMEEEAVRLFIEWLKNGGPSSGAPPPS

SEQ ID NO: 6 - protein, 30 aa, Artificial sequence
  Exendin-4 peptide with Gly to Aib2 substitution at position 2 and 9 amino acid carboxy truncation
HXEGTFTSDLSKQMEEEAVRLFIEWLKNGG

SEQ ID NO: 7 - protein, 28 aa, Artificial sequence
  Exendin-4 peptide with Gly to Aib2 substitution at position 2 and 11 amino acid carboxy truncation
HXEGTFTSDLSKQMEEEAVRLFIEWLKN

SEQ ID NO: 8 - protein, 28 aa, Artificial sequence
  Exendin-4 peptide with Gly to Aib2 substitution at position 2, Met to Leu substitution at position 14, Trp to Phe substitution at position 25, and 11 amino acid carboxy truncation
HXEGTFTSDLSKQLEEEAVRLFIEFLKN

SEQ ID NO: 9 - protein, 28 aa, Artificial sequence
  Exendin-4 peptide with Gly to Aib2 substitution at position 2, Met to Leu substitution at position 14, Phe to Ala substitution at position 22, Trp to Phe substitution at position 25, and 11 amino acid carboxy truncation
HXEGTFTSDLSKQLEEEAVRLAIEFLKN

SEQ ID NO: 10 - protein, 39 aa, Artificial sequence
  Exendin-4 peptide with Gly to Aib2 substitution at position 2, Met to Leu substitution at position 14, Phe to Ala substitution at position 22, and Trp to Phe substitution at position 25
HXEGTFTSDLSKQLEEEAVRLAIEFLKNGGPSSGAPPPS

SEQ ID NO: 11 - protein, 39 aa, Artificial sequence
  Exendin-4 peptide with Gly to Aib2 substitution at position 2, Met to Leu substitution at position 14, and Trp to Phe substitution at position 25
HXEGTFTSDLSKQLEEEAVRLFIEFLKNGGPSSGAPPPS

SEQ ID NO: 12 - protein, 39 aa, Artificial sequence
  Exendin-4 peptide with Gly to Aib2 substitution at position 2, K to K(Ac) substitution at position 12, and K to K(Ac) substitution at position 27
HXEGTFTSDLSXQMEEEAVRLFIEWLXNGGPSSGAPPPS

SEQ ID NO: 13 - protein, 39 aa, Artificial sequence; identical to SEQ ID NO: 12
  Exendin-4 peptide with Gly to Aib2 substitution at position 2, K to K(benzoyl) substitution at position 12, and K to K(benzoyl) substitution at position 27

SEQ ID NO: 14 - protein, 39 aa, Artificial sequence
  Exendin-4 peptide with Gly to Aib2 substitution at position 2, Pro to Lys substitution at position 38
HXEGTFTSDLSKQMEEEAVRLFIEWLKNGGPSSGAPPKS

SEQ ID NO: 15 - protein, 39 aa, Artificial sequence
  Exendin-4 peptide with Gly to Aib2 substitution at position 2, Pro to Lys substitution at position 36
HXEGTFTSDLSKQMEEEAVRLFIEWLKNGGPSSGAKPPS

SEQ ID NO: 16 - protein, 39 aa, Artificial sequence
  Exendin-4 peptide with Gly to Aib2 substitution at position 2, Gly to Lys substitution at position 34
HXEGTFTSDLSKQMEEEAVRLFIEWLKNGGPSSKAPPPS

SEQ ID NO: 17 - protein, 39 aa, Artificial sequence
  Exendin-4 peptide with Gly to Aib2 substitution at position 2, Ser to Lys substitution at position 32
HXEGTFTSDLSKQMEEEAVRLFIEWLKNGGPKSGAPPPS

SEQ ID NO: 18 - protein, 39 aa, Artificial sequence
  Exendin-4 peptide with Gly to Aib2 substitution at position 2, Asn to Lys substitution at position 28
HXEGTFTSDLSKQMEEEAVRLFIEWLKKGGPSSGAPPPS

SEQ ID NO: 19 - protein, 39 aa, Artificial sequence
  Exendin-4 peptide with Gly to Aib2 substitution at position 2, Leu to Lys substitution at position 26
HXEGTFTSDLSKQMEEEAVRLFIEWKKNGGPSSGAPPPS

SEQ ID NO: 20 - protein, 39 aa, Artificial sequence
  Exendin-4 peptide with Gly to Aib2 substitution at position 2, Glu to Lys substitution at position 24
HXEGTFTSDLSKQMEEEAVRLFIKWLKNGGPSSGAPPPS

SEQ ID NO: 21 - protein, 39 aa, Artificial sequence
  Exendin-4 peptide with Gly to Aib2 substitution at position 2, Ile to Lys substitution at position 23
HXEGTFTSDLSKQMEEEAVRLFKEWLKNGGPSSGAPPPS

SEQ ID NO: 22 - protein, 39 aa, Artificial sequence
  Exendin-4 peptide with Gly to Aib2 substitution at position 2, Leu to Lys substitution at position 21
HXEGTFTSDLSKQMEEEAVRKFIEWLKNGGPSSGAPPPS

SEQ ID NO: 23 - protein, 39 aa, Artificial sequence
  Exendin-4 peptide with Gly to Aib2 substitution at position 2, Arg to Lys substitution at position 20
HXEGTFTSDLSKQMEEEAVKLFIEWLKNGGPSSGAPPPS

SEQ ID NO: 24 - protein, 39 aa, Artificial sequence
  Exendin-4 peptide with Gly to Aib2 substitution at position 2, Val to Lys substitution at position 19
HXEGTFTSDLSKQMEEEAKRLFIEWLKNGGPSSGAPPPS

SEQ ID NO: 25 - protein, 39 aa, Artificial sequence
  Exendin-4 peptide with Gly to Aib2 substitution at position 2, Glu to Lys substitution at position 17
HXEGTFTSDLSKQMEEKAVRLFIEWLKNGGPSSGAPPPS

SEQ ID NO: 26 - protein, 39 aa, Artificial sequence
  Exendin-4 peptide with Gly to Aib2 substitution at position 2, Glu to Lys substitution at position 16
HXEGTFTSDLSKQMEKEAVRLFIEWLKNGGPSSGAPPPS

SEQ ID NO: 27 - protein, 39 aa, Artificial sequence
  Exendin-4 peptide with Gly to Aib2 substitution at position 2, Met to Lys substitution at position 14
HXEGTFTSDLSKQKEEEAVRLFIEWLKNGGPSSGAPPPS

SEQ ID NO: 28 - protein, 39 aa, Artificial sequence
  Exendin-4 peptide with Gly to Aib2 substitution at position 2, Gln to Lys substitution at position 13
HXEGTFTSDLSKKMEEEAVRLFIEWLKNGGPSSGAPPPS

SEQ ID NO: 29 - protein, 39 aa, Artificial sequence
  Exendin-4 peptide with Gly to Aib2 substitution at position 2, Ser to Lys substitution at position 11
HXEGTFTSDLKKQMEEEAVRLFIEWLKNGGPSSGAPPPS

SEQ ID NO: 30 - protein, 31 aa, Artificial sequence
  Exendin-4 peptide with Gly to Aib2 substitution at position 2, Pro to Lys substitution at position 31, and 8 amino acid carboxy truncation
HXEGTFTSDLSKQMEEEAVRLFIEWLKNGGK

SEQ ID NO: 31 - protein, 33 aa, Artificial sequence
  Exendin-4 peptide with Gly to Aib2 substitution at position 2, Leu to Lys substitution at position 21, and 6 amino acid carboxy truncation
HXEGTFTSDLSKQMEEEAVRKFIEWLKNGGPSS

SEQ ID NO: 32 - protein, 30 aa, Artificial sequence
  GLP-1 (7-36) peptide with Ala to Aib2 substitution at position 2
HXEGTFTSDVSSYLEGQAAKEFIAWLVKGR

SEQ ID NO: 33 - protein, 31 aa, Artificial sequence
  GLP-1 (7-36) peptide with C-terminal lysine addition
HAEGTFTSDVSSYLEGQAAKEFIAWLVKGRK

SEQ ID NO: 34 - protein, 31 aa, Artificial sequence
  GLP-1 (7-36) peptide with Ala to Gly substitution at position 2, C-terminal lysine addition
HGEGTFTSDVSSYLEGQAAKEFIAWLVKGRK

SEQ ID NO: 35 - protein, 30 aa, Artificial sequence
  GLP-1 (7-36) peptide with Ala to Aib2 substitution at position 2, Gly to Aib2 substitution at position 29
HXEGTFTSDVSSYLEGQAAKEFIAWLVKXR

SEQ ID NO: 36 - protein, 31 aa, Artificial sequence
  GLP-1 (7-36) peptide with Ala to Aib2 substitution at position 2, Gly to Aib2 substitution at position 29, and C-terminal lysine addition
HXEGTFTSDVSSYLEGQAAKEFIAWLVKXRK

SEQ ID NO: 37 - protein, 31 aa, Artificial sequence
  GLP-1 (7-36) peptide with Ala to Aib2 substitution at position 2, C-terminal lysine addition
HXEGTFTSDVSSYLEGQAAKEFIAWLVKGRK

SEQ ID NO: 38 - protein, 31 aa, Artificial sequence
  GLP-1 (7-36) peptide with Ala to Aib2 substitution at position 2, C-terminal Lys(SH) addition
HXEGTFTSDVSSYLEGQAAKEFIAWLVKGRX

SEQ ID NO: 39 - protein, 30 aa, Artificial sequence
  GLP-1 (7-36) peptide with Ala to Aib2 substitution at position 2, Lys to Lys(SH) substitution at position 20
HXEGTFTSDVSSYLEGQAAXEFIAWLVKGR

SEQ ID NO: 40 - protein, 30 aa, Artificial sequence
  GLP-1 (7-36) peptide with Ala to Aib2 substitution at position 2, Leu to Gly substitution at position 14, and Gly to Aib2 at position 29
HXEGTFTSDVSSYGEGQAAKEFIAWLVKXR

SEQ ID NO: 41 - protein, 30 aa, Artificial sequence
  GLP-1 (7-36) peptide with Ala to Aib2 substitution at position 2, Leu to Cys substitution at position 14, and Gly to Aib2 at position 29
HXEGTFTSDVSSYCEGQAAKEFIAWLVKXR

SEQ ID NO: 42 - protein, 30 aa, Artificial sequence
  GLP-1 (7-36) peptide with Ala to Aib2 substitution at position 2, Leu to Phe substitution at position 14, and Gly to Aib2 at position 29
HXEGTFTSDVSSYFEGQAAKEFIAWLVKXR

SEQ ID NO: 43 - protein, 30 aa, Artificial sequence
  GLP-1 (7-36) peptide with Ala to Aib2 substitution at position 2, Leu to Tyr substitution at position 14, and Gly to Aib2 at position 29
HXEGTFTSDVSSYYEGQAAKEFIAWLVKXR

SEQ ID NO: 44 - protein, 30 aa, Artificial sequence
  GLP-1 (7-36) peptide with Ala to Aib2 substitution at position 2, Leu to Trp substitution at position 14, and Gly to Aib2 at position 29
HXEGTFTSDVSSYWEGQAAKEFIAWLVKXR

SEQ ID NO: 45 - protein, 30 aa, Artificial sequence
  GLP-1 (7-36) peptide with Ala to Aib2 substitution at position 2, Gly to Glu substitution at position 16, and Gly to Aib2 at position 29
HXEGTFTSDVSSYLEEQAAKEFIAWLVKXR

SEQ ID NO: 46 - protein, 30 aa, Artificial sequence
  GLP-1 (7-36) peptide with Ala to Aib2 substitution at position 2, Gly to Asp substitution at position 16, and Gly to Aib2 at position 29
HXEGTFTSDVSSYLEDQAAKEFIAWLVKXR

SEQ ID NO: 47 - protein, 30 aa, Artificial sequence
  GLP-1 (7-36) peptide with Ala to Aib2 substitution at position 2, Gly to Lys substitution at position 16, and Gly to Aib2 at position 29
HXEGTFTSDVSSYLEKQAAKEFIAWLVKXR

SEQ ID NO: 48 - protein, 30 aa, Artificial sequence
  GLP-1 (7-36) peptide with Ala to Aib2 substitution at position 2, Ala to Val substitution at position 19, and Gly to Aib2 at position 29
HXEGTFTSDVSSYLEGQAVKEFIAWLVKXR

SEQ ID NO: 49 - protein, 30 aa, Artificial sequence
  GLP-1 (7-36) peptide with Ala to Aib2 substitution at position 2, Ala to Ile substitution at position 19, and Gly to Aib2 at position 29
HXEGTFTSDVSSYLEGQAIKEFIAWLVKXR

SEQ ID NO: 50 - protein, 30 aa, Artificial sequence
  GLP-1 (7-36) peptide with Ala to Aib2 substitution at position 2, Ala to Leu substitution at position 19, and Gly to Aib2 at position 29
HXEGTFTSDVSSYLEGQALKEFIAWLVKXR

SEQ ID NO: 51 - protein, 30 aa, Artificial sequence
  GLP-1 (7-36) peptide with Ala to Aib2 substitution at position 2, Lys to Arg substitution at position 20, and Gly to Aib2 at position 29
HXEGTFTSDVSSYLEGQAAREFIAWLVKXR

SEQ ID NO: 52 - protein, 30 aa, Artificial sequence
  GLP-1 (7-36) peptide with Ala to Aib2 substitution at position 2, Lys to Orn substitution at position 20, and Gly to Aib2 at position 29
HXEGTFTSDVSSYLEGQAAXEFIAWLVKXR

SEQ ID NO: 53 - protein, 30 aa, Artificial sequence
  GLP-1 (7-36) peptide with Ala to Aib2 substitution at position 2, Trp to Phe substitution at position 25, and Gly to Aib2 at position 29
HXEGTFTSDVSSYLEGQAAKEFIAFLVKXR

SEQ ID NO: 54 - protein, 30 aa, Artificial sequence
  GLP-1 (7-36) peptide with Ala to Aib2 substitution at position 2, Val to Ile substitution at position 27, and Gly to Aib2 at position 29
HXEGTFTSDVSSYLEGQAAKEFIAWLIKXR

SEQ ID NO: 55 - protein, 30 aa, Artificial sequence
  GLP-1 (7-36) peptide with Ala to Aib2 substitution at position 2, Lys to Arg substitution at position 28, and Gly to Aib2 at position 29
HXEGTFTSDVSSYLEGQAAKEFIAWLVRXR

SEQ ID NO: 56 - protein, 30 aa, Artificial sequence
  GLP-1 (7-36) peptide with Ala to Aib2 substitution at position 2, Lys to Orn substitution at position 28, and Gly to Aib2 at position 29
HXEGTFTSDVSSYLEGQAAKEFIAWLVXXR

SEQ ID NO: 57 - protein, 30 aa, Artificial sequence
  GLP-1 (7-36) peptide with Ala to Aib2 substitution at position 2, Gly to Glu substitution at position 16, Ala to Val substitution at position 19, Val to Ile substitution at position 27, and Gly to Aib2 at position 29
HXEGTFTSDVSSYLEEQAVKEFIAWLIKXR

SEQ ID NO: 58 - protein, 30 aa, Artificial sequence
  GLP-1 (7-36) peptide with Ala to Aib2 substitution at position 2, Leu to Phe substitution at position 14, Gly to Glu substitution at position 16, Ala to Val substitution at position 19, Val to Ile substitution at position 27, and Gly to Aib2 at position 29
HXEGTFTSDVSSYFEEQAVKEFIAWLIKXR

SEQ ID NO: 59 - protein, 30 aa, Artificial sequence
  GLP-1 (7-36) peptide with Ala to Aib2 substitution at position 2, Leu to Tyr substitution at position 14, Gly to Glu substitution at position 16, Ala to Val substitution at position 19, Val to Ile substitution at position 27, and Gly to Aib2 at position 29
HXEGTFTSDVSSYYEEQAVKEFIAWLIKXR

SEQ ID NO: 60 - protein, 30 aa, Artificial sequence
  GLP-1 (7-36) peptide with Ala to Aib2 substitution at position 2, Leu to Trp substitution at position 14, Gly to Glu substitution at position 16, Ala to Val substitution at position 19, Val to Ile substitution at position 27, and Gly to Aib2 at position 29
HXEGTFTSDVSSYWEEQAVKEFIAWLIKXR

SEQ ID NO: 61 - protein, 30 aa, Artificial sequence
  GLP-1 (7-36) peptide with Ala to Aib2 substitution at position 2, Gly to Glu substitution at position 16, Ala to Val substitution at position 19, Val to Ile substitution at position 27, Lys to Arg substitution at position 28, and Gly to Aib2 at position 29
HXEGTFTSDVSSYLEEQAVKEFIAWLIRXR

SEQ ID NO: 62 - protein, 30 aa, Artificial sequence
  GLP-1 (7-36) peptide with Ala to Aib2, Gly to Glu, Ala to Val, Lys to Arg, Val to Ile, Lys to Arg, and Gly to Aib2 substitutions at positions 2, 16, 19, 20, 27, 28, and 29, respectively
HXEGTFTSDVSSYLEEQAVREFIAWLIRXR

SEQ ID NO: 63 - protein, 39 aa, Artificial sequence
  GLP-1 (7-36)/exendin-4 fusion peptide
HXEGTFTSDVSSYLEEQAVKEFIAWLIKXRPSSGAPPPS

SEQ ID NO: 64 - protein, 30 aa, Artificial sequence
  GLP-1 (7-36) peptide with Ala to Aib2 substitution at position 2, Val to Lys at position 10, Gly to Glu substitution at position 16, Ala to Val substitution at position 19, Val to Ile substitution at position 27, and Gly to Aib2 at position 29
HXEGTFTSDKSSYLEEQAVKEFIAWLIKXR

SEQ ID NO: 65 - protein, 30 aa, Artificial sequence
  GLP-1 (7-36) peptide with Ala to Aib2 substitution at position 2, Ser to Lys at position 12, Gly to Glu substitution at position 16, Ala to Val substitution at position 19, Val to Ile substitution at position 27, and Gly to Aib2 at position 29
HXEGTFTSDVSKYLEEQAVKEFIAWLIKXR

SEQ ID NO: 66 - protein, 30 aa, Artificial sequence
  GLP-1 (7-36) peptide with Ala to Aib2 substitution at position 2, Leu to Lys at position 14, Gly to Glu substitution at position 16, Ala to Val substitution at position 19, Val to Ile substitution at position 27, and Gly to Aib2 at position 29
HXEGTFTSDVSSYKEEQAVKEFIAWLIKXR

SEQ ID NO: 67 - protein, 30 aa, Artificial sequence
  GLP-1 (7-36) peptide with Ala to Aib2 substitution at position 2, Gly to Lys substitution at position 16, Ala to Val substitution at position 19, Val to Ile substitution at position 27, and Gly to Aib2 at position 29
HXEGTFTSDVSSYLEKQAVKEFIAWLIKXR

SEQ ID NO: 68 - protein, 30 aa, Artificial sequence
  GLP-1 (7-36) peptide with Ala to Aib2 substitution at position 2, Gly to Glu at position 16, Ala to Lys substitution at position 18, Ala to Val substitution at position 19, Val to Ile substitution at position 27, and Gly to Aib2 at position 29
HXEGTFTSDVSSYLEEQKVKEFIAWLIKXR

SEQ ID NO: 69 - protein, 30 aa, Artificial sequence
  GLP-1 (7-36) peptide with Ala to Aib2 substitution at position 2, Gly to Glu at position 16, Ala to Val substitution at position 19, Phe to Lys substitution at position 22, Val to Ile substitution at position 27, and Gly to Aib2 at position 29
HXEGTFTSDVSSYLEEQAVKEKIAWLIKXR

SEQ ID NO: 70 - protein, 30 aa, Artificial sequence
  GLP-1 (7-36) peptide with Ala to Aib2 substitution at position 2, Gly to Glu at position 16, Ala to Val substitution at position 19, Ala to Lys substitution at position 24, Val to Ile substitution at position 27, and Gly to Aib2 at position 29
HXEGTFTSDVSSYLEEQAVKEFIKWLIKXR

SEQ ID NO: 71 - protein, 30 aa, Artificial sequence
  GLP-1 (7-36) peptide with Ala to Aib2 substitution at position 2, Gly to Glu at position 16, Ala to Val substitution at position 19, Leu to Lys substitution at position 26, Val to Ile substitution at position 27, and Gly to Aib2 at position 29
HXEGTFTSDVSSYLEEQAVKEFIAWKIKXR

SEQ ID NO: 72 - protein, 40 aa, Artificial sequence
  GLP-1 (7-36)/exendin-4 fusion peptide
HXEGTFTSDVSSYLEEQAVKEFIAWLIKXRPSSGAPPPSK

SEQ ID NO: 73 - protein, 30 aa, Artificial sequence
  GLP-1 (7-36) peptide with Ala to Aib2 substitution at position 2, Gly to Glu at position 16, Ala to Val substitution at position 19, Val to Ile substitution at position 27, Lys to Lys(Ac) substitution at position 28, and Gly to Aib2 at position 29
HXEGTFTSDVSSYLEEQAVKEFIAWLIXXR

SEQ ID NO: 74 - protein, 30 aa, Artificial sequence; identical to SEQ ID NO: 73
  GLP-1 (7-36) peptide with Ala to Aib2 substitution at position 2, Gly to Glu at position 16, Ala to Val substitution at position 19, Val to Ile substitution at position 27, Lys to Lys(Benzoyl) substitution at position 28, and Gly to Aib2 at position 29

SEQ ID NO: 75 - protein, 30 aa, Artificial sequence
  GLP-1 (7-36) peptide with trans 3-hexanoyl linked to N-terminal via His residue, Ala to Aib2 substitution at position 2, Gly to Glu at position 16, Ala to Val substitution at position 19, Val to Ile substitution at position 27, and Gly to Aib2 at position
XXEGTFTSDVSSYLEEQAVKEFIAWLIKXR

SEQ ID NO: 76 - protein, 30 aa, Artificial sequence; identical to SEQ ID NO: 75
  GLP-1 (7-36) peptide with 3-aminophenylacetyl linked to N-terminal His residue, Ala to Aib2 substitution at position 2, Gly to Glu at position 16, Ala to Val substitution at position 19, Val to Ile substitution at position 27, and Gly to Aib2 at

SEQ ID NO: 77 - protein, 112 aa, Mus musculus
  MISC_FEATURE (1)..(23): FR1
DVVMTQTPLSLPVRLGDQASISCRSSQSLLHTYGSPYLNWYLQKPGQSPKLLIYKVSNRFSGVPDRFSGSGSGTDFTLRISRVEAEDLGVYFCSQGTHLPYTFGGGTKLEIK

SEQ ID NO: 78 - protein, 118 aa, Mus musculus
  MISC_FEATURE (1)..(30): FR1
EVKLVESGGGLVQPGGTMKLSCEISGLTFRNYWMSWVRQSPEKGLEWVAEIRLRSDNYATHYAESVKGKFTISRDDSKSRLYLQMNSLRTEDTGIYYCKTYFYSFSYWGQGTLVTVSA

SEQ ID NO: 79 - protein, 112 aa, Homo sapiens
  MISC_FEATURE (1)..(23): FR1
ELQMTQSPSSLSASVGDRVTITCRSSQSLLHTYGSPYLNWYLQKPGQSPKLLIYKVSNRFSGVPSRFSGSGSGTDFTLTISSLQPEDFAVYFCSQGTHLPYTFGGGTKVEIK

SEQ ID NO: 80 - protein, 118 aa, Homo sapiens
  MISC_FEATURE (1)..(30): FR1
EVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCKTYFYSFSYWGQGTLVTVSS

SEQ ID NO: 81 - protein, 95 aa, Homo sapiens
  MISC_FEATURE (1)..(23): FR1
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 82 - protein, 98 aa, Homo sapiens
  MISC_FEATURE (1)..(30): FR1
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 83 - protein, 12 aa, Homo sapiens
  MISC_FEATURE (1)..(2): CDR3
LTFGGGTKVEIK

SEQ ID NO: 84 - protein, 15 aa, Homo sapiens
  MISC_FEATURE (1)..(4): CDR3
YFDYWGQGTLVTVSS

SEQ ID NO: 85 - DNA, 68 bp, Artificial sequence
  PCR primer for generating humanized Vk
gagctccagatgacccagtctccatcctccctgtctgcatctgtaggtgaccgcgtcaccatcacttg

SEQ ID NO: 86 - DNA, 69 bp, Artificial sequence
  PCR primer for generating humanized Vk
attcagatatgggctgccataagtgtgcaggaggctctgactggagcggcaagtgatggtgacgcggtc

SEQ ID NO: 87 - DNA, 66 bp, Artificial sequence
  PCR primer for generating humanized Vk
tatggcagcccatatctgaattggtatctccagaaaccaggccagtctcctaagctcctgatctat

SEQ ID NO: 88 - DNA, 65 bp, Artificial sequence
  PCR primer for generating humanized Vk
ctgaaacgtgatgggacaccactgaaacgattggacactttatagatcaggagcttaggagactg

SEQ ID NO: 89 - DNA, 90 bp, Artificial sequence
  PCR primer for generating humanized Vk
agtggtgtcccatcacgtttcagtggcagtggttctggcacagatttcactctcaccatcagcagtctgcaacctgaagattttgcagtg

SEQ ID NO: 90 - DNA, 87 bp, Artificial sequence
  PCR primer for generating humanized Vk
gatctccaccttggtccctccgccgaaagtataagggaggtgggtgccctgactacagaagtacactgcaaaatcttcaggttgcag

SEQ ID NO: 91 - DNA, 79 bp, Artificial sequence
  PCR primer for generating humanized VH
gaggtgcagctggtggagtctggcggtggcttggtacagcctggcggttccctgcgcctctcctgtgcagcctctggct

SEQ ID NO: 92 - DNA, 81 bp, Artificial sequence
  PCR primer for generating humanized VH
ctccaggcccttctctggagactggcggacccagctcatccaatagttgctaaaggtgaagccagaggctgcacaggagag

SEQ ID NO: 93 - DNA, 81 bp, Artificial sequence
  PCR primer for generating humanized VH
tctccagagaagggcctggagtgggtctcagagattcgtctgcgcagtgacaactacgccacgcactatgcagagtctgtc

SEQ ID NO: 94 - DNA, 73 bp, Artificial sequence
  PCR primer for generating humanized VH
cagatacagcgtgttcttggaattgtcacgggagatggtgaagcggcccttgacagactctgcatagtgcgtg

SEQ ID NO: 95 - DNA, 76 bp, Artificial sequence
  PCR primer for generating humanized VH
caattccaagaacacgctgtatctgcaaatgaacagcctgcgcgccgaggacacgggcatttattactgtaaaacg

SEQ ID NO: 96 - DNA, 79 bp, Artificial sequence
  PCR primer for generating humanized VH
tgaggagacggtgaccagggtgccctggccccagtagctgaaactgtagaagtacgttttacagtaataaatgcccgtg

SEQ ID NO: 97 - DNA, 48 bp, Artificial sequence
  PCR primer for amplifying h38C2 VH coding sequence
gaggaggaggaggaggagctcactccgaggtgcagctggtggagtctg

SEQ ID NO: 98 - DNA, 72 bp, Artificial sequence
  PCR primer for amplifying h38C2 VL coding sequence
gaggaggaggaggagaagcttgttgctctggatctctggtgcctacggggagctccagatgacccagtctcc

SEQ ID NO: 99 - protein, 39 aa, Artificial sequence
  Exendin-4 peptide with Gly to Aib2 substitution at position 2, Pro to K(SH) substitution at position 38
HXEGTFTSDLSKQMEEEAVRLFIEWLKNGGPSSGAPPXS

SEQ ID NO: 100 - protein, 39 aa, Artificial sequence
  Exendin-4 peptide with Gly to Aib2 substitution at position 2, Pro to K(SH) substitution at position 36
HXEGTFTSDLSKQMEEEAVRLFIEWLKNGGPSSGAXPPS

SEQ ID NO: 101 - protein, 39 aa, Artificial sequence
  Exendin-4 peptide with Gly to Aib2 substitution at position 2, Gly to K(SH) substitution at position 34
HXEGTFTSDLSKQMEEEAVRLFIEWLKNGGPSSXAPPPS

SEQ ID NO: 102 - protein, 39 aa, Artificial sequence
  Exendin-4 peptide with Gly to Aib2 substitution at position 2, Asn to K(SH) substitution at position 28
HXEGTFTSDLSKQMEEEAVRLFIEWLKXGGPSSGAPPPS

SEQ ID NO: 103 - protein, 39 aa, Artificial sequence
  Exendin-4 peptide with Gly to Aib2 substitution at position 2, Leu to K(SH) substitution at position 26
HXEGTFTSDLSKQMEEEAVRLFIEWXKNGGPSSGAPPPS

SEQ ID NO: 104 - protein, 39 aa, Artificial sequence
  Exendin-4 peptide with Gly to Aib2 substitution at position 2, Glu to K(SH) substitution at position 24
HXEGTFTSDLSKQMEEEAVRLFIXWLKNGGPSSGAPPPS

SEQ ID NO: 105 - protein, 39 aa, Artificial sequence
  Exendin-4 peptide with Gly to Aib2 substitution at position 2, Ile to K(SH) substitution at position 23
HXEGTFTSDLSKQMEEEAVRLFXEWLKNGGPSSGAPPPS

SEQ ID NO: 106 - protein, 39 aa, Artificial sequence
  Exendin-4 peptide with Gly to Aib2 substitution at position 2, Leu to K(SH) substitution at position 21
HXEGTFTSDLSKQMEEEAVRXFIEWLKNGGPSSGAPPPS

SEQ ID NO: 107 - protein, 39 aa, Artificial sequence
  Exendin-4 peptide with Gly to Aib2 substitution at position 2, Arg to K(SH) substitution at position 20
HXEGTFTSDLSKQMEEEAVXLFIEWLKNGGPSSGAPPPS

SEQ ID NO: 108 - protein, 39 aa, Artificial sequence
  Exendin-4 peptide with Gly to Aib2 substitution at position 2, Val to K(SH) substitution at position 19
HXEGTFTSDLSKQMEEEAXRLFIEWLKNGGPSSGAPPPS

SEQ ID NO: 109 - protein, 39 aa, Artificial sequence
  Exendin-4 peptide with Gly to Aib2 substitution at position 2, Glu to K(SH) substitution at position 17
HXEGTFTSDLSKQMEEXAVRLFIEWLKNGGPSSGAPPPS

SEQ ID NO: 110 - protein, 39 aa, Artificial sequence
  Exendin-4 peptide with Gly to Aib2 substitution at position 2, Glu to K(SH) substitution at position 16
HXEGTFTSDLSKQMEXEAVRLFIEWLKNGGPSSGAPPPS

SEQ ID NO: 111 - protein, 39 aa, Artificial sequence
  Exendin-4 peptide with Gly to Aib2 substitution at position 2, Met to K(SH) substitution at position 14
HXEGTFTSDLSKQXEEEAVRLFIEWLKNGGPSSGAPPPS

SEQ ID NO: 112 - protein, 39 aa, Artificial sequence
  Exendin-4 peptide with Gly to Aib2 substitution at position 2, Gln to K(SH) substitution at position 13
HXEGTFTSDLSKXMEEEAVRLFIEWLKNGGPSSGAPPPS

SEQ ID NO: 113 - protein, 39 aa, Artificial sequence
  Exendin-4 peptide with Gly to Aib2 substitution at position 2, Ser to K(SH) substitution at position 11
HXEGTFTSDLXKQMEEEAVRLFIEWLKNGGPSSGAPPPS

SEQ ID NO: 114 - protein, 31 aa, Artificial sequence
  Exendin-4 peptide with Gly to Aib2 substitution at position 2, Pro to Lys(SH) substitution at position 31, and 8 amino acid carboxy truncation
HXEGTFTSDLSKQMEEEAVRLFIEWLKNGGX

SEQ ID NO: 115 - protein, 33 aa, Artificial sequence
  Exendin-4 peptide with Gly to Aib2 substitution at position 2, Leu to Lys substitution at position 21, Lys to Lys(SH) substitution at position 27, and 6 amino acid carboxy truncation
HXEGTFTSDLSKQMEEEAVRKFIEWLXNGGPSS

SEQ ID NO: 116 - protein, 30 aa, Artificial sequence
  GLP-1 (7-36) peptide with Ala to Aib2 substitution at position 2, Val to Lys(SH) at position 10, Gly to Glu substitution at position 16, Ala to Val substitution at position 19, Val to Ile substitution at position 27, and Gly to Aib2 at position 29
HXEGTFTSDXSSYLEEQAVKEFIAWLIKXR

SEQ ID NO: 117 - protein, 30 aa, Artificial sequence
  GLP-1 (7-36) peptide with Ala to Aib2 substitution at position 2, Ser to Lys(SH) at position 12, Gly to Glu substitution at position 16, Ala to Val substitution at position 19, Val to Ile substitution at position 27, and Gly to Aib2 at position 29
HXEGTFTSDVSXYLEEQAVKEFIAWLIKXR

SEQ ID NO: 118 - protein, 30 aa, Artificial sequence
  GLP-1 (7-36) peptide with Ala to Aib2 substitution at position 2, Leu to Lys(SH) at position 14, Gly to Glu substitution at position 16, Ala to Val substitution at position 19, Val to Ile substitution at position 27, and Gly to Aib2 at position 29
HXEGTFTSDVSSYXEEQAVKEFIAWLIKXR

SEQ ID NO: 119 - protein, 30 aa, Artificial sequence
  GLP-1 (7-36) peptide with Ala to Aib2 substitution at position 2, Gly to Lys(SH) substitution at position 16, Ala to Val substitution at position 19, Val to Ile substitution at position 27, and Gly to Aib2 at position 29
HXEGTFTSDVSSYLEXQAVKEFIAWLIKXR

SEQ ID NO: 120 - protein, 30 aa, Artificial sequence
  GLP-1 (7-36) peptide with Ala to Aib2 substitution at position 2, Gly to Glu at position 16, Ala to Lys(SH) substitution at position 18, Ala to Val substitution at position 19, Val to Ile substitution at position 27, and Gly to Aib2 at position 29
HXEGTFTSDVSSYLEEQXVKEFIAWLIKXR

SEQ ID NO: 121 - protein, 30 aa, Artificial sequence
  GLP-1 (7-36) peptide with Ala to Aib2 substitution at position 2, Gly to Glu at position 16, Ala to Val at position 19, Lys to Lys(SH) at position 20, Phe to Lys at position 22, Val to Ile at position 27, and Gly to Aib2 at position 29
HXEGTFTSDVSSYLEEQAVXEKIAWLIKXR

SEQ ID NO: 122 - protein, 30 aa, Artificial sequence
  GLP-1 (7-36) peptide with Ala to Aib2 substitution at position 2, Gly to Glu at position 16, Ala to Val substitution at position 19, Ala to Lys(SH) substitution at position 24, Val to Ile substitution at position 27, and Gly to Aib2 at position 29
HXEGTFTSDVSSYLEEQAVKEFIXWLIKXR

SEQ ID NO: 123 - protein, 30 aa, Artificial sequence
  GLP-1 (7-36) peptide with Ala to Aib2 substitution at position 2, Gly to Glu at position 16, Ala to Val substitution at position 19, Leu to Lys(SH) substitution at position 26, Val to Ile substitution at position 27, and Gly to Aib2 at position 29
HXEGTFTSDVSSYLEEQAVKEFIAWXIKXR

SEQ ID NO: 124 - protein, 40 aa, Artificial sequence
  GLP-1 (7-36)/exendin-4 fusion peptide
HXEGTFTSDVSSYLEEQAVKEFIAWLIKXRPSSGAPPPSX

SEQ ID NO: 125 - protein, 30 aa, Artificial sequence
  GLP-1 (7-36) peptide with Lys to Arg substitution at position 28
HAEGTFTSDVSSYLEGQAAKEFIAWLVRGR

SEQ ID NO: 126 - protein, 31 aa, Artificial sequence
  GLP-1 (7-36) peptide with Lys to Arg substitution at position 20, Lys to Arg substitution at position 28, and C-terminal Lys addition
HAEGTFTSDVSSYLEGQAAREFIAWLVRGRK

SEQ ID NO: 127 - protein, 30 aa, Artificial sequence
  GLP-1 (7-36) peptide with Lys to Arg substitution at position 20, Lys to Arg substitution at position 28, and Arg to Lys substitution at position 30
HAEGTFTSDVSSYLEGQAAREFIAWLVRGK

SEQ ID NO: 128 - protein, 30 aa, Artificial sequence
  GLP-1 (7-36) peptide with Ala to Aib2 substitution at position 2, Gly to Aib2 substitution at position 16, and Gly to Aib2 substitution at position 29
HXEGTFTSDVSSYLEXQAAKEFIAWLVKXR

SEQ ID NO: 129 - protein, 30 aa, Artificial sequence
  GLP-1 (7-36) peptide with Ala to Aib2 substitution at position 2 and Gly to Aib2 substitution at position 16
HXEGTFTSDVSSYLEXQAAKEFIAWLVKGR

SEQ ID NO: 130 - protein, 31 aa, Artificial sequence
  GLP-1 (7-36) peptide with Ala to Aib2 substitution at position 2, Gly to Aib2 substitution at position 16, Lys to Arg substitution at position 20, Lys to Arg substitution at position 28, Gly to Aib2 substitution at position 29, and C-terminal Lys addition
HXEGTFTSDVSSYLEXQAAREFIAWLVRXRK

SEQ ID NO: 131 - protein, 31 aa, Artificial sequence
  GLP-1 (7-36) peptide with Ala to Aib2 substitution at position 2, Lys to Arg substitution at position 20, Lys to Arg substitution at position 28, Gly to Aib2 substitution at position 29, and C-terminal Lys addition
HXEGTFTSDVSSYLEGQAAREFIAWLVRXRK

SEQ ID NO: 132 - protein, 31 aa, Artificial sequence
  GLP-1 (7-36) peptide with Ala to Aib2 substitution at position 2, Gly to Aib2 substitution at position 16, Lys to Arg substitution at position 20, Lys to Arg substitution at position 28, and C-terminal Lys addition
HXEGTFTSDVSSYLEXQAAREFIAWLVRGRK

SEQ ID NO: 133 - protein, 30 aa, Artificial sequence
  GLP-1 (7-36) peptide with Ala to Aib2 substitution at position 2, Gly to Aib2 substitution at position 16, Lys to Arg substitution at position 20, and Gly to Aib2 substitution at position 29 FEATURE
HXEGTFTSDVSSYLEXQAAREFIAWLVKXR

SEQ ID NO: 134 - protein, 31 aa, Artificial sequence
  GLP-1 (7-36) peptide with Ala to Aib2 substitution at position 2, Lys to Arg substitution at position 20, and C-terminal Lys addition
HXEGTFTSDVSSYLEGQAAREFIAWLVKGRK

SEQ ID NO: 135 - protein, 31 aa, Artificial sequence
  GLP-1 (7-36) peptide with Ala to Aib2 substitution at position 2, Gly to Aib2 substitution at position 16, Lys to Arg substitution at position 20, and C-terminal Lys addition
HXEGTFTSDVSSYLEXQAAREFIAWLVKGRK

SEQ ID NO: 136 - protein, 30 aa, Artificial sequence
  GLP-1 (7-36) peptide with Ala to Aib2 substitution at position 2, Gly to Aib2 substitution at position 16, Lys to Arg substitution at position 28, and Gly to Aib2 substitution at position 29
HXEGTFTSDVSSYLEXQAAKEFIAWLVRXR

SEQ ID NO: 137 - protein, 31 aa, Artificial sequence
  GLP-1 (7-36) peptide with Ala to Aib2 substitution at position 2, Lys to Arg substitution at position 28, Gly to Aib2 substitution at position 29, and C-terminal Lys addition
HXEGTFTSDVSSYLEGQAAKEFIAWLVRXRK

SEQ ID NO: 138 - protein, 31 aa, Artificial sequence
  GLP-1 (7-36) peptide with Ala to Aib2 substitution at position 2, Gly to Aib2 substitution at position 16, Lys to Arg substitution at position 28, and C-terminal Lys addition
HXEGTFTSDVSSYLEXQAAKEFIAWLVRGRK

SEQ ID NO: 139 - protein, 32 aa, Artificial sequence
  GLP-1 (7-36) peptide with Ala to Aib2 substitution at position 2, Gly to Aib2 substitution at position 16, Gly to Aib2 substitution at position 29, and C-terminal Ala-Lys addition
HXEGTFTSDVSSYLEXQAAKEFIAWLVKXRAK

SEQ ID NO: 140 - protein, 32 aa, Artificial sequence
  GLP-1 (7-36) peptide with Ala to Aib2 substitution at position 2, Gly to Aib2 substitution at position 29, and C-terminal Ala-Lys addition
HXEGTFTSDVSSYLEGQAAKEFIAWLVKXRAK

SEQ ID NO: 141 - protein, 32 aa, Artificial sequence
  GLP-1 (7-36) peptide with Ala to Aib2 substitution at position 2, Gly to Aib2 substitution at position 16, and C-terminal Ala-Lys addition
HXEGTFTSDVSSYLEXQAAKEFIAWLVKGRAK

SEQ ID NO: 142 - protein, 31 aa, Artificial sequence
  GLP-1 (7-36) peptide with Ala to Aib2 substitution at position 2, Gly to Aib2 substitution at position 16, Gly to Aib2 substitution at position 29, and C-terminal Lys addition
HXEGTFTSDVSSYLEXQAAKEFIAWLVKXRK

SEQ ID NO: 143 - protein, 31 aa, Artificial sequence
  GLP-1 (7-36) peptide with Ala to Aib2 substitution at position 2, Gly to Aib2 substitution at position 16, and C-terminal Lys addition
HXEGTFTSDVSSYLEXQAAKEFIAWLVKGRK

SEQ ID NO: 144 - protein, 31 aa, Artificial sequence; identical to SEQ ID NO: 37
  GLP-1 (7-36) peptide with Ala to D-Ala substitution at position 2 and C-terminal Lys addition

SEQ ID NO: 145 - protein, 31 aa, Artificial sequence
  GLP-1 (7-36) peptide with Ala to Aib2 substitution at position 2, Gly to Aib2 substitution at position 16, Ala to Aib2 substitution at position 19, and C-terminal Lys addition
HXEGTFTSDVSSYLEXQAXKEFIAWLVKGRK

SEQ ID NO: 146 - protein, 31 aa, Artificial sequence
  GLP-1 (7-36) peptide with Ala to Aib2, Gly to Glu, Gln to Glu, Lys to Arg, Ala to Glu, and Lys to Arg substitutions at positions 2, 16, 17, 20, 24, and 28, respectively, and C-terminal Lys addition
HXEGTFTSDVSSYLEEEAAREFIEWLVRGRK

SEQ ID NO: 147 - protein, 45 aa, Artificial sequence
  Exendin-4 peptide with six Lys C-terminal addition
HGEGTFTSDLSKQMEEEAVRLFIEWLKNGGPSSGAPPPSKKKKKK

SEQ ID NO: 148 - protein, 39 aa, Artificial sequence
  Exendin-4 peptide with Gly to Aib2 substitution at position 2 and Lys at position 27 attached to linker L
HXEGTFTSDLSKQMEEEAVRLFIEWLXNGGPSSGAPPPS

SEQ ID NO: 149 - protein, 39 aa, Artificial sequence; identical to SEQ ID NO: 106
  Exendin-4 peptide with Gly to Aib2 substitution at position 2 and Leu to Lys substitution at position 21, where Lys at position 21 is attached to linker L

SEQ ID NO: 150 - protein, 39 aa, Artificial sequence; identical to SEQ ID NO: 107
  Exendin-4 peptide with Gly to Aib2 substitution at position 2 and Arg to Lys substitution at position 20, where Lys at position 20 is attached to linker L

SEQ ID NO: 151 - protein, 39 aa, Artificial sequence; identical to SEQ ID NO: 108
  Exendin-4 peptide with Gly to Aib2 substitution at position 2 and Val to Lys substitution at position 19, where Lys at position 19 is attached to linker L

SEQ ID NO: 152 - protein, 39 aa, Artificial sequence; identical to SEQ ID NO: 111
  Exendin-4 peptide with Gly to Aib2 substitution at position 2 and Met to Lys substitution at position 14, where Lys at position 14 is attached to linker L

SEQ ID NO: 153 - protein, 40 aa, Artificial sequence; identical to SEQ ID NO: 124
  GLP-1 (7-36)/exendin-4 fusion peptide

SEQ ID NO: 154 - protein, 39 aa, Artificial sequence
  GLP-1 (7-36)/exendin-4 fusion peptide
HXEGTFTSDVSSYLEEQAVXEFIAWLIKXRPSSGAPPPS

SEQ ID NO: 155 - protein, 30 aa, Artificial sequence; identical to SEQ ID NO: 39
  GLP-1 (7-36) peptide with Ala to Aib2, Lys at position 20 attached to linker L

SEQ ID NO: 156 - protein, 39 aa, Artificial sequence; identical to SEQ ID NO: 148
  Exendin-4 peptide with Gly to Aib2 substitution at position 2 and Lys at position 27 attached to linker L′

SEQ ID NO: 157 - protein, 39 aa, Artificial sequence; identical to SEQ ID NO: 106
  Exendin-4 peptide with Gly to Aib2 substitution at position 2 and Lys at position 21 attached to linker L′

SEQ ID NO: 158 - protein, 39 aa, Artificial sequence; identical to SEQ ID NO: 107
  Exendin-4 peptide with Gly to Aib2 substitution at position 2 and Lys at position 20 attached to linker L′

SEQ ID NO: 159 - protein, 39 aa, Artificial sequence; identical to SEQ ID NO: 108
  Exendin-4 peptide with Gly to Aib2 substitution at position 2 and Lys at position 19 attached to linker L′

SEQ ID NO: 160 - protein, 39 aa, Artificial sequence; identical to SEQ ID NO: 111
  Exendin-4 peptide with Gly to Aib2 substitution at position 2 and Lys at position 14 attached to linker L′

SEQ ID NO: 161 - protein, 40 aa, Artificial sequence; identical to SEQ ID NO: 124
  GLP-1 (7-36)/exendin-4 fusion peptide

SEQ ID NO: 162 - protein, 39 aa, Artificial sequence; identical to SEQ ID NO: 154
  GLP-1 (7-36)/exendin-4 fusion peptide

SEQ ID NO: 163 - protein, 30 aa, Artificial sequence; identical to SEQ ID NO: 39
  GLP-1 (7-36) peptide with Ala to Aib2, Lys at position 20 attached to linker L′

SEQ ID NO: 164 - protein, 39 aa, Artificial sequence; identical to SEQ ID NO: 110
  Exendin-4 peptide with Gly to Aib2 substitution at position 2 and Glu to Lys substitution at position 16, where Lys at position 16 is attached to linker L

SEQ ID NO: 165 - protein, 39 aa, Artificial sequence; identical to SEQ ID NO: 110
  Exendin-4 peptide with Gly to Aib2 substitution at position 2 and Glu to Lys substitution at position 16, where Lys at position 16 is attached to linker L′

SEQ ID NO: 166 - protein, 40 aa, Artificial sequence
  Exendin-4 peptide with C-terminus Lys addition, where Lys at position 40 is attached to linker L
HGEGTFTSDLSKQMEEEAVRLFIEWLKNGGPSSGAPPPSX

SEQ ID NO: 167 - protein, 40 aa, Artificial sequence
  Exendin-4 peptide with Gly to Aib2 substitution at position 2 and C-terminal Lys addition, where Lys at position 40 is attached to linker L
HXEGTFTSDLSKQMEEEAVRLFIEWLKNGGPSSGAPPPSX

SEQ ID NO: 168 - protein, 39 aa, Artificial sequence
  Exendin-4 peptide with Gly to Aib2 substitution at position 2, Ser to K(SH) substitution at position 32
HXEGTFTSDLSKQMEEEAVRLFIEWLKNGGPXSGAPPPS

SEQ ID NO: 169 - protein, 39 aa, Artificial sequence
  Exendin-4 peptide with Gly to Aib2 substitution at position 2, Lys at position 12 is K(SH)
HXEGTFTSDLSXQMEEEAVRLFIEWLKNGGPSSGAPPPS

SEQ ID NO: 170 - protein, 39 aa, Artificial sequence; identical to SEQ ID NO: 148
  Exendin-4 peptide with Gly to Aib2 substitution at position 2, Lys at position 27 is K(SH)

SEQ ID NO: 171 - protein, 39 aa, Artificial sequence; identical to SEQ ID NO: 154
  GLP-1 (7-36)/exendin-4 fusion peptide

SEQ ID NO: 172 - protein, 40 aa, Artificial sequence; identical to SEQ ID NO: 166
  Exendin-4 peptide with C-terminus Lys(SH) addition

SEQ ID NO: 173 - protein, 40 aa, Artificial sequence; identical to SEQ ID NO: 167
  Exendin-4 peptide with Gly to Aib2 substitution at position 2 and C-terminal Lys(SH) addition

(19) United States
(12) Patent Application Publication
(10) Pub. No.: US 2009/0098130 A1
(43) Pub. Date: Apr. 16, 2009

(54) GLUCAGON-LIKE PROTEIN-1 RECEPTOR (GLP-1R) AGONIST COMPOUNDS

(76) Inventors: Curt W. BRADSHAW, San Diego, CA (US); Sukumar Sakamuri, Indira Nagar (IN); Yanwen Fu, San Diego, CA (US); Bryan Oates, Carlsbad, CA (US); Joel Desharnais, San Diego, CA (US); David Tumelty, San Diego, CA (US)

Correspondence Address: PERKINS COIE LLP, POST OFFICE BOX 1208, SEATTLE, WA, 98111-1208, US

(21) Appl. No.: 11/969,850

(22) Filed: Jan. 4, 2008

Related U.S. Application Data
(60) Provisional application No. 60/879,048, filed on Jan. 5, 2007.
(60) Provisional application No. 60/939,831, filed on May 23, 2007.
(60) Provisional application No. 60/945,319, filed on Jun. 20, 2007.

(51) Int. Cl. A61K 39/44; A61P 3/10; C07K 19/00

(52) U.S. Cl. 424/139.1; 530/387.9

(57) ABSTRACT

The present invention provides GA targeting compounds which comprise GA targeting agent-linker conjugates linked to a combining site of an antibody. Various uses of the compounds are provided, including methods to prevent or treat diabetes or diabetes-related conditions.

### RELATED APPLICATIONS

The present application claims priority to U.S. Provisional Application No. 60/879,048, filed Jan. 5, 2007, U.S. Provisional Application No. 60/939,831, filed May 23, 2007, and U.S. Provisional Application No. 60/945,319, filed Jun. 20, 2007, the disclosures of which are incorporated by reference herein in their entirety.

### FIELD OF THE DISCLOSURE

The present invention relates to novel compounds that promoting insulin secretion and lower blood glucose levels, and methods of making and using these compounds. In particular, the present invention relates to compounds that bind to and activate the glucagon-like protein 1 receptor (GLP-1R).

### BACKGROUND

Type II diabetes is the most prevalent form of diabetes. The disease is caused by insulin resistance and pancreatic β cell failure, which results in decreased glucose-stimulated insulin secretion. Incretins, which are compounds that stimulate glucose-dependent insulin secretion and inhibit glucagon secretion, have emerged as attractive candidates for the treatment of type II diabetes. Two incretins that have been found to improve β cell function in vitro are glucose insulinotropic polypeptide (GIP) and glucagon-like peptide (7-36) amide (GLP-1). GIP does not appear to be an attractive therapeutic candidate, because diabetic β cells are relatively resistant to its action. However, diabetic β cells are sensitive to the effects of GLP-1.

In addition to increasing insulin secretion and decreasing glucagon secretion, the 30-amino acid GLP-1 peptide stimulates pro-insulin gene transcription, slows down gastric emptying time, and reduces food intake. GLP-1 exerts its physiological effects by binding to the glucagon-like peptide 1 receptor (GLP-1R), a putative seven-transmembrane domain receptor.

A drawback to the therapeutic use of GLP-1 is its short in vivo half-life (1-2 minutes). This short half-life is the result of rapid degradation of the peptide by dipeptidyl peptidase 4 (DPP-IV). This has led to the identification or development of GLP-1 analogs that exhibit increased half lives while maintaining the ability to agonize GLP-1R activity. Examples of these analogs include exendin-4 and GLP-1-Gly8.

Although several GLP-1 analogs have been developed that maintain insulinotropic activities while displaying increased half-lives, there is still a need for GLP-1R agonists with improved pharmacokinetic profiles.

The reference to any art in this specification is not, and should not be taken as, an acknowledgement of any form or suggestion that the referenced art forms part of the common general knowledge.

### SUMMARY

Disclosed herein are compositions formed by covalently linking one or more GLP-1R agonist peptides to a combining site of one or more antibodies, and methods of making and using these compositions. In certain embodiments, GLP-1R agonist (GA) compounds with improved in vivo half-lives are provided. GA targeting compounds are formed by covalently linking a GA targeting agent, either directly or via an intervening linker, to a combining site of an antibody. Pharmaceutical compositions comprising targeting compounds of the invention and a pharmaceutically acceptable carrier are also provided.

In certain embodiments, GLP-1R agonist (GA) peptides are provided. In some aspects, the present invention provides a GA targeting agent, wherein a GA targeting agent is a peptide agonist of the GLP-1 receptor, comprising a peptide comprising a sequence substantially homologous to:

R¹—H¹x²E³G⁴T⁵F⁶T⁷S⁸D⁹x¹⁰S¹¹x¹²x¹³x¹⁴E¹⁵x¹⁶x¹⁷A¹⁸x¹⁹x²⁰x²¹F²²x²³x²⁴x²⁵x²⁶x²⁷x²⁸x²⁹x³⁰x³¹x³²x³³x³⁴x³⁵x³⁶x³⁷x³⁸x³⁹-R²,
wherein:

R¹ is absent, CH₃, C(O)CH3, C(O)CH₂CH₃, C(O)CH₂CH₂CH₃, or C(O)CH(CH₃)CH₃;

R² is absent, OH, NH₂, NH(CH₃), NHCH₂CH₃, NHCH₂CH₂CH₃, NHCH(CH₃)CH₃, NHCH₂CH₂CH₂CH₃, NHCH(CH₃)CH₂CH₃, NHC₆H₅, NHCH₂CH₂OCH₃, NHOCH₃, NHOCH₂CH₃, a carboxy protecting group, a lipid fatty acid group, or a carbohydrate, and

x² is a blocking group such as Aib, A, S, T, V, L, I, or D-Ala, (wherein the term “blocking group” in the context of position x² refers to a residue or group that can block certain cleavage reactions, such as DPP-4 cleavage), x¹⁰ is V, L, I, or A, x¹² is S or K, x¹³ is Q or Y, x¹⁴ is G, C, F, Y, W, M, or L, x¹⁶ is K, D, E, or G, x¹⁷ is E or Q, x¹⁹ is L, I, V, or A, x²⁰ is Orn, K(SH), R, or K, x²¹ is L or E, x²³ is I or L, x²⁴ is A or E, x²⁵ is W or F, x²⁶ is L or I, x²⁷ is I, K, or V, x²⁸ is R, Orn, N, or K, x²⁹ is Aib or G, x³⁰ is any amino acid, preferably G or R, x³¹ is P or absent, x³² is S or absent, x³³ is S or absent, x³⁴ is G or absent, x³⁵ is A or absent, x³⁶ is P or absent, x³⁷ is P or absent, x³⁸ is P or absent, x³⁹ is S or absent, x⁴⁰ is a linking residue or absent, and in addition, wherein one of x¹⁰, x¹¹, x¹², x¹³, x¹⁴, x¹⁶, x¹⁷, x¹⁹, x²⁰, x²¹, x²⁴, x²⁶, x²⁷, x²⁸, x³², x³³, x³⁴, x³⁵, x³⁶, x³⁷, x³⁸, x³⁹, or x⁴⁰ is substituted with a linking residue (-[LR]-) comprising a nucleophilic sidechain covalently linkable to the combining site of an antibody via an intermediate linker, wherein the linking residue is K(SH). In these embodiments, x² may be Aib.

Compounds of the invention may comprise a peptide comprising a sequence substantially homologous to one or more compounds selected from the group consisting of:

(SEQ ID NO:172)

R¹-HGEGTFTSDLSKQMEEEAVRLFIEWLKLNGGPSSGAPPPSK(SH)-
R²,

(SEQ ID NO:173)

R¹-HAibEGTFTSDLSKQMEEEAVRLFIEWLKNGGPSSGAPPPSK(SH)-
R²,

(SEQ ID NO:99)

R¹-HAibEGTFTSDLSKQMEEEAVRLFIEWLKNGGPSSGAPPK(SH)S-
R²,

(SEQ ID NO:100)

R¹-HAibEGTFTSDLSKQMEEEAVRLFIEWLKNGGPSSGAK(SH)PPS-
R²,

(SEQ ID NO:110)

R¹-HAibEGTFTSDLSKQMEEEAVRLFIEWLKNGGPSSK(SH)APPPS-
R²,

(SEQ ID NO:168)

R¹-HAibEGTFTSDLSKQMEEEAVRLFIEWLKNGGPK(SH)SGAPPPS-
R²,

(SEQ ID NO:102)

R¹-HAibEGTFTSDLSKQMEEEAVRLFIEWLKK(SH)GGPSSGAPPPS-
R²,

(SEQ ID NO:170)

R¹-HAibEGTFTSDLSKQMEEEAVRLFIEWLK(SH)NGGPSSGAPPPS-
R²,

(SEQ ID NO:103)

R¹-HAibEGTFTSDLSKQMEEEAVRLFIEWK(SH)KNGGPSSGAPPPS-
R²,

(SEQ ID NO:104)

R¹-HAibEGTFTSDLSKQMEEEAVRLFIK(SH)WLKNGGPSSGAPPPS-
R²,

(SEQ ID NO:105)

R¹-HAibEGTFTSDLSKQMEEEAVRLFK(SH)EWLKNGGPSSGAPPPS-
R²,

(SEQ ID NO:106)

R¹-HAibEGTFTSDLSKQMEEEAVRK(SH)FIEWLKNGGPSSGAPPPS-
R²,

(SEQ ID NO:107)

R¹-HAibEGTFTSDLSKQMEEEAVK(SH)LFIEWLKNGGPSSGAPPPS-
R²,

(SEQ ID NO:108)

R¹-HAibEGTFTSDLSKQMEEEAK(SH)RLFIEWLKNGGPSSGAPPPS-
R²,

(SEQ ID NO:109)

R¹-HAibEGTFTSDLSKQMEEK(SH)AVRLFIEWLKNGGPSSGAPPPS-
R²,

(SEQ ID NO:110)

R¹-HAibEGTFTSDLSKQMEK(SH)EAVRLFIEWLKNGGPSSGAPPPS-
R²,

(SEQ ID NO:111)

R¹-HAibEGTFTSDLSKQK(SH)EEEAVRLFIEWLKNGGPSSGAPPPS-
R²,

(SEQ ID NO:112)

R¹-HAibEGTFTSDLSKK(SH)MEEEAVRLFIEWLKNGGPSSGAPPPS-
R²,

(SEQ ID NO:169)

R¹-HAibEGTFTSDLSK(SH)QMEEEAVRLFIEWLKNGGPSSGAPPPS-
R²,

(SEQ ID NO:113)

R¹-HAibEGTFTSDLK(SH)KQMEEEAVRLFIEWLKNGGPSSGAPPPS-
R²,

(SEQ ID NO:114)

R¹-HAibEGTFTSDLSKQMEEEAVRLFIEWLKNGGK(SH)-R²,

(SEQ ID NO:115)

R¹-HAibEGTFTSDLSKQMEEEAVRKFIEWLK(SH)NGGPSS-R²,

(SEQ ID NO:38)

R¹-HAibEGTFTSDVSSYLEGQAAKEFIAWLVKGRK(SH)-R²,

(SEQ ID NO:39)

R¹-HAibEGTFTSDVSSYLEGQAAK(SH)EFIAWLVKGR-R²,

(SEQ ID NO:171)

R¹-HAibEGTFTSDVSSYLEEQAVK(SH)EFIAWLIKAibRPSSGAPPP
S-R²,

(SEQ ID NO:116)

R¹-HAibEGTFTSDK(SH)SSYLEEQAVKEFIAWLIKIAibR-R²,

(SEQ ID NO:117)

R¹-HAibEGTFTSDVSK(SH)YLEEQAVKEFIAWLIKAibR-R²,

(SEQ ID NO:118)

R¹-HAibEGTFTSDVSSYK(SH)EEQAVKEFIAWLIKAibR-R²,

(SEQ ID NO:119)

R¹-HAibEGTFTSDVSSYLEK(SH)QAVKEFIAWLIKAibR-R²,

(SEQ ID NO:120)

R¹-HAibEGTFTSDVSSYLEEQK(SH)VKEFIAWLIKAibR-R²,

(SEQ ID NO:121)

R¹-HAibEGTFTSDVSSYLEEQAVK(SH)EKIAWLIKAibR-R²,

(SEQ ID NO:122)

R¹-HAibEGTFTSDVSSYLEEQAVKEFIK(SH)WLIKAibR-R²,

(SEQ ID NO:123)

R¹-HAibEGTFTSDVSSYLEEQAVKEFIAWK(SH)IKAibR-R²,
and

(SEQ ID NO:124)

R¹-HAibEGTFTSDVSSYLEEQAVKEFIAWLIKAibRPSSGAPPPSK
(SH)-R².

Compounds of the invention may comprise a peptide comprising a sequence substantially homologous to one or more compounds selected from the group consisting of:

(SEQ ID NO:38)

R¹-HAibEGTFTSDVSSYLEGQAAKEFIAWLVKGRK(SH)-R²,

(SEQ ID NO:39)

R¹-HAibEGTFTSDVSSYLEGQAAK(SH)EFIAWLVKGR-R²,

(SEQ ID NO:116)

R¹-HAibEGTFTSDK(SH)SSYLEEQAVKEFIAWLIKAibR-R²,

(SEQ ID NO:117)

R¹-HAibEGTFTSDVSK(SH)YLEEQAVKEFIAWLIKAibR-R²,

(SEQ ID NO:118)

R¹-HAibEGTFTSDVSSYK(SH)EEQAVKEFIAWLIKAibR-R²,

(SEQ ID NO:119)

R¹-HAibEGTFTSDVSSYLEK(SH)QAVKEFIAWLIKAibR-R²,

(SEQ ID NO:120)

R¹-HAibEGTFTSDVSSYLEEQK(SH)VKEFIAWLIKAibR-R²,

(SEQ ID NO:121)

R¹-HAibEGTFTSDVSSYLEEQAVK(SH)EKIAWLIKAibR-R²,

(SEQ ID NO:122)

R¹-HAibEGTFTSDVSSYLEEQAVKEFIK(SH)WLIKAibR-R²,
and

(SEQ ID NO:123)

R¹-HAibEGTFTSDVSSYLEEQAVKEFIAWK(SH)IKAibR-R².

Compounds of the invention may comprise a peptide comprising a sequence substantially homologous to one or more compounds selected from the group consisting of:

(SEQ ID NO:172)

R¹-HGEGTFTSDLSKQMEEEAVRLFIEWLKNGGPSSGAPPPSK(SH)-
R²,

(SEQ ID NO:173)

R¹-HAibEGTFTSDLSKQMEEEAVRLFIEWLKNGGPSSGAPPPSK(SH)-
R²,

(SEQ ID NO:99)

R¹-HAibEGTFTSDLSKQMEEEAVRLFIEWLKNGGPSSGAPPK(SH)S-
R²,

(SEQ ID NO:100)

R¹-HAibEGTFTSDLSKQMEEEAVRLFIEWLKNGGPSSGAK(SH)PPS-
R²,

(SEQ ID NO:101)

R¹-HAibEGTFTSDLSKQMEEEAVRLFIEWLKNGGPSSK(SH)APPPS-
R²,

(SEQ ID NO:168)

R¹-HAibEGTFTSDLSKQMEEEAVRLFIEWLKNGGPK(SH)SGAPPPS-
R²,

(SEQ ID NO:170)

R¹-HAibEGTFTSDLSKQMEEEAVRLFIEWLKK(SH)GGPSSGAPPPS-
R²,

(SEQ ID NO:170)

R¹-HAibEGTFTSDLSKQMEEEAVRLFIEWLK(SH)NGGPSSGAPPPS-
R²,

(SEQ ID NO:103)

R¹-HAibEGTFTSDLSKQMEEEAVRLFIEWK(SH)KNGGPSSGAPPPS-
R²,

(SEQ ID NO:104)

R¹-HAibEGTFTSDLSKQMEEEAVRLFIK(SH)WLKNGGPSSGAPPPS-
R²,

(SEQ ID NO:105)

R¹-HAibEGTFTSDLSKQMEEEAVRLFK(SH)EWLKNGGPSSGAPPPS-
R²,

(SEQ ID NO:106)

R¹-HAibEGTFTSDLSKQMEEEAVRK(SH)FIEWLKNGGPSSGAPPPS-
R²,

(SEQ ID NO:107)

R¹-HAibEGTFTSDLSKQMEEEAVK(SH)LFIEWLKNGGPSSGAPPPS-
R²,

(SEQ ID NO:108)

R¹-HAibEGTFTSDLSKQMEEEAK(SH)RLFIEWLKNGGPSSGAPPPS-
R²,

(SEQ ID NO:109)

R¹-HAibEGTFTSDLSKQMEEK(SH)AVRLFIEWLKNGGPSSGAPPPS-
R²,

(SEQ ID NO:110)

R¹-HAibEGTFTSDLSKQMEK(SH)EAVRLFIEWLKNGGPSSGAPPPS-
R²,

(SEQ ID NO:111)

R¹-HAibEGTFTSDLSKQK(SH)EEEAVRLFIEWLKNGGPSSGAPPPS-
R²,

(SEQ ID NO:112)

R¹-HAibEGTFTSDLSKK(SH)MEEEAVRLFIEWLKNGGPSSGAPPPS-
R²,

(SEQ ID NO:169)

R¹-HAibEGTFTSDLSK(SH)QMEEEAVRLFIEWLKNGGPSSGAPPPS-
R²,

(SEQ ID NO:113)

R¹-HAibEGTFTSDLK(SH)KQMEEEAVRLFIEWLKNGGPSSGAPPPS-
R²,

(SEQ ID NO:1114)

R¹-HAibEGTFTSDLSKQMEEEAVRLFIEWLKNGGK(SH)-R²,
and

(SEQ ID NO:115)

R¹-HAibEGTFTSDLSKQMEEEAVRKFIEWLK(SH)NGGPSS-R².

Compounds of the invention may comprise a peptide comprising a sequence substantially homologous to one or more compounds selected from the group consisting of:

(SEQ ID NO:99)

R¹-HAibEGTFTSDLSKQMEEEAVRLFIEWLKNGGPSSGAPPK(SH)S-
R²,

(SEQ ID NO:100)

R¹-HAibEGTFTSDLSKQMEEEAVRLFIEWLKNGGPSSGAK(SH)PPS-
R²,

(SEQ ID NO:101)

R¹-HAibEGTFTSDLSKQMEEEAVRLFIEWLKNGGPSSK(SH)APPPS-
R²,

(SEQ ID NO:168)

R¹-HAibEGTFTSDLSKQMEEEAVRLFIEWLKNGGPK(SH)SGAPPPS-
R²,

(SEQ ID NO:102)

R¹-HAibEGTFTSDLSKQMEEEAVRLFIEWLKK(SH)GGPSSGAPPPS-
R²,

(SEQ ID NO:170)

R¹-HAibEGTFTSDLSKQMEEEAVRLFIEWLK(SH)NGGPSSGAPPPS-
R²,

(SEQ ID NO:103)

R¹-HAibEGTFTSDLSKQMEEEAVRLFIEWK(SH)KNGGPSSGAPPPS-
R²,

(SEQ ID NO:104)

R¹-HAibEGTFTSDLSKQMEEEAVRLFIK(SH)WLKNGGPSSGAPPPS-
R²,

(SEQ ID NO:105)

R¹-HAibEGTFTSDLSKQMEEEAVRLFK(SH)EWLKNGGPSSGAPPPS-
R²,

(SEQ ID NO:106)

R¹-HAibEGTFTSDLSKQMEEEAVRK(SH)FIEWLKNGGPSSGAPPPS-
R²,

(SEQ ID NO:107)

R¹-HAibEGTFTSDLSKQMEEEAVK(SH)LFIEWLKNGGPSSGAPPPS-
R²,

(SEQ ID NO:108)

R¹-HAibEGTFTSDLSKQMEEEAK(SH)RLFIEWLKNGGPSSGAPPPS-
R²,

(SEQ ID NO:109)

R¹-HAibEGTFTSDLSKQMEEK(SH)AVRLFIEWLKNGGPSSGAPPPS-
R²,

(SEQ ID NO:110)

R¹-HAibEGTFTSDLSKQMEK(SH)EAVRLFIEWLKNGGPSSGAPPPS-
R²,

(SEQ ID NO:111)

R¹-HAibEGTFTSDLSKQK(SH)EEEAVRLFIEWLKNGGPSSGAPPPS-
R²,

(SEQ ID NO:112)

R¹-HAibEGTFTSDLSKK(SH)MEEEAVRLFIEWLKNGGPSSGAPPPS-
R²,

(SEQ ID NO:169)

R¹-HAibEGTFTSDLSK(SH)QMEEEAVRLFIEWLKNGGPSSGAPPPS-
R²,

(SEQ ID NO:113)

R¹-HAibEGTFTSDLK(SH)KQMEEEAVRLFIEWLKNGGPSSGAPPPS-
R²,
and

(SEQ ID NO:115)

R¹-HAibEGTFTSDLSKQMEEEAVRKFIEWLK(SH)NGGPSS-R².

Compounds of the invention may comprise a peptide comprising a sequence selected from the group consisting of:

(SEQ ID NO:99)

R¹-HAibEGTFTSDLSKQMEEEAVRLFIEWLKNGGPSSGAPPK(SH)S-
R²,

(SEQ ID NO:100)

R¹-HAibEGTFTSDLSKQMEEEAVRLFIEWLKNGGPSSGAK(SH)PPS-
R²,

(SEQ ID NO:101)

R¹-HAibEGTFTSDLSKQMEEEAVRLFIEWLKNGGPSSK(SH)APPPS-
R²,

(SEQ ID NO:168)

R¹-HAibEGTFTSDLSKQMEEEAVRLFIEWLKNGGPK(SH)SGAPPPS-
R²,

(SEQ ID NO:102)

R¹-HAibEGTFTSDLSKQMEEEAVRLFIEWLKK(SH)GGPSSGAPPPS-
R²,

(SEQ ID NO:170)

R¹-HAibEGTFTSDLSKQMEEEAVRLFIEWLK(SH)NGGPSSGAPPPS-
R²,

(SEQ ID NO:106)

R¹-HAibEGTFTSDLSKQMEEEAVRK(SH)FIEWLKNGGPSSGAPPPS-
R²,

(SEQ ID NO:107)

R¹-HAibEGTFTSDLSKQMEEEAVK(SH)LFIEWLKNGGPSSGAPPPS-
R²,

(SEQ ID NO:108)

R¹-HAibEGTFTSDLSKQMEEEAK(SH)RLFIEWLKNGGPSSGAPPPS-
R²,

(SEQ ID NO:110)

R¹-HAibEGTFTSDLSKQMEK(SH)EAVRLFIEWLKNGGPSSGAPPPS-
R²,

(SEQ ID NO:111)

R¹-HAibEGTFTSDLSKQK(SH)EEEAVRLFIEWLKNGGPSSGAPPPS-
R²,

(SEQ ID NO:112)

R¹-HAibEGTFTSDLSKK(SH)MEEEAVRLFIEWLKNGGPSSGAPPPS-
R²,

(SEQ ID NO:169)

R¹-HAibEGTFTSDLSK(SH)QMEEEAVRLFIEWLKNGGPSSGAPPPS-
R²,

(SEQ ID NO:113)

R¹-HAibEGTFTSDLK(SH)KQMEEEAVRLFIEWLKNGGPSSGAPPPS-
R²,
and

(SEQ ID NO:115)

R¹-HAibEGTFTSDLSKQMEEEAVRKFIEWLK(SH)NGGPSS-R².

Compounds of the invention may comprise a peptide comprising a sequence selected from the group consisting of:

(SEQ ID NO:106)

R¹-HAibEGTFTSDLSKQMEEEAVRK(SH)FIEWLKNGGPSSGAPPPS-
R²,

(SEQ ID NO:107)

R¹-HAibEGTFTSDLSKQMEEEAVK(SH)LFIEWLKNGGPSSGAPPPS-
R²,

(SEQ ID NO:108)

R¹-HAibEGTFTSDLSKQMEEEAK(SH)RLFIEWLKNGGPSSGAPPPS-
R²,

(SEQ ID NO:110)

R¹-HAibEGTFTSDLSKQMEK(SH)EAVRLFIEWLKNGGPSSGAPPPS-
R²,

(SEQ ID NO:111)

R¹-HAibEGTFTSDLSKQK(SH)EEEAVRLFIEWLKNGGPSSGAPPPS-
R²,
and

(SEQ ID NO:112)

R¹-HAibEGTFTSDLSKK(SH)MEEEAVRLFIEWLKNGGPSSGAPPPS-
R².

In certain aspects, the present invention provides a GA targeting agent, wherein a GA targeting agent is a peptide agonist of the GLP-1 receptor, comprising a peptide comprising a sequence substantially homologous to:

R¹—H¹x²E³G⁴T⁵F⁶T⁷S⁸D⁹x¹⁰S¹¹x¹²x¹³x¹⁴E¹⁵x¹⁶x¹⁷A¹⁸x¹⁹x²⁰x²¹F²²x²³x²⁴x²⁵x²⁶x²⁷x²⁸x²⁹x³⁰x³¹x³²x³³x³⁴x³⁵x³⁶x³⁷x³⁸x³⁹-R²

wherein

R¹ is absent, CH₃, C(O)CH3, C(O)CH₂CH₃, C(O)CH₂CH₂CH₃, or C(O)CH(CH₃)CH₃; and

R² is absent, OH, NH₂, NH(CH₃), NHCH₂CH₃, NHCH₂CH₂CH₃, NHCH(CH₃)CH₃, NHCH₂CH₂CH₂CH₃, NHCH(CH₃)CH₂CH₃, NHC₆H₅, NHCH₂CH₂OCH₃, NHOCH₃, NHOCH₂CH₃, a carboxy protecting group, a lipid fatty acid group or a carbohydrate,

x² is a blocking group such as Aib, A, S, T, V, L, I, or D-Ala, x¹⁰ is V, L, I, or A, x¹² is S or K, x¹³ is Q or Y, x¹⁴ is G, C, F, Y, W, M, or L, x¹⁶ is K, D, E, or G, x¹⁷ is E or Q, x¹⁹ is L, I, V, or A, x²⁰ is Orn, K(SH), R, or K, x²¹ is L or E, x²³ is I or L, x²⁴ is A or E, x²⁵ is W or F, x²⁶ is L or I, x²⁷ is I, K, or V, x²⁸ is R, Orn, N, or K, x²⁹ is Aib or G, x³⁰ is any amino acid, preferably G or R, x³¹ is P or absent, x³² is S or absent, x³³ is S or absent, x³⁴ is G or absent, x³⁵ is A or absent, x³⁶ is P or absent, x³⁷ is P or absent, x³⁸ is P or absent, x³⁹ is S or absent, x⁴⁰ is a linking residue or absent,

and wherein the peptide is covalently linked to the combining site of an antibody via an intermediate linker (L′), and L′ is covalently linked to either the C-terminus or a nucleophilic sidechain of a Linking Residue (-[LR]-), such that [LR]- is selected from the group comprising K, R, Y, C, T, S, homologs of lysine (including K(SH)), homocysteine, and homoserine, and when present, substitutes one of x¹⁰, x¹¹, x¹², x¹³, x¹⁴, x¹⁶, x¹⁷, x¹⁹, x²⁰, x²¹, x²⁴, x²⁶, x²⁷, x²⁸, x³², x³³, x³⁴, x³⁵, x³⁶, x³⁷, x³⁸, or x³⁹, or x⁴⁰.

In certain aspects, the invention provides a GA targeting agent comprising a peptide comprising a sequence substantially homologous to:

R¹—H¹Aib²E³G⁴T⁵F⁶T⁷S⁸D⁹V¹⁰S¹¹S¹²Y¹³x¹⁴E¹⁵x¹⁶Q¹⁷A¹⁸x¹⁹x²⁰E²¹F²²I²³A²⁴x²⁵L²⁶x²⁷x²⁸x²⁹R³⁰—R²

wherein x¹⁴ is G, C, F, Y, W, or L, x¹⁶ is K, D, E, or G, x¹⁹ is L, I, V, or A, x²⁰ is Orn, R, or K, x²⁵ is W or F, x²⁷ is I or V, x²⁸ is R or K, and x²⁹ is Aib or G.

In certain aspects, the invention provides a GA targeting agent comprising a peptide comprising a sequence substantially homologous to:

R¹—H¹Aib²E³G⁴T⁵F⁶T⁷S⁸D⁹L¹⁰S¹¹K¹²Q¹³M¹⁴E¹⁵E¹⁶E¹⁷A¹⁸V¹⁹R²⁰L²¹F²²I²³E²⁴W²⁵L²⁶K²⁷N²⁸G²⁹G³⁰P³¹S³²S³³G³⁴A³⁵P³⁶P³⁷P³⁸S³⁹—R².

In certain aspects the linking residue is selected from the group consisting of K, Y, T, and homologs of lysine (including K(SH)). The linking residue may be K(L), wherein K(L) is a lysine reside attached to a linker L wherein L is capable of forming a covalent bond with an amino acid sidechain in a combining site of an antibody.

Throughout this specification, claims and accompanying figures and sequence listings, “(L)” is employed to indicate a Linker covalently connected to the preceding residue. When describing the amino acid residue leucine, the single amino acid code “L” is used. The use of parentheses for linker: “(L)”, and absence of parentheses for leucine: “L”, as well as the context of the usage will enable the skilled person to avoid confusion between the two terms.

The linking residue may be selected from the group consisting of x¹¹, x¹², x¹³, x¹⁴, x¹⁵, x¹⁶, x¹⁷, x¹⁹, x²⁰, x²¹, x²⁴, x²⁷, x²⁸, x³², x³⁴, x³⁸, and C-terminus. The linking residue may be selected from the group consisting of x¹¹, x¹², x¹³, x¹⁴, x¹⁶, x¹⁹, x²⁰, x²¹, x²⁷, x²⁸, x³²x and x³⁴. The linking residue may be selected from the group consisting of x¹¹, x¹², x¹³, x¹⁴, x¹⁶, x¹⁹, x²⁰, and x²¹. The linking residue may be selected from the group consisting of x¹³, x¹⁴, x¹⁶, x¹⁹, x²⁰, and x²¹. x¹⁴ may be the linking residue. In some aspects of the invention, R¹ is C(O)CH₃, thus acetylating the amino terminus of a GA targeting agent.

In some aspects of the invention, R² is NH₂, thus amidating the carboxy terminus of a GA targeting agent.

In some embodiments, the present invention provides a GA targeting compound comprising a peptide comprising a sequence substantially homologous to:

Hx²EGTFTSDx¹⁰x¹¹x¹²x¹³x¹⁴Ex¹⁶x¹⁷Ax¹⁹x²⁰x²¹Fx²³x²⁴x²⁵x²⁶x²⁷x²⁸x²⁹x³⁰x³¹x³²x³³x³⁴x³⁵x³⁶x³⁷x³⁸x³⁹x⁴⁰
wherein:

x² is a blocking group such as Aib, A, S, T, V, L, I, or D-Ala, x¹⁰ is V, L, I, or A, x¹¹ is a linking residue or S, x¹² is a linking residue, S, or K, x¹³ is a linking residue, Q, or Y, x¹⁴ is a linking residue, G, C, F, Y, W, M, or L, x¹⁶ is a linking residue, K, D, E, or G, x¹⁷ is a linking residue, E, or Q, x¹⁹ is a linking residue, L, I, V, or A, x²⁰ is a linking residue, Orn, K(SH), R, or K, x²¹ is a linking residue, L, or E, x²³ is a linking residue, I, or L, x²⁴ is a linking residue, A, or E, x²⁵ is a linking residue or aromatic residue, x²⁶ is a linking residue, L, or I, x²⁷ is a linking residue, I, K, or V, x²⁸ is a linking residue, R, Orn, N, or K, x²⁹ is a linking residue, Aib, or G, x³⁰ is a linking residue, any amino acid, or G, x³¹ is a linking residue, P, K(SH), or absent, x³² is a linking residue, S, or absent, x³³ is a linking residue, S, or absent, x³⁴ is a linking residue, G, or absent, x³⁵ is a linking residue, A, or absent, x³⁶ is a linking residue, P, or absent, x³⁷ is a linking residue, P, or absent, x³⁸ is a linking residue, P, or absent, x³⁹ is a linking residue, S, or absent, x⁴⁰ is a linking residue or absent, such that the GA targeting compound contains one linking residue comprising a nucleophilic sidechain, the linking residue being selected from the group comprising K, R, C, T, and S.

The linking residue may be K.

The N-terminus may be uncapped.

The sidechain of the linking residue may be covalently linkable to the combining site of an antibody directly or via an intermediate linker. In some embodiments, the sidechain of the linking residue is covalently linked to the combining site of an antibody directly or via an intermediate linker

In some embodiments x²⁶ is L. In some embodiments x¹¹ is S. In some embodiments x²⁵ is W or F. In some embodiments x²⁵ is W. x² may be Aib.

In some aspects of the invention, the invention comprises a GA targeting compound comprising a peptide comprising a sequence substantially homologous to:

HAibEGTFTSDx¹⁰Sx¹²x¹³x¹⁴ Ex¹⁶x¹⁷Ax¹⁹x²⁰x²¹Fx²³x²⁴x²⁵Lx²⁷x²⁸x²⁹x³⁰x³¹x³²x³³x³⁴x³⁵x³⁶x³⁷x³⁸x³⁹

In some aspects, the invention comprises a GA targeting compound comprising a peptide comprising a sequence substantially homologous to:

H¹x²E³G⁴T⁵F⁶T⁷S⁸D⁹x¹⁰S¹¹K¹²Q¹³M¹⁴E¹⁵E¹⁶E¹⁷A¹⁸V¹⁹R²⁰L²¹F²²I²³E²⁴x²⁵L²⁶K²⁷N²⁸G²⁹G³⁰P³¹S³²S³³G³⁴A³⁵P³⁶P³⁷P³⁸S³⁹x⁴⁰
wherein:

x² is a blocking group such as Aib, A, S, T, V, L, I, or D-Ala,

x²⁵ is a linking residue, or aromatic residue,

one or more of the residues P³¹ through to S³⁹ may be absent,

x⁴⁰ is a linking residue, or absent, and

one of residues S¹¹ to x⁴⁰ is a linking residue comprising a sidechain suitable for forming covalent linkages, the linking residue being selected from the group comprising K, R, C, T, and S.

In some embodiments, the GA targeting agent of the invention comprises a trp-cage, comprising a peptide sequence substantially homologous to comprising at least the residues P³¹ S³² S³³ G³⁴ A³⁵ P³⁶ P³⁷P³⁸ and S³⁹. In other embodiments, one or more of the residues comprising the trp-cage, or all of the trp-cage is absent from the GA targeting agent.

The linking residue may be substituted for one of S¹¹, K¹², Q¹³, M¹⁴, E¹⁶, E¹⁷, V¹⁹, R²⁰, L²¹, I²³, E²⁴, L²⁶, K²⁷, N²⁸, G²⁹ and G³⁰, or one of P³¹, S³², S³³, G³⁴, A³⁵, P³⁶, P³⁷, P³, or S³⁹ or x⁴⁰ Such embodiments are exemplified by: SEQ ID NO:3, SEQ ID NO:172, SEQ ID NO:4, SEQ ID NO:173, SEQ ID NO:115, SEQ ID NO:114, SEQ ID NO:113, SEQ ID NO:169 SEQ ID NO:112, SEQ ID NO:111, SEQ ID NO:110, SEQ ID NO:109, SEQ ID NO:108, SEQ ID NO:107, SEQ ID NO:106, SEQ ID NO:105, SEQ ID NO:104, SEQ ID NO:103, SEQ ID NO:170, SEQ ID NO:102, SEQ ID NO:168, SEQ ID NO:101, SEQ ID NO:100, SEQ ID NO:99, SEQ ID NO:31, SEQ ID NO:30, SEQ ID NO:29, SEQ ID NO:28, SEQ ID NO:27, SEQ ID NO:26, SEQ ID NO:25, SEQ ID NO:24, SEQ ID NO:23, SEQ ID NO:22, SEQ ID NO:21, SEQ ID NO:20, SEQ ID NO:19, SEQ ID NO:18, SEQ ID NO:17, SEQ ID NO:16, SEQ ID NO:15, SEQ ID NO:14, and SEQ ID NO:5.

Such embodiments are also exemplified by SEQ ID NO:32, SEQ ID NO:34, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, and SEQ ID NO:76.

In some embodiments, the linking residue may be substituted for one of K¹², Q¹³, M¹⁴, E¹⁶, E¹⁷, V¹⁹, R²⁰, L²¹, I²³, E²⁴, L²⁶, K²⁷, and N²⁸. Such embodiments are exemplified by: SEQ ID NO:169, SEQ ID NO:112, SEQ ID NO:111, SEQ ID NO:110, SEQ ID NO:109, SEQ ID NO:108, SEQ ID NO:107, SEQ ID NO:106, SEQ ID NO:105, SEQ ID NO:104, SEQ ID NO:103, SEQ ID NO:170, SEQ ID NO:102, SEQ ID NO:28, SEQ ID NO:27, SEQ ID NO:26, SEQ ID NO:25, SEQ ID NO:24, SEQ ID NO:23, SEQ ID NO:22, SEQ ID NO:21, SEQ ID NO:20, SEQ ID NO:19, SEQ ID NO:18, and SEQ ID NO:5.

The linking residue may be substituted for 123. Such embodiments are exemplified by SEQ ID NO:21 and SEQ ID NO:105.

The linking residue may be substituted for L²⁶. Such embodiments are exemplified by SEQ ID NO:19 and SEQ ID NO:103.

The linking residue may be K¹². Such embodiments are exemplified by SEQ ID NO:5.

In some embodiments, the linking residue may be substituted for one of Q¹³, M¹⁴, E¹⁶, E¹⁷, V¹⁹, R²⁰, L²¹, and E²⁴. Such embodiments are exemplified by: SEQ ID NO:112, SEQ ID NO:111, SEQ ID NO:110, SEQ ID NO:109, SEQ ID NO:108, SEQ ID NO:107, SEQ ID NO:106, SEQ ID NO:104, SEQ ID NO:28, SEQ ID NO:27, SEQ ID NO:26, SEQ ID NO:25, SEQ ID NO:24, SEQ ID NO:23, SEQ ID NO:22, and SEQ ID NO:20.

The linking residue may be substituted for Q¹³. Such embodiments are exemplified by SEQ ID NO:28 and SEQ ID NO:112.

In some embodiments, the linking residue may be substituted for one of M¹⁴, E¹⁶, E¹⁷, V¹⁹, R²⁰, L²¹, and E²⁴. Such embodiments are exemplified by: SEQ ID NO:111, SEQ ID NO:110, SEQ ID NO:109, SEQ ID NO:108, SEQ ID NO:107, SEQ ID NO:106, SEQ ID NO:104, SEQ ID NO:27, SEQ ID NO:26, SEQ ID NO:25, SEQ ID NO:24, SEQ ID NO:23, SEQ ID NO:22, and SEQ ID NO:20.

The linking residue may be E²⁴. Such embodiments are exemplified by SEQ ID NO:20 and SEQ ID NO:104.

In some embodiments, the linking residue may be substituted for one of M¹⁴, E¹⁶, E¹⁷, V¹⁹, R²⁰, and L²¹. Such embodiments are exemplified by: SEQ ID NO:111, SEQ ID NO:110, SEQ ID NO:109, SEQ ID NO:108, SEQ ID NO:107, SEQ ID NO:106, SEQ ID NO:27, SEQ ID NO:26, SEQ ID NO:25, SEQ ID NO:24, SEQ ID NO:23, and SEQ ID NO:22.

The linking residue may be substituted for M¹⁴. Such embodiments are exemplified by SEQ ID NO:27 and SEQ ID NO:111.

The linking residue may be substituted for E⁶. Such embodiments are exemplified by SEQ ID NO:26 and SEQ ID NO:1110.

The linking residue may be substituted for E¹⁷. Such embodiments are exemplified by SEQ ID NO:25 and SEQ ID NO:109.

The linking residue may be substituted for V¹⁹. Such embodiments are exemplified by SEQ ID NO:24 and SEQ ID NO:108.

The linking residue may be substituted for R²⁰. Such embodiments are exemplified by SEQ ID NO:23 and SEQ ID NO:107.

The linking residue may be substituted for L²¹. Such embodiments are exemplified by SEQ ID NO:22 and SEQ ID NO:106.

In some embodiments, the GA targeting agent of the invention comprises a trp-cage, comprising a peptide sequence substantially homologous to comprising at least the residues P³¹ S³² S³³ G³⁴ A³⁵ P³⁶ P³⁷ P³⁸ and S³⁹. In other embodiments, one or more or all of the trp-cage is absent from the GA targeting agent.

In some embodiments, the present invention provides a GA targeting compound comprising a peptide comprising a sequence substantially homologous to:

H¹x²E³G⁴T⁵F⁶T⁷S⁸D⁹x¹⁰x¹¹x¹²x¹³x¹⁴E¹⁵x¹⁶x¹⁷Ax¹⁹x²⁰x²¹F²²x²³x²⁴x²⁵x²⁶x²⁷x²⁸x²⁹x³⁰x³¹x³²x³³x³⁴x³⁵x³⁶x³⁷x³⁸x³⁹x⁴⁰,
wherein:

x² is a blocking group such as Aib, A, S, T, V, L, or I, x¹⁰ is V, L, I, or A, x¹¹ is a linking residue or S, x¹² is a linking residue, S, or K, x¹³ is a linking residue or Y, x¹⁴ is a linking residue, G, C, F, Y, W, or L, x¹⁶ is a linking residue, K, D, E, or G, x¹⁷ is a linking residue or Q, x¹⁹ is a linking residue, L, F, V, or A, x²⁰ is a linking residue, Orn, K(SH), R, or K, x²¹ is a linking residue or E, x²³ is a linking residue or I, x²⁴ is a linking residue or A, x²⁵ is a linking residue or aromatic residue, x²⁶ is a linking residue or L, x²⁷ is a linking residue, I, or V, x²⁸ is a linking residue, R, Orn, or K, x²⁹ is a linking residue, Aib, or G, x³⁰ is a linking residue, or G, x³¹ is a linking residue, P, K(SH), or absent, x³² is a linking residue, S, or absent, x is a linking residue, S, or absent, x³⁴ is a linking residue, G, or absent, x³⁵ is a linking residue, A, or absent, x³⁶ is a linking residue, P, or absent, x³⁷ is a linking residue, P, or absent, x³⁸ is a linking residue, P, or absent, x³⁹ is a linking residue, S, or absent, and x⁴⁰ is a linking residue, or absent,

such that the GA targeting compound contains one linking residue comprising a nucleophilic sidechain, the linking residue being selected from the group comprising K, R, C, T, and S.

In some embodiments, x² is Aib. In some embodiments, x³¹ is Aib.

In some embodiments, x¹⁶ is E. In some embodiments, x¹⁹ is V.

In some embodiments, the present invention provides a GA targeting compound comprising a peptide comprising a sequence substantially homologous to:

H¹Aib²E³G⁴T⁵F⁶T⁷S⁸D⁹L¹⁰x¹¹x¹²x¹³x¹⁴E¹⁵E¹⁶x¹⁷AV¹⁹x²⁰x²¹F²²x²³x²⁴x²⁵x²⁶x²⁷x²⁸x²⁹x³⁰Aib³¹x³²x³³x³⁴x³⁵x³⁶x³⁷x³⁸x³⁹x⁴⁰

In some embodiments, the invention comprises a GA targeting compound comprising a sequence substantially homologous to the sequence:

H¹Aib²E³G⁴T⁵F⁶T⁷S⁸D⁹V¹⁰S¹¹S¹²Y¹³L¹⁴E¹⁵E¹⁶Q¹⁷A¹⁸V¹⁹K²⁰E²¹F²²I²³A²⁴W²⁵L²⁶I²⁷K²⁸G²⁹R³⁰Aib³¹S³²S³³G³⁴A³⁵P³⁶P³⁷P³⁸S³⁹x⁴⁰,
wherein one or more of the residues Aib³¹ through to S³⁹ may be absent, and x⁴⁰ is a linking residue, or absent, and wherein one of residues from S¹¹ to x⁴⁰ is a linking residue comprising a sidechain suitable for forming covalent linkages, the linking residue being selected from the group comprising K, R, C, T, and S. Such embodiments are exemplified by SEQ ID NO:57, SEQ ID NO:64, SEQ ID NO:65, SEQ ID NO:66, SEQ ID NO:67, SEQ ID NO:68, SEQ ID NO:69, SEQ ID NO:70, SEQ ID NO:71, and SEQ ID NO:72.

In some embodiments, the invention comprises a GA targeting compound comprising a sequence substantially homologous to the sequence:

H¹x²E³G⁴T⁵F⁶T⁷S⁸D⁹V¹⁰S¹¹S¹²Y¹³L¹⁴E¹⁵E¹⁶Q¹⁷A¹⁸A¹⁹K²⁰E²¹F²²I²³A²⁴x²⁵L²⁶V²⁷K²⁸G²⁹R³⁰P³¹S³²S³³G³⁴A³⁵P³⁶P³⁷P³⁸S³⁹x⁴⁰
wherein:

x is a blocking group such as Aib, A, S, T, V, L, or I,

x²⁵ is a linking residue or aromatic residue,

one or more of the residues P³¹ to S³⁹ may be absent,

x⁴⁰ is a linking residue or absent, and

wherein one of residues from S¹¹ to x⁴⁰ is a linking residue comprising a nucleophilic sidechain, the linking residue being selected from the group comprising K, R, C, T, and S.

Such embodiments are exemplified by SEQ ID NO:32, SEQ ID NO:33, SEQ ID NO:34, SEQ ID NO:35, SEQ ID NO:36, and SEQ ID NO:37.

In some embodiments, the GA targeting agent of the invention comprises a trp-cage, comprising a peptide sequence substantially homologous to a sequence comprising at least the residues P³¹ S³²S³³G³⁴A³⁵P³⁶P³⁷P³³ and S³⁹. In other embodiments, one or more of the residues comprising the trp-cage or all of the trp-cage are absent from the GA targeting agent.

The linking residue may be K.

The N-terminus may be uncapped.

The sidechain of the linking residue may be covalently linkable to the combining site of an antibody directly or via an intermediate linker. In some embodiments, the sidechain of the linking residue is covalently linked to the combining site of an antibody directly or via an intermediate linker.

In certain embodiments, these peptides are selected from the group consisting of: a GA targeting compound as described herein, including but not limited to

(SEQ ID NO:3)

R¹-HGEGTFTSDLSKQMEEEAVRLFIEWLKNGGPSSGAPPPSK-R²

(SEQ ID NO:172)

R¹-HGEGTFTSDLSKQMEEEAVRLFIEWLKNGGPSSGAPPPSK(SH)-R²

(SEQ ID NO:4)

R¹-HAibEGTFTSDLSKQMEEEAVRLFIEWLKNGGPSSGAPPPSK-R²

(SEQ ID NO:173)

R¹-HAibEGTFTSDLSKQMEEEAVRLFIEWLKNGGPSSGAPPPSK(SH)-
R²

(SEQ ID NO:5)

R¹-HAibEGTFTSDLSKQMEEEAVRLFIEWLKNGGPSSGAPPPS-R²

(SEQ ID NO:6)

R¹-HAibEGTFTSDLSKQMEEEAVRLFIEWLKNGG-R²

(SEQ ID NO:7)

R¹-HAibEGTFTSDLSKQMEEEAVRLFIEWLKN-R²

(SEQ ID NO:8)

R¹-HAibEGTFTSDLSKQLEEEAVRLFIEFLKLN-R²

(SEQ ID NO:9)

R¹-HAibEGTFTSDLSKQLEEEAVRLAIEFLKLN-R²

(SEQ ID NO:10)

R¹-HAibEGTFTSDLSKQLEEEAVRLAIEFLKINGGPSSGAPPPS-R²

(SEQ ID NO11)

R¹-HAibEGTFTSDLSKQLEEEAVRLFIEFLKINGGPSSGAPPPS-R²

(SEQ ID NO:12)

R¹-HAibEGTFTSDLSK(Ac)QMEEEAVRLFIEWLK(Ac)NGGPSSGAPP
PS-R²

(SEQ ID NO:13)

R¹-HAibEGTFTSDLSK(benzoyl)QMEEEAVRLFIEWLK(benzoyl)
NGGPSSGAPPPS-R²

(SEQ ID NO:14)

R¹-HAibEGTFTSDLSKQMEEEAVRLFIEWLKNGGPSSGAPPKS-R²

(SEQ ID NO:99)

R¹-HAibEGTFTSDLSKQMEEEAVRLFIEWLKNGGPSSGAPPK(SH)S-
R²

(SEQ ID NO:15)

R¹-HAibEGTFTSDLSKQMEEEAVRLFIEWLKNGGPSSGAKPPS-R²

(SEQ ID NO:100)

R¹-HAibEGTFTSDLSKQMEEEAVRLFIEWLKNGGPSSGAK(SH)PPS-
R²

(SEQ ID NO:16)

R¹-HAibEGTFTSDLSKQMEEEAVRLFIEWLKNGGPSSKAPPPS-R²

(SEQ ID NO:101)

R¹-HAibEGTFTSDLSKQMEEEAVRLFIEWLKNGGPSSK(SH)APPPS-
R²

(SEQ ID NO:17)

R¹-HAibEGTFTSDLSKQMEEEAVRLFIEWLKNGGPKSGAPPPS-R²

(SEQ ID NO:168)

R¹-HAibEGTFTSDLSKQMEEEAVRLFIEWLKNGGPK(SH)SGAPPPS-

(SEQ ID NO:18)

R¹-HAibEGTFTSDLSKQMEEEAVRLFIEWLKKGGPSSGAPPPS-R²

(SEQ ID NO:102)

R¹-HAibEGTFTSDLSKQMEEEAVRLFIEWLKK(SH)GGPSSGAPPPS-
R²

(SEQ ID NO:19)

R¹-HAibEGTFTSDLSKQMEEEAVRLFIEWKKNGGPSSGAPPPS-R²

(SEQ ID NO:170)

R¹-HAibEGTFTSDLSKQMEEEAVRLFIEWLK(SH)NGGPSSGAPPPS-
R²

(SEQ ID NO:103)

R¹-HAibEGTFTSDLSKQMEEEAVRLFIEWK(SH)KNGGPSSGAPPPS-
R²

(SEQ ID NO:20)

R¹-HAibEGTFTSDLSKQMEEEAVRLFIKWLKNGGPSSGAPPPS-R²

(SEQ ID NO:104)

R¹-HAibEGTFTSDLSKQMEEEAVRLFIK(SH)WLKNGGPSSGAPPPS-
R²

(SEQ ID NO:21)

R¹-HAibEGTFTSDLSKQMEEEAVRLFKEWLIKINGGPSSGAPPPS-R²

(SEQ ID NO:105)

R¹-HAibEGTFTSDLSKQMEEEAVRLFK(SH)EWLKNGGPSSGAPPPS-
R²

(SEQ ID NO:22)

R¹-HAibEGTFTSDLSKQMEEEAVRKFIEWLKNGGPSSGAPPPS-R²

(SEQ ID NO:106)

R¹-HAibEGTFTSDLSKQMEEEAVRK(SH)FIEWLKNGGPSSGAPPPS-
R²

(SEQ ID NO:23)

R¹-HAibEGTFTSDLSKQMEEEAVKLFIEWLKINGGPSSGAPPPS-R²

(SEQ ID NO:107)

R¹-HAibEGTFTSDLSKQMEEEAVK(SH)LFIEWLKNGGPSSGAPPPS-
R²

(SEQ ID NO:24)

R¹-HAibEGTFTSDLSKQMEEEAKRLFIEWLKNGGPSSGAPPPS-R²

(SEQ ID NO:108)

R¹-HAibEGTFTSDLSKQMEEEAK(SH)RLFIEWLKNGGPSSGAPPPS-
R²

(SEQ ID NO:25)

R¹-HAibEGTFTSDLSKQMEEKAVRLFIEWLKNGGPSSGAPPPS-R²

(SEQ ID NO:109)

R¹-HAibEGTFTSDLSKQMEEK(SH)AVRLFIEWLKNGGPSSGAPPPS-
R²

(SEQ ID NO:26)

R¹-HAibEGTFTSDLSKQMEKEAVRLFIEWLKNGGPSSGAPPPS-R²

(SEQ ID NO:110)

R¹-HAibEGTFTSDLSKQMEK(SH)EAVRLFIEWLKNGGPSSGAPPPS-
R²

(SEQ ID NO:27)

R¹-HAibEGTFTSDLSKQKEEEAVRLFIEWLKNGGPSSGAPPPS-R²

(SEQ ID NO:111)

R¹-HAibEGTFTSDLSKQK(SH)EEEAVRLFIEWLKNGGPSSGAPPPS-
R²

(SEQ ID NO:28)

R¹-HAibEGTFTSDLSKKMEEEAVRLFIEWLKNGGPSSGAPPPS-R²

(SEQ ID NO:112)

R¹-HAibEGTFTSDLSKK(SH)MEEEAVRLFIEWLKNGGPSSGAPPPS-
R²

(SEQ ID NO:29)

R¹-HAibEGTFTSDLKKQMEEEAVRLFIEWLKNGGPSSGAPPPS-R²

(SEQ ID NO:169)

R¹-HAibEGTFTSDLSK(SH)QMEEEAVRLFIEWLKNGGPSSGAPPPS-
R²

(SEQ ID NO:113)

R¹-HAibEGTFTSDLK(SH)KQMEEEAVRLFIEWLKNGGPSSGAPPPS-
R²

(SEQ ID NO:30)

R¹-HAibEGTFTSDLSKQMEEEAVRLFIEWLKNGGK-R²

(SEQ ID NO:114)

R¹-HAibEGTFTSDLSKQMEEEAVRLFIEWLKNGGK(SH)-R²

(SEQ ID NO:31)

R¹-HAibEGTFTSDLSKQMEEEAVRKFIEWLKNGGPSS-R²

(SEQ ID NO:115)

R¹-HAibEGTFTSDLSKQMEEEAVRKFIEWLK(SH)NGGPSS-R²

(SEQ ID NO:32)

R¹-HAibEGTFTSDVSSYLEGQAAKEFIAWLVKGR-R²

(SEQ ID NO:33)

R¹-HAEGTFTSDVSSYLEGQAAKEFIAWLVKGRK-R²

(SEQ ID NO:34)

R¹-HGEGTFTSDVSSYLEGQAAKEFIAWLVKGRK-R²

(SEQ ID NO:35)

R¹-HAibEGTFTSDVSSYLEGQAAKEFIAWLVKAibR-R²

(SEQ ID NO:36)

R¹-HAibEGTFTSDVSSYLEGQAAKEFIAWLVKAibRK-R²

(SEQ ID NO:37)

R¹-HAibEGTFTSDVSSYLEGQAAKEFIAWLVKGRK-R²

(SEQ ID NO:38)

R¹-HAibEGTFTSDVSSYLEGQAAKEFIAWLVKGRK(SH)-R²

(SEQ ID NO:39)

R¹-HAibEGTFTSDVSSYLEGQAAK(SH)EFIAWLVKGR-R²

(SEQ ID NO:40)

R¹-HAibEGTFTSDVSSYGEGQAAKEFIAWLVKAibR-R²

(SEQ ID NO:41)

R¹-HAibEGTFTSDVSSYCEGQAAKEFIAWLVKAibR-R²

(SEQ ID NO:42)

R¹-HAibEGTFTSDVSSYFEGQAAKEFIAWLVKAibR-R²

(SEQ ID NO:43)

R¹-HAibEGTFTSDVSSYYEGQAAKEFIAWLVKAibR-R²

(SEQ ID NO:44)

R¹-HAibEGTFTSDVSSYWEGQAAKEFIAWLVKAibR-R²

(SEQ ID NO:45)

R¹-HAibEGTFTSDVSSYLEEQAAKEFIAWLVKAibR-R²

(SEQ ID NO:46)

R¹-HAibEGTFTSDVSSYLEDQAAKEFIAWLVKAibR-R²

(SEQ ID NO:47)

R¹-HAibEGTFTSDVSSYLEKQAAKEFIAWLVKAibR-R²

(SEQ ID NO:48)

R¹-HAibEGTFTSDVSSYLEGQAVKEFIAWLVKAibR-R²

(SEQ ID NO:49)

R¹-HAibEGTFTSDVSSYLEGQAIKEFIAWLVKAibR-R²

(SEQ ID NO:50)

R¹-HAibEGTFTSDVSSYLEGQALKEFIAWLVKAibR-R²

(SEQ ID NO:51)

R¹-HAibEGTFTSDVSSYLEGQAAREFIAWLVKAibR-R²

(SEQ ID NO:52)

R¹-HAibEGTFTSDVSSYLEGQAAOrnEFIAWLVKAibR-R²

(SEQ ID NO:53)

R¹-HAibEGTFTSDVSSYLEGQAAKEFIAFLVKAibR-R²

(SEQ ID NO:54)

R¹-HAibEGTFTSDVSSYLEGQAAKEFIAWLIKAibR-R²

(SEQ ID NO:55)

R¹-HAibEGTFTSDVSSYLEGQAAKEFIAWLVRAibR-R²

(SEQ ID NO:56)

R¹-HAibEGTFTSDVSSYLEGQAAKEFIAWLVOrnAibR-R²

(SEQ ID NO:57)

R¹-HAibEGTFTSDVSSYLEEQAVKEFIAWLIKAibR-R²

(SEQ ID NO:58)

R¹-HAibEGTFTSDVSSYFEEQAVIKIEFIAWLIKAibR-R²

(SEQ ID NO:59)

R¹-HAibEGTFTSDVSSYYEEQAVKEFIAWLIKAibR-R²

(SEQ ID NO:60)

R¹-HAibEGTFTSDVSSYWEEQAVKEFIAWLIKAibR-R²

(SEQ ID NO:61)

R¹-HAibEGTFTSDVSSYLEEQAVKEFIAWLIRAibR-R²

(SEQ ID NO:62)

R¹-HAibEGTFTSDVSSYLEEQAVREFIAWLIRAibR-R²

(SEQ ID NO:63)

R¹-HAibEGTFTSDVSSYLEEQAVKEFIAWLIKAibRPSSGAPPPS-R²

(SEQ ID NO:171)

R¹-HAibEGTFTSDVSSYLEEQAVK(SH)EFIAWLIKAibRPSSGAPPP
S-R²

(SEQ ID NO:64)

R¹-HAibEGTFTSDKSSYLEEQAVKEFIAWLIKAibR-R²

(SEQ ID NO:116)

R¹-HAibEGTFTSDK(SH)SSYLEEQAVKEFIAWLIKAibR-R²

(SEQ ID NO:65)

R¹-HAibEGTFTSDVSKYLEEQAVKEFIAWLIKAibR-R²

(SEQ ID NO:117)

R¹-HAibEGTFTSDVSK(SH)YLEEQAVKEFIAWLIKAibR-R²

(SEQ ID NO:66)

R¹-HAibEGTFTSDVSSYKEEQAVKEFIAWLIKAibR-R²

(SEQ ID NO:118)

R¹-HAibEGTFTSDVSSYK(SH)EEQAVKEFIAWLIKAibR-R²

(SEQ ID NO:67)

R¹-HAibEGTFTSDVSSYLEKQAVKEFIAWLIKAibR-R²

(SEQ ID NO:119)

R¹-HAibEGTFTSDVSSYLEK(SH)QAVKEFIAWLIKAibR-R²

(SEQ ID NO:68)

R¹-HAibEGTFTSDVSSYLEEQKVKEFIAWLIKAibR-R²

(SEQ ID NO:120)

R¹-HAibEGTFTSDVSSYLEEQK(SH)VKEFIAWLIKAibR-R²

(SEQ ID NO:69)

R¹-HAibEGTFTSDVSSYLEEQAVKEKIAWLIKAibR-R²

(SEQ ID NO:121)

R¹-HAibEGTFTSDVSSYLEEQAVK(SH)EKIAWLIKAibR-R²

(SEQ ID NO:70)

R¹-HAibEGTFTSDVSSYLEEQAVKEFIKWLIKAibR-R²

(SEQ ID NO:122)

R¹-HAibEGTFTSDVSSYLEEQAVKEFIK(SH)WLIKAibR-R²

(SEQ ID NO:71)

R¹-HAibEGTFTSDVSSYLEEQAVKEFIAWKIKAibR-R²

(SEQ ID NO:123)

R¹-HAibEGTFTSDVSSYLEEQAVKEFIAWK(SH)IKAibR-R²

(SEQ ID NO:72)

R¹-HAibEGTFTSDVSSYLEEQAVKEFIAWLIKAibRPSSGAPPPSK-R²

(SEQ ID NO:124)

R¹-HAibEGTFTSDVSSYLEEQAVKEFIAWLIKAibRPSSGAPPPSK
(SH)-R²

(SEQ ID NO:73)

R¹-HAibEGTFTSDVSSYLEEQAVKEFIAWLIK(Ac)AibR-R²

(SEQ ID NO:74)

R¹-HAibEGTFTSDVSSYLEEQAVKEFIAWLIK(benzoyl)AibR-R²

(SEQ ID NO:75)

R¹-H(Trans 3-hexanoyl)AibEGTFTSDVSSYLEEQAVKEFIAWLI
KAibR-R²

(SEQ ID NO:76)

R¹-H(3-Aminophenylacetyl)AibEGTFTSDVSSYLEEQAVKEFIA
WLIKAibR-R²

R¹ is absent, CH₃, C(O)CH3, C(O)CH₂CH₃, C(O)CH₂CH₂CH₃, or C(O)CH(CH₃)CH₃; and

R² is OH, NH₂, NH(CH₃), NHCH₂CH₃, NHCH₂CH₂CH₃, NHCH(CH₃)CH₃, NHCH₂CH₂CH₂CH₃, NHCH(CH₃)CH₂CH₃, NHC₆H₅, NHCH₂CH₂OCH₃, NHOCH₃, NHOCH₂CH₃, a carboxy protecting group, a lipid fatty acid group or a carbohydrate.

In certain embodiments, these peptides are selected from the group consisting of a GA targeting compound as described herein, including but not limited to:

(SEQ ID NO:125)

R¹-HAFGTFTSDVSSYLFGQAAKFFIAWLVRGR

(SEQ ID NO:126)

R¹-HAEGTFTSDVSSYLEGQAAREFIAWLVRGRK

(SEQ ID NO:127)

R¹-HAEGTFTSDVSSYLEGQAAREFIAWLVRGK

(SEQ ID NO:128)

R¹-HAibEGTFTSDVSSYLEAibQAAKEFIAWLVKAibR

(SEQ ID NO:35)

R¹-HAibEGTFTSDVSSYLEGQAAKEFIAWLVKAibR

(SEQ ID NO:129)

R¹-HAibEGTFTSDVSSYLEAibQAAKEFIAWLVKGR

(SEQ ID NO:130)

R¹-HAibEGTFTSDVSSYLEAibQAAREFIAWLVRAibRK

(SEQ ID NO:131)

R¹-HAibEGTFTSDVSSYLEGQAAREFIAWLVRAibRK

(SEQ ID NO:132)

R¹-HAibEGTFTSDVSSYLEAibQAAREFIAWLVRGRK

(SEQ ID NO:133)

R¹-HAibEGTFTSDVSSYLEAibQAAREFIAWLVKAibR

(SEQ ID NO:134)

R¹-HAibEGTFTSDVSSYLEGQAAREFIAWLVKGRK

(SEQ ID NO:135)

R¹-HAibEGTFTSDVSSYLEAibQAAREFIAWLVKGRK

(SEQ ID NO:136)

R¹-HAibEGTFTSDVSSYLEAibQAAKEFIAWLVRAibR

(SEQ ID NO:137)

R¹-HAibEGTFTSDVSSYLEGQAAKEFIAWLVRAibRK

(SEQ ID NO:138)

R¹-HAibEGTFTSDVSSYLEAibQAAKEFIAWLVRGRK

(SEQ ID NO:139)

R¹-HAibEGTFTSDVSSYLEAibQAAKEFIAWLVKAibRAK

(SEQ ID NO:140)

R¹-HAibEGTFTSDVSSYLEGQAAKEFIAWLVKAibRAK

(SEQ ID NO:141)

R¹-HAibEGTFTSDVSSYLEAibQAAKEFIAWLVKGRAK

(SEQ ID NO:142)

R¹-HAibEGTFTSDVSSYLEAibQAAKEFIAWLVKAibRK

(SEQ ID NO:36)

R¹-HAibEGTFTSDVSSYLEGQAAKEFIAWLVKAibRK

(SEQ ID NO:143)

R¹-HAibEGTFTSDVSSYLEAibQAAKEFIAWLVKGRK

(SEQ ID NO:144)

R¹-H(D-Ala)EGTFTSDVSSYLEGQAAKEFIAWLVKGRK

(SEQ ID NO:145)

R¹-HAibEGTFTSDVSSYLEAibQAAibKEFIAWLVKGRK

(SEQ ID NO:146)

R¹-HAibEGTFTSDVSSYLEEEAAREFIEWLVRGRK

wherein:

R¹ is absent, CH₃, C(O)CH3, C(O)CH₂CH₃, C(O)CH₂CH₂CH₃, or C(O)CH(CH₃)CH₃; and

R² is OH, NH₂, NH(CH₃), NHCH₂CH₃, NHCH₂CH₂CH₃, NHCH(CH₃)CH₃, NHCH₂CH₂CH₂CH₃, NHCH(CH₃)CH₂CH₃, NHC₆H₅, NHCH₂CH₂OCH₃, NHOCH₃, NHOCH₂CH₃, a carboxy protecting group, a lipid fatty acid group or a carbohydrate.

In one embodiment of the invention the GA targeting compound comprises a sequence with at least an 80% amino acid homology with either SEQ ID NO 1 or SEQ ID NO 2

### R¹-HAEGTFTSDVSSYLEGQAAKEFIAWLVKGR (SEQ ID NO:1) [Glp-1]

### R¹-HGEGTFTSDLSKQMEEEAVRLFIEWLKNGGPSSGAPPPS (SEQ ID NO:2) [Exendin-4]

The GA targeting compound may comprise an amino acid sequence of the formula:

X¹X²E³G⁴T⁵F⁶T⁷S⁸D⁹X¹⁰S¹¹X¹²X¹³X¹⁴E¹⁵X¹⁶X¹⁷A¹⁸X¹⁹X²⁰X²¹F²²X²³X²⁴X²⁵X²⁶X²⁷X²⁸X²⁹X³⁰X³¹X³²X³³X³⁴X³⁵X³⁶X³⁷X³⁸X³⁹X⁴⁰
wherein:

x¹ is L-histidine, D-histidine, desamino-histidine, 2-amino-histidine, β-hydroxy-histidine, homohistidine, Nα-acetyl-histidine, α-fluoromethyl-histidine, α-methyl-histidine, 3-pyridylalanine, 2-pyridylalanine, or 4-pyridylalanine; x² is A, D-Ala, G, V, L, I, K, Aib, (1-aminocyclopropyl)carboxylic acid, (1-aminocyclobutyl)carboxylic acid, 1-aminocyclopentyl)carboxylic acid, (1-aminocyclohexyl)carboxylic acid, (1-aminocycloheptyl)carboxylic acid, or (1-aminocyclooctyl)carboxylic acid; X¹⁰ is V or L; X¹² is S, K or R, X¹³ is Y or Q; X¹⁴ is L or M; X¹⁶ is G, E or Aib; X¹⁷ is Q, E, K, or R; X¹⁹ is A or V; X²⁰ is K, E or A; X²¹ is E or L; X²⁴ is A, E or R; X²⁷ is V or K; X²⁸ is K, E, N, or R; X²⁹ is G or R; X³⁰ is R, G or K; X³¹ is G, A, E, P, K, amide, or absent; X³² is K, S, amide or absent. X³³ is S, K, amide, or absent; X³⁴ is G, amide, or absent; X³⁵ is A, amide, or absent; X³⁶ is P, amide, or absent; X³⁷ is P, amide, or absent; X³⁸ is P, amide, or absent; X³⁹ is S, amide, or absent; X⁴⁰ is amide or absent;

provided that if X³², X³³, X³⁴, X³⁵, X³⁶,X³⁷, X³⁸, X³⁹ or X⁴⁰ is absent then each amino acid residue downstream is also absent.

In another embodiment of the invention the GA targeting compound of the invention may comprise the amino acid sequence of formula:

X¹X²E³G⁴T⁵F⁶T⁷S⁸D⁹V¹⁰S¹¹X¹²Y¹³L¹⁴E¹⁵X¹⁶X¹⁷A¹⁸A¹⁹X²⁰E²¹F²²I²³X²⁴W²⁵L²⁶V²⁷X²⁸X²⁹X³⁰X³¹X³²
wherein X¹ is L-histidine, D-histidine, desamino-histidine, 2-amino-histidine, β-hydroxy-histidine, homohistidine, N α-acetyl-histidine, α-fluoromethyl-histidine, α-methyl-histidine, 3-pyridylalanine, 2-pyridylalanine, or 4-pyridylalanine; X² is A, D-Ala, G, V, L, I, K, Aib, (1-aminocyclopropyl)carboxylic acid, (1-aminocyclobutyl)carboxylic acid, 1-aminocyclopentyl)carboxylic acid, or (1-aminocyclohexyl)carboxylic acid, (1-aminocycloheptyl)carboxylic acid, or (1-aminocyclooctyl)carboxylic acid; X¹² is S, K, or R; X¹⁶ is G, E, or Aib; X¹⁷ is Q, E, K, or R; X²⁰ is K, E, or T; X²⁴ is A, E, or R; X²⁸ is K, E, or R; X²⁹ is G or Aib; X³⁰ is R or K; X³¹ is G, A, E, or K; X³² is K, amide, or absent.

In another embodiment of the invention the GA targeting agent is dipeptidyl aminopeptidase IV protected. In another embodiment of the invention the GA targeting agent is hydrolysed by DPP-IV at a rate lower than the rate of hydrolysis of SEQ ID NO:1 using the DPP-IV hydrolysis assay disclosed herein. In another embodiment of the invention A² of the GA targeting agent has been substituted by another amino acid residue (X²). In some embodiments, X² is Aib. In another embodiment of the invention X¹ is selected from the group consisting of D-histidine, desamino-histidine, 2-amino-histidine, [beta]-hydroxy-histidine, homohistidine, N α-acetyl-histidine, α-fluoromethyl-histidine, α-methyl-histidine, 3-pyridylalanine, 2-pyridylalanine, and 4-pyridylalanine.

In another embodiment of the invention the GA targeting agent comprises no more than twelve amino acid residues which have been exchanged, added or deleted as compared to SEQ ID NO:1 or SEQ ID NO:2. In another embodiment of the invention the GA targeting agent comprises no more than six amino acid residues which have been exchanged, added or deleted as compared to SEQ ID NO:1 or SEQ ID NO:2. In another embodiment of the invention the GA targeting agent comprises no more than four amino acid residues which have been exchanged, added or deleted as compared to SEQ ID NO:1 or SEQ ID NO:2. In another embodiment of the invention the GA targeting agent comprises no more than two amino acid residues which have been exchanged, added or deleted as compared to SEQ ID NO:1 or SEQ ID NO:2. In another embodiment of the invention the GA targeting agent comprises no more than 4 amino acid residues which are not encoded by the genetic code.

In another embodiment of the invention the GA targeting compound is: HGEGTFTSDLSKQMEEEAVRLFIEWLKNGGPSSGAPPPSKKKKKK-amide (SEQ ID NO:147).

In some embodiments, the invention provides a GA targeting agent that is substantially homologous to GLP-1. GA targeting agents of the invention may be at least 95% homologous to GLP-1 (SEQ ID NO:1). GA targeting agents of the invention may be at least 90% homologous to GLP-1. GA targeting agents of the invention may be at least 80% homologous to GLP-1. GA targeting agents of the invention may be at least 70% homologous to GLP-1. GA targeting agents of the invention may be at least 60% homologous to GLP-1. GA targeting agents of the invention may be at least 53% homologous to GLP-1. GA targeting agents of the invention may be at least 50% homologous to GLP-1.

In some embodiments, the invention provides a GA targeting agent that is substantially homologous to Exendin-4 (SEQ ID NO:2). GA targeting agents of the invention may be at least 95% homologous to Exendin-4. GA targeting agents of the invention may be at least 90% homologous to Exendin-4. GA targeting agents of the invention may be at least 80% homologous to Exendin-4. GA targeting agents of the invention may be at least 70% homologous to Exendin-4. GA targeting agents of the invention may be at least 60% homologous to Exendin-4. GA targeting agents of the invention may be at least 53% homologous to Exendin-4. GA targeting agents of the invention may be at least 50% homologous to Exendin-4.

In certain embodiments, a GA targeting agent-linker conjugate is provided having Formula I:

L-[GA targeting agent] (I)

wherein:

[GA targeting agent] is a peptide agonist of GLP-1R. In certain embodiments, [GA targeting agent] is a peptide selected from the group consisting of: a GA targeting compound as described herein, including but not limited to:

(SEQ ID NO:3)

R¹-HGEGTFTSDLSKQMEEEAVRLFIEWLKNGGPSSGAPPPSK-R²

(SEQ ID NO:172)

R¹-HGEGTFTSDLSKQMEEEAVRLFIEWLKNGGPSSGAPPPSK(SH)-R²

(SEQ ID NO:4)

R¹-HAibEGTFTSDLSKQMEEEAVRLFIEWLKNGGPSSGAPPPSK-R²

(SEQ ID NO:173)

R¹-HAibEGTFTSDLSKQMEEEAVRLFIEWLKNGGPSSGAPPPSK(SH)-
R²

(SEQ ID NO:5)

R¹-HAibEGTFTSDLSKQMEEEAVRLFIEWLKNGGPSSGAPPPS-R²

(SEQ ID NO:6)

R¹-HAibEGTFTSDLSKQMEEEAVRLFIEWLKNGG-R²

(SEQ ID NO:7)

R¹-HAibEGTFTSDLSKQMEEEAVRLFIEWLKN-R²

(SEQ ID NO:8)

R¹-HAibEGTFTSDLSKQLEEEAVRLFIEFLKIN-R²

(SEQ ID NO:9)

R¹-HAibEGTFTSDLSKQLEEEAVRLAIEFLKIN-R²

(SEQ ID NO:10)

R¹-HAibEGTFTSDLSKQLEEEAVRLAIEFLKINGGPSSGAPPPS-R²

(SEQ ID NO:11)

R¹-HAibEGTFTSDLSKQLEEEAVRLFIEFLKINGGPSSGAPPPS-R²

(SEQ ID NO:12)

R¹-HAibEGTFTSDLSK(Ac)QMEEEAVRLFIEWLK(Ac)NGGPSSGAPP
PS-R²

(SEQ ID NO:13)

R¹-HAibEGTFTSDLSK(benzoyl)QMEEEAVRLFIEWLK(benzoyl)
NGGPSSGAPPPS-R²

(SEQ ID NO:14)

R¹-HAibEGTFTSDLSKQMEEEAVRLFIEWLKNGGPSSGAPPKS-R²

(SEQ ID NO:99)

R¹-HAibEGTFTSDLSKQMEEEAVRLFIEWLKNGGPSSGAPPK(SH)S-
R²

(SEQ ID NO:15)

R¹-HAibEGTFTSDLSKQMEEEAVRLFIEWLKNGGPSSGAKPPS-R²

(SEQ ID NO:100)

R¹-HAibEGTFTSDLSKQMEEEAVRLFIEWLKNGGPSSGAK(SH)PPS-
R²

(SEQ ID NO:16)

R¹-HAibEGTFTSDLSKQMEEEAVRLFIEWLKNGGPSSKAPPPS-R²

(SEQ ID NO:101)

R¹-HAibEGTFTSDLSKQMEEEAVRLFIEWLKNGGPSSK(SH)APPPS-
R²

(SEQ ID NO:17)

R¹-HAibEGTFTSDLSKQMEEEAVRLFIEWLKNGGPKSGAPPPS-R²

(SEQ ID NO:168)

R¹-HAibEGTFTSDLSKQMEEEAVRLFIEWLKNGGPK(SH)SGAPPPS-
R²

(SEQ ID NO:18)

R¹-HAibEGTFTSDLSKQMEEEAVRLFIEWLKKGGPSSGAPPPS-R²

(SEQ ID NO:102)

R¹-HAibEGTFTSDLSKQMEEEAVRLFIEWLKK(SH)GGPSSGAPPPS-
R²

(SEQ ID NO:19)

R¹-HAibEGTFTSDLSKQMEEEAVRLFIEWKKNGGPSSGAPPPS-R²

(SEQ ID NO:170)

R¹-HAibEGTFTSDLSKQMEEEAVRLFIEWLK(SH)NGGPSSGAPPPS-
R²

(SEQ ID NO:103)

R¹-HAibEGTFTSDLSKQMEEEAVRLFIEWK(SH)KNGGPSSGAPPPS-
R²

(SEQ ID NO:20)

R¹-HAibEGTFTSDLSKQMEEEAVRLFIKWLKNGGPSSGAPPPS-R²

(SEQ ID NO:104)

R¹-HAibEGTFTSDLSKQMEEEAVRLFIK(SH)WLKNGGPSSGAPPPS-
R²

(SEQ ID NO:21)

R¹-HAibEGTFTSDLSKQMEEEAVRLFKEWLIKINGGPSSGAPPPS-R²

(SEQ ID NO:105)

R¹-HAibEGTFTSDLSKQMEEEAVRLFK(SH)EWLKNGGPSSGAPPPS-
R²

(SEQ ID NO:22)

R¹-HAibEGTFTSDLSKQMEEEAVRKFIEWLKNGGPSSGAPPPS-R²

(SEQ ID NO:106)

R¹-HAibEGTFTSDLSKQMEEEAVRK(SH)FIEWLKNGGPSSGAPPPS-
R²

(SEQ ID NO:23)

R¹-HAibEGTFTSDLSKQMEEEAVKLFIEWLKINGGPSSGAPPPS-R²

(SEQ ID NO:107)

R¹-HAibEGTFTSDLSKQMEEEAVK(SH)LFIEWLKNGGPSSGAPPPS-
R²

(SEQ ID NO:24)

R¹-HAibEGTFTSDLSKQMEEEAKRLFIEWLKNGGPSSGAPPPS-R²

(SEQ ID NO:108)

R¹-HAibEGTFTSDLSKQMEEEAK(SH)RLFIEWLKNGGPSSGAPPPS-
R²

(SEQ ID NO:25)

R¹-HAibEGTFTSDLSKQMEEKAVRLFIEWLKNGGPSSGAPPPS-R²

(SEQ ID NO:109)

R¹-HAibEGTFTSDLSKQMEEK(SH)AVRLFIEWLKNGGPSSGAPPPS-
R²

(SEQ ID NO:26)

R¹-HAibEGTFTSDLSKQMEKEAVRLFIEWLKNGGPSSGAPPPS-R²

(SEQ ID NO:110)

R¹-HAibEGTFTSDLSKQMEK(SH)EAVRLFIEWLKNGGPSSGAPPPS-
R²

(SEQ ID NO:27)

R¹-HAibEGTFTSDLSKQKEEEAVRLFIEWLKNGGPSSGAPPPS-R²

(SEQ ID NO:111)

R¹-HAibEGTFTSDLSKQK(SH)EEEAVRLFIEWLKNGGPSSGAPPPS-
R²

(SEQ ID NO:28)

R¹-HAibEGTFTSDLSKKMEEEAVRLFIEWLKNGGPSSGAPPPS-R²

(SEQ ID NO:112)

R¹-HAibEGTFTSDLSKK(SH)MEEEAVRLFIEWLKNGGPSSGAPPPS-
R²

(SEQ ID NO:29)

R¹-HAibEGTFTSDLKKQMEEEAVRLFIEWLKNGGPSSGAPPPS-R²

(SEQ ID NO:169)

R¹-HAibEGTFTSDLSK(SH)QMEEEAVRLFIEWLKNGGPSSGAPPPS-
R²

(SEQ ID NO:113)

R¹-HAibEGTFTSDLK(SH)KQMEEEAVRLFIEWLKNGGPSSGAPPPS-
R²

(SEQ ID NO:30)

R¹-HAibEGTFTSDLSKQMEEEAVRLFIEWLKNGGK-R²

(SEQ ID NO:114)

R¹-HAibEGTFTSDLSKQMEEEAVRLFIEWLKNGGK(SH)-R²

(SEQ ID NO:31)

R¹-HAibEGTFTSDLSKQMEEEAVRKFIEWLKNGGPSS-R²

(SEQ ID NO:115)

R¹-HAibEGTFTSDLSKQMEEEAVRKFIEWLK(SH)NGGPSS-R²

(SEQ ID NO:32)

R¹-HAibEGTFTSDVSSYLEGQAAKEFIAWLVKGR-R²

(SEQ ID NO:33)

R¹-HAEGTFTSDVSSYLEGQAAKEFIAWLVKGRK-R²

(SEQ ID NO:34)

R¹-HGEGTFTSDVSSYLEGQAAKEFIAWLVKGRK-R²

(SEQ ID NO:35)

R¹-HAibEGTFTSDVSSYLEGQAAKEFIAWLVKAibR-R²

(SEQ ID NO:36)

R¹-HAibEGTFTSDVSSYLEGQAAKEFIAWLVKAibRK-R²

(SEQ ID NO:37)

R¹-HAibEGTFTSDVSSYLEGQAAKEFIAWLVKGRK-R²

(SEQ ID NO:38)

R¹-HAibEGTFTSDVSSYLEGQAAKEFIAWLVKGRK(SH)-R²

(SEQ ID NO:39)

R¹-HAibEGTFTSDVSSYLEGQAAK(SH)EFIAWLVKGR-R²

(SEQ ID NO:40)

R¹-HAibEGTFTSDVSSYGEGQAAKEFIAWLVKAibR-R²

(SEQ ID NO:41)

R¹-HAibEGTFTSDVSSYCEGQAAKEFIAWLVKAibR-R²

(SEQ ID NO:42)

R¹-HAibEGTFTSDVSSYFEGQAAKEFIAWLVKAibR-R²

(SEQ ID NO:43)

R¹-HAibEGTFTSDVSSYYEGQAAKEFIAWLVKAibR-R²

(SEQ ID NO:44)

R¹-HAibEGTFTSDVSSYWEGQAAKEFIAWLVKAibR-R²

(SEQ ID NO:45)

R¹-HAibEGTFTSDVSSYLEEQAAKEFIAWLVKAibR-R²

(SEQ ID NO:46)

R¹-HAibEGTFTSDVSSYLEDQAAKEFIAWLVKAibR-R²

(SEQ ID NO:47)

R¹-HAibEGTFTSDVSSYLEKQAAKEFIAWLVKAibR-R²

(SEQ ID NO:48)

R¹-HAibEGTFTSDVSSYLEGQAVKEFIAWLVKAibR-R²

(SEQ ID NO:49)

R¹-HAibEGTFTSDVSSYLEGQAIKEFIAWLVKAibR-R²

(SEQ ID NO:50)

R¹-HAibEGTFTSDVSSYLEGQALKEFIAWLVKAibR-R²

(SEQ ID NO:51)

R¹-HAibEGTFTSDVSSYLEGQAAREFIAWLVKAibR-R²

(SEQ ID NO:52)

R¹-HAibEGTFTSDVSSYLEGQAAOrnEFIAWLVKAibR-R²

(SEQ ID NO:53)

R¹-HAibEGTFTSDVSSYLEGQAAKEFIAFLVKAibR-R²

(SEQ ID NO:54)

R¹-HAibEGTFTSDVSSYLEGQAAKEFIAWLIKAibR-R²

(SEQ ID NO:55)

R¹-HAibEGTFTSDVSSYLEGQAAKEFIAWLVRAibR-R²

(SEQ ID NO:56)

R¹-HAibEGTFTSDVSSYLEGQAAKEFIAWLVOrnAibR-R²

(SEQ ID NO:57)

R¹-HAibEGTFTSDVSSYLEEQAVKEFIAWLIKAibR-R²

(SEQ ID NO:58)

R¹-HAibEGTFTSDVSSYFEEQAVIKIEFIAWLIKAibR-R²

(SEQ ID NO:59)

R¹-HAibEGTFTSDVSSYYEEQAVKEFIAWLIKAibR-R²

(SEQ ID NO:60)

R¹-HAibEGTFTSDVSSYWEEQAVKEFIAWLIKAibR-R²

(SEQ ID NO:61)

R¹-HAibEGTFTSDVSSYLEEQAVKEFIAWLIRAibR-R²

(SEQ ID NO:62)

R¹-HAibEGTFTSDVSSYLEEQAVREFIAWLIRAibR-R²

(SEQ ID NO:63)

R¹-HAibEGTFTSDVSSYLEEQAVKEFIAWLIKAibRPSSGAPP
PS-R²

(SEQ ID NO:117)

R¹-HAibEGTFTSDVSSYLEEQAVK(SH)EFIAWLIKAibRPSSGAPPP
S-R²

(SEQ ID NO:64)

R¹-HAibEGTFTSDKSSYLEEQAVKEFIAWLIKAibR-R²

(SEQ ID NO:116)

R¹-HAibEGTFTSDK(SH)SSYLEEQAVKEFIAWLIKAibR-R²

(SEQ ID NO:65)

R¹-HAibEGTFTSDVSKYLEEQAVKEFIAWLIKAibR-R²

(SEQ ID NO:117)

R¹-HAibEGTFTSDVSK(SH)YLEEQAVKEFIAWLIKAibR-R²

(SEQ ID NO:66)

R¹-HAibEGTFTSDVSSYKEEQAVKEFIAWLIKAibR-R²

(SEQ ID NO:118)

R¹-HAibEGTFTSDVSSYK(SH)EEQAVKEFIAWLIKAibR-R²

(SEQ ID NO:67)

R¹-HAibEGTFTSDVSSYLEKQAVKEFIAWLIKAibR-R²

(SEQ ID NO:119)

R¹-HAibEGTFTSDVSSYLEK(SH)QAVKEFIAWLIKAibR-R²

(SEQ ID NO:68)

R¹-HAibEGTFTSDVSSYLEEQKVKEFIAWLIKAibR-R²

(SEQ ID NO:120)

R¹-HAibEGTFTSDVSSYLEEQK(SH)VKEFIAWLIKAibR-R²

(SEQ ID NO:69)

R¹-HAibEGTFTSDVSSYLEEQAVKEKIAWLIKAibR-R²

(SEQ ID NO:121)

R¹-HAibEGTFTSDVSSYLEEQAVK(SH)EKIAWLIKAibR-R²

(SEQ ID NO:70)

R¹-HAibEGTFTSDVSSYLEEQAVKEFIKWLIKAibR-R²

(SEQ ID NO:122)

R¹-HAibEGTFTSDVSSYLEEQAVKEFIK(SH)WLIKAibR-R²

(SEQ ID NO:71)

R¹-HAibEGTFTSDVSSYLEEQAVKEFIAWKIKAibR-R²

(SEQ ID NO:123)

R¹-HAibEGTFTSDVSSYLEEQAVKEFIAWK(SH)IKAibR-R²

(SEQ ID NO:72)

R¹-HAibEGTFTSDVSSYLEEQAVKEFIAWLIKAibRPSSGAPPPSK-R²

(SEQ ID NO:124)

R¹-HAibEGTFTSDVSSYLEEQAVKEFIAWLIKAibRPSSGAPPPSK
(SH)-R²

(SEQ ID NO:73)

R¹-HAibEGTFTSDVSSYLEEQAVKEFIAWLIK(Ac)AibR-R²

(SEQ ID NO:74)

R¹-HAibEGTFTSDVSSYLEEQAVKEFIAWLIK(benzoyl)AibR-R²

(SEQ ID NO:75)

R¹-H(Trans 3-hexanoyl)AibEGTFTSDVSSYLEEQAVKEFIAWLI
KAibR-R²

(SEQ ID NO:76)

R¹-H(3-Aminophenylacetyl)AibEGTFTSDVSSYLEEQAVKEFIA
WLIKAibR-R²

wherein:

R¹ is absent, CH₃, C(O)CH3, C(O)CH₂CH₃, C(O)CH₂CH₂CH₃, or C(O)CH(CH₃)CH₃; and

R² is OH, NH₂, NH(CH₃), NHCH₂CH₃, NHCH₂CH₂CH₃, NHCH(CH₃)CH₃, NHCH₂CH₂CH₂CH₃, NHCH(CH₃)CH₂CH₃, NHC₆H₅, NHCH₂CH₂OCH₃, NHOCH₃, NHOCH₂CH₃, a carboxy protecting group, a lipid fatty acid group or a carbohydrate;

and any of the C-terminus truncations, and analogs that may be formed from these peptides; and

L is a linker moiety having the formula —X—Y-Z, wherein:
X is optionally present, and is a biologically compatible polymer, block copolymer C, H, N, O, P, S, halogen (F, Cl, Br, I), or a salt thereof, alkyl, alkenyl, alkynyl, oxoalkyl, oxoalkenyl, oxoalkynyl, aminoalkyl, aminoalkenyl, aminoalkynyl, sulfoalkyl, sulfoalkenyl, sulfoalkynyl, phosphoalkyl, phosphoalkenyl, or phosphoalkynyl group, attached to one of the residues that comprises a GA targeting agent;
Y is an optionally present recognition group comprising at least a ring structure; and
Z is a reactive group that is capable of covalently linking to a sidechain in a combining site of an antibody; and

pharmaceutically acceptable salts, stereoisomers, tautomers, solvates, and prodrugs thereof.

In some embodiments X is attached to the carboxy terminus, a S sidechain, a K sidechain, a K(SH) sidechain, a T sidechain, or a Y sidechain of a GA targeting agent.

In other aspects, the invention provides compounds having the formula selected from the group consisting of

(SEQ ID NO:148)

R¹-HAibEGTFTSDLSKQMEEEAVRLFIEWLK(L)NGGPSSGAPPPS-R²

(SEQ ID NO:149)

R¹-HAibEGTFTSDLSKQMEEEAVRK(L)FIEWLKINGGPSSGAPPPS-
R²

(SEQ ID NO:150)

R¹-HAibEGTFTSDLSKQMEEEAVK(L)LFIEWLKNGGPSSGAPPPS-R²

(SEQ ID NO:151)

R¹-HAibEGTFTSDLSKQMEEEAK(L)RLFIEWLKNGGPSSGAPPPS-R²

(SEQ ID NO:164)

R¹-HAibEGTFTSDLSKQMEK(L)EAVRLFIEWLKINGGPSSGAPPPS-
R²

(SEQ ID NO:152)

R¹-HAibEGTFTSDLSKQK(L)EEEAVRLFIEWLKNGGPSSGAPPPS-R²

(SEQ ID NO:153)

R¹-HAibEGTFTSDVSSYLEEQAVKEFIAWLIKAibRPSSGAPPPSK
(L)-R²

(SEQ ID NO:154)

R¹-HAibEGTFTSDVSSYLEEQAVK(L)EFIAWLIKAibRPSSGAPPPS-
R²

(SEQ ID NO:155)

R¹-HAibEGTFTSDVSSYLEGQAAK(L)EFIAWLVKGR-R²

wherein

R¹ is absent, CH₃, C(O)CH3, C(O)CH₂CH₃, C(O)CH₂CH₂CH₃, or C(O)CH(CH₃)CH₃; and

R² is OH, NH₂, NH(CH₃), NHCH₂CH₃, NHCH₂CH₂CH₃, NHCH(CH₃)CH₃, NHCH₂CH₂CH₂CH₃, NHCH(CH₃)CH₂CH₃, NHC₆H₅, NHCH₂CH₂OCH₃, NHOCH₃, NHOCH₂CH₃, a carboxy protecting group, a lipid fatty acid group or a carbohydrate, and

K(L) is a lysine residue covalently linked to a linker L. In certain embodiments, K(L) is:

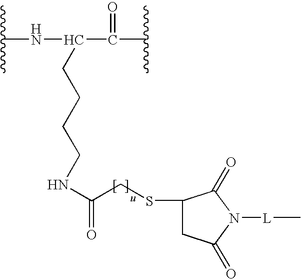

wherein u is 1, 2 or 3;

-L- is a linker moiety having the formula —X—Y-Z-, wherein:

X is:

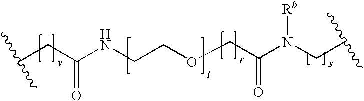

wherein v is 0, 1, 2, or 3; t is 1, 2, or 3, r is 1 or 2; s is 0, 1 or 2;
R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl;
Y is a recognition group comprising at least a ring structure; and
Z is a reactive group that is capable of forming a covalent bond with an amino acid sidechain in a combining site of an antibody.

In some embodiments Y has the optionally substituted structure:

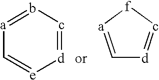

wherein a, b, c, d, and e are independently carbon or nitrogen; f is carbon, nitrogen, oxygen, or sulfur; Y is attached to X and Z independently at any two ring positions of sufficient valence; and no more than four of a, b, c, d, e, or f are simultaneously nitrogen.

In some embodiments, Z is selected from the group consisting of substituted 1,3-diketones or acyl beta-lactams

In some embodiments Z has the structure:

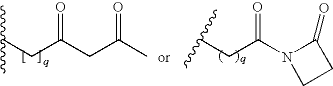

wherein q=0, 1, 2, 3, 4, or 5. In other embodiments, q=1, 2, or 3.

In some embodiments of compounds of Formula I, X is:

—R²²—P—R²³— or —R²²—P—R²¹—P′—R²³—

wherein:

P and P′ are independently selected from the group consisting of polyoxyalkylene oxides such as polyethylene oxide, polyethyloxazoline, poly-N-vinyl pyrrolidone, polyvinyl alcohol, polyhydroxyethyl acrylate, polyhydroxy ethylmethacrylate and polyacrylamide, polyamines having amine groups on either the polymer backbone or the polymer sidechains, such as polylysine, polyornithine, polyarginine, and polyhistidine, nonpeptide polyamines such as polyaminostyrene, polyaminoacrylate, poly(N-methyl aminoacrylate), poly(N-ethylaminoacrylate), poly(N,N-dimethyl aminoacrylate), poly(N,N-diethylaminoacrylate), poly(aminomethacrylate), poly(N-methyl amino-methacrylate), poly(N-ethyl aminomethacrylate), poly(N,N-dimethyl aminomethacrylate), poly(N,N-diethyl aminomethacrylate), poly(ethyleneimine), polymers of quaternary amines, such as poly(N,N,N-trimethylaminoacrylate chloride), poly(methyacrylamidopropyltrimethyl ammonium chloride), proteoglycans such as chondroitin sulfate-A (4-sulfate) chondroitin sulfate-C (6-sulfate) and chondroitin sulfate-B, polypeptides such as polyserine, polythreonine, polyglutamine, natural or synthetic polysaccharides such as chitosan, hydroxy ethyl cellulose, and lipids;

R²¹, R²², and R²³ are each independently a covalent bond, —O—, —S—, —NR^{b}—, amide, substituted or unsubstituted straight or branched chain C₁₋₅₀ alkylene, or substituted or unsubstituted straight or branched chain C₁₋₅₀ heteroalkylene;

R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl; and

R²¹, R²², and R²³ are selected such that the backbone length of X remains about 200 atoms or less.

In some embodiments of compounds of Formula I, X is attached to an amino acid residue in [GA targeting agent], and is an optionally substituted —R²²—[CH₂—CH₂—O]ₜ—R²³—R²²-cycloalkyl-R²³—, —R²²-aryl-R²³—, or —R²²-heterocyclyl-R²³—, wherein t is 0 to 50.

In some embodiments X is attached to the carboxy terminus, a S sidechain, a K sidechain, a K(SH) sidechain, a T sidechain, or a Y sidechain of a GA targeting agent

In some embodiments of compounds of Formula I, R²² is —(CH₂)ᵥ—, —(CH₂)ᵤ—C(O)—(CH₂)ᵥ—, —(CH₂)ᵤ—C(O)—O—(CH₂)ᵥ—, —(CH₂)ᵤ—C(S)—NR^{b}—(CH₂)ᵥ—, —(CH₂)ᵤ—C(O)—NR^{b}—(CH₂)ᵥ—, —(CH₂)ᵤ—NR^{b}—(CH₂)ᵥ—, —(CH₂)ᵤ—O—(CH₂)ᵥ—, —(CH₂)ᵤ—S(O)₀₋₂—(CH₂)ᵥ—, —(CH₂)ᵤ—S(O)₀₋₂—NR^{b}—(CH₂)ᵥ—, or —(CH₂)ᵤ—P(O)(OR^{b})—O—(CH₂)ᵥ—, wherein u and v are independently 0, 1, 2, 3, 4, 5, 6, 7, 8, 9, 10, 11, 12, 13, 14, 15, 16, 17, 18, 19 or 20.

In some embodiments of compounds of Formula I, R²¹ and R²³ are independently —(CH₂)ₛ—, —(CH₂)ᵣ—C(O)—(CH₂)ₛ—, —(CH₂)ᵣ—C(O)—O—(CH₂)ᵥ—, —(CH₂)ᵣ—C(S)—NR^{b}—(CH₂)—, —(CH₂)ᵣ—C(O)—NR^{b}—(CH₂)ₛ—, —(CH₂)ᵣ—NR^{b}—(CH₂)ₛ—, —(CH₂)—O—(CH₂)ₛ—, —(CH₂)ᵣ—S(O)₀₋₂—(CH₂)—, —(CH₂)ᵣ—S(O)₀₋₂—NR^{b}—(CH₂)ₛ—, or —(CH₂)ᵣ—P(O)(OR^{b})—O—(CH₂)ₛ—, wherein r, s, and v are independently 0, 1, 2, 3, 4, 5, 6, 7, 8, 9, 10, 11, 12, 13, 14, 15, 16, 17, 18, 19 or 20.

In some embodiments of Formula I, if t>1 or if X is —R²²—[CH₂—CH₂—O]ₜ—R²³—, —R²²-cycloalkyl-R²³, —R²²-aryl-R²³—, or —R²²-heterocyclyl-R²³—, Y is present.

Exemplary compounds in accordance with Formula I are illustrated in Formula 1 and 3.

Another aspect of the invention, illustrated in Formula II, is a GA targeting compound comprising a GA targeting agent covalently linked to a combining site of an Antibody via an intervening linker L′. The Antibody portion of a GA targeting compound can include whole (full length) antibody, unique antibody fragments, or any other forms of an antibody as this term is used herein. In one embodiment, the Antibody is a humanized version of a murine aldolase antibody comprising a constant region from a human IgG, IgA, IgM, IgD, or IgE antibody. In another embodiment, the Antibody is a chimeric antibody comprising the variable region from a murine aldolase antibody and a constant region from a human IgG, IgA, IgM, IgD, or IgE antibody. In a further embodiment, the Antibody is a fully human version of a murine aldolase antibody comprising a polypeptide sequence from natural or native human IgG, IgA, IgM, IgD, or IgE antibody

Antibody-L′-[GA targeting agent] (II)

wherein:
[GA targeting agent] is a peptide agonist of GLP-1R. In certain embodiments, [GA targeting agent] is a peptide selected from the group consisting of a GA targeting compound as described herein, including but not limited to:

(SEQ ID NO:3)

R¹-HGEGTFTSDLSKQMEEEAVRLFIEWLKNGGPSSGAPPPSK-R²

(SEQ ID NO:172)

R¹-HGEGTFTSDLSKQMEEEAVRLFIEWLKNGGPSSGAPPPSK(SH)-R²

(SEQ ID NO:4)

R¹-HAibEGTFTSDLSKQMEEEAVRLFIEWLKNGGPSSGAPPPSK-R²

(SEQ ID NO:173)

R¹-HAibEGTFTSDLSKQMEEEAVRLFIEWLKNGGPSSGAPPPSK(SH)-
R²

(SEQ ID NO:5)

R¹-HAibEGTFTSDLSKQMEEEAVRLFIEWLKNGGPSSGAPPPS-R²

(SEQ ID NO:6)

R¹-HAibEGTFTSDLSKQMEEEAVRLFIEWLKNGG-R²

(SEQ ID NO:7)

R¹-HAibEGTFTSDLSKQMEEEAVRLFIEWLKN-R²

(SEQ ID NO:8)

R¹-HAibEGTFTSDLSKQLEEEAVRLFIEFLKIN-R²

(SEQ ID NO:9)

R¹-HAibEGTFTSDLSKQLEEEAVRLAIEFLKIN-R²

(SEQ ID NO:10)

R¹-HAibEGTFTSDLSKQLEEEAVRLAIEFLKINGGPSSGAPPPS-R²

(SEQ ID NO:11)

R¹-HAibEGTFTSDLSKQLEEEAVRLFIEFLKINGGPSSGAPPPS-R²

(SEQ ID NO:12)

R¹-HAibEGTFTSDLSK(Ac)QMEEEAVRLFIEWLK(Ac)NGGPSSGAPP
PS-R²

(SEQ ID NO:13)

R¹-HAibEGTFTSDLSK(benzoyl)QMEEEAVRLFIEWLK(benzoyl)
NGGPSSGAPPPS-R²

(SEQ ID NO:14)

R¹-HAibEGTFTSDLSKQMEEEAVRLFIEWLKNGGPSSGAPPKS-R²

(SEQ ID NO:99)

R¹-HAibEGTFTSDLSKQMEEEAVRLFIEWLKNGGPSSGAPPK(SH)S-
R²

(SEQ ID NO:15)

R¹-HAibEGTFTSDLSKQMEEEAVRLFIEWLKNGGPSSGAKPPS-R²

(SEQ ID NO:100)

R¹-HAibEGTFTSDLSKQMEEEAVRLFIEWLKNGGPSSGAK(SH)PPS-
R²

(SEQ ID NO:16)

R¹-HAibEGTFTSDLSKQMEEEAVRLFIEWLKNGGPSSKAPPPS-R²

(SEQ ID NO:101)

R¹-HAibEGTFTSDLSKQMEEEAVRLFIEWLKNGGPSSK(SH)APPPS-
R²

(SEQ ID NO:17)

R¹-HAibEGTFTSDLSKQMEEEAVRLFIEWLKNGGPKSGAPPPS-R²

(SEQ ID NO:168)

R¹-HAibEGTFTSDLSKQMEEEAVRLFIEWLKNGGPK(SH)SGAPPPS-
R²

(SEQ ID NO:18)

R¹-HAibEGTFTSDLSKQMEEEAVRLFIEWLKKGGPSSGAPPPS-R²

(SEQ ID NO:102)

R¹-HAibEGTFTSDLSKQMEEEAVRLFIEWLKK(SH)GGPSSGAPPPS-
R²

(SEQ ID NO:19)

R¹-HAibEGTFTSDLSKQMEEEAVRLFIEWKKNGGPSSGAPPPS-R²

(SEQ ID NO:170)

R¹-HAibEGTFTSDLSKQMEEEAVRLFIEWLK(SH)NGGPSSGAPPPS-
R²

(SEQ ID NO:103)

R¹-HAibEGTFTSDLSKQMEEEAVRLFIEWK(SH)KNGGPSSGAPPPS-
R²

(SEQ ID NO:20)

R¹-HAibEGTFTSDLSKQMEEEAVRLFIKWLKNGGPSSGAPPPS-R²

(SEQ ID NO:104)

R¹-HAibEGTFTSDLSKQMEEEAVRLFIK(SH)WLKNGGPSSGAPPPS-
R²

(SEQ ID NO:21)

R¹-HAibEGTFTSDLSKQMEEEAVRLFKEWLIKINGGPSSGAPPPS-R²

(SEQ ID NO:105)

R¹-HAibEGTFTSDLSKQMEEEAVRLFK(SH)EWLKNGGPSSGAPPPS-
R²

(SEQ ID NO:22)

R¹-HAibEGTFTSDLSKQMEEEAVRKFIEWLKNGGPSSGAPPPS-R²

(SEQ ID NO:106)

R¹-HAibEGTFTSDLSKQMEEEAVRK(SH)FIEWLKNGGPSSGAPPPS-
R²

(SEQ ID NO:23)

R¹-HAibEGTFTSDLSKQMEEEAVKLFIEWLKINGGPSSGAPPPS-R²

(SEQ ID NO:107)

R¹-HAibEGTFTSDLSKQMEEEAVK(SH)LFIEWLKNGGPSSGAPPPS-
R²

(SEQ ID NO:24)

R¹-HAibEGTFTSDLSKQMEEEAKRLFIEWLKNGGPSSGAPPPS-R²

(SEQ ID NO:108)

R¹-HAibEGTFTSDLSKQMEEEAK(SH)RLFIEWLKNGGPSSGAPPPS-
R²

(SEQ ID NO:25)

R¹-HAibEGTFTSDLSKQMEEKAVRLFIEWLKNGGPSSGAPPPS-R²

(SEQ ID NO:109)

R¹-HAibEGTFTSDLSKQMEEK(SH)AVRLFIEWLKNGGPSSGAPPPS-
R²

(SEQ ID NO:26)

R¹-HAibEGTFTSDLSKQMEKEAVRLFIEWLKNGGPSSGAPPPS-R²

(SEQ ID NO:110)

R¹-HAibEGTFTSDLSKQMEK(SH)EAVRLFIEWLKNGGPSSGAPPPS-
R²

(SEQ ID NO:27)

R¹-HAibEGTFTSDLSKQKEEEAVRLFIEWLKNGGPSSGAPPPS-R²

(SEQ ID NO:111)

R¹-HAibEGTFTSDLSKQK(SH)EEEAVRLFIEWLKNGGPSSGAPPPS-
R²

(SEQ ID NO:28)

R¹-HAibEGTFTSDLSKKMEEEAVRLFIEWLKNGGPSSGAPPPS-R²

(SEQ ID NO:112)

R¹-HAibEGTFTSDLSKK(SH)MEEEAVRLFIEWLKNGGPSSGAPPPS-
R²

(SEQ ID NO:29)

R¹-HAibEGTFTSDLKKQMEEEAVRLFIEWLKNGGPSSGAPPPS-R²

(SEQ ID NO:169)

R¹-HAibEGTFTSDLSK(SH)QMEEEAVRLFIEWLKNGGPSSGAPPPS-
R²

(SEQ ID NO:113)

R¹-HAibEGTFTSDLK(SH)KQMEEEAVRLFIEWLKNGGPSSGAPPPS-
R²

(SEQ ID NO:30)

R¹-HAibEGTFTSDLSKQMEEEAVRLFIEWLKNGGK-R²

(SEQ ID NO:114)

R¹-HAibEGTFTSDLSKQMEEEAVRLFIEWLKNGGK(SH)-R²

(SEQ ID NO:31)

R¹-HAibEGTFTSDLSKQMEEEAVRKFIEWLKNGGPSS-R²

(SEQ ID NO:115)

R¹-HAibEGTFTSDLSKQMEEEAVRKFIEWLK(SH)NGGPSS-R²

(SEQ ID NO:32)

R¹-HAibEGTFTSDVSSYLEGQAAKEFIAWLVKGR-R²

(SEQ ID NO:33)

R¹-HAEGTFTSDVSSYLEGQAAKEFIAWLVKGRK-R²

(SEQ ID NO:34)

R¹-HGEGTFTSDVSSYLEGQAAKEFIAWLVKGRK-R²

(SEQ ID NO:35)

R¹-HAibEGTFTSDVSSYLEGQAAKEFIAWLVKAibR-R²

(SEQ ID NO:36)

R¹-HAibEGTFTSDVSSYLEGQAAKEFIAWLVKAibRK-R²

(SEQ ID NO:37)

R¹-HAibEGTFTSDVSSYLEGQAAKEFIAWLVKGRK-R²

(SEQ ID NO:38)

R¹-HAibEGTFTSDVSSYLEGQAAKEFIAWLVKGRK(SH)-R²

(SEQ ID NO:39)

R¹-HAibEGTFTSDVSSYLEGQAAK(SH)EFIAWLVKGR-R²

(SEQ ID NO:40)

R¹-HAibEGTFTSDVSSYGEGQAAKEFIAWLVKAibR-R²

(SEQ ID NO:41)

R¹-HAibEGTFTSDVSSYCEGQAAKEFIAWLVKAibR-R²

(SEQ ID NO:42)

R¹-HAibEGTFTSDVSSYFEGQAAKEFIAWLVKAibR-R²

(SEQ ID NO:43)

R¹-HAibEGTFTSDVSSYYEGQAAKEFIAWLVKAibR-R²

(SEQ ID NO:44)

R¹-HAibEGTFTSDVSSYWEGQAAKEFIAWLVKAibR-R²

(SEQ ID NO:45)

R¹-HAibEGTFTSDVSSYLEEQAAKEFIAWLVKAibR-R²

(SEQ ID NO:46)

R¹-HAibEGTFTSDVSSYLEDQAAKEFIAWLVKAibR-R²

(SEQ ID NO:47)

R¹-HAibEGTFTSDVSSYLEKQAAKEFIAWLVKAibR-R²

(SEQ ID NO:48)

R¹-HAibEGTFTSDVSSYLEGQAVKEFIAWLVKAibR-R²

(SEQ ID NO:49)

R¹-HAibEGTFTSDVSSYLEGQAIKEFIAWLVKAibR-R²

(SEQ ID NO:50)

R¹-HAibEGTFTSDVSSYLEGQALKEFIAWLVKAibR-R²

(SEQ ID NO:51)

R¹-HAibEGTFTSDVSSYLEGQAAREFIAWLVKAibR-R²

(SEQ ID NO:52)

R¹-HAibEGTFTSDVSSYLEGQAAOrnEFIAWLVKAibR-R²

(SEQ ID NO:53)

R¹-HAibEGTFTSDVSSYLEGQAAKEFIAFLVKAibR-R²

(SEQ ID NO:54)

R¹-HAibEGTFTSDVSSYLEGQAAKEFIAWLIKAibR-R²

(SEQ ID NO:55)

R¹-HAibEGTFTSDVSSYLEGQAAKEFIAWLVRAibR-R²

(SEQ ID NO:56)

R¹-HAibEGTFTSDVSSYLEGQAAKEFIAWLVOrnAibR-R²

(SEQ ID NO:57)

R¹-HAibEGTFTSDVSSYLEEQAVKEFIAWLIKAibR-R²

(SEQ ID NO:58)

R¹-HAibEGTFTSDVSSYFEEQAVIKIEFIAWLIKAibR-R²

(SEQ ID NO:59)

R¹-HAibEGTFTSDVSSYYEEQAVKEFIAWLIKAibR-R²

(SEQ ID NO:60)

R¹-HAibEGTFTSDVSSYWEEQAVKEFIAWLIKAibR-R²

(SEQ ID NO:61)

R¹-HAibEGTFTSDVSSYLEEQAVKEFIAWLIRAibR-R²

(SEQ ID NO:62)

R¹-HAibEGTFTSDVSSYLEEQAVREFIAWLIRAibR-R²

(SEQ ID NO:63)

R¹-HAibEGTFTSDVSSYLEEQAVKEFIAWLIKAibRPSSGAPP
PS-R²

(SEQ ID NO:117)

R¹-HAibEGTFTSDVSSYLEEQAVK(SH)EFIAWLIKAibRPSSGAPPP
S-R²

(SEQ ID NO:64)

R¹-HAibEGTFTSDKSSYLEEQAVKEFIAWLIKAibR-R²

(SEQ ID NO:116)

R¹-HAibEGTFTSDK(SH)SSYLEEQAVKEFIAWLIKAibR-R²

(SEQ ID NO:65)

R¹-HAibEGTFTSDVSKYLEEQAVKEFIAWLIKAibR-R²

(SEQ ID NO:117)

R¹-HAibEGTFTSDVSK(SH)YLEEQAVKEFIAWLIKAibR-R²

(SEQ ID NO:66)

R¹-HAibEGTFTSDVSSYKEEQAVKEFIAWLIKAibR-R²

(SEQ ID NO:118)

R¹-HAibEGTFTSDVSSYK(SH)EEQAVKEFIAWLIKAibR-R²

(SEQ ID NO:67)

R¹-HAibEGTFTSDVSSYLEKQAVKEFIAWLIKAibR-R²

(SEQ ID NO:119)

R¹-HAibEGTFTSDVSSYLEK(SH)QAVKEFIAWLIKAibR-R²

(SEQ ID NO:68)

R¹-HAibEGTFTSDVSSYLEEQKVKEFIAWLIKAibR-R²

wherein:

R¹ is absent, CH₃, C(O)CH3, C(O)CH₂CH₃, C(O)CH₂CH₂CH₃, or C(O)CH(CH₃)CH₃; and

R² is OH, NH₂, NH(CH₃), NHCH₂CH₃, NHCH₂CH₂CH₃, NHCH(CH₃)CH₃, NHCH₂CH₂CH₂CH₃, NHCH(CH₃)CH₂CH₃, NHC₆H₅, NHCH₂CH₂OCH₃, NHOCH₃, NHOCH₂CH₃, a carboxy protecting group, a lipid fatty acid group or a carbohydrate,

and any of the C-terminus truncations, and analogs that may be formed from these peptides; and

L′ is a linker moiety having the formula —X—Y-Z′, wherein:
X is a biologically compatible polymer or block copolymer attached to one of the residues that comprises a GA targeting agent;
Y is an optionally present recognition group comprising at least a ring structure; and
Z is a group that is covalently linked to a sidechain in a combining site of an antibody;
   and pharmaceutically acceptable salts, stereoisomers, tautomers, solvates, and prodrugs thereof.

In some embodiments of compounds of Formula II, X is:

—R²²—P—R²³— or —R²²—P—R²¹—P′—R²³—

wherein:

P and P′ are independently selected from the group consisting of polyoxyalkylene oxides such as polyethylene oxide, polyethyloxazoline, poly-N-vinyl pyrrolidone, polyvinyl alcohol, polyhydroxyethyl acrylate, polyhydroxy ethylmethacrylate and polyacrylamide, polyamines having amine groups on either the polymer backbone or the polymer sidechains, such as polylysine, polyornithine, polyarginine, and polyhistidine, nonpeptide polyamines such as polyaminostyrene, polyaminoacrylate, poly(N-methyl aminoacrylate), poly(N-ethylaminoacrylate), poly(N,N-dimethyl aminoacrylate), poly(N,N-diethylaminoacrylate), poly(aminomethacrylate), poly(N-methyl amino-methacrylate), poly(N-ethyl aminomethacrylate), poly(N,N-dimethyl aminomethacrylate), poly(N,N-diethyl aminomethacrylate), poly(ethyleneimine), polymers of quaternary amines, such as poly(N,N,N-trimethylaminoacrylate chloride), poly(methyacrylamidopropyltrimethyl ammonium chloride), proteoglycans such as chondroitin sulfate-A (4-sulfate) chondroitin sulfate-C (6-sulfate) and chondroitin sulfate-B, polypeptides such as polyserine, polythreonine, polyglutamine, natural or synthetic polysaccharides such as chitosan, hydroxy ethyl cellulose, and lipids;

R²¹, R²², and R²³ are each independently a covalent bond, —O—, —S—, —NR^{b}—, substituted or unsubstituted straight or branched chain C₁₋₅₀ alkylene, or substituted or unsubstituted straight or branched chain C₁₋₅₀ heteroalkylene;

R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl; and

R²¹, R²², and R²³ are selected such that the backbone length of X remains about 200 atoms or less.

In some embodiments of compounds of Formula II, X is attached to an amino acid residue in [GA targeting agent], and is an optionally substituted —R²²—[CH₂—CH₂—O]ₜ—R²³—, —R²²-cycloalkyl-R²³—, —R²²-aryl-R²³—, or —R²²-heterocyclyl-R²³—, wherein t is 0 to 50.

In some embodiments of compounds of Formula II, R²² is —(CH₂)ᵥ—, —(CH₂)ᵤ—C(O)—(CH₂)ᵥ—, —(CH₂)ᵤ—C(O)—O—(CH₂)ᵥ—, —(CH₂)ᵤ—C(S)—NR^{b}—(CH₂)ᵥ—, —(CH₂)ᵤ—C(O)—NR^{b}—(CH₂)ᵥ—, —(CH₂)ᵤ—NR^{b}—(CH₂)ᵥ—, —(CH₂)ᵤ—O—(CH₂)ᵥ—, —(CH₂)ᵤ—S(O)₀₋₂—(CH₂)ᵥ—, —(CH₂)ᵤ—S(O)₀₋₂—NR^{b}—(CH₂)ᵥ—, or —(CH₂)ᵤ—P(O)(OR^{b})—O—(CH₂)ᵥ—, wherein u and v are independently 0, 1, 2, 3, 4, 5, 6, 7, 8, 9, 10, 11, 12, 13, 14, 15, 16, 17, 18, 19 or 20.

In some embodiments of compounds of Formula II, R²¹ and R²³ are independently —(CH₂)ₛ—, —(CH₂)ᵣ—C(O)—(CH₂)ₛ—, —(CH₂)ᵣ—C(O)—O—(CH₂)ᵥ—, —(CH₂)ᵣ—C(S)—NR^{b}—(CH₂)ₛ—, —(CH₂)—C(O)—NR^{b}—(CH₂)ₛ—, —(CH₂)ᵣ—NR^{b}—(CH₂)ₛ—, —(CH₂)ᵣ—O—(CH₂)ₛ—, —(CH₂)ᵣ—S(O)₀₋₂—(CH₂)—, —(CH₂)ᵣ—S(O)₀₋₂—NR^{b}—(CH₂)ₛ—, or —(CH₂)ᵣ—P(O)(OR^{b})—O—(CH₂)ₛ—, wherein r, s, and v are independently 0, 1, 2, 3, 4, 5, 6, 7, 8, 9, 10, 11, 12, 13, 14, 15, 16, 17, 18, 19 or 20.

In some embodiments of compounds of Formula II, [GA targeting agent] is a peptide selected from the group consisting of:

(SEQ ID NO:156)

R¹-HAibEGTFTSDLSKQMEEEAVRLFIEWLK(L′)NGGPSSGAPPPS-
R²

(SEQ ID NO:157)

R¹-HAibEGTFTSDLSKQMEEEAVRK(L′)FIEWLKNGGPSSGAPPPS-
R²

(SEQ ID NO:158)

R¹-HAibEGTFTSDLSKQMEEEAVK(L′)LFIEWLKNGGPSSGAPPPS-
R²

(SEQ ID NO:159)

R¹-HAibEGTFTSDLSKQMEEEAK(L′)RLFIEWLKNGGPSSGAPPPS-
R²

(SEQ ID NO:160)

R¹-HAibEGTFTSDLSKQK(L′)EEEAVRLFIEWLKNGGPSSGAPPPS-
R²

(SEQ ID NO:161)

R¹-HAibEGTFTSDVSSYLEEQAVKEFIAWLIKAibRPSSGAPPPSK
(L′)-R²

(SEQ ID NO:162)

R¹-HAibEGTFTSDVSSYLEEQAVK(L′)EFIAWLIKAibRPSSGAPPP
S-R²

(SEQ ID NO:163)

R¹-HAibEGTFTSDVSSYLEGQAAK(L′)EFIAWLVKGR-R²

wherein

R¹ is absent, CH₃, C(O)CH3, C(O)CH₂CH₃, C(O)CH₂CH₂CH₃, or C(O)CH(CH₃)CH₃; and

R² is OH, NH₂, NH(CH₃), NHCH₂CH₃, NHCH₂CH₂CH₃, NHCH(CH₃)CH₃, NHCH₂CH₂CH₂CH₃, NHCH(CH₃)CH₂CH₃, NHC₆H₅, NHCH₂CH₂OCH₃, NHOCH₃, NHOCH₂CH₃, a carboxy protecting group, a lipid fatty acid group or a carbohydrate, and

K(L) is a lysine residue covalently linked to a linker L′. In certain embodiments, K(L′) is:

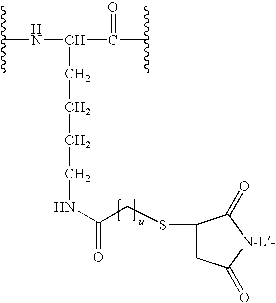

wherein u is 1, 2 or 3;

-L′- is a linker moiety having the formula —X—Y-Z-, wherein:

X is:

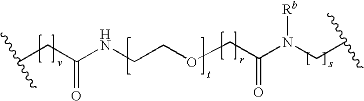

wherein v is 0, 1, 2, or 3; t is 1, 2, or 3, r is 1 or 2; s is 0, 1 or 2;
R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl;
Y is a recognition group comprising at least a ring structure; and
Z′ is an attachment moiety comprising a covalent link to an amino acid sidechain in a combining site of an antibody.
   and pharmaceutically acceptable salts, stereoisomers, tautomers, solvates, and prodrugs thereof.

In some embodiments Y has the optionally substituted structure:

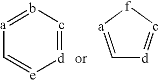

wherein a, b, c, d, and e are independently carbon or nitrogen; f is carbon, nitrogen, oxygen, or sulfur; Y is attached to X and Z independently at any two ring positions of sufficient valence; and no more than four of a, b, c, d, e, or f are simultaneously nitrogen.

In some embodiments Z′ has the structure:

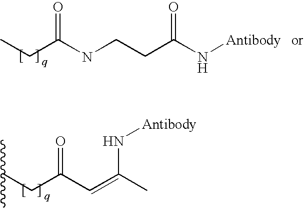

wherein q=0, 1, 2, 3, 4, or 5 and N-Antibody refers to a covalent link to an amino acid sidechain in a combining site of an antibody bearing an amino group. In other aspects, q=1, 2 or 3.

Another aspect of the invention, illustrated in Formula III, is a GA targeting compound in which two GA targeting agents, which may be the same or different, are each covalently linked to a combining site of an antibody. The Antibody portion of a GA targeting compound can include whole (full length) antibody, unique antibody fragments, or any other forms of an antibody as this term is used herein. In one embodiment, the Antibody is a humanized version of a murine aldolase antibody comprising a constant region from a human IgG, IgA, IgM, IgD, or IgE antibody. In another embodiment, the Antibody is a chimeric antibody comprising the variable region from a murine aldolase antibody and a constant region from a human IgG, IgA, IgM, IgD, or IgE antibody. In a further embodiment, the Antibody is a fully human version of a murine aldolase antibody comprising a polypeptide sequence from natural or native human IgG, IgA, IgM, IgD, or IgE antibody:

Antibody[-L′-[GA targeting agent]]₂ (III)

wherein [GA targeting agent], Antibody, and L′ are as defined according to Formula II.

Exemplary compounds in accordance with Formula I are illustrated in FIGS. 2 and 4.

In certain embodiments, methods are provided for treating diabetes or a diabetes-related condition in a subject comprising administering to the subject a therapeutically effective amount of a GA targeting compound or a pharmaceutical derivative thereof.

In certain embodiments, methods are provided for increasing insulin secretion in a subject comprising administering to the subject a therapeutically effective amount of a GA targeting compound or a pharmaceutical derivative thereof.

In certain embodiments, methods are provided for decreasing blood glucose levels in a subject comprising administering to the subject a therapeutically effective amount of a GA targeting compound or a pharmaceutical derivative thereof.

In certain embodiments, the use of GA targeting compounds and pharmaceutical derivatives thereof for generating a medicament for treating diabetes or a diabetes-related condition, or for increasing insulin secretion or decreasing blood glucose levels, are provided.

Some GA targeting compounds of the invention include:

(SEQ ID NO:22)

R¹-HAibFGTFTSDLSKQMFFFAVRKFIFWLKNGGPSSGAPPPS-R²

(SEQ ID NO:23)

R¹-HAibFGTFTSDLSKQMFFFAVKLFIFWLKNGGPSSGAPPPS-R²

(SEQ ID NO:24)

R¹-HAibFGTFTSDLSKQMFFFAKRLFIFWLKNGGPSSGAPPPS-R²

(SEQ ID NO:25)

R¹-HAibFGTFTSDLSKQMFKFAVRLFIFWLKNGGPSSGAPPPS-R²

(SEQ ID NO:26)

R¹-HAibFGTFTSDLSKQKFFFAVRLFIFWLKNGGPSSGAPPPS-R²

Some GA targeting compounds of the invention include:

(SEQ ID NO:106)

R¹-HAibEGTFTSDLSKQMEEEAVRK(SH)FIEWLKNGGPSSGAPPPS-
R²

(SEQ ID NO:107)

R¹-HAibFGTFTSDLSKQMFFFAVK(SH)LFIFWLKNGGPSSGAPPPS-
R²

(SEQ ID NO:108)

R¹-HAibFGTFTSDLSKQMFFFAK(SH)RLFIFWLKNGGPSSGAPPPS-
R²

(SEQ ID NO:110)

R¹-HAibFGTFTSDLSKQMFK(SH)FAVRLFIFWLKNGGPSSGAPPPS-
R²

(SEQ ID NO:111)

R¹-HAibEGTFTSDLSKQK(SH)EEEAVRLFIEWLKNGGPSSGAPPPS-
R²

Some GA targeting compounds of the invention include:

(SEQ ID NO:149)

R¹-HAibFGTFTSDLSKQMFFFAVRK(L)FIFWLKNGGPSSGAPPPS-R²

(SEQ ID NO:150)

R¹-HAibFGTFTSDLSKQMFFFAVK(L)LFIFWLKNGGPSSGAPPPS-R²

(SEQ ID NO:151)

R¹-HAibFGTFTSDLSKQMFFFAK(L)RLFIFWLKINGGPSSGAPPPS-
R²

(SEQ ID NO:164)

R¹-HAibFGTFTSDLSKQMFK(L)FAVRLFIFWLKNGGPSSGAPPPS-R²

(SEQ ID NO:152)

R¹-HAibEGTFTSDLSKQK(L)EEEAVRLFIEWLKINGGPSSGAPPPS-
R²

wherein K(L) is a lysine reside attached to a linker L wherein L is capable of forming a covalent bond with an amino acid sidechain in a combining site of an antibody.

Some compounds of the invention include:

(SEQ ID NO:157)

R¹-HAibEGTFTSDLSKQMEEEAVRK(L′)FIEWLKNGGPSSGAPPPS-
R²

(SEQ ID NO:158)

R¹-HAibFGTFTSDLSKQMFFFAVK(L′)LFIFWLKNGGPSSGAPPPS-
R²

(SEQ ID NO:159)

R¹-HAibFGTFTSDLSKQMFFFAK(L′)RLFIFWLKNGGPSSGAPPPS-
R²

(SEQ ID NO:165)

R¹-HAibFGTFTSDLSKQMFK(L′)FAVRLFIFWLKNGGPSSGAPPPS-
R²

(SEQ ID NO:160)

R¹-HAibEGTFTSDLSKQK(L′)EEEAVRLFIEWLKNGGPSSGAPPPS-
R²

wherein K(L′) is a lysine reside attached to a linker L′ wherein L′ is capable of forming a covalent bond with an amino acid sidechain in a combining site of an antibody. In certain embodiments, K(L′) is:

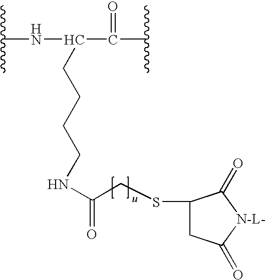

wherein u is 1, 2 or 3;

-L- is a linker having one of the formula —X—Y-Z- or X—Y-Z′ wherein:

X is:

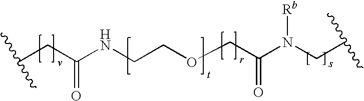

wherein v is 0, 1, 2, or 3; t is 1, 2, or 3, r is 1 or 2; s is 0, 1 or 2;
R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl;
Y is an optionally present recognition group comprising at least a ring structure; and
Z is a reactive group that is capable of forming a covalent bond with an amino acid sidechain in a combining site of an antibody.

### BRIEF DESCRIPTION OF THE DRAWINGS

FIG. 1a and FIG. 1b respectively illustrate one embodiment according to Formula I and one embodiment according to either Formula II or Formula III.

FIG. 2a and FIG. 2b respectively illustrate one embodiment according to Formula I and one embodiment according to either Formula II or Formula III.

FIG. 3 illustrates one embodiment according to Formula I.

FIG. 4 illustrates one embodiment according to Formula II or Formula III.

FIGS. 5A and 5B illustrate the solid phase synthesis of targeting agent-linker conjugates of the present invention.

FIG. 6 illustrates the amino acid sequence alignment of the variable domains of m38c2 (SEQ ID NOs:77 and 78), h38c2 (SEQ ID NOs:79 and 80), and human germlines DPK-9 (SEQ ID NO:81), DP-47 (SEQ ID NO:82), JK4 (SEQ ID NO:83), and JH4 (SEQ ID NO:84). Framework regions (FR) and complementarity determining regions (CDR) are defined according to Kabat et al. Asterisks mark differences between m38c2 and h38c2 or between h38c2 and the human germlines.

FIG. 7 shows various structures that may serve as linker reactive groups. X in structure A may be N, C, or any other heteroatom. R′₁, R′₂, R₃, and R₄ in structures A-C represent substituents which include, for example, C, H, N, O, P, S, halogen (F, Cl, Br, I), or a salt thereof. These substituents may also include a group such as an alkyl, alkenyl, alkynyl, oxoalkyl, oxoalkenyl, oxoalkynyl, aminoalkyl, aminoalkenyl, aminoalkynyl, sulfoalkyl, sulfoalkenyl, sulfoalkynyl, phosphoalkyl, phosphoalkenyl, or phosphoalkynyl group. R′₂ and R₃ may be part of a cyclic structure, as exemplified in structures B and C. Structures A-C form reversible covalent bonds with surface accessible reactive nucleophilic groups (e.g., lysine or cysteine sidechains) of a combining site of an antibody. For example, structure A may form an irreversible covalent bond with a reactive nucleophile if X is N and if R′₁, and R₃ form part of a cyclic structure. Structures D-G may form nonreversible covalent bonds with reactive nucleophilic groups in a combining site of an antibody. In these structures, R″₁, and R″₂ represent C, O, N, halide or leaving groups such as mesyl or tosyl.

FIG. 8 shows various electrophiles that are suitable for reactive modification with a reactive amino acid sidechain in a combining site of an antibody and thus may serve as linker reactive groups. Key: (A) acyl beta-lactam; (B) simple diketone; (C) succinimide active ester; (D) maleimide; (E) haloacetamide with linker; (F) haloketone; (G) cyclohexyl diketone; and (H) aldehyde. The squiggle line indicates the point of attachment to the rest of the linker or targeting agent. X refers to a halogen.

FIG. 9 shows the addition of a nucleophilic (“nu”) sidechain in an antibody combining site to compounds A-G in FIG. 7.

FIG. 10 shows the addition of a nucleophilic sidechain in an antibody combining to compounds A-H in FIG. 8.

FIG. 11 shows the synthesis of:

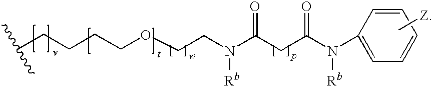

FIG. 12 shows a synthesis of:

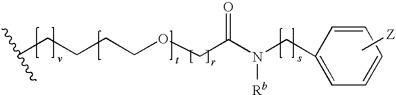

FIG. 13 shows a synthesis of:

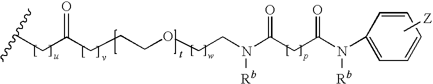

FIG. 14 shows a synthesis of:

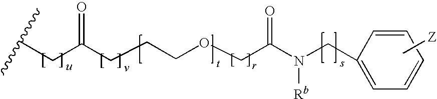

FIG. 15 shows a synthesis of:

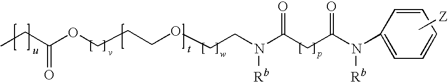

FIG. 16 shows a synthesis of:

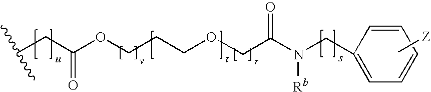

FIG. 17 shows a synthesis of:

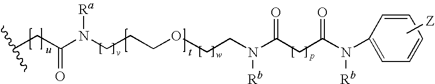

FIG. 18 shows a synthesis of:

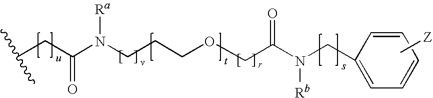

FIG. 19 shows a synthesis of:

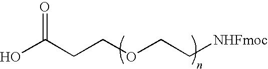

FIG. 20 shows syntheses of:

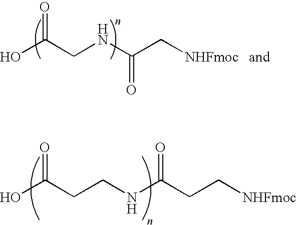

FIG. 21 shows a synthesis of:

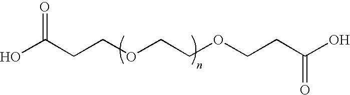

FIG. 22 shows a synthesis of:

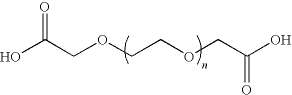

FIG. 23 shows a synthesis of:

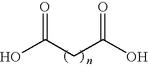

FIG. 24 shows a synthesis of:

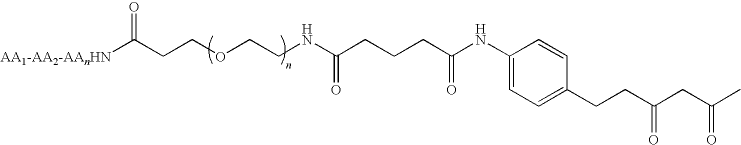

FIG. 25 shows a synthesis of:

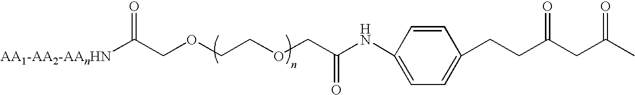

FIG. 26 shows a synthesis of

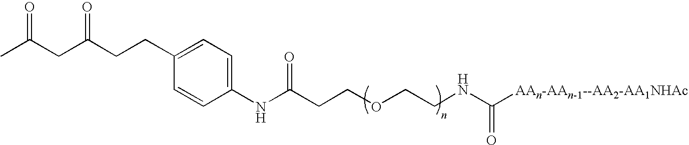

FIG. 27 illustrates the mammalian expression vector PIGG-h38c2. The 9 kb vector comprises heavy chain γ1 and light chain κ expression cassettes driven by a bidirectional CM promoter construct.

FIG. 28 shows a synthesis of:

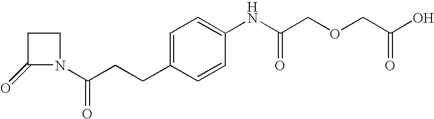

FIG. 29 shows a synthesis of the 20-atom AZD maleimide linker:

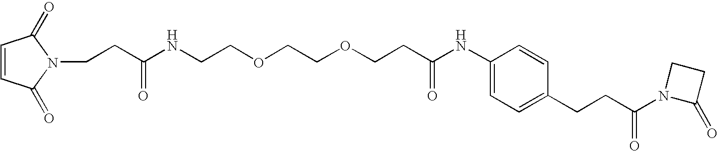

FIG. 30 shows a synthesis of side-chain modified lysine for use in GA targeting peptides.

FIG. 31 shows a synthesis of a GA targeting agent-linker conjugate comprising the GA targeting peptide of SEQ ID NO:22 linked to the 20-atom AZD maleimide linker set forth in FIG. 29 via a side-chain modified Lys residue in the peptide.

FIG. 32 shows a synthesis of a GA targeting agent-linker conjugate comprising the GA targeting peptide of SEQ ID NO:32 linked to the 20-atom AZD maleimide linker set forth in FIG. 29 via a side-chain modified Lys residue in the peptide.

FIG. 33 illustrates the amino acid sequence of the light and heavy chains of one embodiment of a humanized 38c2 IgG1.

FIG. 34 shows subcutaneous half-life (SC T1/2) and percentage bioavailability of compounds of the invention comprising a peptide according to one of the SEQ IDs of the invention as follows: K11: SEQ ID NO:29, K12: SEQ ID NO:5, K13: SEQ ID NO:28, K14: SEQ ID NO:27, K16: SEQ ID NO:26, K17: SEQ ID NO:25, K19: SEQ ID NO:24, K20: SEQ ID NO:23, K21: SEQ ID NO:22, K23: SEQ ID NO:21, K24: SEQ ID NO:20, K26: SEQ ID NO:19, K27: SEQ ID NO:132, K28: SEQ ID NO:18, K38: SEQ ID NO:14, C: SEQ ID NO:3, C1-30: SEQ ID NO:30, K21 1-33: SEQ ID NO:31, linked through a lysine residue or a K(SH) residue (at the position indicated, e.g., K11 means linked though a K or K(SH) residue at position 11) via the linker of FIGS. 1-4 to the h38c2 antibody. SC T1/2 (subcutaneous half-life) was obtained by SC injection of compound into mice and detecting compound concentration by ELISA. SC Bioavailability represents a ratio between the area under the curve of compound from IV injected mice and area under the curve of the same compound from SC injected mice.

FIG. 35 shows results of Glucose Tolerance Test (GTT). Single 0.3 mg/kg SC Dose Compounds of the invention used in FIG. 35 are referred to as the specific SEQ ID NO: that was conjugated to the humanized aldolase antibody h38c2 via the linker structures shown in FIGS. 1-4. Compounds were dosed as indicated above, at 0.3 mg/kg SC (again, linked through a K or K(SH) substitution at the respective amino acid position). A: Amalgamation of data from 48 hour test and 72 hour test. B: 48 hr data. C: 72 hr data.

FIG. 36 A-D Daily Food Intake: shows results of Body Weight Change analysis for the same animals as tested in FIG. 35.

FIG. 37 A-D Cumulative body weight change for the same animals as tested in FIGS. 35 and 36.

FIG. 38a and FIG. 38b respectively illustrate one embodiment according to Formula I and one embodiment according to either Formula II or Formula III.

FIG. 39a and FIG. 29b respectively illustrate one embodiment according to Formula I and one embodiment according to either Formula II or Formula III.

FIG. 40a and FIG. 40b respectively illustrate one embodiment according to Formula I and one embodiment according to either Formula II or Formula III.

FIG. 41a and FIG. 41b respectively illustrate one embodiment according to Formula I and one embodiment according to either Formula II or Formula III.

FIG. 42a and FIG. 42b respectively illustrate one embodiment according to Formula I and one embodiment according to either Formula II or Formula III.

FIG. 43a and FIG. 43b respectively illustrate one embodiment according to Formula I and one embodiment according to either Formula II or Formula III.

FIG. 44a and FIG. 44b respectively illustrate one embodiment according to Formula I and one embodiment according to either Formula II or Formula III.

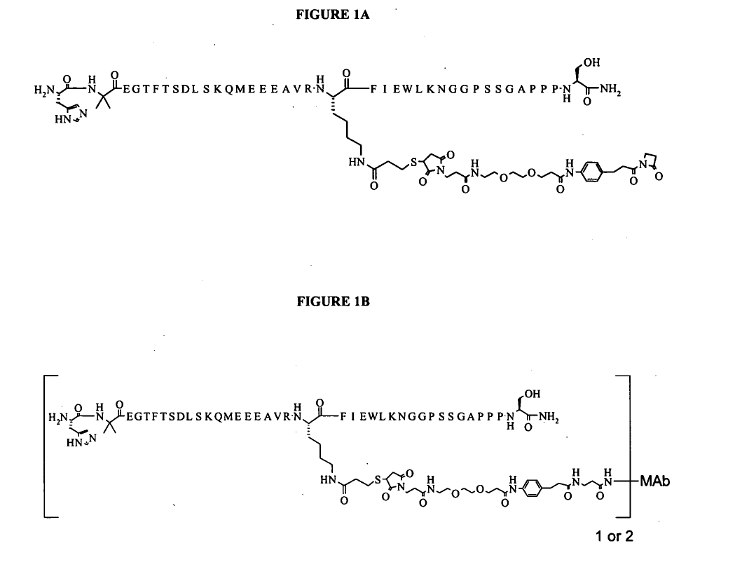

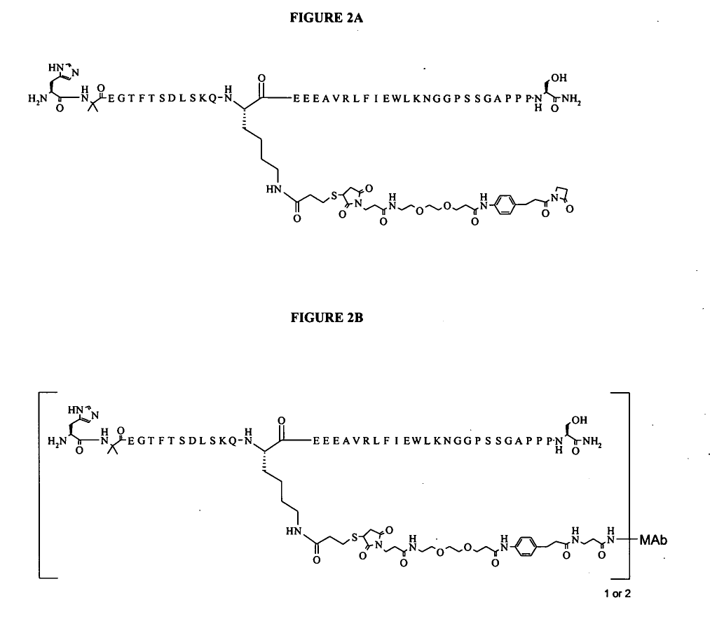

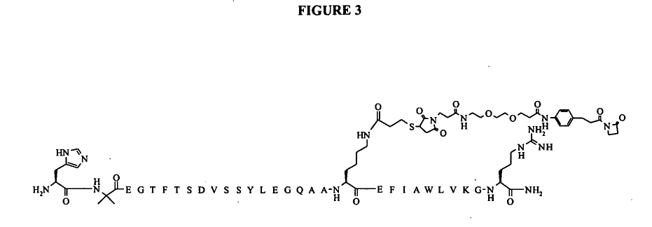

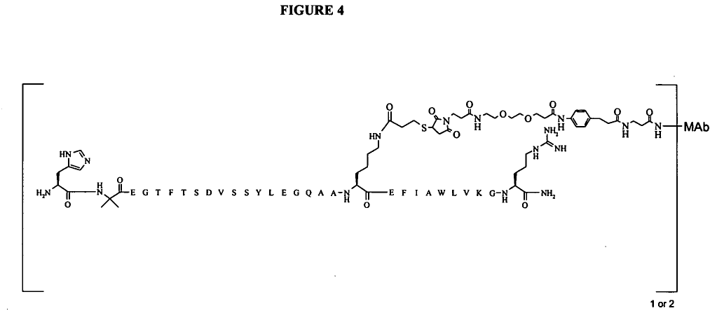

### DETAILED DESCRIPTION

### Definitions

The following abbreviations, terms and phrases are used herein as defined below.

| Amino acid | One letter abbreviation | Three letter abbreviation |
|---|---|---|
| 2-aminoisobutyric acid | — | Aib2 or Aib |
| Alanine | A | Ala |
| Arginine | R | Arg |
| Asparagine | N | Asn |
| Aspartic acid | D | Asp |
| Cysteine | C | Cys |
| Glutamic acid | E | Glu |
| Glutamine | Q | Gln |
| Glycine | G | Gly |
| Histidine | H | His |
| Isoleucine | I | Ile |
| Leucine | L | Leu |
| Lysine | K | Lys |
| Methionine | M | Met |
| Norleucine | — | Nle |
| Ornithine | — | Orn |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

Unless indicated otherwise by a “D” prefix, e.g., D-Ala or N-Me-D-Ile, the stereochemistry of the alpha-carbon of the amino acids and aminoacyl residues in peptides described herein is the natural or “L” configuration. The Cahn-Ingold-Prelog “R” and “S” designations are used to specify the stereochemistry of chiral centers in certain acyl substituents at the N-terminus of the peptides. The designation “R,S” is meant to indicate a racemic mixture of the two enantiomeric forms. This nomenclature follows that described in R. S. Cahn, et al., Angew. Chem. Int. Ed. Engl., 5:385-415 (1966).

D-H refers to D Histidine.

2-aminoisobutyric acid as used herein has the following structure:

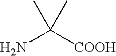

“Polypeptide,” “peptide,” and “protein” are used interchangeably to refer to a polymer of amino acid residues. As used herein, these terms may apply to amino acid polymers in which one or more amino acid residues is an artificial chemical analog of a corresponding naturally occurring amino acid. These terms also apply to naturally occurring amino acid polymers. Amino acids can be in the L or D form as long as the binding function of the peptide is maintained. Peptides may be cyclic, having an intramolecular bond between two non-adjacent amino acids within the peptide, e.g., backbone to backbone, side-chain to backbone and side-chain to side-chain cyclization. Cyclic peptides can be prepared by methods well know in the art. See, e.g., U.S. Pat. No. 6,013,625; S. Cheng et al., J. Med. Chem. 37:1-8 (1994).

All peptide sequences are written according to the generally accepted convention whereby the alpha-N-terminal amino acid residue is on the left and the alpha-C-terminal amino acid residue is on the right. As used herein, the term “N-terminus” refers to the free alpha-amino group of an amino acid in a peptide, and the term “C-terminus” refers to the free carboxylic acid terminus of an amino acid in a peptide. A peptide which is N-terminated with a group refers to a peptide bearing a group on the alpha-amino nitrogen of the N-terminal amino acid residue. An amino acid which is N-terminated with a group refers to an amino acid bearing a group on the alpha-amino nitrogen.

In general, “substituted” refers to a group as defined below in which one or more bonds to a hydrogen atom contained therein are replaced by a bond to non-hydrogen or non-carbon atoms such as, but not limited to, a halogen atom such as F, Cl, Br, and I; an oxygen atom in groups such as hydroxyl, alkoxy, aryloxy, and ester groups; a sulfur atom in groups such as thiol, alkyl sulfide, aryl sulfide, sulfone, sulfonyl, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl, dialkylarylsilyl, alkyldiarylsilyl, and triarylsilyl groups; and other heteroatoms in various other groups. Substituted alkyl groups, substituted cycloalkyl groups, and other substituted groups also include groups in which one or more bonds to a carbon(s) or hydrogen(s) atom is replaced by a bond to a heteroatom such as oxygen in carbonyl, carboxyl, and ester groups or nitrogen in groups such as imines, oximes, hydrazones, and nitriles. As employed herein, a group which is “optionally substituted” may be substituted or unsubstituted. Thus, e.g., “optionally substituted alkyl” refers to both substituted alkyl groups and unsubstituted alkyl groups.

The phrase “unsubstituted alkyl” refers to alkyl groups that do not contain heteroatoms. Thus, the phrase includes straight chain alkyl groups such as methyl, ethyl, propyl, butyl, pentyl, hexyl, heptyl, octyl, nonyl, decyl, undecyl, dodecyl and the like. The phrase also includes branched chain isomers of straight chain alkyl groups, including but not limited to, the following which are provided by way of example: —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃)CH(CH₃)CH(CH₃)₂, —CH(CH₂CH₃)CH(CH₃)CH(CH₃)(CH₂CH₃), and others. The phrase does not include cycloalkyl groups. Thus, the phrase unsubstituted alkyl group includes primary alkyl groups, secondary alkyl groups, and tertiary alkyl groups. Unsubstituted alkyl groups may be bonded to one or more carbon atom(s), oxygen atom(s), nitrogen atom(s), and/or sulfur atom(s) in the parent compound. Possible unsubstituted alkyl groups include straight and branched chain alkyl groups having 1 to 20 carbon atoms. Alternatively, such unsubstituted alkyl groups have from 1 to 10 carbon atoms or are lower alkyl groups having from 1 to about 6 carbon atoms. Other unsubstituted alkyl groups include straight and branched chain alkyl groups having from 1 to 3 carbon atoms and include methyl, ethyl, propyl, and —CH(CH₃)₂.

The phrase “substituted alkyl” refers to an unsubstituted alkyl group as defined herein in which one or more bonds to a carbon(s) or hydrogen(s) are replaced by a bond to non-hydrogen and non-carbon atoms such as, but not limited to, a halogen atom in halides such as F, Cl, Br, and I; an oxygen atom in groups such as hydroxyl, alkoxy, aryloxy, and ester groups; a sulfur atom in groups such as thiol, alkyl sulfide, aryl sulfide, sulfone, sulfonyl, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl, dialkylarylsilyl, alkyldiarylsilyl, and triarylsilyl groups; and other heteroatoms in various other groups. Substituted alkyl groups also include groups in which one or more bonds to a carbon(s) or hydrogen(s) atom is replaced by a bond to a heteroatom such as oxygen in carbonyl, carboxyl, and ester groups or nitrogen in groups such as imines, oximes, hydrazones, and nitriles. Substituted alkyl groups include, among others, alkyl groups in which one or more bonds to a carbon or hydrogen atom is/are replaced by one or more bonds to fluorine atoms. One example of a substituted alkyl group is the trifluoromethyl group and other alkyl groups that contain the trifluoromethyl group. Other alkyl groups include those in which one or more bonds to a carbon or hydrogen atom is replaced by a bond to an oxygen atom such that the substituted alkyl group contains a hydroxyl, alkoxy, aryloxy group, or heterocyclyloxy group. Still other alkyl groups include alkyl groups that have an amine, alkylamine, dialkylamine, arylamine, (alkyl)(aryl)amine, diarylamine, heterocyclylamine, (alkyl)(heterocyclyl)amine, (aryl)(heterocyclyl)amine, or diheterocyclylamine group.

The phrase “unsubstituted alkylene” refers to a divalent unsubstituted alkyl group as defined herein. Thus, methylene, ethylene, and propylene are each examples of unsubstituted alkylenes. The phrase “substituted alkylene” refers to a divalent substituted alkyl group as defined herein. Substituted or unsubstituted lower alkylene groups have from 1 to about 6 carbons.

The phrase “unsubstituted cycloalkyl” refers to cyclic alkyl groups such as cyclopropyl, cyclobutyl, cyclopentyl, cyclohexyl, cycloheptyl, and cyclooctyl, and such rings substituted with straight and branched chain alkyl groups as defined herein. The phrase also includes polycyclic alkyl groups such as, but not limited to, adamantyl norbornyl, bicyclo[2.2.2]octyl, and the like, as well as such rings substituted with straight and branched chain alkyl groups as defined herein. Thus, the phrase would include methylcyclohexyl groups, among others. The phrase does not include cyclic alkyl groups containing heteroatoms. Unsubstituted cycloalkyl groups may be bonded to one or more carbon atom(s), oxygen atom(s), nitrogen atom(s), and/or sulfur atom(s) in the parent compound. In some embodiments unsubstituted cycloalkyl groups have from 3 to 20 carbon atoms. In other embodiments, such unsubstituted alkyl groups have from 3 to 8 carbon atoms, while in others such groups have from 3 to 7 carbon atoms.

The phrase “substituted cycloalkyl”” has the same meaning with respect to unsubstituted cycloalkyl groups that “substituted alkyl” has with respect to unsubstituted alkyl groups. Thus, the phrase includes, but is not limited to, oxocyclohexyl, chlorocyclohexyl, hydroxycyclopentyl, and chloromethylcyclohexyl groups.

The phrase “unsubstituted aryl” refers to aryl groups that do not contain heteroatoms. Thus, the phrase includes, but is not limited to, groups such as phenyl, biphenyl, anthracenyl, and naphthenyl. Although the phrase “unsubstituted aryl” includes groups containing condensed rings such as naphthalene, it does not include aryl groups that have other groups such as alkyl or halo groups bonded to one of the ring members, as aryl groups such as tolyl are considered herein to be substituted aryl groups as described below. Typically, an unsubstituted aryl may be a lower aryl, having from 6 to about 10 carbon atoms. One unsubstituted aryl group is phenyl. Unsubstituted aryl groups may be bonded to one or more carbon atom(s), oxygen atom(s), nitrogen atom(s), and/or sulfur atom(s) in the parent compound.

The phrase “substituted aryl” has the same meaning with respect to unsubstituted aryl groups that “substituted alkyl” has with respect to unsubstituted alkyl groups. However, a substituted aryl group also includes aryl groups in which one of the aromatic carbons is bonded to one of the non-carbon or non-hydrogen atoms described herein, and also includes aryl groups in which one or more aromatic carbons of the aryl group is bonded to a substituted and/or unsubstituted alkyl, alkenyl, or alkynyl group as defined herein. This includes bonding arrangements in which two carbon atoms of an aryl group are bonded to two atoms of an alkyl, alkenyl, or alkynyl group to define a fused ring system (e.g., dihydronaphthyl or tetrahydronaphthyl). Thus, the phrase “substituted aryl” includes, but is not limited to tolyl and hydroxyphenyl, among others.

The phrase “unsubstituted alkenyl” refers to straight and branched chain and cyclic groups such as those described with respect to unsubstituted alkyl groups as defined herein, except that at least one double bond exists between two carbon atoms. Examples include, but are not limited to vinyl, —CH═C(H)(CH₃), —CH═C(CH₃)₂, —C(CH₃)═C(H)₂, —C(CH₃)═C(H)(CH₃), —C(CH₂CH₃)═CH₂, cyclohexenyl, cyclopentenyl, cyclohexadienyl, butadienyl, pentadienyl, and hexadienyl, among others. Lower unsubstituted alkenyl groups have from 1 to about 6 carbons.

The phrase “substituted alkenyl” has the same meaning with respect to unsubstituted alkenyl groups that “substituted alkyl” has with respect to unsubstituted alkyl groups. A substituted alkenyl group includes alkenyl groups in which a non-carbon or non-hydrogen atom is bonded to a carbon that is double bonded to another carbon, and those in which one of the non-carbon or non-hydrogen atoms is bonded to a carbon not involved in a double bond to another carbon. For example, —CH═CH—OCH₃ and —CH═CH—CH₂—OH are both substituted alkenyls. Oxoalkenyls wherein a CH₂ group is replaced by a carbonyl, such as —CH═CH—C(O)—CH₃, are also substituted alkenyls.

The phrase “unsubstituted alkenylene” refers to a divalent unsubstituted alkenyl group as defined herein. Thus, —CH═CH— is an example of an unsubstituted alkenylene. The phrase “substituted alkenylene” refers to a divalent substituted alkenyl group as defined herein.

The phrase “unsubstituted alkynyl” refers to straight and branched chain groups such as those described with respect to unsubstituted alkyl groups as defined herein, except that at least one triple bond exists between two carbon atoms. Examples include, but are not limited to —C—C(H), —C—C(CH₃), —C≡C(CH₂CH₃), —C(H₂)C≡C(H), —C(H)₂C≡C(CH₃), and —C(H)₂C≡C(CH₂CH₃), among others. Unsubstituted lower alkynyl groups have from 1 to about 6 carbons.

The phrase “substituted alkynyl” has the same meaning with respect to unsubstituted alkynyl groups that “substituted alkyl” has with respect to unsubstituted alkyl groups. A substituted alkynyl group includes alkynyl groups in which a non-carbon or non-hydrogen atom is bonded to a carbon that is triple bonded to another carbon, and those in which a non-carbon or non-hydrogen atom is bonded to a carbon not involved in a triple bond to another carbon. Examples include, but are not limited to, oxoalkynyls wherein a CH₂ group is replaced by a carbonyl, such as in —C(O)—CH≡CH—CH₃ and —C(O)—CH₂—CH≡CH, among others.

The phrase “unsubstituted alkynylene” refers to a divalent unsubstituted alkynyl group as defined herein. Thus, —C≡C— is an example of an unsubstituted alkynylene. The phrase “substituted alkynylene” refers to a divalent substituted alkynyl group as defined herein.

The phrase “unsubstituted aralkyl” refers to unsubstituted alkyl groups as defined herein in which a hydrogen or carbon bond of the unsubstituted alkyl group is replaced with a bond to an aryl group as defined herein. For example, methyl (—CH₃) is an unsubstituted alkyl group. If a hydrogen atom of the methyl group is replaced by a bond to a phenyl group, such as if the carbon of the methyl were bonded to a carbon of benzene, then the compound is an unsubstituted aralkyl group (i.e., a benzyl group). Thus, the phrase includes, but is not limited to, groups such as benzyl, diphenylmethyl, and 1-phenylethyl (—CH(C₆H₅)(CH₃)), among others.

The phrase “substituted aralkyl” has the same meaning with respect to unsubstituted aralkyl groups that “substituted aryl” has with respect to unsubstituted aryl groups. However, a substituted aralkyl group also includes groups in which a carbon or hydrogen bond of the alkyl part of the group is replaced by a bond to a non-carbon or a non-hydrogen atom. Examples of substituted aralkyl groups include, but are not limited to, —CH₂C(═O)(C₆H₅), and —CH₂(2-methylphenyl), among others.

The phrase “unsubstituted aralkenyl” refers to unsubstituted alkenyl groups as defined herein in which a hydrogen or carbon bond of the unsubstituted alkenyl group is replaced with a bond to an aryl group as defined herein. For example, vinyl is an unsubstituted alkenyl group. If a hydrogen atom of the vinyl group is replaced by a bond to a phenyl group, such as if a carbon of the vinyl were bonded to a carbon of benzene, then the compound is an unsubstituted aralkenyl group (i.e., a styryl group). Thus, the phrase includes, but is not limited to, groups such as styryl, diphenylvinyl, and 1-phenylethenyl (—C(C₆H₅)(CH₂)), among others.

The phrase “substituted aralkenyl” has the same meaning with respect to unsubstituted aralkenyl groups that “substituted aryl” has with respect to unsubstituted aryl groups. However, a substituted aralkenyl group also includes groups in which a carbon or hydrogen bond of the alkenyl part of the group is replaced by a bond to a non-carbon or a non-hydrogen atom. Examples of substituted aralkenyl groups include, but are not limited to, —CH═C(Cl)(C₆H₅), and CH═CH(2-methylphenyl), among others.

The phrase “unsubstituted aralkynyl” refers to unsubstituted alkynyl groups as defined herein in which a hydrogen or carbon bond of the unsubstituted alkynyl group is replaced with a bond to an aryl group as defined herein. For example, acetylene is an unsubstituted alkynyl group. If a hydrogen atom of the acetylene group is replaced by a bond to a phenyl group, such as if a carbon of the acetylene were bonded to a carbon of benzene, then the compound is an unsubstituted aralkynyl group. Thus, the phrase includes, but is not limited to, groups such as —C≡C-phenyl, and —CH₂—C≡C-phenyl, among others.

The phrase “substituted aralkynyl” has the same meaning with respect to unsubstituted aralkynyl groups that “substituted aryl” has with respect to unsubstituted aryl groups. However, a substituted aralkynyl group also includes groups in which a carbon or hydrogen bond of the alkynyl part of the group is replaced by a bond to a non-carbon or a non-hydrogen atom. Examples of substituted aralkynyl groups include, but are not limited to, —C≡C—C(Br)(C₆H₅), and —C≡C(2-methylphenyl), among others.

The phrase “unsubstituted heteroalkyl” refers to unsubstituted alkyl groups as defined herein in which the carbon chain is interrupted by one or more heteroatoms chosen from N, O, and S. Unsubstituted heteroalkyls containing N may have NH or N(unsubstituted alkyl) in the carbon chain. Thus, unsubstituted heteroalkyls include alkoxy, alkoxyalkyl, alkoxyalkoxy, thioether, alkylaminoalkyl, aminoalkyloxy, and other such groups. Typically, unsubstituted heteroalkyl groups contain 1-5 heteroatoms, and particularly 1-3 heteroatoms. In some embodiments unsubstituted heteroalkyls include, for example, alkoxyalkoxyalkoxy groups such as ethyloxyethyloxyethyloxy.

The phrase “substituted heteroalkyl” has the same meaning with respect to unsubstituted heteroalkyl groups that “substituted alkyl” has with respect to unsubstituted alkyl groups.

The phrase “unsubstituted heteroalkylene” refers to a divalent unsubstituted heteroalkyl group as defined herein. Thus, CH₂—O—CH₂— and CH₂—NH—CH₂CH₂— are both examples of unsubstituted heteroalkylenes. The phrase “substituted heteroalkylene” refers to a divalent substituted heteroalkyl group as defined herein.

The phrase “unsubstituted heteroalkenyl” refers to unsubstituted alkene groups as defined herein in which the carbon chain is interrupted by one or more heteroatoms chosen from N, O, and S. Unsubstituted heteroalkenyls containing N may have NH or N(unsubstituted alkyl or alkene) in the carbon chain. The phrase “substituted heteroalkenyl” has the same meaning with respect to unsubstituted heteroalkenyl groups that “substituted heteroalkyl” has with respect to unsubstituted heteroalkyl groups.

The phrase “unsubstituted heteroalkenylene” refers to a divalent unsubstituted heteroalkenyl group as defined herein. Thus CH₂—O—CH═CH— is an example of an unsubstituted heteroalkenylene. The phrase “substituted heteroalkenylene” refers to a divalent substituted heteroalkenyl group as defined herein.

The phrase “unsubstituted heteroalkynyl” refers to unsubstituted alkynyl groups as defined herein in which the carbon chain is interrupted by one or more heteroatoms chosen from N, O, and S. Unsubstituted heteroalkynyls containing N may have NH or N(unsubstituted alkyl, alkene, or alkyne) in the carbon chain. The phrase “substituted heteroalkynyl” has the same meaning with respect to unsubstituted heteroalkynyl groups that “substituted heteroalkyl” has with respect to unsubstituted heteroalkyl groups.

The phrase “unsubstituted heteroalkynylene” refers to a divalent unsubstituted heteroalkynyl group as defined herein. Thus, —CH₂—O—CH₂—C≡C— is an example of an unsubstituted heteroalkynylene. The phrase “substituted heteroalkynylene” refers to a divalent substituted heteroalkynyl group as defined herein.

The phrase “unsubstituted heterocyclyl” refers to both aromatic and nonaromatic ring compounds, including monocyclic, bicyclic, and polycyclic ring compounds such as, for example, quinuclidyl, which contain three or more ring members, of which one or more is a heteroatom such as, but not limited to, N, O, and S. Although the phrase “unsubstituted heterocyclyl” includes condensed heterocyclic rings such as benzimidazolyl, it does not include heterocyclyl groups that have other groups such as alkyl or halo groups bonded to one of the ring members, as compounds such as 2-methylbenzimidazolyl are substituted heterocyclyl groups. Examples of heterocyclyl groups include, but are not limited to: unsaturated 3 to 8 member rings containing 1 to 4 nitrogen atoms such as, but not limited to pyrrolyl, pyrrolinyl, imidazolyl, pyrazolyl, pyridyl, dihydropyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.), tetrazolyl, (e.g., 1H-tetrazolyl, 2H tetrazolyl, etc.); saturated 3 to 8 membered rings containing 1 to 4 nitrogen atoms such as, but not limited to, pyrrolidinyl, imidazolidinyl, piperidinyl, piperazinyl; condensed unsaturated heterocyclic groups containing 1 to 4 nitrogen atoms such as, but not limited to, indolyl, isoindolyl, indolinyl, indolizinyl, benzimidazolyl, quinolyl, isoquinolyl, indazolyl, benzotriazolyl; unsaturated 3 to 8 membered rings containing 1 to 3 oxygen atoms and 1 to 3 nitrogen atoms such as, but not limited to, oxazolyl, isoxazolyl, oxadiazolyl (e.g., 1,2,4-oxadiazolyl, 1,3,4-oxadiazolyl, 1,2,5-oxadiazolyl, etc.); saturated 3 to 8 membered rings containing 1 to 2 oxygen atoms and 1 to 3 nitrogen atoms such as, but not limited to, morpholinyl; unsaturated condensed heterocyclic groups containing 1 to 2 oxygen atoms and 1 to 3 nitrogen atoms, for example, benzoxazolyl, benzoxadiazolyl, benzoxazinyl (e.g., 2H-1,4-benzoxazinyl etc.); unsaturated 3 to 8 membered rings containing 1 to 3 sulfur atoms and 1 to 3 nitrogen atoms such as, but not limited to, thiazolyl, isothiazolyl, thiadiazolyl (e.g., 1,2,3-thiadiazolyl, 1,2,4-thiadiazolyl, 1,3,4-thiadiazolyl, 1,2,5-thiadiazolyl, etc.); saturated 3 to 8 membered rings containing 1 to 2 sulfur atoms and 1 to 3 nitrogen atoms such as, but not limited to, thiazolodinyl; saturated and unsaturated 3 to 8 membered rings containing 1 to 2 sulfur atoms such as, but not limited to, thienyl, dihydrodithiinyl, dihydrodithionyl, tetrahydrothiophene, tetrahydrothiopyran; unsaturated condensed heterocyclic rings containing 1 to 2 sulfur atoms and 1 to 3 nitrogen atoms such as, but not limited to, benzothiazolyl, benzothiadiazolyl, benzothiazinyl (e.g., 2H-1,4-benzothiazinyl, etc.), dihydrobenzothiazinyl (e.g., 2H-3,4-dihydrobenzothiazinyl, etc.), unsaturated 3 to 8 membered rings containing oxygen atoms such as, but not limited to furyl; unsaturated condensed heterocyclic rings containing 1 to 3 oxygen atoms such as benzodioxolyl (e.g., 1,3-benzodioxoyl, etc.); unsaturated 3 to 8 membered rings containing an oxygen atom and 1 to 2 sulfur atoms such as, but not limited to, dihydrooxathiinyl; saturated 3 to 8 membered rings containing 1 to 3 oxygen atoms and 1 to 2 sulfur atoms such as 1,4-oxathiane; unsaturated condensed rings containing 1 to 2 sulfur atoms such as benzothienyl, benzodithiinyl; and unsaturated condensed heterocyclic rings containing an oxygen atom and 1 to 3 oxygen atoms such as benzoxathiinyl. Heterocyclyl group also include those described above in which one or more S atoms in the ring are double-bonded to one or two oxygen atoms (sulfoxides and sulfones). For example, heterocyclyl groups include tetrahydrothiophene, tetrahydrothiophene oxide, and tetrahydrothiophene 1,1-dioxide. In some embodiments heterocyclyl groups contain 5 or 6 ring members. In other embodiments heterocyclyl groups include morpholine, piperazine, piperidine, pyrrolidine, imidazole, pyrazole, 1,2,3-triazole, 1,2,4-triazole, tetrazole, thiomorpholine, thiomorpholine in which the S atom of the thiomorpholine is bonded to one or more O atoms, pyrrole, homopiperazine, oxazolidin-2-one, pyrrolidin-2-one, oxazole, quinuclidine, thiazole, isoxazole, furan, and tetrahydrofuran.

The phrase “substituted heterocyclyl” refers to an unsubstituted heterocyclyl group as defined herein in which one of the ring members is bonded to a non-hydrogen atom, such as described above with respect to substituted alkyl groups and substituted aryl groups. Examples include, but are not limited to, 2-methylbenzimidazolyl, 5-methylbenzimidazolyl, 5-chlorobenzthiazolyl, 1-methyl piperazinyl, and 2-chloropyridyl, among others.

The phrase “unsubstituted heteroaryl” refers to unsubstituted aromatic heterocyclyl groups as defined herein. Thus, unsubstituted heteroaryl groups include but are not limited to furyl, imidazolyl, oxazolyl, isoxazolyl, pyridinyl, benzimidazolyl, and benzothiazolyl. The phrase “substituted heteroaryl” refers to substituted aromatic heterocyclyl groups as defined herein.

The phrase “unsubstituted heterocyclylalkyl” refers to unsubstituted alkyl groups as defined herein in which a hydrogen or carbon bond of the unsubstituted alkyl group is replaced with a bond to a heterocyclyl group as defined herein. For example, methyl (—CH₃) is an unsubstituted alkyl group. If a hydrogen atom of the methyl group is replaced by a bond to a heterocyclyl group, such as if the carbon of the methyl is bonded to carbon 2 of pyridine (one of the carbons bonded to the N of the pyridine) or carbons 3 or 4 of the pyridine, then the compound is an unsubstituted heterocyclylalkyl group.

The phrase “substituted heterocyclylalkyl” has the same meaning with respect to unsubstituted heterocyclylalkyl groups that “substituted aralkyl” has with respect to unsubstituted aralkyl groups. However, a substituted heterocyclylalkyl group also includes groups in which a non-hydrogen atom is bonded to a heteroatom in the heterocyclyl group of the heterocyclylalkyl group such as, but not limited to, a nitrogen atom in the piperidine ring of a piperidinylalkyl group.

The phrase “unsubstituted heterocyclylalkenyl” refers to unsubstituted alkenyl groups as defined herein in which a hydrogen or carbon bond of the unsubstituted alkenyl group is replaced with a bond to a heterocyclyl group as defined herein. For example, vinyl is an unsubstituted alkenyl group. If a hydrogen atom of the vinyl group is replaced by a bond to a heterocyclyl group, such as if the carbon of the vinyl is bonded to carbon 2 of pyridine or carbons 3 or 4 of the pyridine, then the compound is an unsubstituted heterocyclylalkenyl group.

The phrase “substituted heterocyclylalkenyl” has the same meaning with respect to unsubstituted heterocyclylalkenyl groups that “substituted aralkenyl” has with respect to unsubstituted aralkenyl groups. However, a substituted heterocyclylalkenyl group also includes groups in which a non-hydrogen atom is bonded to a heteroatom in the heterocyclyl group of the heterocyclylalkenyl group such as, but not limited to, a nitrogen atom in the piperidine ring of a piperidinylalkenyl group.

The phrase “unsubstituted heterocyclylalkynyl” refers to unsubstituted alkynyl groups as defined herein in which a hydrogen or carbon bond of the unsubstituted alkynyl group is replaced with a bond to a heterocyclyl group as defined herein. For example, acetylene is an unsubstituted alkynyl group. If a hydrogen atom of the acetylene group is replaced by a bond to a heterocyclyl group, such as if the carbon of the acetylene is bonded to carbon 2 of pyridine or carbons 3 or 4 of the pyridine, then the compound is an unsubstituted heterocyclylalkynyl group.

The phrase “substituted heterocyclylalkynyl” has the same meaning with respect to unsubstituted heterocyclylalkynyl groups that “substituted aralkynyl” has with respect to unsubstituted aralkynyl groups. However, a substituted heterocyclylalkynyl group also includes groups in which a non-hydrogen atom is bonded to a heteroatom in the heterocyclyl group of the heterocyclylalkynyl group such as, but not limited to, a nitrogen atom in the piperidine ring of a piperidinylalkynyl group.

The phrase “unsubstituted alkoxy” refers to a hydroxyl group (—OH) in which the bond to the hydrogen atom is replaced by a bond to a carbon atom of an otherwise unsubstituted alkyl group as defined herein.

The phrase “substituted alkoxy” refers to a hydroxyl group (—OH) in which the bond to the hydrogen atom is replaced by a bond to a carbon atom of an otherwise substituted alkyl group as defined herein.

A “pharmaceutically acceptable salt” includes a salt with an inorganic base, organic base, inorganic acid, organic acid, or basic or acidic amino acid. Salts of inorganic bases include, for example, alkali metals such as sodium or potassium; alkaline earth metals such as calcium and magnesium or aluminum; and ammonia. Salts of organic bases include, for example, trimethylamine, triethylamine, pyridine, picoline, ethanolamine, diethanolamine, and triethanolamine. Salts of inorganic acids include, for example, hydrochloric acid, hydroboric acid, nitric acid, sulfuric acid, and phosphoric acid. Salts of organic acids include, for example, formic acid, acetic acid, trifluoroacetic acid, fumaric acid, oxalic acid, tartaric acid, maleic acid, citric acid, succinic acid, malic acid, methanesulfonic acid, benzenesulfonic acid, and p-toluenesulfonic acid. Salts of basic amino acids include, for example, arginine, lysine and ornithine. Acidic amino acids include, for example, aspartic acid and glutamic acid.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, ketones are typically in equilibrium with their enol forms. Thus, ketones and their enols are referred to as tautomers of each other. As readily understood by one skilled in the art, a wide variety of functional groups and other structures may exhibit tautomerism, and all tautomers of compounds having Formulas I, II, and III are within the scope of the present invention.

The compounds according to the invention may be solvated, especially hydrated. Hydration may occur during manufacturing of the compounds or compositions comprising the compounds, or the hydration may occur over time due to the hygroscopic nature of the compounds.

Certain embodiments are derivatives referred to as prodrugs. The expression “prodrug” denotes a derivative of a pharmaceutically or therapeutically active drug, e.g., esters and amides, wherein the derivative has enhanced delivery characteristics and therapeutic value as compared to the drug and is transformed into the drug by an enzymatic or chemical process. See, for example, R. E. Notari, Methods Enzymol. 112:309-323 (1985); N. Bodor, Drugs of the Future 6:165-182 (1981); H. Bundgaard, Chapter 1 in Design of Prodrugs (H. Bundgaard, ed.), Elsevier, New York (1985); and A. G. Gilman et al., Goodman And Gilman's The Pharmacological Basis of Therapeutics, 8th ed., McGraw-Hill (1990). Thus, the prodrug may be designed to alter the metabolic stability or transport characteristics of a drug, mask side effects or toxicity of a drug, improve the flavor of a drug, or to alter other characteristics or properties of a drug.

Compounds of the present invention include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners. All such stereoisomers are within the scope of the invention.

The term “carboxy protecting group” as used herein refers to a carboxylic acid protecting ester group employed to block or protect the carboxylic acid functionality while the reactions involving other functional sites of the compound are carried out. Carboxy protecting groups are disclosed in, for example, Greene, Protective Groups in Organic Synthesis, pp. 152-186, John Wiley & Sons, New York (1981), which is hereby incorporated herein by reference. In addition, a carboxy protecting group can be used as a prodrug, whereby the carboxy protecting group can be readily cleaved in vivo by, for example, enzymatic hydrolysis, to release the biologically active parent. T. Higuchi and V. Stella provide a thorough discussion of the prodrug concept in “Pro-drugs as Novel Delivery Systems”, Vol 0.14 of the A.C. S. Symposium Series, American Chemical Society (1975), which is hereby incorporated herein by reference. Such carboxy protecting groups are well known to those skilled in the art, having been extensively used in the protection of carboxyl groups in the penicillin and cephalosporin fields, as described in U.S. Pat. Nos. 3,840,556 and 3,719,667, S. Kukolja, J. Am. Chem. Soc. 93:6267-6269 (1971), and G. E. Gutowski, Tetrahedron Lett. 21:1779-1782 (1970), the disclosures of which are hereby incorporated herein by reference. Examples of esters useful as prodrugs for compounds containing carboxyl groups can be found, for example, at pp. 14-21 in Bioreversible Carriers in Drug Design. Theory and Application (E. B. Roche, ed.), Pergamon Press, New York (1987), which is hereby incorporated herein by reference. Representative carboxy protecting groups are C₁ to C₈ alkyl (e.g., methyl, ethyl or tertiary butyl and the like); haloalkyl; alkenyl; cycloalkyl and substituted derivatives thereof such as cyclohexyl, cyclopentyl and the like; cycloalkylalkyl and substituted derivatives thereof such as cyclohexylmethyl, cyclopentylmethyl and the like; arylalkyl, for example, phenethyl or benzyl and substituted derivatives thereof such as alkoxybenzyl or nitrobenzyl groups and the like; arylalkenyl, for example, phenylethenyl and the like; aryl and substituted derivatives thereof, for example, 5-indanyl and the like; dialkylaminoalkyl (e.g., dimethylaminoethyl and the like); alkanoyloxyalkyl groups such as acetoxymethyl, butyryloxymethyl, valeryloxymethyl, isobutyryloxymethyl, isovaleryloxymethyl, 1-(propionyloxy)-1-ethyl, 1-(pivaloyloxyl)-1-ethyl, 1-methyl-1-(propionyloxy)-1-ethyl, pivaloyloxymethyl, propionyloxymethyl and the like; cycloalkanoyloxyalkyl groups such as cyclopropylcarbonyloxymethyl, cyclobutylcarbonyloxymethyl, cyclopentylcarbonyloxymethyl, cyclohexylcarbonyloxymethyl and the like; aroyloxyalkyl, such as benzoyloxymethyl, benzoyloxyethyl and the like; arylalkylcarbonyloxyalkyl, such as benzylcarbonyloxymethyl, 2-benzylcarbonyloxyethyl and the like; alkoxycarbonylalkyl, such as methoxycarbonylmethyl, cyclohexyloxycarbonylmethyl, 1-methoxycarbonyl-1-ethyl, and the like; alkoxycarbonyloxyalkyl, such as methoxycarbonyloxymethyl, t-butyloxycarbonyloxymethyl, 1-ethoxycarbonyloxy-1-ethyl, 1-cyclohexyloxycarbonyloxy-1-ethyl and the like; alkoxycarbonylaminoalkyl, such as t-butyloxycarbonylaminomethyl and the like; alkylaminocarbonylaminoalkyl, such as methylaminocarbonylaminomethyl and the like; alkanoylaminoalkyl, such as acetylaminomethyl and the like; heterocycliccarbonyloxyalkyl, such as 4-methylpiperazinylcarbonyloxymethyl and the like; dialkylaminocarbonylalkyl, such as dimethylaminocarbonylmethyl, diethylaminocarbonylmethyl and the like; (5-(alkyl)-2-oxo-1,3-dioxolen-4-yl)alkyl, such as (5-t-butyl-2-oxo-1,3-dioxolen-4-yl)methyl and the like; and (5-phenyl-2-oxo-1,3-dioxolen-4-yl)alkyl, such as (5-phenyl-2-oxo-1,3-dioxolen-4-yl)methyl and the like.

The term “N-protecting group” or “N-protected” as used herein refers to those groups intended to protect the N-terminus of an amino acid or peptide or to protect an amino group against undesirable reactions during synthetic procedures. Commonly used N-protecting groups are disclosed in, for example, Greene, Protective Groups in Organic Synthesis, John Wiley & Sons, New York (1981), which is hereby incorporated by reference. N-protecting groups comprise acyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, a-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-di methyl-3,5-dimethoxybenzyloxycarbonyl, benzhydryloxycarbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxycarbonyl, fluorenyl-9-methoxycarbonyl, cyclopentyloxycarbonyl, adamantyloxycarbonyl, cyclohexyloxycarbonyl, phenylthiocarbonyl and the like; alkyl groups such as benzyl, triphenylmethyl, benzyloxymethyl and the like; and silyl groups such as trimethylsilyl and the like. In some embodiments N-protecting groups are formyl, acetyl, benzoyl, pivaloyl, t-butylacetyl, phenylsulfonyl, benzyl, 9-fluorenylmethyloxycarbonyl (Fmoc), t-butyloxycarbonyl (Boc), and benzyloxycarbonyl (Cbz).

As used herein, “halo,” “halogen,” or “halide” refers to F, Cl, Br or I.

As used herein, the abbreviations for any protective groups, amino acids, or other compounds are, unless indicated otherwise, in accord with their common usage, recognized abbreviations, or the IUPAC-IUB Commission on Biochemical Nomenclature, Biochem. 11:942-944 (1972).

As used herein, “substantially pure” means sufficiently homogeneous to appear free of readily detectable impurities as determined by standard methods of analysis, such as thin layer chromatography (TLC), gel electrophoresis, and high performance liquid chromatography (HPLC), used by those of skill in the art to assess such purity, or sufficiently pure such that further purification would not detectably alter the physical and chemical properties, such as enzymatic and biological activities, of the substance. Methods for purification of compounds to produce substantially chemically pure compounds are known to those of skill in the art. A substantially chemically pure compound may, however, be a mixture of stereoisomers. In such instances, further purification may increase the specific activity of the compound.

As used herein, “biological activity” refers to the in vivo activities of a compound, composition, or other mixture, or physiological responses that result upon in vivo administration of a compound, composition or other mixture. Biological activity thus encompasses therapeutic effects and pharmaceutical activity of such compounds, compositions, and mixtures.

As used herein, “pharmacokinetics” refers to the concentration of an administered compound in the serum over time. Pharmacodynamics refers to the concentration of an administered compound in target and nontarget tissues over time and the effects on the target tissue (efficacy) and the non-target tissue (toxicity). Improvements in, for example, pharmacokinetics or pharmacodynamics can be designed for a particular targeting agent or biological agent, such as by using labile linkages or by modifying the chemical nature of any linker (changing solubility, charge, etc.).

As employed herein, the phrases “an effective amount” and “therapeutically effective amount” refer to a dose sufficient to provide concentrations high enough to impart a beneficial effect, e.g., an amelioration of symptoms, on the recipient thereof. The specific therapeutically effective dose level for any particular subject will depend upon a variety of factors including the disorder being treated, the severity of the disorder, the activity of the specific compound, the route of administration, the rate of clearance of the compound, the duration of treatment, the drugs used in combination or coincident with the compound, the age, body weight, sex, diet, and general health of the subject, and like factors well known in the medical arts and sciences. Various general considerations taken into account in determining the “therapeutically effective amount” are known to those of skill in the art and are described, e.g., in Gilman, A. G., et al., Goodman And Gilman's The Pharmacological Basis of Therapeutics, 8th ed., McGraw-Hill (1990); and Remington's Pharmaceutical Sciences, 17th ed., Mack Publishing Co., Easton, Pa. (1990).

In one aspect, the present invention provides various targeting compounds in which GA targeting agents are covalently linked to a combining site of an antibody.

In another aspect, the present invention includes methods of altering at least one physical or biological characteristic of a GA targeting agent. The methods include covalently linking a GA targeting agent to a combining site of an antibody, either directly or though a linker. Characteristics of an GA targeting agent that may be modified include, but are not limited to, binding affinity, susceptibility to degradation (e.g., by proteases), pharmacokinetics, pharmacodynamics, immunogenicity, solubility, lipophilicity, hydrophilicity, hydrophobicity, stability (either more or less stable, as well as planned degradation), rigidity, flexibility, modulation of antibody binding, and the like. Also, the biological potency of a particular GA targeting agent may be increased by the addition of the effector function(s) provided by the antibody. For example, an antibody provides effector functions such as complement mediated effector functions. Without wishing to be bound by any theory, the antibody portion of a GA targeting compound may generally extend the half-life of a smaller sized GA targeting agent in vivo. Thus, in one aspect, the invention provides a method for increasing the effective circulating half-life of a GA targeting agent.

In another aspect, the present invention provides methods for modulating the binding activity of an antibody by covalently attaching a GA targeting agent to a combining site of the antibody. Although not wishing to be bound by any theory, substantially reduced antibody binding to an antigen may result from the linked GA targeting agent(s) sterically hindering the antigen from contacting the antibody combining site. Alternatively, substantially reduced antigen binding may result if the amino acid sidechain of the antibody combining site that is modified by covalent linkage is important for binding to the antigen. By contrast, substantially increased antibody binding to an antigen may result when a linked GA targeting agent(s) does not sterically hinder the antigen from contacting the antibody combining site and/or when the amino acid sidechain of the antibody combining site modified by covalent linkage is not important for binding to the antigen.

In another aspect, the present invention includes methods of modifying a combining site of an antibody to generate binding specificity for GLP-1R. Such methods include covalently linking a reactive amino acid sidechain in a combining site of an antibody to a chemical moiety on a linker of a GA targeting agent-linker compound as described herein, where the GA targeting agent is specific for GLP-1R. The chemical moiety of the linker is sufficiently distanced from the GA targeting agent so that the GA targeting agent can bind to GLP-1R when the GA targeting agent-linker compound is covalently linked to the antibody combining site. In one embodiment, the antibody prior to covalent linking would have an affinity for GLP-1R of less than about 1×10⁻⁵ moles/liter. However, after the antibody is covalently linked to the GA targeting agent-linker compound, the modified antibody preferably has an affinity for the target molecule of at least about 1×10⁻⁶ moles/liter, alternatively, at least about 1×10⁻⁷ moles/liter, alternatively, at least 1×10⁻³ moles/liter, alternatively at least 1×10⁻⁹ moles/liter, or alternatively, at least about 1×1⁻¹⁰ moles/liter.

### GA Targeting Agents

In one embodiment, a GA targeting agent is:

R¹-HAEGTFTSDVSSYLEGQAAKEFIAWLVKGR-R² (SEQ ID NO:1),

### Wherein:

R¹ is absent, CH₃, C(O)CH3, C(O)CH₂CH₃, C(O)CH₂CH₂CH₃, or C(O)CH(CH₃)CH₃; and

R² is OH, NH₂, NH(CH₃), NHCH₂CH₃, NHCH₂CH₂CH₃, NHCH(CH₃)CH₃, NHCH₂CH₂CH₂CH₃, NHCH(CH₃)CH₂CH₃, NHC₆H₅, NHCH₂CH₂OCH₃, NHOCH₃, NHOCH₂CH₃, a carboxy protecting group, a lipid fatty acid group or a carbohydrate.

SEQ ID NO:1 is the 30 amino acid GLP-1 (7-36) generated by cleavage of GLP-1 by dipeptidyl peptidase IV (DPP-IV) at the position 2 alanine. D. J. Drucker. Endocrinology 142:521-527 (2001). GLP-1 (7-36) functions as a GLP-1R agonist, resulting in increased glucose-dependent insulin secretion. However, the half-life of GLP-1 (7-36) is only a few minutes.

In another embodiment, a GA targeting agent is:

R¹-HGEGTFTSDLSKQMEEEAVRLFIEWLKNGGPSSGAPPPS-R² (SEQ ID NO:2);

wherein

R¹ is absent, CH₃, C(O)CH3, C(O)CH₂CH₃, C(O)CH₂CH₂CH₃, or C(O)CH(CH₃)CH₃; and

R² is OH, NH₂, NH(CH₃), NHCH₂CH₃, NHCH₂CH₂CH₃, NHCH(CH₃)CH₃, NHCH₂CH₂CH₂CH₃, NHCH(CH₃)CH₂CH₃, NHC₆H₅, NHCH₂CH₂OCH₃, NHOCH₃, NHOCH₂CH₃, a carboxy protecting group, a lipid fatty acid group or a carbohydrate.

SEQ ID NO:2 is the 39 amino acid peptide exendin-4. Like GLP-1 (7-36), exendin-4 functions as a GLP-1R agonist and stimulates glucose-dependent insulin secretion. Unlike GLP-1 (7-36), however, exendin-4 is resistant to cleavage by DPP-IV. The N-terminal regions of GLP-1 (7-36) and exendin-4 are nearly identical, with the notable difference being the second amino acid residue. This residue is an alanine in GLP-1 (7-36), but a glycine in exendin-4. This single amino acid in the N-terminal region is responsible for the resistance of exendin-4 to DPP-IV digestion. Another notable difference between exendin-4 and DLP-1 (7-36) is the presence of nine additional amino acid residues at the C-terminus of exendin-4, which form a Trp-cage.

In addition to the peptides of SEQ ID NO:1 or SEQ ID NO:2, GA targeting agents as disclosed herein may be analogs of these sequences. Such analogs may possess additional advantageous features, such as, for example, increased bioavailability, increased stability, improved diabetic treatment profile, improved appetite control, improved body weight control, improved glucose tolerance, islet cell assay reactivity, and/or reduced host immune recognition. As used herein, an analog of a peptide of SEQ ID NO:1 or SEQ ID NO:2 is a peptide having essentially the sequence of SEQ ID NO:1 or SEQ ID NO:2, but with one or more amino acid substitutions, insertions, or deletions, or a combination thereof.

In certain embodiments, GA targeting agents as provided herein comprise SEQ ID NO:1 or SEQ ID NO:2, but with one or more amino acid substitutions. One possible class of amino acid substitutions in GA targeting agents would include those amino acid changes that are predicted to stabilize the structure of SEQ ID NO:1 or SEQ ID NO:2. Utilizing SEQ ID NO:1 or SEQ ID NO:2, the skilled artisan can readily compile consensus sequences, and ascertain from these consensus sequences conserved amino acid residues representing preferred amino acid substitutions. The amino acid substitutions may be of a conserved or non-conserved nature. Conserved amino acid substitutions consist of replacing one or more amino acids of SEQ ID NO:1 or SEQ ID NO:2 with amino acids of similar charge, size, and/or hydrophobicity characteristics, such as, for example, a glutamic acid (E) to aspartic acid (D) amino acid substitution. Non-conserved substitutions consist of replacing one or more amino acids of SEQ ID NO:1 or SEQ ID NO:2 with amino acids possessing dissimilar charge, size, and/or hydrophobicity characteristics, such as, for example, a glutamic acid (E) to valine (V) substitution. In certain embodiments, GA targeting agents as provided herein comprise SEQ ID NO:1 or SEQ ID NO:2 analogs, but with 2-aminoisobutyric acid (Aib2) substituted for the glycine residue at position 2 (or alanine, as appropriate). In certain embodiments, GA targeting agents as provided herein comprise SEQ ID NO:1 or SEQ ID NO:2, but with one or more residues substituted with a lysine.

In certain embodiments, GA targeting agents as provided herein comprise SEQ ID NO:1 or SEQ ID NO:2, but with one or more amino acid insertions. Amino acid insertions may consist of single amino acid residues or stretches of residues. The insertions may be made at the carboxy terminal end of the peptide, or at a position internal to the peptide. Such insertions will generally range from 2 to 10 amino acids in length. It is contemplated that insertions made at the carboxy terminus of the peptide of interest may be of a broader size range, with about 2 to about 20 amino acids being possible. One or more such insertions may be introduced into SEQ ID NO:1 or a SEQ ID NO:2 as long as such insertions result in peptides which still exhibit GLP-1R agonist activity.

In certain embodiments, a GA targeting peptide as provided herein comprises the amino acid sequence of SEQ ID NO:2, but with one or more inserted lysine residues. For example, in one embodiment a GA targeting agent is:

R¹-HGEGTFTSDLSKQMEEEAVRLFIEWLKNGGPSSGAPPPSK-R² (SEQ ID NO:3);

wherein:

R¹ is absent, CH₃, C(O)CH3, C(O)CH₂CH₃, C(O)CH₂CH₂CH₃, or C(O)CH(CH₃)CH₃; and

R² is OH, NH₂, NH(CH₃), NHCH₂CH₃, NHCH₂CH₂CH₃, NHCH(CH₃)CH₃, NHCH₂CH₂CH₂CH₃, NHCH(CH₃)CH₂CH₃, NHC₆H₅, NHCH₂CH₂OCH₃, NHOCH₃, NHOCH₂CH₃, a carboxy protecting group, a lipid fatty acid group or a carbohydrate.

The GA targeting agent of SEQ ID NO:3 is identical to SEQ ID NO:2 but for the addition of an extra lysine residue at the carboxy terminus.

In a similar embodiment, a GA targeting agent is:

R¹-HAEGTFTSDVSSYLEGQAAKEFIAWLVKGRK-R² (SEQ ID NO:33);

wherein:

R¹ is absent, CH₃, C(O)CH3, C(O)CH₂CH₃, C(O)CH₂CH₂CH₃, or C(O)CH(CH₃)CH₃; and

R² is OH, NH₂, NH(CH₃), NHCH₂CH₃, NHCH₂CH₂CH₃, NHCH(CH₃)CH₃, NHCH₂CH₂CH₂CH₃, NHCH(CH₃)CH₂CH₃, NHC₆H₅, NHCH₂CH₂OCH₃, NHOCH₃, NHOCH₂CH₃, a carboxy protecting group, a lipid fatty acid group or a carbohydrate.

The GA targeting agent of SEQ ID NO:33 is identical to SEQ ID NO:1 but for the addition of an extra lysine residue at the carboxy terminus.

In certain embodiments, GA targeting agents as provided herein comprise SEQ ID NO:1 or SEQ ID NO:2, but with one or more amino acid deletions. Such deletions may comprise truncations from the carboxy terminus of the peptide, or they may comprise removal of one or more amino acids from a position internal to the peptide. Such deletions may involve a single point deletion, a continuous deletion of two or more consecutive residues, or a combination of point and continuous deletions. One or more such deletions may be introduced into SEQ ID NO:1 or SEQ ID NO:2, so long as such deletions result in peptides that still exhibit GLP-1R agonist activities. In certain embodiments, a GA targeting peptide as provided herein comprises the amino acid sequence of SEQ ID NO:2, but with one or more amino acids deleted from the carboxy terminus of the peptide.

Suitable exemplary SEQ ID NO:1 and SEQ ID NO:2 analogs are set forth in Table I, below, and described herein in general formula format. Peptide sequences in Table I are listed from amino (left) to carboxy (right) terminus.

TABLE I
SEQ ID NO: 1 AND SEQ ID NO:2 ANALOGS

| | |
|---|---|
| R¹-HGEGTFTSDLSKQMEEEAVRLFIEWLKNGGPSSGAPPPSK-R² | (SEQ ID NO:3) |
| R¹-HGEGTFTSDLSKQMEEEAVRLFIEWLKNGGPSSGAPPPSK(SH)-R² | (SEQ ID NO:172) |
| R¹-HAibEGTFTSDLSKQMEEEAVRLFIEWLKNGGPSSGAPPPSK-R² | (SEQ ID NO:4) |
| R¹-HAibEGTFTSDLSKQMEEEAVRLFIEWLKNGGPSSGAPPPSK(SH)-R² | (SEQ ID NO:173) |
| R¹-HAibEGTFTSDLSKQMEEEAVRLFIEWLKNGGPSSGAPPPS-R² | (SEQ ID NO:5) |
| R¹-HAibEGTFTSDLSKQMEEEAVRLFIEWLKNGG-R² | (SEQ ID NO:6) |
| R¹-HAibEGTFTSDLSKQMEEEAVRLFIEWLKN-R² | (SEQ ID NO:7) |
| R¹-HAibEGTFTSDLSKQLEEEAVRLFIEFLKIN-R² | (SEQ ID NO:8) |
| R¹-HAibEGTFTSDLSKQLEEEAVRLAIEFLKIN-R² | (SEQ ID NO:9) |
| R¹-HAibEGTFTSDLSKQLEEEAVRLAIEFLKINGGPSSGAPPPS-R² | (SEQ ID NO:10) |
| R¹-HAibEGTFTSDLSKQLEEEAVRLFIEFLKINGGPSSGAPPPS-R² | (SEQ ID NO:11) |
| R¹-HAibEGTFTSDLSK(Ac)QMEEEAVRLFIEWLK(Ac)NGGPSSGAPPPS-R² | (SEQ ID NO:12) |
| R¹-HAibEGTFTSDLSK(benzoyl)QMEEEAVRLFIEWLK(benzoyl)NGGPSSGAPPPS-R² | (SEQ ID NO:13) |
| R¹-HAibEGTFTSDLSKQMEEEAVRLFIEWLKNGGPSSGAPPKS-R² | (SEQ ID NO:14) |
| R¹-HAibEGTFTSDLSKQMEEEAVRLFIEWLKNGGPSSGAPPK(SH)S-R² | (SEQ ID NO:99) |
| R¹-HAibEGTFTSDLSKQMEEEAVRLFIEWLKNGGPSSGAKPPS-R² | (SEQ ID NO:15) |
| R¹-HAibEGTFTSDLSKQMEEEAVRLFIEWLKNGGPSSGAK(SH)PPS-R² | (SEQ ID NO:100) |
| R¹-HAibEGTFTSDLSKQMEEEAVRLFIEWLKNGGPSSKAPPPS-R² | (SEQ ID NO:16) |
| R¹-HAibEGTFTSDLSKQMEEEAVRLFIEWLKNGGPSSK(SH)APPPS-R² | (SEQ ID NO:101) |
| R¹-HAibEGTFTSDLSKQMEEEAVRLFIEWLKNGGPKSGAPPPS-R² | (SEQ ID NO:17) |
| R¹-HAibEGTFTSDLSKQMEEEAVRLFIEWLKNGGPK(SH)SGAPPPS-R² | (SEQ ID NO:168) |
| R¹-HAibEGTFTSDLSKQMEEEAVRLFIEWLKKGGPSSGAPPPS-R² | (SEQ ID NO:18) |
| R¹-HAibEGTFTSDLSKQMEEEAVRLFIEWLKK(SH)GGPSSGAPPPS-R² | (SEQ ID NO:102) |
| R¹-HAibEGTFTSDLSKQMEEEAVRLFIEWKKNGGPSSGAPPPS-R² | (SEQ ID NO:19) |
| R¹-HAibEGTFTSDLSKQMEEEAVRLFIEWLK(SH)NGGPSSGAPPPS-R² | (SEQ ID NO:170) |
| R¹-HAibEGTFTSDLSKQMEEEAVRLFIEWK(SH)KNGGPSSGAPPPS-R² | (SEQ ID NO:103) |
| R¹-HAibEGTFTSDLSKQMEEEAVRLFIKWLKNGGPSSGAPPPS-R² | (SEQ ID NO:20) |
| R¹-HAibEGTFTSDLSKQMEEEAVRLFIK(SH)WLKNGGPSSGAPPPS-R² | (SEQ ID NO:104) |
| R¹-HAibEGTFTSDLSKQMEEEAVRLFKEWLIKINGGPSSGAPPPS-R² | (SEQ ID NO:21) |
| R¹-HAibEGTFTSDLSKQMEEEAVRLFK(SH)EWLKNGGPSSGAPPPS-R² | (SEQ ID NO:105) |
| R¹-HAibEGTFTSDLSKQMEEEAVRKFIEWLKNGGPSSGAPPPS-R² | (SEQ ID NO:22) |
| R¹-HAibEGTFTSDLSKQMEEEAVRK(SH)FIEWLKNGGPSSGAPPPS-R² | (SEQ ID NO:106) |
| R¹-HAibEGTFTSDLSKQMEEEAVKLFIEWLKINGGPSSGAPPPS-R² | (SEQ ID NO:23) |
| R¹-HAibEGTFTSDLSKQMEEEAVK(SH)LFIEWLKNGGPSSGAPPPS-R² | (SEQ ID NO:107) |
| R¹-HAibEGTFTSDLSKQMEEEAKRLFIEWLKNGGPSSGAPPPS-R² | (SEQ ID NO:24) |
| R¹-HAibEGTFTSDLSKQMEEEAK(SH)RLFIEWLKNGGPSSGAPPPS-R² | (SEQ ID NO:108) |
| R¹-HAibEGTFTSDLSKQMEEKAVRLFIEWLKNGGPSSGAPPPS-R² | (SEQ ID NO:25) |
| R¹-HAibEGTFTSDLSKQMEEK(SH)AVRLFIEWLKNGGPSSGAPPPS-R² | (SEQ ID NO:109) |
| R¹-HAibEGTFTSDLSKQMEKEAVRLFIEWLKNGGPSSGAPPPS-R² | (SEQ ID NO:26) |
| R¹-HAibEGTFTSDLSKQMEK(SH)EAVRLFIEWLKNGGPSSGAPPPS-R² | (SEQ ID NO:110) |
| R¹-HAibEGTFTSDLSKQKEEEAVRLFIEWLKNGGPSSGAPPPS-R² | (SEQ ID NO:27) |
| R¹-HAibEGTFTSDLSKQK(SH)EEEAVRLFIEWLKNGGPSSGAPPPS-R² | (SEQ ID NO:111) |
| R¹-HAibEGTFTSDLSKKMEEEAVRLFIEWLKNGGPSSGAPPPS-R² | (SEQ ID NO:28) |
| R¹-HAibEGTFTSDLSKK(SH)MEEEAVRLFIEWLKNGGPSSGAPPPS-R² | (SEQ ID NO:112) |
| R¹-HAibEGTFTSDLKKQMEEEAVRLFIEWLKNGGPSSGAPPPS-R² | (SEQ ID NO:29) |
| R¹-HAibEGTFTSDLSK(SH)QMEEEAVRLFIEWLKNGGPSSGAPPPS-R² | (SEQ ID NO:169) |
| R¹-HAibEGTFTSDLK(SH)KQMEEEAVRLFIEWLKNGGPSSGAPPPS-R² | (SEQ ID NO:113) |
| R¹-HAibEGTFTSDLSKQMEEEAVRLFIEWLKNGGK-R² | (SEQ ID NO:30) |
| R¹-HAibEGTFTSDLSKQMEEEAVRLFIEWLKNGGK(SH)-R² | (SEQ ID NO:114) |
| R¹-HAibEGTFTSDLSKQMEEEAVRKFIEWLKNGGPSS-R² | (SEQ ID NO:31) |
| R¹-HAibEGTFTSDLSKQMEEEAVRKFIEWLK(SH)NGGPSS-R² | (SEQ ID NO:115) |
| R¹-HAibEGTFTSDVSSYLEGQAAKEFIAWLVKGR-R² | (SEQ ID NO:32) |
| R¹-HAEGTFTSDVSSYLEGQAAKEFIAWLVKGRK-R² | (SEQ ID NO:33) |
| R¹-HGEGTFTSDVSSYLEGQAAKEFIAWLVKGRK-R² | (SEQ ID NO:34) |
| R¹-HAibEGTFTSDVSSYLEGQAAKEFIAWLVKAibR-R² | (SEQ ID NO:35) |
| R¹-HAibEGTFTSDVSSYLEGQAAKEFIAWLVKAibRK-R² | (SEQ ID NO:36) |
| R¹-HAibEGTFTSDVSSYLEGQAAKEFIAWLVKGRK-R² | (SEQ ID NO:37) |
| R¹-HAibEGTFTSDVSSYLEGQAAKEFIAWLVKGRK(SH)-R² | (SEQ ID NO:38) |
| R¹-HAibEGTFTSDVSSYLEGQAAK(SH)EFIAWLVKGR-R² | (SEQ ID NO:39) |
| R¹-HAibEGTFTSDVSSYGEGQAAKEFIAWLVKAibR-R² | (SEQ ID NO:40) |
| R¹-HAibEGTFTSDVSSYCEGQAAKEFIAWLVKAibR-R² | (SEQ ID NO:41) |
| R¹-HAibEGTFTSDVSSYFEGQAAKEFIAWLVKAibR-R² | (SEQ ID NO:42) |
| R¹-HAibEGTFTSDVSSYYEGQAAKEFIAWLVKAibR-R² | (SEQ ID NO:43) |
| R¹-HAibEGTFTSDVSSYWEGQAAKEFIAWLVKAibR-R² | (SEQ ID NO:44) |
| R¹-HAibEGTFTSDVSSYLEEQAAKEFIAWLVKAibR-R² | (SEQ ID NO:45) |
| R¹-HAibEGTFTSDVSSYLEDQAAKEFIAWLVKAibR-R² | (SEQ ID NO:46) |
| R¹-HAibEGTFTSDVSSYLEKQAAKEFIAWLVKAibR-R² | (SEQ ID NO:47) |
| R¹-HAibEGTFTSDVSSYLEGQAVKEFIAWLVKAibR-R² | (SEQ ID NO:48) |
| R¹-HAibEGTFTSDVSSYLEGQAIKEFIAWLVKAibR-R² | (SEQ ID NO:49) |
| R¹-HAibEGTFTSDVSSYLEGQALKEFIAWLVKAibR-R² | (SEQ ID NO:50) |
| R¹-HAibEGTFTSDVSSYLEGQAAREFIAWLVKAibR-R² | (SEQ ID NO:51) |
| R¹-HAibEGTFTSDVSSYLEGQAAOrnEFIAWLVKAibR-R² | (SEQ ID NO:52) |
| R¹-HAibEGTFTSDVSSYLEGQAAKEFIAFLVKAibR-R² | (SEQ ID NO:53) |
| R¹-HAibEGTFTSDVSSYLEGQAAKEFIAWLIKAibR-R² | (SEQ ID NO:54) |
| R¹-HAibEGTFTSDVSSYLEGQAAKEFIAWLVRAibR-R² | (SEQ ID NO:55) |
| R¹-HAibEGTFTSDVSSYLEGQAAKEFIAWLVOrnAibR-R² | (SEQ ID NO:56) |
| R¹-HAibEGTFTSDVSSYLEEQAVKEFIAWLIKAibR-R² | (SEQ ID NO:57) |
| R¹-HAibEGTFTSDVSSYFEEQAVIKIEFIAWLIKAibR-R² | (SEQ ID NO:58) |
| R¹-HAibEGTFTSDVSSYYEEQAVKEFIAWLIKAibR-R² | (SEQ ID NO:59) |
| R¹-HAibEGTFTSDVSSYWEEQAVKEFIAWLIKAibR-R² | (SEQ ID NO:60) |
| R¹-HAibEGTFTSDVSSYLEEQAVKEFIAWLIRAibR-R² | (SEQ ID NO:61) |
| R¹-HAibEGTFTSDVSSYLEEQAVREFIAWLIRAibR-R² | (SEQ ID NO:62) |
| R¹-HAibEGTFTSDVSSYLEEQAVKEFIAWLIKAibRPSSGAPPPS-R² | (SEQ ID NO:63) |
| R¹-HAibEGTFTSDVSSYLEEQAVK(SH)EFIAWLIKAibRPSSGAPPPS-R² | (SEQ ID NO:171) |
| R¹-HAibEGTFTSDKSSYLEEQAVKEFIAWLIKAibR-R² | (SEQ ID NO:64) |
| R¹-HAibEGTFTSDK(SH)SSYLEEQAVKEFIAWLIKAibR-R² | (SEQ ID NO:116) |
| R¹-HAibEGTFTSDVSKYLEEQAVKEFIAWLIKAibR-R² | (SEQ ID NO:65) |
| R¹-HAibEGTFTSDVSK(SH)YLEEQAVKEFIAWLIKAibR-R² | (SEQ ID NO:117) |
| R¹-HAibEGTFTSDVSSYKEEQAVKEFIAWLIKAibR-R² | (SEQ ID NO:66) |
| R¹-HAibEGTFTSDVSSYK(SH)EEQAVKEFIAWLIKAibR-R² | (SEQ ID NO:118) |
| R¹-HAibEGTFTSDVSSYLEKQAVKEFIAWLIKAibR-R² | (SEQ ID NO:67) |
| R¹-HAibEGTFTSDVSSYLEK(SH)QAVKEFIAWLIKAibR-R² | (SEQ ID NO:119) |
| R¹-HAibEGTFTSDVSSYLEEQKVKEFIAWLIKAibR-R² | (SEQ ID NO:68) |
| R¹-HAibEGTFTSDVSSYLEEQK(SH)VKEFIAWLIKAibR-R² | (SEQ ID NO:120) |
| R¹-HAibEGTFTSDVSSYLEEQAVKEKIAWLIKAibR-R² | (SEQ ID NO:69) |
| R¹-HAibEGTFTSDVSSYLEEQAVK(SH)EKIAWLIKAibR-R² | (SEQ ID NO:121) |
| R¹-HAibEGTFTSDVSSYLEEQAVKEFIKWLIKAibR-R² | (SEQ ID NO:70) |
| R¹-HAibEGTFTSDVSSYLEEQAVKEFIK(SH)WLIKAibR-R² | (SEQ ID NO:122) |
| R¹-HAibEGTFTSDVSSYLEEQAVKEFIAWKIKAibR-R² | (SEQ ID NO:71) |
| R¹-HAibEGTFTSDVSSYLEEQAVKEFIAWK(SH)IKAibR-R² | (SEQ ID NO:123) |
| R¹-HAibEGTFTSDVSSYLEEQAVKEFIAWLIKAibRPSSGAPPPSK-R² | (SEQ ID NO:72) |
| R¹-HAibEGTFTSDVSSYLEEQAVKEFIAWLIKAibRPSSGAPPPSK(SH)-R² | (SEQ ID NO:124) |
| R¹-HAibEGTFTSDVSSYLEEQAVKEFIAWLIK(Ac)AibR-R² | (SEQ ID NO:73) |
| R¹-HAibEGTFTSDVSSYLEEQAVKEFIAWLIK(benzoyl)AibR-R² | (SEQ ID NO:74) |
| R¹-H(Trans 3-hexanoyl)AibEGTFTSDVSSYLEEQAVKEFIAWLIKAibR-R² | (SEQ ID NO:75) |
| R¹-H(3-Aminophenylacetyl)AibEGTFTSDVSSYLEEQAVKEFIAWLIKAibR-R² | (SEQ ID NO:76) |

K(SH) as used herein refers to:

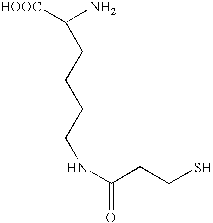

K(benzoyl) as used herein refers to a lysine residue linked to a benzoyl cap having the following structure:

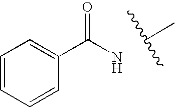

“Trans-3-hexanoyl” as used herein refers to a cap linked to a GA targeting peptide and having the following structure:

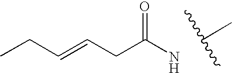

“3-aminophenyl acetyl” as used herein refers to a cap linked to a GA targeting peptide and having the following structure:

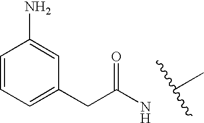

A GA targeting compound can be prepared using techniques well known in the art. Typically, synthesis of the peptidyl GA targeting agent is the first step, and is carried out as described herein. The targeting agent is then derivatized for linkage to a connecting component (the linker), which is then combined with the antibody. One of skill in the art will readily appreciate that the specific synthetic steps used depend upon the exact nature of the three components. Thus, the GA targeting agent linker conjugates and GA targeting compounds described herein can be readily synthesized.

GA targeting agent peptides may be synthesized by many techniques that are known to those skilled in the art. For solid phase peptide synthesis, a summary of the many techniques may be found in Chemical Approaches to the Synthesis of Peptides and Proteins (Williams et al., eds.), CRC Press, Boca Raton, Fla. (1997).

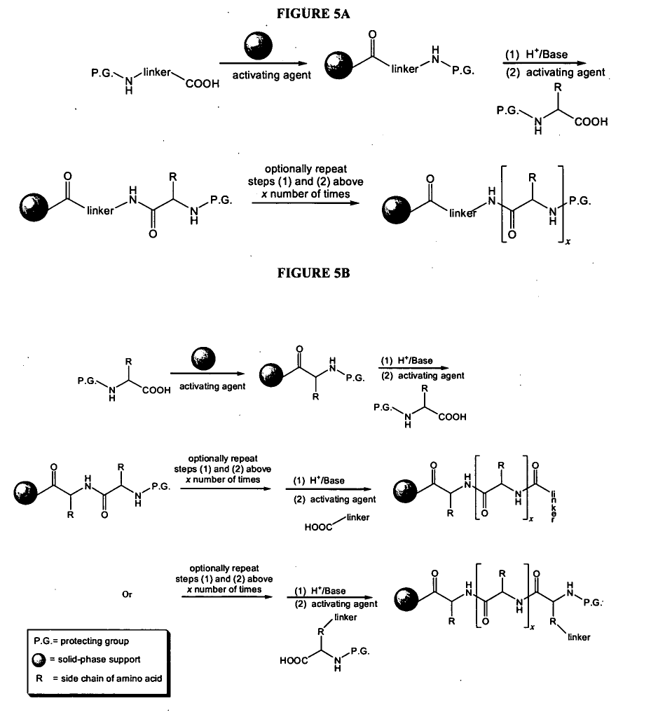

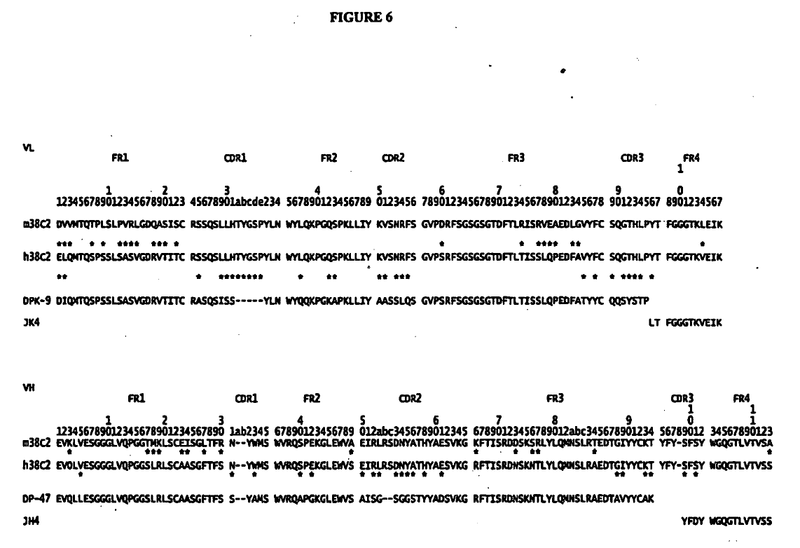

Typically, the desired GA targeting agent peptide is synthesized sequentially on solid phase according to procedures well known in the art. See, e.g., U.S. patent application Ser. No. 10/205,924 (Publication No. 2003/0045477A1). The linker may be attached to the peptide in part or in full on the solid phase, or may be added using solution phase techniques after the removal of the peptide from the resin (see FIGS. 5A and 5B). For example, an N-protected amino and carboxylic acid-containing linking moiety may be attached to a resin such as 4-hydroxymethyl-phenoxymethyl-poly(styrene-1% divinylbenzene). The N-protecting group may be removed by the appropriate acid (e.g., TFA for Boc) or base (e.g., piperidine for Fmoc), and the peptide sequence developed in the normal C-terminus to N-terminus fashion (see FIG. 5A). Alternatively, the peptide sequence may be synthesized first and the linker added to the peptide on the column (see FIG. 5B). Yet another method entails deprotecting an appropriate sidechain during synthesis and derivatizing with a suitably reactive linker. For example, a lysine sidechain may be deprotected and reacted with a linker having an active ester. Alternatively, an amino acid derivative with a suitably protected linker moiety already attached to the sidechain or, in some cases, the alpha-amino nitrogen, may be added as part of the growing peptide sequence.

At the end of the solid phase synthesis, the targeting agent-linker conjugate is removed from the resin and deprotected, either in succession or in a single operation. Removal of the targeting agent-linker conjugate and deprotection can be accomplished in a single operation by treating the resin-bound peptide-linker conjugate with a cleavage reagent, for example, trifluoroacetic acid containing scavengers such as thianisole, water, or ethanedithiol. After deprotection and release of the targeting agent, further derivatization of the targeting agent peptide may be carried out.

The fully deprotected targeting agent-linker conjugate is purified by a sequence of chromatographic steps employing any or all of the following types: ion exchange on a weakly basic resin in the acetate form; hydrophobic adsorption chromatography on underivatized polystyrene-divinylbenzene (e.g., AMBERLITE XAD); silica gel adsorption chromatography; ion exchange chromatography on carboxymethylcellulose; partition chromatography, e.g., on SEPHADEX G-25, LH-20 or countercurrent distribution; high performance liquid chromatography (HPLC), especially reverse-phase HPLC on octyl- or octadecylsilyl-silica bonded phase column packing.

### Antibodies

“Antibody” as used herein includes immunoglobulins which are the product of B cells and variants thereof as well as the T cell receptor (TcR) which is the product of T cells and variants thereof. An immunoglobulin is a protein comprising one or more polypeptides substantially encoded by the immunoglobulin kappa and lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. Subclasses of heavy chains are also known. For example, IgG heavy chains in humans can be any of IgG1, IgG2, IgG3, and IgG4 subclasses.

A typical immunoglobulin structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively. The amino acids of an antibody may be natural or nonnatural.

Antibodies that contain two heavy chains and two light chains are bivalent in that they have two combining sites. A typical natural bivalent antibody is an IgG. Antibodies may be multi-valent, as in the case of dimeric forms of IgA and the pentameric IgM molecule. Antibodies may also be univalent, such as, for example, in the case of Fab or Fab′ fragments.

Antibodies exist as full length intact antibodies or as a number of well-characterized fragments produced by digestion with various peptidases or chemicals. Thus, for example, pepsin digests an antibody below the disulfide linkages in the hinge region to produce F(ab′)₂, a dimer of Fab which itself is a light chain joined to V_{H}—CH₁ by a disulfide bond. The F(ab′)₂ may be reduced under mild conditions to break the disulfide linkage in the hinge region, thereby converting the F(ab′)₂ dimer into a Fab′ monomer. The Fab′ monomer is essentially a Fab fragment with part of the hinge region (see, e.g., Fundamental Immunology (W. E. Paul, ed.), Raven Press, N.Y. (1993) for a more detailed description of other antibody fragments). While various antibody fragments are defined in terms of the digestion of an intact antibody, one of skill in the art will appreciate that any of a variety of antibody fragments may be synthesized de novo either chemically or by utilizing recombinant DNA methodology. Thus, the term antibody as used herein also includes antibody fragments produced by the modification of whole antibodies, synthesized de novo, or obtained from recombinant DNA methodologies. Antibody fragments produced by recombinant techniques may include fragments known by proteolytic processing or may be unique fragments not available or previously unknown by proteolytic processing. Whole antibody and antibody fragments may contain natural as well as unnatural amino acids. One skilled in the art will recognize that there are circumstances in which it is advantageous to use antibody fragments rather than whole antibodies. For example, the smaller size of the antibody fragments allows for rapid clearance, and may lead to improved access to solid tumors.

The T cell receptor (TcR) is a disulfide linked heterodimer composed of two chains. The two chains are generally disulfide-bonded just outside the T cell plasma membrane in a short extended stretch of amino acids resembling the antibody hinge region. Each TcR chain is composed of one antibody-like variable domain and one constant domain. The full TcR has a molecular mass of about 95 kD, with the individual chains varying in size from 35 to 47 kD. Also encompassed within the meaning of TcR are portions of the receptor, such as, for example, the variable region, which can be produced as a soluble protein using methods well known in the art. For example, U.S. Pat. No. 6,080,840 and A. E. Slanetz and A. L. Bothwell, Eur. J. Immunol. 21:179-183 (1991) describe a soluble T cell receptor prepared by splicing the extracellular domains of a TcR to the glycosyl phosphatidylinositol (GPI) membrane anchor sequences of Thy-1. The molecule is expressed in the absence of CD3 on the cell surface, and can be cleaved from the membrane by treatment with phosphatidylinositol specific phospholipase C(PI-PLC). The soluble TcR also may be prepared by coupling the TcR variable domains to an antibody heavy chain CH₂ or CH₃ domain, essentially as described in U.S. Pat. No. 5,216,132 and G. S. Basi et al., J. Immunol. Methods 155:175-191 (1992), or as soluble TcR single chains, as described by E. V. Shusta et al., Nat. Biotechnol. 18:754-759 (2000) or P. D. Holler et al., Proc. Natl. Acad. Sci. U.S.A. 97:5387-5392 (2000). One embodiment of the invention uses TcR “antibodies” as a soluble antibody. The combining site of the TcR can be identified by reference to CDR regions and other framework residues using the same methods discussed above for antibodies.

Recombinant antibodies may be conventional full length antibodies, antibody fragments known from proteolytic digestion, antibody fragments such as Fv or single chain Fv (scFv), single domain fragments such as V_{H} or V_{L}, diabodies, domain deleted antibodies, minibodies, and the like. An Fv antibody is about 50 kD in size and comprises the variable regions of the light and heavy chain. The light and heavy chains may separately be expressed in bacteria where they assemble into an Fv fragment. Alternatively, the two chains can be engineered to form an interchain disulfide bond to give a dsFv. A single chain Fv (“scFv”) is a single polypeptide comprising V_{H} and V_{L} sequence domains linked by an intervening linker sequence, such that when the polypeptide folds the resulting tertiary structure mimics the structure of the antigen binding site. See J. S. Huston et al., Proc. Nat. Acad. Sci. U.S.A. 85:5879-5883 (1988). Single domain antibodies are the smallest functional binding units of antibodies (approximately 13 kD in size), corresponding to the variable regions of either the heavy V_{H} or light V_{L} chains. See U.S. Pat. No. 6,696,245, WO04/058821, WO04/003019 and WO03/002609. Single domain antibodies are well expressed in bacteria, yeast, and other lower eukaryotic expression systems. Domain deleted antibodies have a domain, such as CH2, deleted relative to the full length antibody. In many cases such domain deleted antibodies, particularly CH2 deleted antibodies, offer improved clearance relative to their full length counterparts. Diabodies are formed by the association of a first fusion protein comprising two V_{H} domains with a second fusion protein comprising two V_{L} domains. Diabodies, like full length antibodies, are bivalent. Minibodies are fusion proteins comprising a V_{H}, V_{L}, or scFv linked to CH3, either directly or via an intervening IgG hinge. See T. Olafsen et al., Protein Eng. Des. Sel. 17:315-323 (2004). Minibodies, like domain deleted antibodies, are engineered to preserve the binding specificity of full-length antibodies but with improved clearance due to their smaller molecular weight.

Various techniques have been developed for the production of antibody fragments. Traditionally, these fragments were derived via proteolytic digestion of intact antibodies (see, e.g., K. Morimoto and K. Inouye, J. Biochem. Biophys. Methods 24:107-117 (1992); M. Brennan et al., Science 229:81-83 (1985)). However, these fragments can now be produced directly by recombinant host cells. Fab, Fv and scFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (P. Carter et al., Biotechnology 10: 163-167 (1992)). According to another approach, F(ab′)₂ fragments can be isolated directly from recombinant host cell culture. Fab and F(ab′)₂ fragments with increased in vivo half-life comprising a salvage receptor binding epitope are described in U.S. Pat. No. 5,869,046. Other techniques for the production of antibody fragments will be apparent to the skilled practitioner.

The combining site refers to the part of an antibody molecule that participates in antigen binding. The antigen binding site is formed by amino acid residues of the N-terminal variable (“V”) regions of the heavy (“H”) and light (“L”) chains. The antibody variable regions comprise three highly divergent stretches referred to as “hypervariable regions” or “complementarity determining regions” (CDRs), which are interposed between more conserved flanking stretches known as “framework regions” (FRs). In an antibody molecule, the three hypervariable regions of a light chain (LCDR1, LCDR2, and LCDR3) and the three hypervariable regions of a heavy chain (HCDR1, HCDR2, and HCDR3) are disposed relative to each other in three dimensional space to form an antigen binding surface or pocket. The antibody combining site therefore represents the amino acids that make up the CDRs of an antibody and any framework residues that make up the binding site pocket.

The identity of the amino acid residues in a particular antibody that make up a combining site can be determined using methods well known in the art. For example, antibody CDRs may be identified as the hypervariable regions originally defined by Kabat et al. See E. A. Kabat et al., Sequences of Proteins of Immunological Interest, 5th ed., Public Health Service, NIH, Washington D.C. (1992). The positions of the CDRs may also be identified as the structural loop structures originally described by Chothia and others. See, e.g., C. Chothia and A. M. Lesk, J. Mol. Biol. 196:901-917 (1987); C. Chothia et al., Nature 342:877-883 (1989); and A. Tramontano et al., J. Mol. Biol. 215:175-182 (1990). Other methods include the “AbM definition,” which is a compromise between Kabat and Chothia and is derived using Oxford Molecular's AbM antibody modeling software (now Accelrys), or the “contact definition” of CDRs set forth in R. M. MacCallum et al., J. Mol. Biol. 262:732-745 (1996). The following chart identifies CDRs based upon various known definitions:

| CDR | Kabat | AbM | Chothia | Contact |
|---|---|---|---|---|
| L1 | L24-L34 | L24-L34 | L24-L34 | L30-L36 |
| L2 | L50-L56 | L50-L56 | L50-L56 | L46-L55 |
| L3 | L89-L97 | L89-L97 | L89-L97 | L89-L96 |
| H1 (Kabat | H31-H35B | H26-H35B | H26-H32 . . . | H30-H35B |
| numbering) | | | H34 | |
| H1 (Chothia | H31-H35 | H26-H35 | H26-H32 | H30-H35 |
| numbering) | | | | |
| H2 | H50-H56 | H50-H58 | H52-H56 | H47-H58 |
| H3 | H95-H102 | H95-H102 | H95-H102 | H93-H101 |

General guidelines by which one may identify the CDRs in an antibody from sequence alone are as follows:

LCDR1:

Start—Approximately residue 24.
Residue before is always a Cys.
Residue after is always a Trp, typically followed by Tyr-Gln, but also followed by Leu-Gln, Phe-Gln, or Tyr-Leu.

Length is 10 to 17 residues.

LCDR2:

Start 16 residues after the end of L1.
Sequence before is generally Ile-Tyr, but also may be Val-Tyr, Ile-Lys, or Ile-Phe.
Length is generally 7 residues.

LCDR3:

Start 33 residues after end of L2.
Residue before is a Cys.
Sequence after is Phe-Gly-X-Gly.
Length is 7 to 11 residues.

HCDR1:

Start—approximately residue 26, four residues after a Cys under Chothia/AbM definitions; start is 5 residues later under Kabat definition.
Sequence before is Cys-X-X-X.
Residue after is a Trp, typically followed by Val, but also followed by Ile or Ala.
Length is 10 to 12 residues under AbM definition; Chothia definition excludes the last 4 residues.

HCDR2:

Start 15 residues after the end of Kabat/AbM definition of CDR-H1.
Sequence before is typically Leu-Glu-Trp-Ile-Gly, but a number of variations are possible.
Sequence after is Lys/Arg-Leu/Ile/VaUPhe/Thr/Ala-Thr/Ser/Ile/Ala.
Length is 16 to 19 residues under Kabat definition; AbM definition excludes the last 7 residues.

HCDR3:

Start 33 residues after end of CDR-H2 (two residues after a Cys).
Sequence before is Cys-X-X (typically Cys-Ala-Arg).
Sequence after is Trp-Gly-X-Gly.
Length is 3 to 25 residues.

The identity of the amino acid residues in a particular antibody that are outside the CDRs, but nonetheless make up part of the combining site by having a sidechain that is part of the lining of the combining site (i.e., that is available to linkage through the combining site), can be determined using methods well known in the art, such as molecular modeling and X-ray crystallography. See, e.g., L. Riechmann et al., Nature 332:323-327 (1988).

As discussed, antibodies that can be used in preparing antibody-based GA targeting compounds require a reactive sidechain in the antibody combining site. A reactive sidechain may be present naturally or may be placed in an antibody by mutation. The reactive residue of the antibody combining site may be associated with the antibody, such as when the residue is encoded by nucleic acid present in the lymphoid cell first identified to make the antibody. Alternatively, the amino acid residue may arise by purposely mutating the DNA so as to encode the particular residue (see, e.g., WO 01/22922). The reactive residue may be a non-natural residue arising, for example, by biosynthetic incorporation using a unique codon, tRNA, and aminoacyl-tRNA as discussed herein. In another approach, the amino acid residue or its reactive functional groups (e.g., a nucleophilic amino group or sulfhydryl group) may be attached to an amino acid residue in the antibody combining site. Thus, covalent linkage with the antibody occurring “through an amino acid residue in a combining site of an antibody” as used herein means that linkage can be directly to an amino acid residue of an antibody combining site or through a chemical moiety that is linked to a sidechain of an amino acid residue of an antibody combining site.

Catalytic antibodies are one source of antibodies with combining sites that comprise one or more reactive amino acid sidechains. Such antibodies include aldolase antibodies, beta lactamase antibodies, esterase antibodies, amidase antibodies, and the like.

One embodiment comprises an aldolase antibody such as the mouse monoclonal antibody mAb 38C2 or mAb 33F12, as well as suitably humanized and chimeric versions of such antibodies. Mouse mAb 38C2 has a reactive lysine near to but outside HCDR3, and is the prototype of a new class of catalytic antibodies that were generated by reactive immunization and mechanistically mimic natural aldolase enzymes. See C. F. Barbas 3^{rd} et al., Science 278:2085-2092 (1997)). Other aldolase catalytic antibodies that may be used include the antibodies produced by the hybridoma 85A2, having ATCC accession number PTA-1015; hybridoma 85C7, having ATCC accession number PTA-1014; hybridoma 92F9, having ATCC accession number PTA-1017; hybridoma 93F3, having ATCC accession number PTA-823; hybridoma 84G3, having ATCC accession number PTA-824; hybridoma 84G11, having ATCC accession number PTA-1018; hybridoma 84H9, having ATCC accession number PTA-1019; hybridoma 85H6, having ATCC accession number PTA-825; hybridoma 90G8, having ATCC accession number PTA-1016. Through a reactive lysine, these antibodies catalyze aldol and retro-aldol reactions using the enamine mechanism of natural aldolases. See, e.g., J. Wagner et al., Science 270:1797-1800 (1995); C.F. Barbas 3^{rd} et al., Science 278:2085-2092 (1997); G. Zhong et al., Angew. Chem. Int. Ed. Engl. 38:3738-3741 (1999); A. Karlstrom et al., Proc. Natl. Acad. Sci. U.S.A., 97:3878-3883 (2000). Aldolase antibodies and methods of generating aldolase antibodies are disclosed in U.S. Pat. Nos. 6,210,938, 6,368,839, 6,326,176, 6,589,766, 5,985,626, and 5,733,757.

GA targeting compounds may also be formed by linking a GA targeting agent to a reactive cysteine, such as those found in the combining sites of thioesterase and esterase catalytic antibodies. Suitable thioesterase catalytic antibodies are described by K. D. Janda et al., Proc. Natl. Acad. Sci. U.S.A. 91:2532-2536 (1994). Suitable esterase antibodies are described by P. Wirsching et al., Science 270:1775-1782 (1995). Reactive amino acid-containing antibodies may be prepared by means well known in the art, including mutating an antibody combining site residue to encode for the reactive amino acid or chemically derivatizing an amino acid sidechain in an antibody combining site with a linker that contains the reactive group.

Antibodies suitable for use herein may be obtained by conventional immunization, reactive immunization in vivo, or by reactive selection in vitro, such as with phage display. Antibodies may be produced in humans or in other animal species. Antibodies from one species of animal may be modified to reflect another species of animal. For example, human chimeric antibodies are those in which at least one region of the antibody is from a human immunoglobulin. A human chimeric antibody is typically understood to have variable regions from a non-human animal, e.g., a rodent, with the constant regions from a human. In contrast, a humanized antibody uses CDRs from the non-human antibody with most or all of the variable framework regions and all the constant regions from a human immunoglobulin. Chimeric and humanized antibodies may be prepared by methods well known in the art including CDR grafting approaches (see, e.g., N. Hardman et al., Int. J. Cancer 44:424-433 (1989); C. Queen et al., Proc. Natl. Acad. Sci. U.S.A. 86:10029-10033 (1989)), chain shuffling strategies (see, e.g., Rader et al., Proc. Natl. Acad. Sci. U.S.A. 95:8910-8915 (1998), molecular modeling strategies (see, e.g., M. A. Roguska et al., Proc. Natl. Acad. Sci. U.S.A. 91:969-973 (1994)), and the like.

Methods for humanizing non-human antibodies have been described in the art. Preferably, a humanized antibody has one or more amino acid residues introduced into it from a source which is non-human. These non-human amino acid residues are often referred to as “import” residues, which are typically taken from an “import” variable domain. Humanization can be essentially performed following the methods of Winter and colleagues (see, e.g., P. T. Jones et al., Nature 321:522-525 (1986); L. Riechmann et al., Nature 332:323-327 (1988); M. Verhoeyen et al., Science 239:1534-1536 (1988)) by substituting hypervariable region sequences for the corresponding sequences of a human antibody. Accordingly, such “humanized” antibodies are chimeric antibodies (S. Cabilly et al., Proc. Natl. Acad. Sci. U.S.A. 81:3273-3277 (1984)), wherein substantially less than an intact human variable domain has been substituted by the corresponding sequence from a non-human species. In practice, humanized antibodies are typically human antibodies in which some hypervariable region residues and possibly some framework (FR) residues are substituted by residues from analogous sites in rodent antibodies.

The choice of human variable domains, both light and heavy, to be used in making humanized antibodies is very important to reduce antigenicity and human anti-mouse antibody (HAMA) response when the antibody is intended for human therapeutic use. According to the so-called “best-fit” method, the human variable domain utilized for humanization is selected from a library of known domains based on a high degree of homology with the rodent variable region of interest (M. J. Sims et al., J. Immunol., 151:2296-2308 (1993); M. Chothia and A.M. Lesk, J. Mol. Biol. 196:901-917 (1987)). Another method uses a framework region derived from the consensus sequence of all human antibodies of a particular subgroup of light or heavy chains. The same framework may be used for several different humanized antibodies (see, e.g., P. Carter et al., Proc. Natl. Acad. Sci. U.S.A. 89:4285-4289 (1992); L. G. Presta et al., J. Immunol., 151:2623-2632 (1993)).

It is further important that antibodies be humanized with retention of high linking affinity for the Z group. To achieve this goal, according to one method, humanized antibodies are prepared by analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence with respect to linking to the Z group. In this way, FR residues can be selected and combined from the recipient and import sequences so that the desired antibody characteristic, such as increased affinity for the target antigen(s), is achieved.

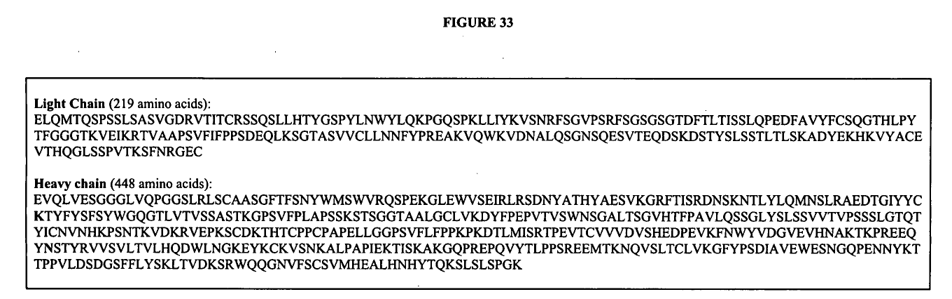

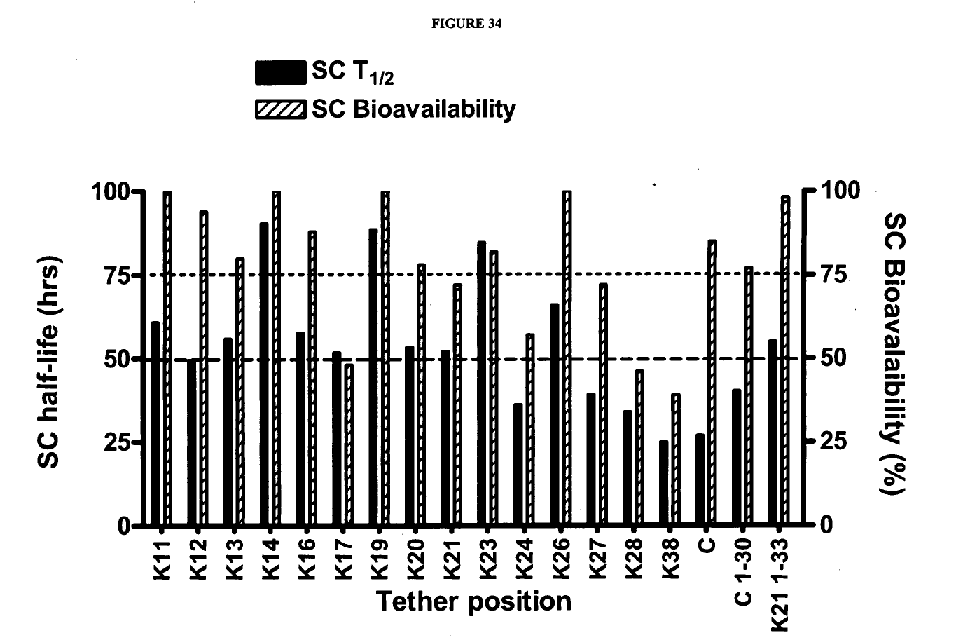

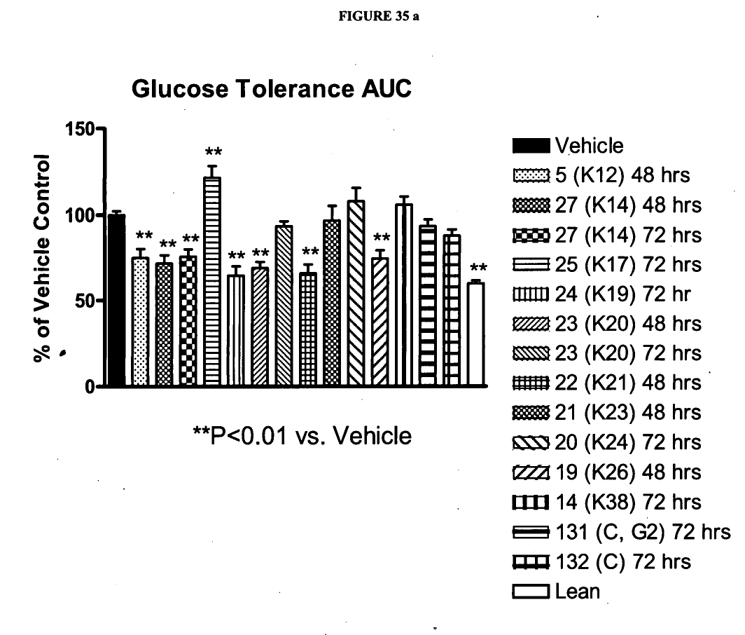

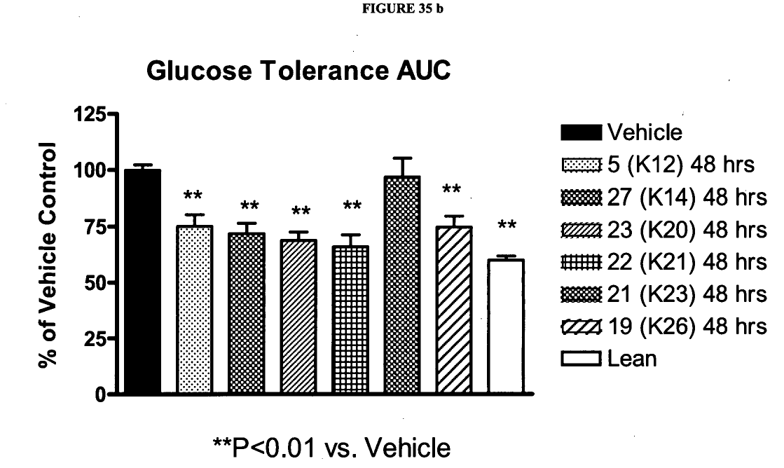

Various forms of humanized murine aldolase antibodies are contemplated. One embodiment uses the humanized aldolase catalytic antibody h38c2 IgG1 or h38c2 Fab with human constant domains C_{κ} and C_{γ1}1. C. Rader et al., J. Mol. Bio. 332:889-899 (2003) discloses the gene sequences and vectors that may be used to produce h38c2 Fab and h38c2 IgG1. The light and heavy chain sequences of h38c2 IgG1 are shown in FIG. 33. FIG. 6 illustrates a sequence alignment between the variable light and heavy chains in m38c2 (SEQ ID NO:77 and 78, respectively), h38c2 (SEQ ID NOs:79 and 80, respectively), and human germlines. Human germline Vₖ gene DPK-9 (SEQ ID NO: 81) and human Jₖ gene JK4 (SEQ ID NO: 83) were used as frameworks for the humanization of the kappa light chain variable domain, and human germline gene DP-47 (SEQ ID NO:82) and human J_{H} gene JH4 (SEQ ID NO:84) were used as frameworks for the humanization of the heavy chain variable domain of m38c2. h38c2 may also use the IgG2, IgG3, or IgG4 constant domains, including any of the allotypes thereof. One embodiment of h38c2 IgG1 uses the Glm(f) allotype. Another embodiment uses a chimeric antibody comprising the variable domains (V_{L} and V_{H}) of h38c2 and the constant domains from an IgG1, IgG2, IgG3, or IgG4.

Various forms of humanized aldolase antibody fragments are also contemplated. One embodiment uses h38c2 F(ab′)₂. h38c2 F(ab′)₂ may be produced by the proteolytic digestion of h38c2 IgG1. Another embodiment uses an h38c2 scFv comprising the V_{L} and V_{H} domains from h38c2 which are optionally connected by the intervening linker (Gly₄Ser)₃.

As an alternative to humanization, human antibodies can be generated. For example, it is now possible to produce transgenic animals (e.g., mice) that are capable, upon immunization (or reactive immunization in the case of catalytic antibodies), of producing a full repertoire of human antibodies in the absence of endogenous immunoglobulin production. For example, it has been described that the homozygous deletion of the antibody heavy-chain joining region (J_{H}) gene in chimeric and germ-line immunoglobulin gene array into such germ-line mutant mice will result in the production of human antibodies upon antigen challenge. See, e.g., A. Jakobovits et al., Proc. Natl. Acad. Sci. U.S.A. 90:2551-2555 (1993); A. Jakobovits et al., Nature 362:255-258 (1993); M. Bruggemann et al., Year Immunol. 7:33-40 (1993); L. D. Taylor, et al. Nucleic Acids Res. 20:6287-6295 (1992); M. Bruggemann et al., Proc. Natl. Acad. Sci. U.S.A. 86:6709-6713 (1989)); and WO 97/17852.

Unlike typical chemical derivatization of antibodies, those derived from immunization can be specifically labeled in their binding site at a defined position, facilitating the rapid and controlled preparation of a homogeneous product. In addition, unlike chemical derivatization of antibodies, those derived from reactive immunization with 1,3-diketones are reversible. Due to this reversibility, a diketone derivative of an GA targeting compound bound to mAb 38C2 can be released from the antibody through competition with the covalent binding hapten JW (see J. Wagner et al., Science 270:1797-1800 (1995)) or related compounds. This allows one to immediately neutralize the conjugate in vivo in case of an adverse reaction. Alternatively, non-reversible covalent linkage is possible, such as with aldolase antibodies and beta lactam derivatives of the targeting compound. Unlike typical anti-hapten antibodies, covalent diketone binding antibodies have the advantage that the covalent linkage that is formed between the diketone and the antibody is between pH 3 and pH 11. The added stability of antibodies covalently linked to their targeting agent should provide additional advantages in terms of formulation, delivery, and long term storage.

Alternatively, phage display technology (see, e.g., J. McCafferty et al., Nature 348:552-553 (1990)) can be used to produce human antibodies and antibody fragments in vitro using immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, and is reviewed in, e.g., K. S. Johnson and D. J. Chiswell, Curr. Opin. Struct. Biol. 3:564-571 (1993). Several sources of V-gene segments can be used for phage display. T. Clackson et al., Nature 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by J. D. Marks et al., J. Mol. Biol. 222:581-597 (1991) or A. D. Griffiths et al., EMBO J. 12:725-734 (1993). See also U.S. Pat. Nos. 5,565,332 and 5,573,905; and L. S. Jespers et al., Biotechnology 12:899-903 (1994).

As indicated above, human antibodies may also be generated by in vitro activated B cells. See, e.g., U.S. Pat. Nos. 5,567,610 and 5,229,275; and C. A. K. Borrebaeck et al., Proc. Natl. Acad. Sci. U.S.A. 85:3995-3999 (1988).

Amino acid sequence modification(s) of the antibodies described herein are contemplated. For example, it may be desirable to improve the binding affinity and/or other biological properties of the antibody. Amino acid sequence variants of an antibody are prepared by introducing appropriate nucleotide changes into the antibody nucleic acid, or by peptide synthesis. Such modifications include, for example, deletions from, insertions into, and/or substitutions of residues within the amino acid sequences of the antibody. Any combination of deletion, insertion, and substitution is made to arrive at the final construct, provided that the final construct possesses the desired characteristics. The amino acid changes also may alter post-translational processes of the antibody, such as changing the number or position of glycosylation sites.

A useful method for identification of certain residues or regions of an antibody that are preferred locations for mutagenesis is called “alanine scanning mutagenesis,” as described in B. C. Cunningham and J. A. Wells, Science 244:1081-1085 (1989). Here, a residue or group of target residues are identified (e.g., charged residues such as Arg, Asp, His, Lys, and Glu) and replaced by a neutral or negatively charged amino acid (most preferably Ala or Polyalanine) to affect the interaction of the amino acids with the Z group of the linker. Those amino acid locations demonstrating functional sensitivity to the substitutions are then refined by introducing further or other variants at, or for, the sites of substitution. Thus, while the site for introducing an amino acid sequence variation is predetermined, the nature of the mutation per se need not be predetermined. For example, to analyze the performance of a mutation at a given site, alanine scanning or random mutagenesis is conducted at the target codon or region and the expressed antibody variants are screened for the ability to form a covalent bond with Z.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of an antibody molecule include the fusion to the N- or C-terminus of an antibody to an enzyme or a polypeptide which increases the serum half-life of the antibody.

Another type of variant is an amino acid substitution variant. These variants have at least one amino acid residue in an antibody molecule replaced by a different residue. The sites of greatest interest for substitutional mutagenesis include the hypervariable regions, but FR alterations are also contemplated. Conservative substitutions are shown in the table below under the heading of “preferred substitutions.” If such substitutions result in a change in biological activity, then more substantial changes, denominated “exemplary substitutions” as further described below in reference to amino acid classes, may be introduced and the products screened.

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the sidechain. Naturally occurring residues are divided into groups based on common side-chain properties:

(1) hydrophobic: Nle, Met, Ala, Val, Leu, Ile;

(2) neutral hydrophilic: Cys, Ser, Thr;

(3) acidic: Asp, Glu;

(4) basic: Asn, Gln, His, Lys, Arg;

(5) residues that influence chain orientation: Gly, Pro; and

(6) aromatic: Trp, Tyr, Phe.

Non-conservative substitutions will entail exchanging a member of one of these classes for a member of another class.

Any cysteine residue not involved in maintaining the proper conformation of the antibody may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

One type of substitutional variant involves substituting one or more hypervariable region residues of a parent antibody (e.g., a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a structure of the antibody conjugate complex to identify contact points between the antibody and the Z group. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody by deleting one or more carbohydrate moieties found in the antibody and/or adding one or more glycosylation sites that are not present in the antibody.

Glycosylation of antibodies is typically either N-linked or O-linked. N-linked refers to the attachment of the carbohydrate moiety to the sidechain of an asparagine residue. The tripeptide sequences Asn-X-Ser and Asn-X-Thr, where X is any amino acid except proline, are the recognition sequences for enzymatic attachment of the carbohydrate moiety to the asparagine sidechain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of or substitution by one or more serine or threonine residues to the sequence of the original antibody (for O-linked glycosylation sites).

It may be desirable to modify an antibody of the invention with respect to effector function, for example to enhance or decrease antigen-dependent cell-mediated cytotoxicity (ADCC) and/or complement dependent cytotoxicity (CDC) of the antibody. This may be achieved by introducing one or more amino acid substitutions in an Fc region of the antibody. Alternatively, an antibody can be engineered which has dual Fc regions and may thereby have enhanced complement lysis and ADCC capabilities. See G. T. Stevenson et al., Anticancer Drug Des. 3:219-230 (1989).

To increase the serum half life of an antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

Amino Acid Substitutions

| Original Residue | Exemplary Substitutions | Preferred Substitutions |
|---|---|---|
| Ala (A) | Val; Leu; Ile | Val |
| Arg (R) | Lys; Gln; Asn | Lys |
| Asn (N) | Gln; His; Asp; Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Nle | Leu |
| Leu (L) | Nle; Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Tyr |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Nle | Leu |

### Linkers and Linked Compounds

A GA targeting agent as herein described may be covalently linked to a combining site in an antibody either directly or via a linker. An appropriate linker can be chosen to provide sufficient distance between the targeting agent and the antibody. The general design of one embodiment of a linker for use in preparing GA targeting compounds is shown in the formula: X—Y-Z, wherein X is a connecting chain, Y is a recognition group and Z is a reactive group. The linker may be linear or branched, and optionally includes one or more carbocyclic or heterocyclic groups. Linker length may be viewed in terms of the number of linear atoms, with cyclic moieties such as aromatic rings and the like to be counted by taking the shortest route around the ring. In certain embodiments, the linker has a linear stretch of between 5-15 atoms, in other embodiments 15-30 atoms, in still other embodiments 30-50 atoms, in still other embodiments 50-100 atoms, and in still other embodiments 100-200 atoms. Other linker considerations include the effect on physical or pharmacokinetic properties of the resulting GA targeting compound or GA targeting agent-linker, solubility, lipophilicity, hydrophilicity, hydrophobicity, stability (more or less stable as well as planned degradation), rigidity, flexibility, immunogenicity, modulation of antibody binding, the ability to be incorporated into a micelle or liposome, and the like.

The connecting chain X of the linker includes any atom from the group C, H, N, O, P, S, halogen (F, Cl, Br, I), or a salt thereof. X also may include a group such as an alkyl, alkenyl, alkynyl, oxoalkyl, oxoalkenyl, oxoalkynyl, aminoalkyl, aminoalkenyl, aminoalkynyl, sulfoalkyl, sulfoalkenyl, sulfoalkynyl, phosphoalkyl, phosphoalkenyl, or phosphoalkynyl group. In some embodiments, X may include one or more ring structures. In some embodiments, the linker is a repeating polymer such as polyethylene glycol comprising 2-100 units.

The recognition group Y of the linker is optional, and if present is located between the reactive group and the connecting chain. In some embodiments, Y is located from 1-20 atoms from Z.

Although not wishing to be bound by any theory, it is believed that the recognition group acts to properly position the reactive group into the antibody combining site so that it may react with a reactive amino acid sidechain. Exemplary recognition groups include carbocyclic and heterocyclic rings, preferably having five or six atoms. However, larger ring structures also may be used. In some embodiments, a GA targeting agent is linked directly to Y without the use of an intervening linker.

Z is capable of forming a covalent bond with a reactive sidechain in an antibody combining site. In some embodiments, Z includes one or more C═O groups arranged to form a diketone, an acyl beta-lactam, an active ester, a haloketone, a cyclohexyl diketone group, an aldehyde, a maleimide, an activated alkene, an activated alkyne or, in general, a molecule comprising a leaving group susceptible to nucleophilic or electrophilic displacement. Other groups may include a lactone, an anhydride, an alpha-haloacetamide, an imine, a hydrazide, or an epoxide. Exemplary linker electrophilic reactive groups that can covalently bond to a reactive nucleophilic group (e.g., a lysine or cysteine sidechain) in a combining site of antibody include acyl beta-lactam, simple diketone, succinimide active ester, maleimide, haloacetamide with linker, haloketone, cyclohexyl diketone, aldehyde, amidine, guanidine, imine, eneamine, phosphate, phosphonate, epoxide, aziridine, thioepoxide, a masked or protected diketone (a ketal for example), lactam, sulfonate, and the like, masked C═O groups such as imines, ketals, acetals, and any other known electrophilic group. In one embodiment, the reactive group includes one or more C═O groups arranged to form an acyl beta-lactam, simple diketone, succinimide active ester, maleimide, haloacetamide with linker, haloketone, cyclohexyl diketone, or aldehyde.

The linker reactive group or similar such reactive group is chosen for use with a reactive residue in a particular combining site. For example, a chemical moiety for modification by an aldolase antibody may be a ketone, diketone, beta lactam, active ester haloketone, lactone, anhydride, maleimide, alpha-haloacetamide, cyclohexyl diketone, epoxide, aldehyde, amidine, guanidine, imine, eneamine, phosphate, phosphonate, epoxide, aziridine, thioepoxide, masked or protected diketone (ketal for example), lactam, haloketone, aldehyde, and the like.

A linker reactive group chemical moiety suitable for covalent modification by a reactive sulfhydryl group in an antibody may be a disulfide, aryl halide, maleimide, alpha-haloacetamide, isocyanate, epoxide, thioester, active ester, amidine, guanidine, imine, eneamine, phosphate, phosphonate, epoxide, aziridine, thioepoxide, masked or protected diketone (ketal for example), lactam, haloketone, aldehyde, and the like.

One of skill in the art will readily appreciate that reactive amino acid sidechains in antibody combining sites may possess an electrophilic group that reacts with a nucleophilic group on a GA targeting agent or its linker, whereas in other embodiments a reactive nucleophilic group in an amino acid sidechain reacts with an electrophilic group in an GA targeting agent or linker.

A GA targeting compound may be prepared by several approaches. In one approach, a GA targeting agent-linker compound is synthesized with a linker that includes one or more reactive groups designed for covalent reaction with a sidechain of an amino acid in a combining site of an antibody. The targeting agent-linker compound and antibody are combined under conditions where the linker reactive group forms a covalent bond with the amino acid sidechain.

In another approach, linking can be achieved by synthesizing an antibody-linker compound comprising an antibody and a linker wherein the linker includes one or more reactive groups designed for covalent reaction with an appropriate chemical moiety of a GA targeting agent. A GA targeting agent may need to be modified to provide the appropriate moiety for reaction with the linker reactive group. The antibody-linker and GA targeting agent are combined under conditions where the linker reactive group covalently links to the targeting and/or biological agent.

A further approach for forming an antibody-GA targeting compound uses a dual linker design. In one embodiment, a GA targeting agent-linker compound is synthesized which comprises a GA targeting agent and a linker with a reactive group. An antibody-linker compound is synthesized which comprises an antibody and a linker with a chemical group susceptible to reactivity with the reactive group of the GA targeting agent-linker of the first step. These two linker containing compounds are then combined under conditions whereby the linkers covalently link, forming the antibody-GA-targeting compound.

Exemplary functional groups that can be involved in the linkage include, for example, esters, amides, ethers, phosphates, amino, keto, amidine, guanidine, imines, eneamines, phosphates, phosphonates, epoxides, aziridines, thioepoxides, masked or protected diketones (ketals for example), lactams, haloketones, aldehydes, thiocarbamate, thioamide, thioester, sulfide, disulfide, phosphoramide, sulfonamide, urea, thioruea, carbamate, carbonate, hydroxamide, and the like.

The linker includes any atom from the group C, H, N, O, P, S, halogen (F, Cl, Br, I), or a salt thereof. The linker also may include a group such as an alkyl, alkenyl, alkynyl, oxoalkyl, oxoalkenyl, oxoalkynyl, aminoalkyl, aminoalkenyl, aminoalkynyl, sulfoalkyl, sulfoalkenyl, sulfoalkynyl group, phosphoalkyl, phosphoalkenyl, or phosphoalkynyl group. The linker also may include one or more ring structures. As used herein a “ring structure” includes saturated, unsaturated, and aromatic carbocyclic rings and saturated, unsaturated, and aromatic heterocyclic rings. The ring structures may be mono-, bi-, or polycyclic, and include fused or unfused rings. Further, the ring structures are optionally substituted with functional groups well known in the art, including but not limited to halogen, oxo, —OH, —CHO, —COOH, —NO₂, —CN, —NH₂, —C(O)NH₂, C₁₋₆ alkyl, C₂₋₆ alkenyl, C₂₋₆ alkynyl, C₁₋₆ oxoalkyl, oxoalkenyl, oxoalkynyl, aminoalkyl, aminoalkenyl, aminoalkynyl, sulfoalkyl, sulfoalkenyl, sulfoalkynyl, phosphoalkyl, phosphoalkenyl, or phosphoalkynyl group. Combinations of the above groups and rings may also be present in the linkers of GA targeting compounds.

One aspect of the invention is a GA targeting agent-linker conjugate having Formula I:

L-[GA targeting agent] (I)

wherein [GA targeting agent] is a GA targeting agent peptide. Suitable GA targeting agent peptides include, but are not limited to, SEQ ID NO:1, SEQ ID NO:2, and analogs of SEQ ID NO:1 or SEQ ID NO:2, including for example carboxy truncations or mutations, and GA targeting compounds as herein described.

The linker moiety L may be attached to the carboxy terminus, or any electrophilic or nucleophilic sidechain of an amino acid side of a GA targeting agent. The point of attachment of L to a GA targeting agent is referred to herein as the “tethering point.”

In certain embodiments, L is linked to a nucleophilic or electrophilic sidechain of an amino acid in a GA targeting agent. Exemplary nucleophilic sidechains are Lys, Cys, Ser, Thr, and Tyr. In those embodiments wherein L is linked to a nucleophilic sidechain, L should comprise an electrophilic group susceptible to covalent reaction with the nucleophilic sidechain. Exemplary electrophilic sidechains are Asp and Glu. In those embodiments wherein L is linked to an electrophilic sidechain, L should comprise a nucleophilic group susceptible to covalent reaction with the electrophilic sidechain.

In certain embodiments wherein L is linked to a nucleophilic sidechain of an amino acid (the linking residue) in a GA targeting agent, L is linked to a nucleophilic sidechain of a Lys residue. In certain of these embodiments, the Lys residue is residue 20 or 28 of SEQ ID NO:1, or residue 12 or 27 of SEQ ID NO:2. In certain other embodiments, a Lys residue is inserted at the carboxy terminus of a GA targeting agent of SEQ ID NO:1 or SEQ ID NO:2 or an analog thereof, and the linker L is covalently attached to the sidechain of this additional amino acid. For example, in one embodiment, a GA targeting agent is:

(SEQ ID NO:3)

HGFGTFTSDLSKQMFFFAVRLFIFWLKNGGPSSGAPPPSK,
or

(SEQ ID NO:4)

HAibFGTFTSDLSKQMFFFAVRLFIFWLKNGGPSSGAPPPSK.

SEQ ID NO:3 is identical to SEQ ID NO:2 but for the insertion of a Lys residue at the carboxy terminus of the peptide. SEQ ID NO:4 is identical to SEQ ID NO:3 but for the substitution of the Gly residue at position 2 with Aib2.

Examples of compounds of Formula I comprising SEQ ID NO:3- or SEQ ID NO:4-based targeting agents include, but are not limited to:

(SEQ ID NO:166)

HGFGTFTSDLSKQMFFFAVRLFIFWLKNGGPSSGAPPPSK(L);

(SEQ ID NO:167)

HAibFGTFTSDLSKQMFFFAVRLFIFWLKNGGPSSGAPPPSK(L);
and

(SEQ ID NO:30)

R¹-HAibFGTFTSDLSKQMFFFAVRLFIFWLKNGGK-R².

In certain other embodiments, a Lys residue is inserted or substituted into a position internal to SEQ ID NO:1 or SEQ ID NO:2 or an analog thereof, and the linker L is covalently attached to the sidechain of this additional amino acid. Examples of these embodiments are set forth in Table II, below. Inserted Lys residues, which serve as tethering points for attachment of linker L are underlined.

TABLE II

SEQ ID NO:1 and SEQ ID NO:2-BASED GA
TARGETING AGENTS W/ LYSINE SUBSTITUTIONS

| | |
|---|---|
| R¹-HAibEGTFTSDLSKQMEEEAVRLFIEWLKNGGPSSGAPPKS-R² | (SEQ ID NO:14) |
| R¹-HAibEGTFTSDLSKQMEEEAVRLFIEWLKNGGPSSGAKPPS-R² | (SEQ ID NO:15) |
| R¹-HAibEGTFTSDLSKQMEEEAVRLFIEWLKNGGPSSKAPPPS-R² | (SEQ ID NO:16) |
| R¹-HAibEGTFTSDLSKQMEEEAVRLFIEWLKNGGPKSGAPPPS-R² | (SEQ ID NO:17) |
| R¹-HAibEGTFTSDLSKQMEEEAVRLFIEWLKKGGPSSGAPPPS-R² | (SEQ ID NO:18) |
| R¹-HAibEGTFTSDLSKQMEEEAVRLFIEWKKNGGPSSGAPPPS-R² | (SEQ ID NO:19) |
| R¹-HAibEGTFTSDLSKQMEEEAVRLFIKWLKNGGPSSGAPPPS-R² | (SEQ ID NO:20) |
| R¹-HAibEGTFTSDLSKQMEEEAVRLFKEWLKNGGPSSGAPPPS-R² | (SEQ ID NO:21) |
| R¹-HAibEGTFTSDLSKQMEEEAVRKFIEWLKNGGPSSGAPPPS-R² | (SEQ ID NO:22) |
| R¹-HAibEGTFTSDLSKQMEEEAVKLFIEWLKNGGPSSGAPPPS-R² | (SEQ ID NO:23) |
| R¹-HAibEGTFTSDLSKQMEEEAKRLFIEWLKNGGPSSGAPPPS-R² | (SEQ ID NO:24) |
| R¹-HAibEGTFTSDLSKQMEEKAVRLFIEWLKNGGPSSGAPPPS-R² | (SEQ ID NO:25) |
| R¹-HAibEGTFTSDLSKQMEKEAVRLFIEWLKNGGPSSGAPPPS-R² | (SEQ ID NO:26) |
| R¹-HAibEGTFTSDLSKQKEEEAVRLFIEWLKNGGPSSGAPPPS-R² | (SEQ ID NO:27) |
| R¹-HAibEGTFTSDLSKKMEEEAVRLFIEWLKNGGPSSGAPPPS-R² | (SEQ ID NO:28) |
| R¹-HAibEGTFTSDLKKQMEEEAVRLFIEWLKNGGPSSGAPPPS-R² | (SEQ ID NO:29) |
| R¹-HAibEGTFTSDLSKQMEEEAVRKFIEWLKNGGPSS-R² | (SEQ ID NO:31) |
| R¹-HAibEGTFTSDVSSYLEKQAAKEFIAWLVKAibR-R² | (SEQ ID NO:47) |
| R¹-HAibEGTFTSDKSSYLEEQAVKEFIAWLIKAibR-R² | (SEQ ID NO:64) |
| R¹-HAibEGTFTSDVSKYLEEQAVKEFIAWLIKAibR-R² | (SEQ ID NO:65) |
| R¹-HAibEGTFTSDVSSYKEEQAVKEFIAWLIKAibR-R² | (SEQ ID NO:66) |
| R¹-HAibEGTFTSDVSSYLEKQAVKEFIAWLIKAibR-R² | (SEQ ID NO:67) |
| R¹-HAibEGTFTSDVSSYLEEQKVKEFIAWLIKAibR-R² | (SEQ ID NO:68) |
| R¹-HAibEGTFTSDVSSYLEEQAVKEKIAWLIKAibR-R² | (SEQ ID NO:69) |
| R¹-HAibEGTFTSDVSSYLEEQAVKEFIKWLIKAibR-R² | (SEQ ID NO:70) |
| R¹-HAibEGTFTSDVSSYLEEQAVKEFIAWKIKAibR-R² | (SEQ ID NO:71) |

In those embodiments wherein the linker L is covalently attached to a Lys residue in the GA targeting peptide, the Lys residue may be a sidechain modified Lys. In certain of these embodiments, the sidechain modified Lys is:

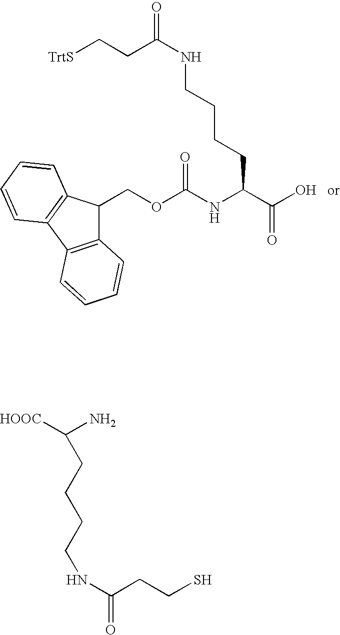

In compounds of Formula I, L is a linker moiety having the formula —X—Y-Z, wherein:

X is a biologically compatible polymer or block copolymer attached to one of the residues that comprises a GA targeting agent;

Y is an optionally present recognition group comprising at least a ring structure; and

Z is a reactive group that is capable of covalently linking to a sidechain in a combining site of an antibody.

In some embodiments of compounds in Formula I, X is:

—R²²—P—R²⁻— or —R²²—P—R²¹—P′—R²³—

wherein:

P and P′ are independently selected from the group consisting of polyoxyalkylene oxides such as polyethylene oxide, polyethyloxazoline, poly-N-vinyl pyrrolidone, polyvinyl alcohol, polyhydroxyethyl acrylate, polyhydroxy ethylmethacrylate and polyacrylamide, polyamines having amine groups on either the polymer backbone or the polymer sidechains, such as polylysine, polyornithine, polyarginine, and polyhistidine, nonpeptide polyamines such as polyaminostyrene, polyaminoacrylate, poly(N-methyl aminoacrylate), poly(N-ethylaminoacrylate), poly(N,N-dimethyl aminoacrylate), poly(N,N-diethylaminoacrylate), poly(aminomethacrylate), poly(N-methyl amino-methacrylate), poly(N-ethyl aminomethacrylate), poly(N,N-dimethyl aminomethacrylate), poly(N,N-diethyl aminomethacrylate), poly(ethyleneimine), polymers of quaternary amines, such as poly(N,N,N-trimethylaminoacrylate chloride), poly(methyacrylamidopropyltrimethyl ammonium chloride), proteoglycans such as chondroitin sulfate-A (4-sulfate) chondroitin sulfate-C (6-sulfate) and chondroitin sulfate-B, polypeptides such as polyserine, polythreonine, polyglutamine, natural or synthetic polysaccharides such as chitosan, hydroxy ethyl cellulose, and lipids;

R²¹, R²², and R²³ are each independently a covalent bond, —O—, —S—, —NR^{b}—, substituted or unsubstituted straight or branched chain C₁₋₅₀ alkylene, or substituted or unsubstituted straight or branched chain C₁₋₅₀ heteroalkylene;

R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl; and

R²¹, R²², and R²³ are selected such that the backbone length of X remains about 200 atoms or less.

In some embodiments of compounds of Formula I, R²² is —(CH₂)ᵥ—, —(CH₂)ᵤ—C(O)—(CH₂)ᵥ—, —(CH₂)ᵤ—C(O)—O—(CH₂)ᵥ—, —(CH₂)ᵤ—C(S)—NR^{b}—(CH₂)ᵥ—, —(CH₂)ᵤ—C(O)—NR^{b}—(CH₂)ᵥ—, —(CH₂)ᵤ—NR^{b}—(CH₂)ᵥ—, —(CH₂)ᵤ—O—(CH₂)ᵥ—, —(CH₂)ᵤ—S(O)₀₋₂—(CH₂)ᵥ—, —(CH₂)ᵤ—S(O)₀₋₂—NR^{b}—(CH₂)ᵥ—, or —(CH₂)ᵤ—P(O)(OR^{b})—O—(CH₂)ᵥ—, wherein u and v are each independently 0, 1, 2, 3, 4, 5, 6, 7, 8, 9, 10, 11, 12, 13, 14, 15, 16, 17, 18, 19 or 20.

In yet other embodiments of compounds of Formula I, R²² is —(CH₂)ᵥ—, —(CH₂)ᵤ—C(O)—(CH₂)ᵥ—, —(CH₂)ᵤ—C(O)—O—(CH₂)ᵥ—, —(CH₂)ᵤ—C(O)—NR^{b}—(CH₂)ᵥ—, or —(CH₂)ᵤ—NR^{b}—(CH₂)ᵥ. In still other embodiments, R⁻² is —(CH₂)ᵤ—C(O)—NR^{b}—(CH₂)ᵥ—.

In some embodiments of compounds of Formula I, R²¹ and R²³ are each independently —(CH₂)ₛ—, —(CH₂)ᵣ—C(O)—(CH₂)ₛ—, —(CH₂)ᵣ—C(O)—O—(CH₂)ᵥ—, —(CH₂)ᵣ—C(S)—NR^{b}—(CH₂)ₛ—, —(CH₂)ᵣ—C(O)—NR^{b}—(CH₂)ₛ—, —(CH₂)ᵣ—NR^{b}—(CH₂)ₛ—, —(CH₂)ᵣ—O—(CH₂)ₛ—, —(CH₂)ᵣ—S(O)₀₋₂—(CH₂)ₛ—, —(CH₂)ᵣ—S(O)₀₋₂—NR^{b}—(CH₂)ₛ—, or —(CH₂)ᵣ—P(O)(OR^{b})—O—(CH₂)ₛ—, wherein r, s, and v are each independently 0, 1, 2, 3, 4, 5, 6, 7, 8, 9, 10, 11, 12, 13, 14, 15, 16, 17, 18, 19 or 20.

In yet other embodiments, R²¹ and R²³ are each independently —(CH₂)ₛ—, —(CH₂)ᵣ—C(O)—(CH₂)ₛ—, —(CH₂)ᵣ—C(O)—O—(CH₂)ₛ—, —(CH₂)—C(O)—NR^{b}—(CH₂)ₛ—, or —(CH₂)ᵣ—NR^{b}—(CH₂)ₛ, and —(CH₂)ᵣ—C(O)—NR^{b}—(CH₂)ₛ—.

In still other embodiments, R²¹ and R²³ each independently have the structure:

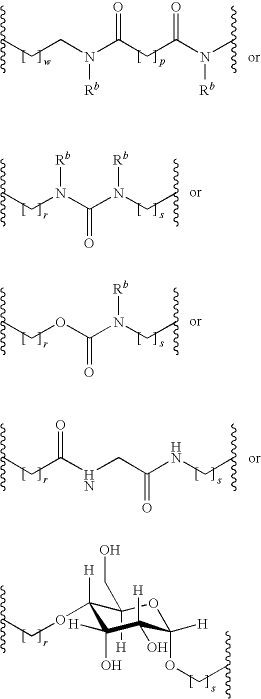

wherein p is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 32, 43, 44, or 45; w, r, and s are each independently 0, 1, 2, 3, 4, 5, 6, 7, 8, 9, 10, 11, 12, 13, 14, 15, 16, 17, 18, 19 or 20; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl.

In certain embodiments of compounds of Formula I, X has the structure:

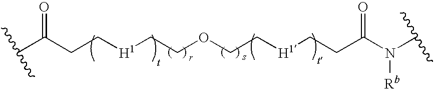

wherein H¹ and H^{1′} at each occurrence are independently N, O, S, or CH₂; r and s are each independently 1, 2, 3, 4, 5, 6, 7, 8, 9, 10, 11, 12, 13, 14, 15, 16, 17, 18, 19, or 20; t and t′ are each independently 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 32, 43, 44, 45, 46, 47, 48, 49 or 50; and R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl.

In certain embodiments of compounds of Formula I, X has the structure:

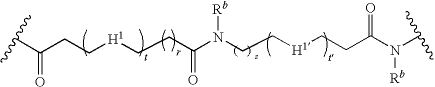

wherein H¹ and H^{1′} at each occurrence are independently N, O, S, or CH₂; r and s are each independently 1, 2, 3, 4, 5, 6, 7, 8, 9, 10, 11, 12, 13, 14, 15, 16, 17, 18, 19, or 20; t and t′ are each independently 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 32, 43, 44, 45, 46, 47, 48, 49 or 50; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl.

In certain embodiments of compounds of Formula I, X has the structure:

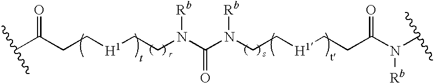

wherein H¹ and H^{1′} at each occurrence are independently N, O, S, or CH₂; r and s are each independently 1, 2, 3, 4, 5, 6, 7, 8, 9, 10, 11, 12, 13, 14, 15, 16, 17, 18, 19, or 20; t and t′ are each independently 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 32, 43, 44, 45, 46, 47, 48, 49 or 50; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl.

In certain embodiments of compounds of Formula I, X has the structure:

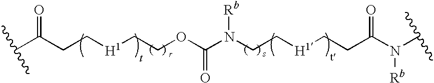

wherein H¹ and H^{1′} at each occurrence are independently N, O, S, or CH₂; r and s are each independently 1, 2, 3, 4, 5, 6, 7, 8, 9, 10, 11, 12, 13, 14, 15, 16, 17, 18, 19, or 20; t and t′ are each independently 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 32, 43, 44, 45, 46, 47, 48, 49 or 50; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl.

In certain embodiments of compounds of Formula I, X has the structure:

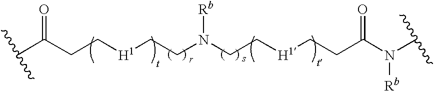

wherein H¹ and H^{1′} at each occurrence are independently N, O, S, or CH₂; r and s are each independently 1, 2, 3, 4, 5, 6, 7, 8, 9, 10, 11, 12, 13, 14, 15, 16, 17, 18, 19, or 20; t and t′ are each independently 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 32, 43, 44, 45, 46, 47, 48, 49 or 50; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl.

In certain embodiments of compounds of Formula I, X has the structure:

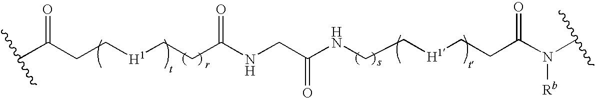

wherein H¹ and H^{1′} at each occurrence are independently N, O, S, or CH₂; r and s are each independently 1, 2, 3, 4, 5, 6, 7, 8, 9, 10, 11, 12, 13, 14, 15, 16, 17, 18, 19, or 20; t and t′ are each independently 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 32, 43, 44, 45, 46, 47, 48, 49 or 50; and R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl.

In certain embodiments of compounds of Formula I, X has the structure:

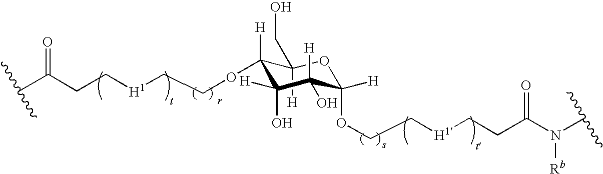

wherein H′ and H″ at each occurrence are independently N, O, S, or CH₂; r and s are each independently 1, 2, 3, 4, 5, 6, 7, 8, 9, 10, 11, 12, 13, 14, 15, 16, 17, 18, 19, or 20; t and t′ are each independently 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 32, 43, 44, 45, 46, 47, 48, 49 or 50; and R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl.

In certain embodiments of compounds of Formula I, X has the structure:

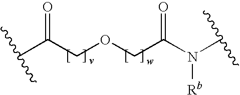

wherein v and w are each independently 1, 2, 3, 4, or 5 and R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain of these embodiments, v is 1, 2 or 3, w is 1, 2, or 3, and R^{b} is hydrogen.

In certain embodiments of Formula I, L is a linker moiety having the formula —X—Y-Z, wherein:

X is attached to one of the residues that comprises a GA targeting agent, and is an optionally substituted —R²²—[CH₂—CH₂—O]ₜ—R²³—, —R²²-cycloalkyl-R²³—, —R²²-aryl-R²³—, or R²²-heterocyclyl-R²³—, wherein;
R²² and R²³ are each independently a covalent bond, —O—, —S—, —NR^{b}—, substituted or unsubstituted straight or branched chain C₁₋₅₀ alkylene, substituted or unsubstituted straight or branched chain C₁₋₅₀ heteroalkylene, substituted or unsubstituted straight or branched chain C₂₋₅₀ alkenylene, or substituted or unsubstituted C₂₋₅₀ heteroalkenylene;
R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl;
t is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 32, 43, 44, 45, 46, 47, 48, 49 or 50;
and the size of R²² and R²³ are such that the backbone length of X remains about 200 atoms or less;

Y is an optionally present recognition group comprising at least a ring structure; and

Z is a reactive group that is capable of covalently linking to a sidechain in a combining site of an antibody.

In some embodiments of compounds of Formula I, if t>1 or if X is —R²²-cycloalkyl-R²³—R²²-aryl-R²³—, or R²²-heterocyclyl-R²³—, Y is present.

In some embodiments of compounds of Formula I, X is:

—R²²—[CH₂—CH₂—O]ₜ—R²³—,

wherein:

R²² is —(CH₂)ᵥ—, —(CH₂)ᵤ—C(O)—(CH₂)ᵥ—, —(CH₂)ᵤ—C(O)—O—(CH₂)ᵥ—, —(CH₂)ᵤ—C(O)—NR^{b}—(CH₂)ᵥ—, —(CH₂)ᵤ—C(S)—NR^{b}—(CH₂)ᵥ—, —(CH₂)ᵤ—NR^{b}—(CH₂)ᵥ—, —(CH₂)ᵤ—O—(CH₂)ᵥ—, —(CH₂)ᵤ—S(O)₀₋₂—(CH₂)ᵥ—, —(CH₂)ᵤ—S(O)₀₋₂—N^{b}—(CH₂)ᵥ—, or —(CH₂)ᵤ—P(O)(OR^{b})—O—(CH₂)ᵥ—;

u and v are each independently 0, 1, 2, 3, 4, 5, 6, 7, 8, 9, 10, 11, 12, 13, 14, 15, 16, 17, 18, 19 or 20 and t is 0 to 50.
R²³ has the structure:

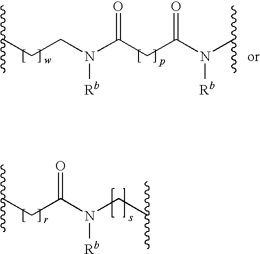

wherein:

p is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 32, 43, 44, or 45;

w and r are each independently 1, 2, 3, 4, 5, 6, 7, 8, 9, 10, 11, 12, 13, 14, 15, 16, 17, 18, 19 or 20;

s is 0, 1, 2, 3, 4, 5, 6, 7, 8, 9, 10, 11, 12, 13, 14, 15, 16, 17, 18, 19 or 20; and

R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl;

and the values of t, u, w, p, v, r and s are such that the backbone length of X remains about 200 atoms or less.

In one embodiment of compounds of Formula I, X has the formula:

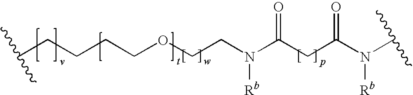

wherein the values of v, t, w, and p are selected such that the backbone length of X is less than 200 atoms, alternatively is less than 100 atoms, alternatively is less than 75 atoms, or alternatively, is less than 50 atoms.

In another embodiment of compounds of Formula I, X has the formula:

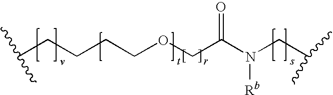

wherein the values of v, t, r, and s are selected such that the backbone length of X is less than 200 atoms, alternatively is less than 100 atoms, alternatively is less than 75 atoms, or alternatively, is less than 50 atoms.

In another embodiment of compounds of Formula I, X has the formula:

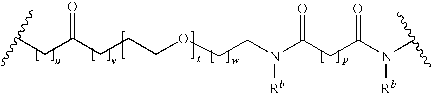

wherein the values of u, v, t, w, and p are selected such that the backbone length of X is less than 200 atoms, alternatively is less than 100 atoms, alternatively is less than 75 atoms, or alternatively, is less than 50 atoms.

In another embodiment of compounds of Formula I, X has the formula:

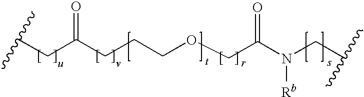

wherein the values of u, v, t, r, and s are selected such that the backbone length of X is less than 200 atoms, alternatively is less than 100 atoms, alternatively is less than 75 atoms, or alternatively, is less than 50 atoms.

In another embodiment of compounds of Formula I, X has the formula:

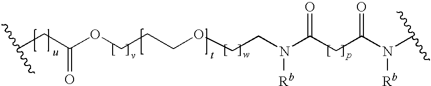

wherein the values of u, v, t, w, and p are selected such that the backbone length of X is less than 200 atoms, alternatively is less than 100 atoms, alternatively is less than 75 atoms, or alternatively, is less than 50 atoms.

In another embodiment of compounds of Formula I, X has the formula:

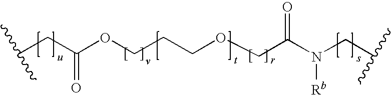

wherein the values of u, v, t, r, and s are selected such that the backbone length of X is less than 200 atoms, alternatively is less than 100 atoms, alternatively is less than 75 atoms, or alternatively, is less than 50 atoms.

In certain embodiments of compounds of Formula I, X has the structure:

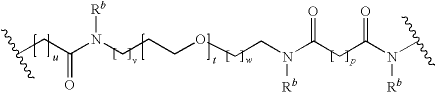

wherein the values of u, v, t, w, and p are selected such that the backbone length of X is less than 200 atoms, alternatively is less than 100 atoms, alternatively is less than 75 atoms, alternatively is less than 50 atoms, alternatively is less than 25 atoms, or alternatively is less than 15 atoms.

In certain embodiments of compounds of Formula I, X has the structure:

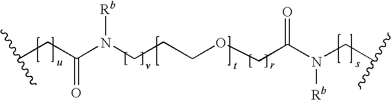

wherein the values of u, v, t, r, and s are selected such that the backbone length of X is less than 200 atoms, alternatively is less than 100 atoms, alternatively is less than 75 atoms, alternatively is less than 50 atoms, alternatively is less than 25 atoms, or alternatively is less than 15 atoms.

In certain embodiments of compounds of Formula I, X has the structure:

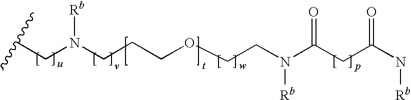

wherein the values of u, v, t, w, and p are selected such that the backbone length of X is less than 200 atoms, alternatively is less than 100 atoms, alternatively is less than 75 atoms, alternatively is less than 50 atoms, alternatively is less than 25 atoms, or alternatively is less than 15 atoms.

In certain embodiments of compounds of Formula I, X has the structure:

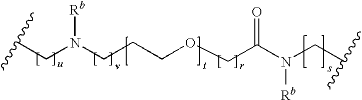

wherein the values of u, v, t, r, and s are selected such that the backbone length of X is less than 200 atoms, alternatively is less than 100 atoms, alternatively is less than 75 atoms, alternatively is less than 50 atoms, alternatively is less than 25 atoms, or alternatively is less than 15 atoms.

In another embodiment of compounds of Formula I, X has the formula:

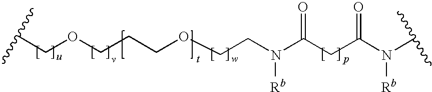

wherein the values of u, v, t, w, and p are selected such that the backbone length of X is less than 200 atoms, alternatively is less than 100 atoms, alternatively is less than 75 atoms, or alternatively, is less than 50 atoms.

In another embodiment of compounds of Formula I, X has the formula:

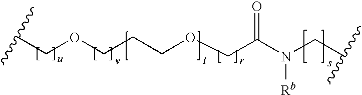

wherein the values of u, v, t, r, and s are selected such that the backbone length of X is less than 200 atoms, alternatively is less than 100 atoms, alternatively is less than 75 atoms, or alternatively, is less than 50 atoms.

In compounds having Formula I wherein L has the formula X—Y-Z, the ring structure of Y includes saturated, unsaturated, and aromatic carbocyclic rings and saturated, unsaturated, and aromatic heterocyclic rings. The ring structure(s) may be mono-, bi-, or polycyclic, and include fused or unfused rings. Further, the ring structure(s) is optionally substituted with functional groups well known in the art including, but not limited to halogen, oxo, —OH, —CHO, —COOH, —NO₂, —CN, —NH₂, amidine, guanidine, hydroxylamine, —C(O)NH₂, secondary and tertiary amides, sulfonamides, substituted or unsubstituted alkyl, substituted or unsubstituted alkenyl, substituted or unsubstituted alkynyl, oxoalkyl, oxoalkenyl, oxoalkynyl, aminoalkyl, aminoalkenyl, aminoalkynyl, sulfoalkyl, sulfoalkenyl, sulfoalkynyl, phosphoalkyl, phosphoalkenyl, and phosphoalkynyl groups.

In some embodiments of compounds having Formula I, the ring structure of Y has the optionally substituted formula:

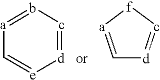

wherein

a, b, c, d, and e are independently carbon or nitrogen; and

f is carbon, nitrogen, oxygen, or sulfur;

Y is attached to X and Z independently at any two ring positions of sufficient valence; and

no more than four of a, b, c, d, e, or f are simultaneously nitrogen.

Any open valences remaining on atoms constituting the ring structure may be filled by hydrogen or other substituents, or by the covalent attachments to X and Z. For example, if b is carbon, its valence may be filled by hydrogen, a substituent such as halogen, a covalent attachment to X, or a covalent attachment to Z. In some embodiments, a, b, c, d, and e are each carbon, while in others, a, c, d and f are each carbon. In other embodiments, at least one of a, b, c, d, or e is nitrogen, and in still others, f is oxygen or sulfur. In yet another embodiment, the ring structure of Y is unsubstituted. In one embodiment, Y is phenyl.

In certain embodiments of compounds of Formula I, X—Y has the structure:

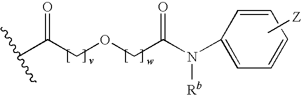

In certain of these embodiments, v is 1, 2, 3, 4, or 5; w is 1, 2, 3, 4, or 5; and R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain other embodiments, v is 1, 2 or 3 and w is 1, 2, or 3. In still other embodiments, v is 1 or 2 and w is 1 or 2.

In certain embodiments of compounds of Formula I, X—Y has the structure:

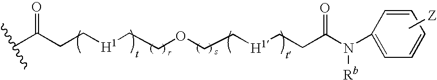

wherein H′ and H″ are each independently N, O, S, or CH₂; r and s are each independently 1, 2, 3, 4, or 5; and t and t′ are each independently 0, 1, 2, 3, 4, or 5. In certain of these embodiments, H′ and H″ are each independently O or CH₂; r and s are each independently 1 or 2; and t and t′ are each independently 0 or 1.

In certain embodiments of compounds of Formula I, X—Y has the structure:

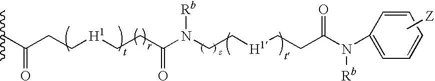

wherein H¹ and H^{1′} are each independently N, O, S, or CH₂; r and s are each independently 1, 2, 3, 4, or 5; t and t′ are each independently 0, 1, 2, 3, 4, or 5, and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain of these embodiments, H¹ and H^{1′} are each independently O or CH₂; r and s are each independently 1 or 2; and t and t′ are each independently 0 or 1.

In certain embodiments of compounds of Formula I, X—Y has the structure:

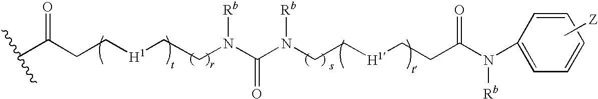

wherein H¹ and H^{1′} are each independently N, O, S, or CH₂; r and s are each independently 1, 2, 3, 4, or 5; t and t′ are each independently 0, 1, 2, 3, 4, or 5, and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain of these embodiments, H¹ and H^{1′} are each independently O or CH₂; r and s are each independently 1 or 2; and t and t′ are each independently 0 or 1.

In certain embodiments of compounds of Formula I, X—Y has the structure:

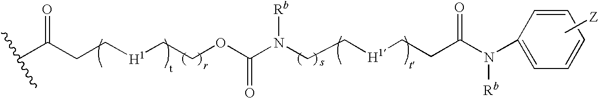

wherein H¹ and H^{1′} are each independently N, O, S, or CH₂; r and s are each independently 1, 2, 3, 4, or 5; t and t′ are each independently 0, 1, 2, 3, 4, or 5, and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain of these embodiments, H¹ and H^{1′} are each independently O or CH₂; r and s are each independently 1 or 2; and t and t′ are each independently 0 or 1.

In certain embodiments of compounds of Formula I, X—Y has the structure:

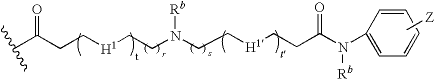

wherein H¹ and H^{1′} are each independently N, O, S, or CH₂; r and s are each independently 1, 2, 3, 4, or 5; t and t′ are each independently 0, 1, 2, 3, 4, or 5, and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain of these embodiments, H¹ and H^{1′} are each independently O or CH₂; r and s are each independently 1 or 2; and t and t′ are each independently 0 or 1.

In certain embodiments of compounds of Formula I, X—Y has the structure:

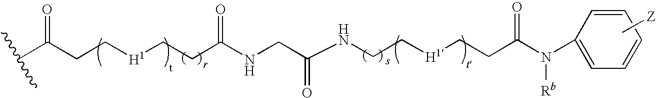

wherein H¹ and H^{1′} are each independently N, O, S, or CH₂; r and s are each independently 1, 2, 3, 4, or 5; t and t′ are each independently 0, 1, 2, 3, 4, or 5, and R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain of these embodiments, H¹ and H^{1′} are each independently O or CH₂; r and s are each independently 1 or 2; and t and t′ are each independently 0 or 1.

In certain embodiments of compounds of Formula I, X—Y has the structure:

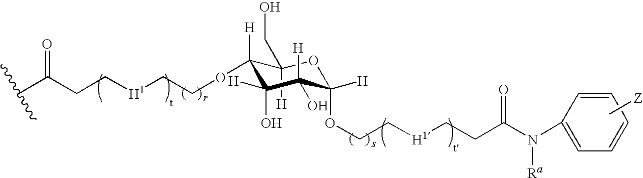

wherein H¹ and H^{1′} are each independently N, O, S, or CH₂; r and s are each independently 1, 2, 3, 4, or 5; and t and t′ are each independently 0, 1, 2, 3, 4, or 5. In certain of these embodiments, H¹ and H^{1′} are each independently O or CH₂; r and s are each independently 1 or 2; and t and t′ are each independently 0 or 1.

In certain of these embodiments of compounds of Formula I, X—Y has the structure:

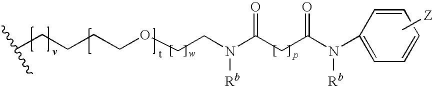

In certain of these embodiments, v is 0, 1, 2, 3, 4, or 5; t is 1, 2, 3, 4, 5, or 6; w is 1, 2, 3, 4, or 5; p is 1, 2, 3, 4, or 5, and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; w is 1; and p is 3. In some embodiments, v is 0; t is 1, 2, or 3,w is 1; and p is 1 or 2.

In certain embodiments of compounds of Formula I, X—Y has the structure:

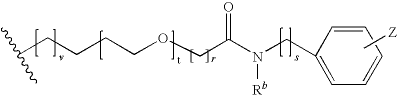

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; and R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; r is 1 or 2; and s is 3. In some embodiments, v is 0; t is 1, 2, or 3, r is 1; and s is 1 or 2.

In certain embodiments of compounds of Formula I, X—Y has the structure:

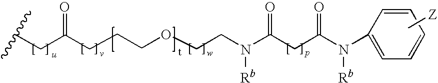

In certain of these embodiments, u is 0, 1, 2, 3, 4, or 5; v is 0, 1, 2, 3, 4, or 5; t is 1, 2, 3, 4, 5, or 6; w is 1, 2, 3, 4, or 5; p is 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; w is 1; and p is 3. In some embodiments, u is 0 or 1; v is 0; t is 1 or 2; w is 1; and p is 1 or 2.

In certain embodiments of compounds of Formula I, X—Y has the structure:

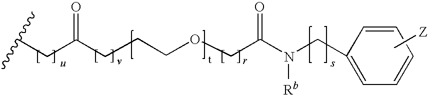

In certain of these embodiments, u is 0, 1, 2, 3, 4, or 5; v is 0, 1, 2, 3, 4, or 5; t is 1, 2, 3, 4, 5, or 6; r is 1, 2, 3, 4, or 5; s is 0, 1, 2, 3, 4, or 5; and R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; r is 1 or 2; and s is 3. In some embodiments, u is 0 or 1; v is 0; t is 1, 2, or 3; r is 1; and s is 1 or 2.

In certain embodiments of compounds of Formula I, X—Y has the structure:

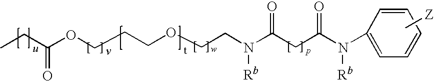

In certain of these embodiments, u is 0, 1, 2, 3, 4, or 5; v is 0, 1, 2, 3, 4, or 5; t is 1, 2, 3, 4, 5, or 6; w is 1, 2, 3, 4, or 5; p is 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; w is 1; and p is 3. In some embodiments, u is 0 or 1; v is 0; t is 1 or 2; w is 1; and p is 1 or 2.

In certain embodiments of compounds of Formula I, X—Y has the structure:

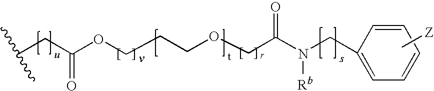

In certain of these embodiments, u is 0, 1, 2, 3, 4, or 5; v is 0, 1, 2, 3, 4, or 5; t is 1, 2, 3, 4, 5, or 6; r is 1, 2, 3, 4, or 5; s is 0, 1, 2, 3, 4, or 5; and R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; r is 1 or 2; and s is 3. In some embodiments, u is 0 or 1; v is 0; t is 1, 2, or 3, r is 1; and s is 1 or 2.

In certain embodiments of compounds of Formula I, X—Y has the structure:

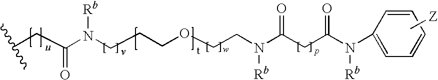

In certain of these embodiments, u is 0, 1, 2, 3, 4, or 5; v is 0, 1, 2, 3, 4, or 5; t is 1, 2, 3, 4, 5, or 6; w is 1, 2, 3, 4, or 5; p is 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; w is 1; and p is 3. In some embodiments, u is 0 or 1; v is 0; t is 1 or 2; w is 1; and p is 1 or 2.

In certain embodiments of compounds of Formula I, X—Y has the structure:

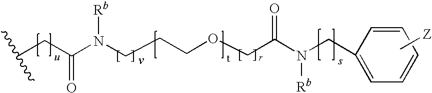

In certain of these embodiments, u is 0, 1, 2, 3, 4, or 5; v is 0, 1, 2, 3, 4, or 5; t is 1, 2, 3, 4, 5, or 6; r is 1, 2, 3, 4, or 5; s is 0, 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; r is 1 or 2; and s is 3. In some embodiments, u is 0 or 1; v is 0; t is 1, 2, or 3, r is 1; and s is 1 or 2.

In certain embodiments of compounds of Formula I, X—Y has the structure:

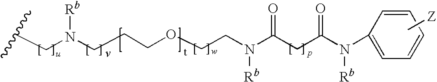

In certain of these embodiments, u is 0, 1, 2, 3, 4, or 5; v is 0, 1, 2, 3, 4, or 5; t is 1, 2, 3, 4, 5, or 6; w is 1, 2, 3, 4, or 5; p is 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; w is 1; and p is 3. In some embodiments, u is 0 or 1; v is 0; t is 1 or 2; w is 1; and p is 1 or 2.

In certain embodiments of compounds of Formula I, X—Y has the structure:

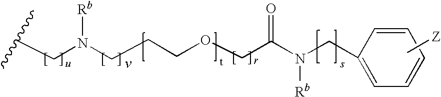

In certain of these embodiments, u is 0, 1, 2, 3, 4, or 5; v is 0, 1, 2, 3, 4, or 5; t is 1, 2, 3, 4, 5, or 6; r is 1, 2, 3, 4, or 5; s is 0, 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; r is 1 or 2; and s is 3. In some embodiments, u is 0 or 1; v is 0; t is 1, 2, or 3, r is 1; and s is 1 or 2.

In certain embodiments of compounds of Formula I, X—Y has the structure:

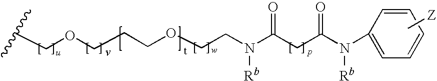

In certain of these embodiments, u is 0, 1, 2, 3, 4, or 5; v is 0, 1, 2, 3, 4, or 5; t is 1, 2, 3, 4, 5, or 6; w is 1, 2, 3, 4, or 5; p is 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; w is 1; and p is 3. In some embodiments, u is 0 or 1; v is 0; t is 1 or 2; w is 1; and p is 1 or 2.

In certain embodiments of compounds of Formula I, X—Y has the structure:

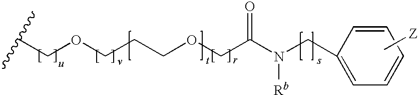

In certain of these embodiments, u is 0, 1, 2, 3, 4, or 5; v is 0, 1, 2, 3, 4, or 5; t is 1, 2, 3, 4, 5, or 6; r is 1, 2, 3, 4, or 5; s is 0, 1, 2, 3, 4, or 5; and R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiment, u is 0; v is 0; t is 1, 2, 3, 4, 5, or 6; r is 1 or 2; and s is 3. In some embodiments, u is o or 1; v is 0; t is 1, 2, or 3, r is 1; and s is 1 or 2.

In one embodiment X—Y has the formula:

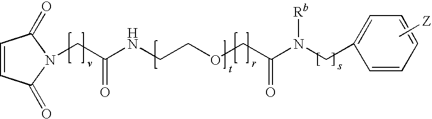

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; and R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; r is 1 or 2; s is 0 or 1.

In compounds having Formula I wherein L has the formula X—Y-Z, the reactive group Z contains a moiety capable of forming a covalent linkage with an amino acid in a combining site of an antibody. For example, Z may be substituted alkyl, substituted cycloalkyl, substituted aryl, substituted arylalkyl, substituted heterocyclyl, or substituted heterocyclylalkyl, wherein at least one substituent is a 1,3-diketone moiety, an acyl beta-lactam, an active ester, an alpha-haloketone, an aldehyde, a maleimide, a lactone, an anhydride, an alpha-haloacetamide, an amine, a hydrazide, or an epoxide. In some such embodiments, Z is substituted alkyl.

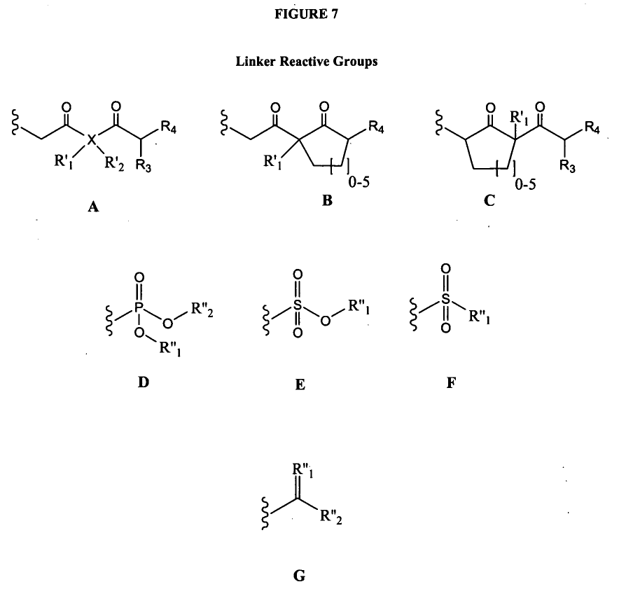

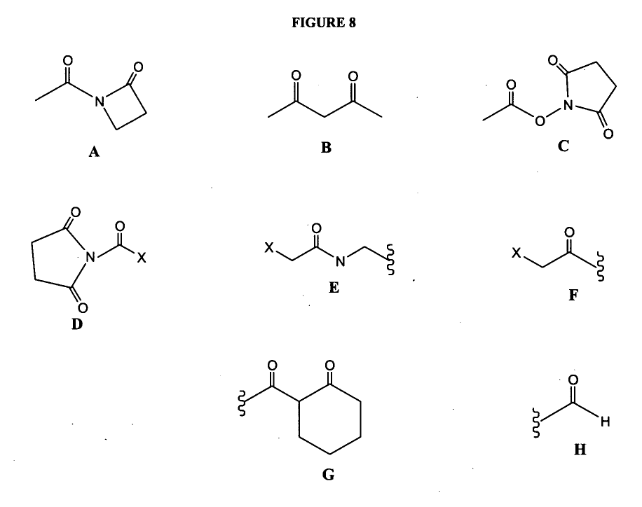

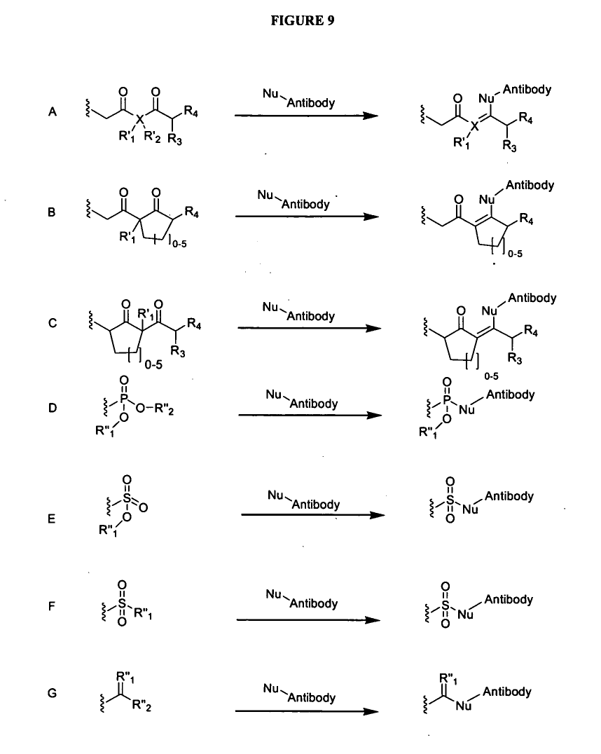

Z may be a group that forms a reversible or irreversible covalent bond. In some embodiments, reversible covalent bonds may be formed using diketone Z groups such as those shown in FIG. 7. Thus, structures A-C may form reversible covalent bonds with reactive nucleophilic groups (e.g., lysine or cysteine sidechains) in a combining site of an antibody. R′₁, R′₂, R₃, and R₄ in structures A-C of FIG. 7 represent substituents which can be C, H, N, O, P, S, halogen (F, Cl, Br, I), or a salt thereof. These substituents may also include a group such as an alkyl, alkenyl, alkynyl, oxoalkyl, oxoalkenyl, oxoalkynyl, aminoalkyl, aminoalkenyl, aminoalkynyl, sulfoalkyl, sulfoalkenyl, or sulfoalkynyl group, phosphoalkyl, phosphoalkenyl, phosphoalkynyl group. R′₂ and R₃ also could form a ring structure as exemplified in structures B and C. X in FIG. 7 could be a heteroatom. Other Z groups that form reversible covalent bonds include the amidine, imine, and other reactive groups encompassed by structure G of FIG. 7. FIG. 8 includes the structures of other linker reactive groups that form reversible covalent bonds, e.g., structures B, G, H, and, where X is not a leaving group, E and F.

Z reactive groups that form an irreversible covalent bond with a combining site of an antibody include structures D-G in FIG. 7 (e.g., when G is an imidate) and structures A, C, and D of FIG. 8. When X is a leaving group, structures E and F of FIG. 8 may also form irreversible covalent bonds. Such structures are useful for irreversibly attaching a targeting agent-linker to a reactive nucleophilic group in a combining site of an antibody.

In other such embodiments, Z is a 1,3-diketone moiety. In still other such embodiments, Z is alkyl substituted by a 1,3-diketone moiety. In one embodiment, Z has the structure:

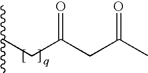

wherein q=0-5. In another embodiment, Z has the structure:

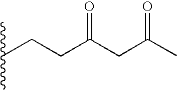

One linker for use in GA targeting compounds and for preparing GA targeting agent-linker compounds includes a 1,3-diketone reactive group as Z. In one embodiment of Formula I, L has the structure:

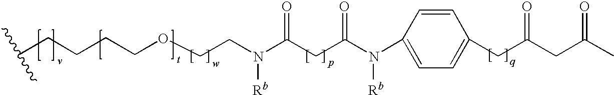

In certain of these embodiments, v is 0, 1, 2, 3, 4, or 5; t is 1, 2, 3, 4, 5, or 6; w is 1, 2, 3, 4, or 5; p is 1, 2, 3, 4, or 5; q is 0, 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments v is 0; t is 1, 2, 3, 4, 5, or 6; w is 1; p is 3; and q is 0, 1, 2, or 3. In some embodiments, v is 0; t is 1 or 2; w is 1; p is 1 or 2; and q is 1 or 2.

In certain embodiments of Formula I, L has the structure:

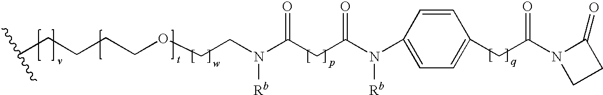

In certain of these embodiments, v is 0, 1, 2, 3, 4, or 5; t is 1, 2, 3, 4, 5, or 6; w is 1, 2, 3, 4, or 5; p is 1, 2, 3, 4, or 5; q is 0, 1, 2, or 3; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; w is 1; p is 3; and q is 0, 1, 2, or 3. In some embodiments, v is 0; t is 1 or 2; w is 1; p is 1 or 2; and q is 2 or 3.

In certain embodiments of Formula I, L has the structure:

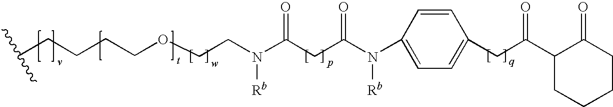

In certain of these embodiments, v is 0, 1, 2, 3, 4, or 5; t is 1, 2, 3, 4, 5, or 6; w is 1, 2, 3, 4, or 5; p is 1, 2, 3, 4, or 5; q is 0, 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; w is 1; p is 3; and q is 0, 1, 2, or 3. In some embodiments, v is 0; t is 1 or 2; w is 1; p is 1 or 2; and q is 1 or 2.

In certain embodiments of Formula I, L has the structure:

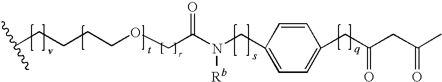

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; r is 1 or 2; s is 3; and q is 0, 1, 2, or 3. In some embodiments, v is 0; t is 1, 2, or 3, r is 1; s is 1 or 2; and q is 1 or 2.

In certain embodiments of Formula I, L has the structure:

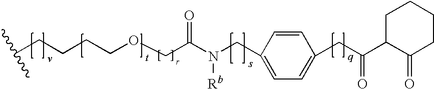

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; r is 1 or 2; s is 3; and q is 0, 1, 2, or 3. In some embodiments, v is 0; t is 1, 2, or 3, r is 1; s is 1 or 2; and q is 1 or 2.

In certain embodiments of Formula I, L has the structure:

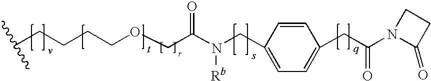

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, or 3; and R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; r is 1 or 2; s is 3; and q is 0, 1, 2, or 3. In some embodiments, v is 0; t is 1, 2, or 3, r is 1; s is 1 or 2; and q is 2 or 3.

In certain embodiments of Formula I, L has the structure:

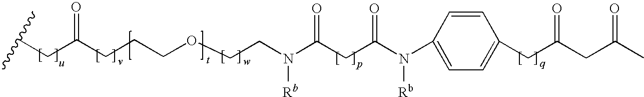

In certain of these embodiments, u is 0, 1, 2, 3, 4, or 5; v is 0, 1, 2, 3, 4, or 5; t is 1, 2, 3, 4, 5, or 6; w is 1, 2, 3, 4, or 5; p is 1, 2, 3, 4, or 5; q is 0, 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; w is 1; p is 3; and q is 0, 1, 2, or 3. In some embodiments, u is 0 or 1; v is 0; t is 1 or 2; w is 1; p is 1 or 2; and q is 1 or 2.

In certain embodiments of Formula I, L has the structure:

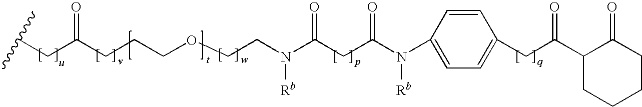

In certain of these embodiments, u is 0, 1, 2, 3, 4, or 5; v is 0, 1, 2, 3, 4, or 5; t is 1, 2, 3, 4, 5, or 6; w is 1, 2, 3, 4, or 5; p is 1, 2, 3, 4, or 5;q is 0, 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; w is 1; p is 3; and q is 0, 1, 2, or 3. In some embodiments, v is 0; t is 1 or 2; w is 1; p is 1 or 2; and q is 1 or 2.

In certain embodiments of Formula I, L has the structure:

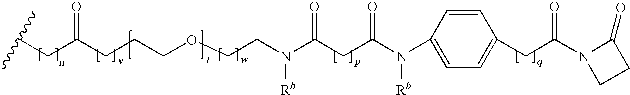

In certain of these embodiments, u is 0, 1, 2, 3, 4, or 5;v is 0, 1, 2, 3, 4, or 5; t is 1, 2, 3, 4, 5, or 6; w is 1, 2, 3, 4, or 5; p is 1, 2, 3, 4, or 5; q is 0, 1, 2, or 3; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; w is 1; p is 3; and q is 0, 1, 2, or 3. In some embodiments, v is 0; t is 1 or 2; w is 1; p is 1 or 2;and q is 2 or 3.

In certain embodiments of Formula I, L has the structure:

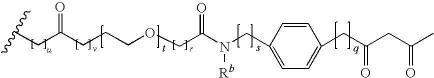

In certain of these embodiments, u is 0, 1, 2, 3, 5, or 5; v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; r is 1 or 2; s is 3; and q is 0, 1, 2, or 3. In some embodiments, u is 0 or 1; v is 0; t is 1 or 2; w is 1; p is 1 or 2; and q is 1 or 2.

In certain embodiments of Formula I, L has the structure:

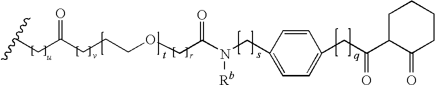

In certain of these embodiments u is 0, 1, 2, 3, 5, or 5; v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; r is 1 or 2; s is 3; and q is 0, 1, 2, or 3. In some embodiments, u is 0 or 1; v is 0; t is 1, 2, or 3, r is 1; s is 1 or 2; and q is 1 or 2.

In certain embodiments of Formula I, L has the structure:

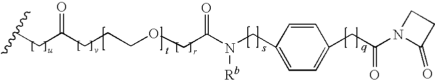

In certain of these embodiments, u is 0, 1, 2, 3, 5, or 5; v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, or 3; and R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; r is 1 or 2; s is 3; and q is 0, 1, 2, or 3. In some embodiments, u is 0 or 1; v is 0; t is 1, 2, or 3r is 1; s is 1 or 2; and q is 2 or 3.

In certain embodiments of Formula I, L has the structure:

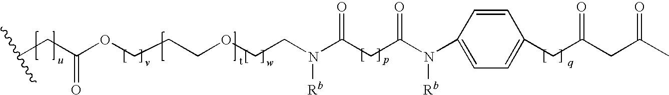

In certain of these embodiments, u is 0, 1, 2, 3, 4, or 5; v is 0, 1, 2, 3, 4, or 5; t is 1, 2, 3, 4, 5, or 6; w is 1, 2, 3, 4, or 5; p is 1, 2, 3, 4, or 5; q is 0, 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; w is 1; p is 3; and q is 0, 1, 2, or 3. In some embodiments, u is 0 or 1; v is 0; t is 1 or 2; w is 1; p is 1 or 2; and q is 1 or 2.

In certain embodiments of Formula I, L has the structure:

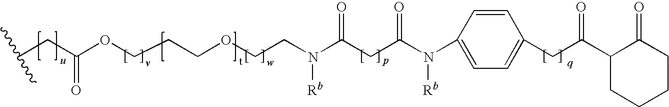

In certain of these embodiments, u is 0, 1, 2, 3, 4, or 5;v is 0, 1, 2, 3, 4, or 5; t is 1, 2, 3, 4, 5, or 6; w is 1, 2, 3, 4, or 5; p is 1, 2, 3, 4, or 5; q is 0, 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; w is 1; p is 3; and q is 0, 1, 2, or 3. In some embodiments, u is 0 or 1; v is 0; t is 1 or 2; w is 1; p is 1 or 2; and q is 1 or 2.

In certain embodiments of Formula I, L has the structure:

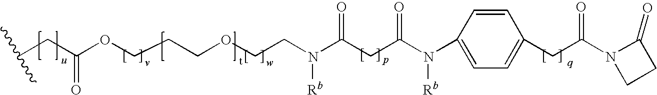

In certain of these embodiments, u is 0, 1, 2, 3, 4, or 5; v is 0, 1, 2, 3, 4, or 5; t is 1, 2, 3, 4, 5, or 6; w is 1, 2, 3, 4, or 5; p is 1, 2, 3, 4, or 5; q is 0, 1, 2, or 3; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; w is 1; p is 3; and q is 0, 1, 2, or 3. In some embodiments, u is 0 or 1; v is 0; t is 1 or 2; w is 1; p is 1 or 2; and q is 2 or 3.

In certain embodiments of Formula I, L has the structure:

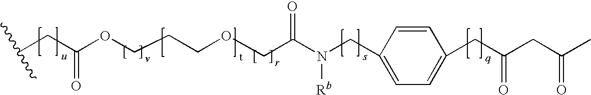

In certain of these embodiments, u is 0, 1, 2, 3, 5, or 5; v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; r is 1 or 2; s is 3; and q is 0, 1, 2, or 3. In some embodiments, u is 0 or 1; v is 0; t is 1, 2, or 3, r is 1; s is 1 or 2; and q is 1 or 2.

In certain embodiments of Formula I, L has the structure:

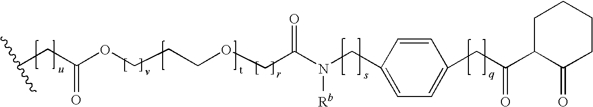

In certain of these embodiments, u is 0, 1, 2, 3, 5, or 5; v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; r is 1 or 2; s is 3; and q is 0, 1, 2, or 3. In some embodiments, u is 0 or 1; v is 0; t is 1, 2, or 3, r is 1; s is 1 or 2; and q is 1 or 2.

In certain embodiments of Formula I, L has the structure:

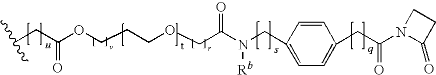

In certain of these embodiments, u is 0, 1, 2, 3, 5, or 5; v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, or 3; and R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; r is 1 or 2; s is 3; and q is 0, 1, 2, or 3. In some embodiments, u is 0 or 1; v is 0; t is 1, 2, or 3, r is 1; s is 1 or 2; and q is 2 or 3.

In certain embodiments of Formula I, L has the structure:

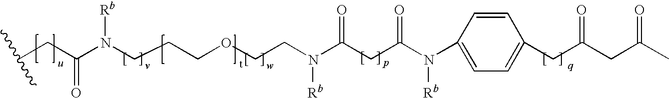

In certain of these embodiments, u is 0, 1, 2, 3, 4, or 5; v is 0, 1, 2, 3, 4, or 5; t is 1, 2, 3, 4, 5, or 6; w is 1, 2, 3, 4, or 5; p is 1, 2, 3, 4, or 5; q is 0, 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; w is 1; p is 3; and q is 0, 1, 2, or 3. In some embodiments, u is 0 or 1; v is 0; t is 1 or 2; w is 1; p is 1 or 2; and q is 1 or 2.

In certain embodiments of Formula I, L has the structure:

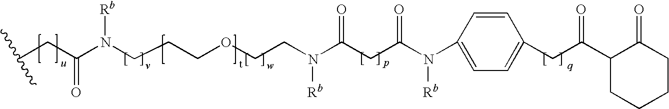

In certain of these embodiments, u is 0, 1, 2, 3, 4, or 5; v is 0, 1, 2, 3, 4, or 5; t is 1, 2, 3, 4, 5, or 6; w is 1, 2, 3, 4, or 5; p is 1, 2, 3, 4, or 5; q is 0, 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; w is 1; p is 3; and q is 0, 1, 2, or 3. In some embodiments, u is 0 or 1; v is 0; t is 1 or 2; w is 1; p is 1 or 2; and q is 1 or 2.

In certain embodiments of Formula I, L has the structure:

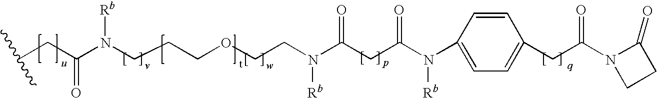

In certain of these embodiments, u is 0, 1, 2, 3, 4, or 5; v is 0, 1, 2, 3, 4, or 5; t is 1, 2, 3, 4, 5, or 6; w is 1, 2, 3, 4, or 5; p is 1, 2, 3, 4, or 5; q is 0, 1, 2, or 3; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; w is 1; p is 3; and q is 0, 1, 2, or 3. In some embodiments, u is 0 or 1; v is 0; t is 1 or 2; w is 1; p is 1 or 2; and q is 2 or 3.

In certain embodiments of Formula I, L has the structure:

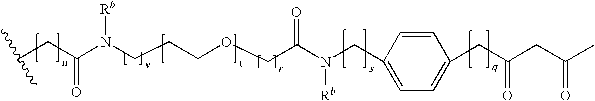

In certain of these embodiments, u is 0, 1, 2, 3, 5, or 5; v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; r is 1 or 2; s is 3;and q is 0, 1, 2, or 3. In some embodiments, u is or 1;v is 0; t is 1, 2, or 3, r is 1; s is 1 or 2;and q is 1 or 2.

In certain embodiments of Formula I, L has the structure:

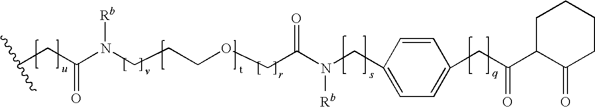

In certain of these embodiments, u is 0, 1, 2, 3, 5, or 5; v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; r is 1 or 2; s is 3; and q is 0, 1, 2, or 3. In some embodiments, u is 0 or 1; v is 0; t is 1, 2, or 3, r is 1; s is 1 or 2; and q is 1 or 2.

In certain embodiments of Formula I, L has the structure:

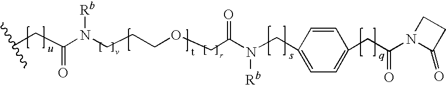

In certain of these embodiments, u is 0, 1, 2, 3, 5, or 5; v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, or 3; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; r is 1 or 2; s is 3; and q is 0, 1, 2, or 3. In some embodiments, u is 0 or 1; v is 0; t is 1, 2, or 3, r is 1; s is 1 or 2; and q is 2 or 3.

In certain embodiments of Formula I, L has the structure:

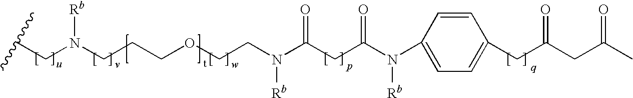

In certain of these embodiments, u is 0, 1, 2, 3, 4, or 5;v is 0, 1, 2, 3, 4, or 5; t is 1, 2, 3, 4, 5, or 6; w is 1, 2, 3, 4, or 5; p is 1, 2, 3, 4, or 5; q is 0, 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; w is 1; p is 3; and q is 0, 1, 2, or 3. In still some embodiments, u is or 1; v is 0; t is 1 or 2; w is 1; p is 1 or 2;and q is 1 or 2.

In certain embodiments of Formula I, L has the structure:

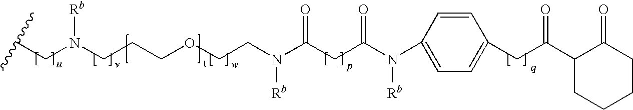

In certain of these embodiments, u is 0, 1, 2, 3, 4, or 5; v is 0, 1, 2, 3, 4, or 5; t is 1, 2, 3, 4, 5, or 6; w is 1, 2, 3, 4, or 5; p is 1, 2, 3, 4, or 5; q is 0, 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; w is 1; p is 3; and q is 0, 1, 2, or 3. In some embodiments, u is 0 or 1; v is 0; t is 1 or 2; w is 1; p is 1 or 2; and q is 1 or 2.

In certain embodiments of Formula I, L has the structure:

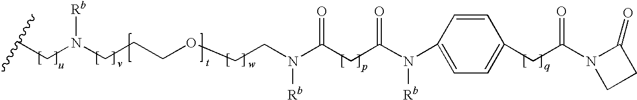

In certain of these embodiments, u is 0, 1, 2, 3, 4, or 5; v is 0, 1, 2, 3, 4, or 5; t is 1, 2, 3, 4, 5, or 6; w is 1, 2, 3, 4, or 5; p is 1, 2, 3, 4, or 5; q is 0, 1, 2, or 3; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; w is 1; p is 3; and q is 0, 1, 2, or 3. In some embodiments, u is 0 or 1; v is 0; t is 1 or 2; w is 1; p is 1 or 2; and q is 2 or 3.

In certain embodiments of Formula I, L has the structure:

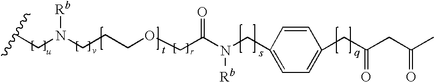

In certain of these embodiments, u is 0, 1, 2, 3, 5, or 5; v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; r is 1 or 2; s is 3; and q is 0, 1, 2, or 3. In some embodiments, u is 0 or 1; v is 0; t is 1, 2, or 3, r is 1; s is 1 or 2; and q is 1 or 2.

In certain embodiments of Formula I, L has the structure:

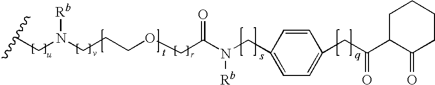

In certain of these embodiments, u is 0, 1, 2, 3, 5, or 5; v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; r is 1 or 2; s is 3; and q is 0, 1, 2, or 3. In some embodiments, u is 0 or 1; v is 0; t is 1, 2, or 3, r is 1; s is 1 or 2; and q is 1 or 2.

In certain embodiments of Formula I, L has the structure:

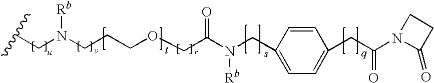

In certain of these embodiments, u is 0, 1, 2, 3, 5, or 5; v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, or 3; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; r is 1 or 2; s is 3; and q is 0, 1, 2, or 3. In some embodiments, u is 0 or 1; v is 0; t is 1, 2, or 3, r is 1; s is 1 or 2; and q is 2 or 3.

In certain embodiments of Formula I, L has the structure:

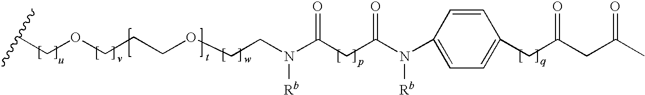

In certain of these embodiments, u is 0, 1, 2, 3, 4, or 5; v is 0, 1, 2, 3, 4, or 5; t is 1, 2, 3, 4, 5, or 6; w is 1, 2, 3, 4, or 5; p is 1, 2, 3, 4, or 5; q is 0, 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; w is 1; p is 3; and q is 0, 1, 2, or 3. In still other embodiments, u is 0 or 1; v is 0; t is 1 or 2; w is 1; p is 1 or 2; and q is 1 or 2.

In certain embodiments of Formula I, L has the structure:

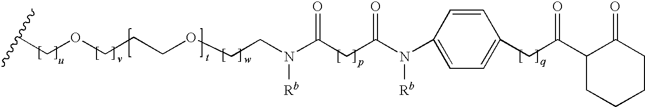

In certain of these embodiments, u is 0, 1, 2, 3, 4, or 5;v is 0, 1, 2, 3, 4, or 5; t is 1, 2, 3, 4, 5, or 6; w is 1, 2, 3, 4, or 5; p is 1, 2, 3, 4, or 5; q is 0, 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; w is 1; p is 3; and q is 0, 1, 2, or 3. In some embodiments, u is 0 or 1; v is 0; t is 1 or 2; w is 1; p is 1 or 2; and q is 1 or 2.

In certain embodiments of Formula I, L has the structure:

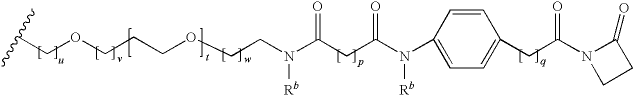

In certain of these embodiments, u is 0, 1, 2, 3, 4, or 5; v is 0, 1, 2, 3, 4, or 5; t is 1, 2, 3, 4, 5, or 6; w is 1, 2, 3, 4, or 5; p is 1, 2, 3, 4, or 5; q is 0, 1, 2, or 3; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; w is 1; p is 3; and q is 0, 1, 2, or 3. In some embodiments, u is 0 or 1; v is 0; t is 1 or 2; w is 1; p is 1 or 2; and q is 2 or 3.

In certain embodiments of Formula I, L has the structure:

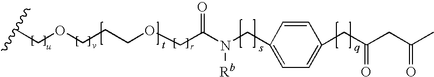

In certain of these embodiments, u is 0, 1, 2, 3, 5, or 5; v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; r is 1 or 2; s is 3; and q is 0, 1, 2, or 3. In some embodiments, u is 0 or 1; v is 0; t is 1, 2, or 3, r is 1; s is 1 or 2; and q is 1 or 2.

In certain embodiments of Formula I, L has the structure:

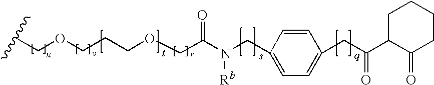

In certain of these embodiments, u is 0, 1, 2, 3, 5, or 5; v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; r is 1 or 2; s is 3; and q is 0, 1, 2, or 3. In some embodiments, u is 0 or 1; v is 0; t is 1, 2, or 3, r is 1; s is 1 or 2; and q is 1 or 2.

In certain embodiments of Formula I, L has the structure:

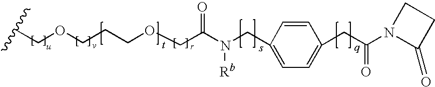

In certain of these embodiments, u is 0, 1, 2, 3, 5, or 5; v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, or 3; and R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 0; v is 0; t is 1, 2, 3, 4, 5, or 6; r is 1 or 2; s is 3; and q is 0, 1, 2, or 3. In some embodiments, u is 0 or 1; v is 0; t is 1, 2, or 3, r is 1; s is 1 or 2; and q is 2 or 3.

In certain embodiments of Formula I, L as the structure:

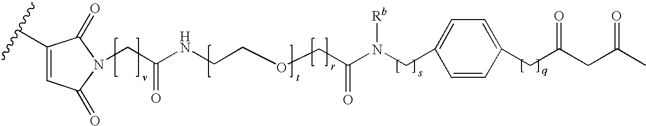

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; r is 1 or 2; s is 0; and q is 0, 1, 2, or 3. In some embodiments, v is 0; t is 1, 2, or 3, r is 1 or 2; s is 0; and q is 1 or 2.

In certain embodiments of Formula I, L as the structure:

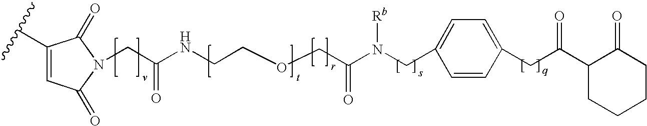

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; r is 1 or 2; s is 0; and q is 0, 1, 2, or 3. In some embodiments, v is 0; t is 1, 2, or 3, r is 1 or 2; s is 0; and q is 1 or 2.

In certain embodiments of Formula I, L as the structure:

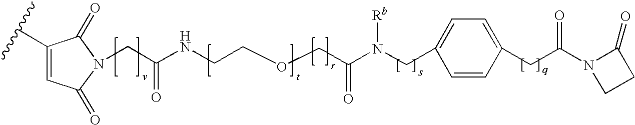

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; r is 1 or 2; s is 0; and q is 0, 1, 2, or 3. In some embodiments, v is 0; t is 1, 2, or 3, r is 1 or 2; s is 0; and q is 1 or 2.

Certain embodiments in accordance with Formula I have the structure:

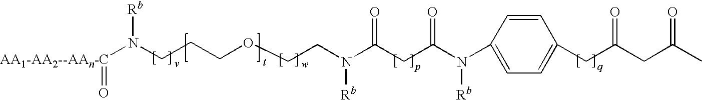

In certain of these embodiments, v is 0, 1, 2, 3, 4, or 5; t is 1, 2, 3, 4, 5, or 6; w is 1, 2, 3, 4, or 5; p is 1, 2, 3, 4, or 5; q is 0, 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; w is 1; p is 3; and q is 0, 1, 2, or 3. In some embodiments, v is 0; t is 1 or 2; w is 1; p is 1 or 2; and q is 1 or 2.

Certain embodiments in accordance with Formula I have the structure:

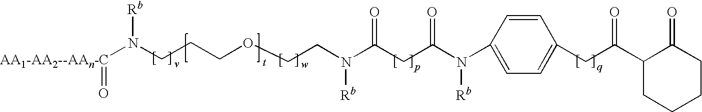

In certain of these embodiments, v is 0, 1, 2, 3, 4, or 5; t is 1, 2, 3, 4, 5, or 6; w is 1, 2, 3, 4, or 5; p is 1, 2, 3, 4, or 5; q is 0, 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; w is 1; p is 3; and q is 0, 1, 2, or 3. In some embodiments, v is 0; t is 1 or 2; w is 1; p is 1 or 2; and q is 1 or 2.

Certain embodiments in accordance with Formula I have the structure:

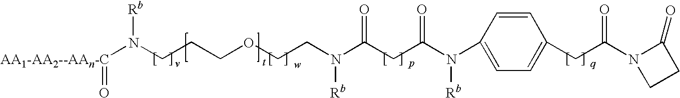

In certain of these embodiments, v is 0, 1, 2, 3, 4, or 5; t is 1, 2, 3, 4, 5, or 6; w is 1, 2, 3, 4, or 5; p is 1, 2, 3, 4, or 5; q is 0, 1, 2, or 3; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; w is 1; p is 3; and q is 0, 1, 2, or 3. In some embodiments, v is 0; t is 1 or 2; w is 1; p is 1 or 2; and q is 2 or 3.

Certain embodiments in accordance with Formula I have the structure:

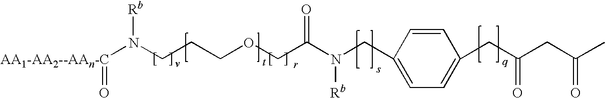

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; r is 1 or 2; s is 3; and q is 0, 1, 2, or 3. In some embodiments, v is 0; t is 1, 2, or 3, r is 1; s is 1 or 2; and q is 1 or 2.

Certain embodiments in accordance with Formula I have the structure:

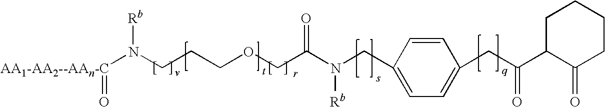

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; r is 1 or 2; s is 3; and q is 0, 1, 2, or 3. In some embodiments, v is 0; t is 1, 2, or 3, r is 1; s is 1 or 2; and q is 1 or 2.

Certain embodiments in accordance with Formula I have the structure:

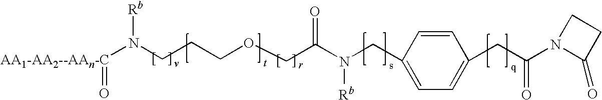

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, or 3; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; r is 1 or 2; s is 3; and q is 0, 1, 2, or 3. In some embodiments, v is 0; t is 1, 2, or 3, r is 1; s is 1 or 2; and q is 2 or 3.

Certain embodiments in accordance with Formula I have the structure:

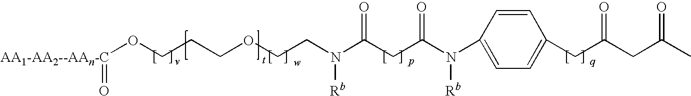

In certain of these embodiments, v is 0, 1, 2, 3, 4, or 5; t is 1, 2, 3, 4, 5, or 6; w is 1, 2, 3, 4, or 5; p is 1, 2, 3, 4, or 5; q is 0, 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; w is 1; p is 3; and q is 0, 1, 2, or 3. In some embodiments, v is 0; t is 1 or 2; w is 1; p is 1 or 2; and q is 1 or 2.

Certain embodiments in accordance with Formula I have the structure:

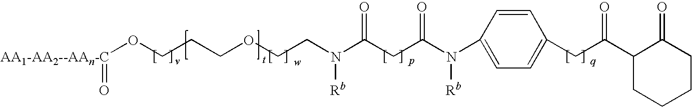

In certain of these embodiments, v is 0, 1, 2, 3, 4, or 5; t is 1, 2, 3, 4, 5, or 6; w is 1, 2, 3, 4, or 5; p is 1, 2, 3, 4, or 5; q is 0, 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; w is 1; p is 3; and q is 0, 1, 2, or 3. In some embodiments, v is 0; t is 1 or 2; w is 1; p is 1 or 2; and q is 1 or 2.

Certain embodiments in accordance with Formula I have the structure:

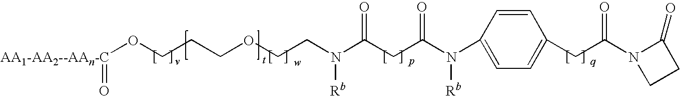

In certain of these embodiments, v is 0, 1, 2, 3, 4, or 5; t is 1, 2, 3, 4, 5, or 6; w is 1, 2, 3, 4, or 5; p is 1, 2, 3, 4, or 5; q is 0, 1, 2, or 3; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; w is 1; p is 3; and q is 0, 1, 2, or 3. In some embodiments, v is 0; t is 1 or 2; w is 1; p is 1 or 2; and q is 2 or 3.

Certain embodiments in accordance with Formula I have the structure:

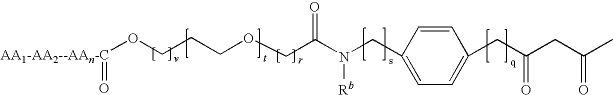

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} is hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl In certain embodiments, v is 0; t is 1, 2, 3, 4, 5, or 6; r is 1 or 2; s is 3; and q is 0, 1, 2, or 3. In some embodiments, v is 0; t is 1, 2, or 3, r is 1; s is 1 or 2; and q is 1 or 2.

Certain embodiments in accordance with Formula I have the structure:

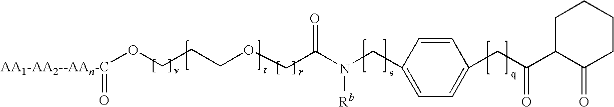

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} is hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, v is 0; t is 1, 2, 3, 4, 5, or 6; r is 1 or 2; s is 3; and q is 0, 1, 2, or 3. In some embodiments, v is 0; t is 1, 2, or 3, r is 1; s is 1 or 2; and q is 1 or 2.

Certain embodiments in accordance with Formula I have the structure:

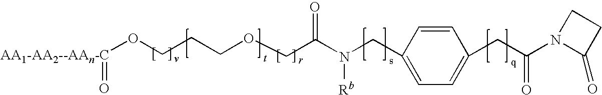

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, or 3; and R^{b} is hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, v is 0; t is 1, 2, 3, 4, 5, or 6; r is 1 or 2; s is 3; and q is 0, 1, 2, or 3. In some embodiments, v is 0; t is 1, 2, or 3, r is 1; s is 1 or 2;and q is 2 or 3.

Another embodiment in accordance with Formula I is:

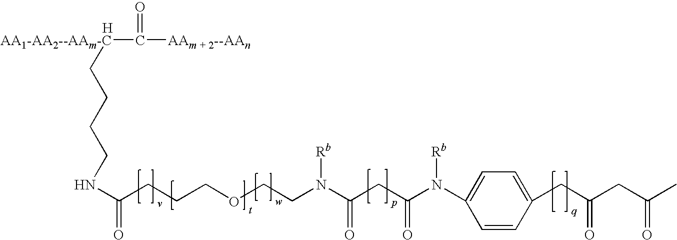

wherein v is 0, 1, 2, 3, 4, or 5; t is 1, 2, 3,4, 5, or 6; w is 1, 2, 3, 4,or 5; p is 1, 2, 3, 4, or 5; and q is 0, 1, 2, 3, 4, or 5. In certain of these embodiments v is 0; t is 1, 2, 3, 4, 5 or 6; w is 1; p is 3; and q is 2.

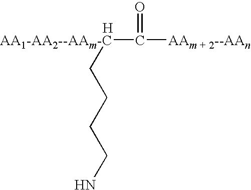

as used herein refers to a GA targeting agent wherein “AA₁” is the first amino acid in a GA targeting agent sequence as measured from the N-terminus, “AA₂” is the second amino acid in a GA targeting agent sequence as measured from the N-terminus, and “AAₙ” is the n h amino acid in a GA targeting agent sequence as measured from the N-terminus. The targeting agent further comprises a Lys residue at arbitrary position m+1 as measured from the N-terminus. It will be appreciated that in addition to linking to a Lys sidechain in the body of a GA targeting agent, it is also possible to link to a Lys sidechain on the N-terminus or C-terminus of a GA targeting agent.

Certain embodiments in accordance with Formula I have the structure:

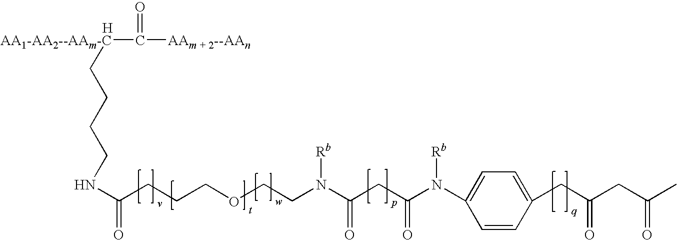

In certain of these embodiments, v is 0, 1, 2, 3, 4, or 5; t is 1, 2, 3, 4, 5, or 6; w is 1, 2, 3, 4, or 5; p is 1, 2, 3, 4, or 5; q is 0, 1, 2, 3, 4, or 5; and R^{b} at each occurrence is independently hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, v is 0; t is 1, 2, 3, 4, 5, or 6; w is 1; p is 3; and q is 0, 1, 2, or 3. In some embodiments, v is 0; t is 1 or 2; w is 1; p is 1 or 2; and q is 1 or 2.

Certain embodiments in accordance with Formula I have the structure:

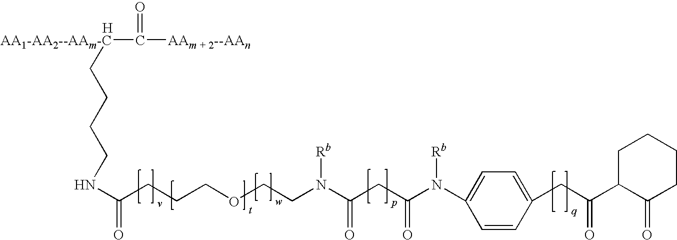

In certain of these embodiments, v is 0, 1, 2, 3, 4, or 5; t is 1, 2, 3, 4, 5, or 6; w is 1, 2, 3, 4, or 5; p is 1, 2, 3, 4, or 5; q is 0, 1, 2, 3, 4, or 5; and R^{b} at each occurrence is independently hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, v is 0; t is 1, 2, 3, 4, 5, or 6; w is 1; p is 3; and q is 0, 1, 2, or 3. In some embodiments, v is 0; t is 1 or 2; w is 1; p is 1 or 2; and q is 1 or 2.

Certain embodiments in accordance with Formula I have the structure:

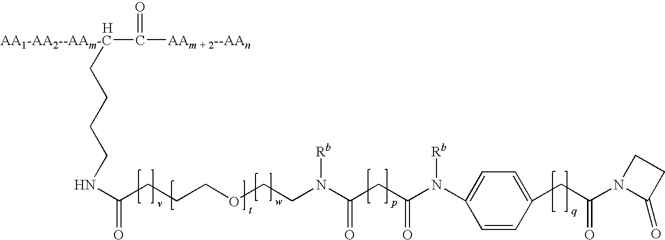

In certain of these embodiments, v is 0, 1, 2, 3, 4, or 5; t is 1, 2, 3, 4, 5, or 6; w is 1, 2, 3, 4, or 5; p is 1, 2, 3, 4, or 5; q is 0, 1, 2, or 3; and R^{b} at each occurrence is independently hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, v is 0; t is 1, 2, 3, 4, 5, or 6; w is 1; p is 3; and q is 0, 1, 2, or 3. In some embodiments, v is 0; t is 1 or 2; w is 1; p is 1 or 2; and q is 2 or 3.

Certain embodiments in accordance with Formula I have the structure:

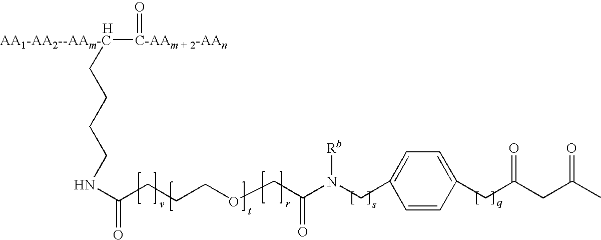

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} is hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, v is 0; t is 1, 2, 3, 4, 5, or 6; r is 1 or 2; s is 3; and q is 0, 1, 2, or 3. In some embodiments, v is 0; t is 1, 2, or 3, r is 1; s is 1 or 2; and q is 1 or 2.

Certain embodiments in accordance with Formula I have the structure:

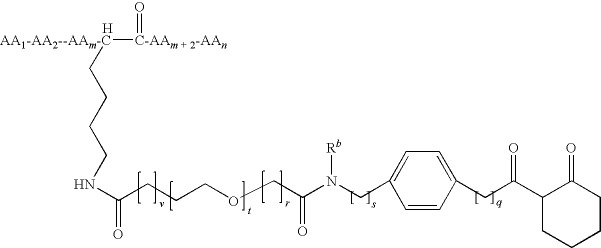

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} is hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, v is 0; t is 1, 2, 3, 4, 5, or 6; r is 1 or 2; s is 3; and q is 0, 1, 2, or 3. In some embodiments, v is 0; t is 1, 2, or 3, r is 1; s is 1 or 2;and q is 1 or 2.

Certain embodiments in accordance with Formula I have the structure:

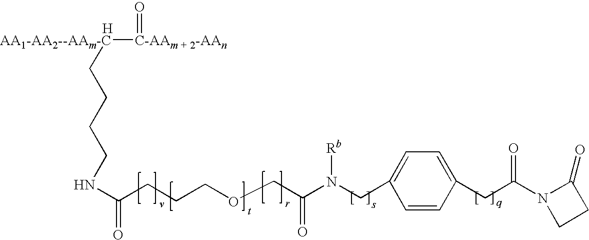

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, or 3; and R^{b} is hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, v is 0; t is 1, 2, 3, 4, 5, or 6; r is 1 or 2; s is 3; and q is 0, 1, 2, or 3. In some embodiments, v is 0; t is 1, 2, or 3, r is 1; s is 1 or 2; and q is 2 or 3.

Certain embodiments in accordance with Formula II have the structure:

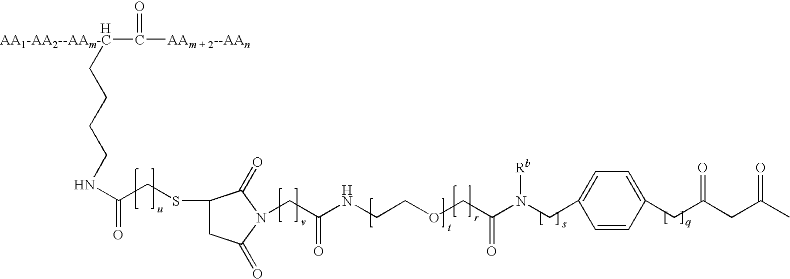

In certain of these embodiments, u is 1, 2, 3, 4, or 5; v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} is hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, u is 1, 2, 3, 4, or 5; v is 0; t is 1, 2, 3, 4, 5, or 6; r is 1 or 2; s is 0; and q is 0, 1, 2, or 3. In some embodiments, u is 1, 2 or 3; v is 0; t is 1, 2, or 3, r is 1 or 2; s is 0; and q is 1 or 2.

Certain embodiments in accordance with Formula I have the structure:

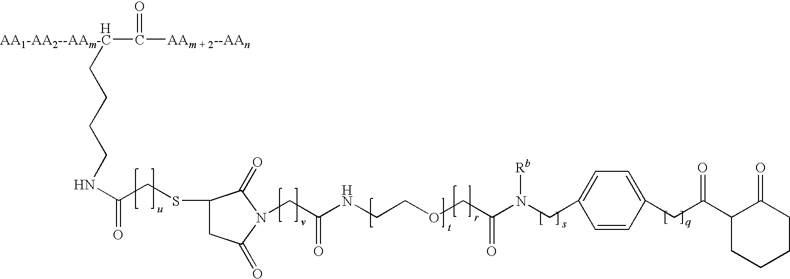

In certain of these embodiments, u is 1, 2, 3, 4, or 5; v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} is hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, u is 1, 2, 3, 4, or 5; v is 0; t is 1, 2, 3, 4, 5, or 6; r is 1 or 2; s is 0; and q is 0, 1, 2, or 3. In some embodiments, u is 1, 2 or 3; v is 0; t is 1, 2, or 3, r is 1 or 2; s is 0; and q is 1 or 2.

Certain embodiments in accordance with Formula I have the structure:

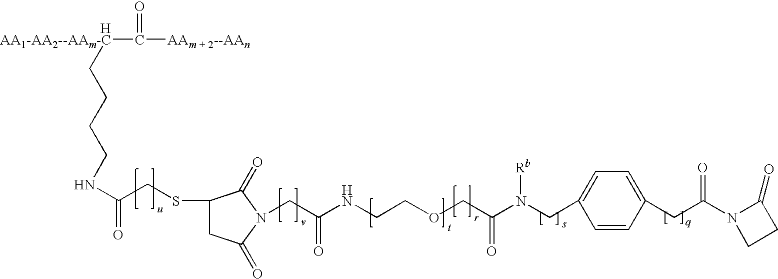

In certain of these embodiments, u is 1, 2, 3, 4, or 5; v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} is hydrogen, substituted or unsubstituted C₁₋₁₀ alkyl, substituted or unsubstituted C₃₋₇ cycloalkyl-C₀₋₆ alkyl or substituted or unsubstituted aryl-C₀₋₆ alkyl. In certain embodiments, u is 1, 2, 3, 4, or 5; v is 0; t is 1, 2, 3, 4, 5, or 6; r is 1 or 2; s is 0; and q is 0, 1, 2, or 3. In some embodiments, u is 1, 2 or 3; v is 0; t is 1, 2, or 3, r is 1 or 2; s is 0; and q is 1 or 2.

Certain embodiments in accordance with Formula I have the structure:

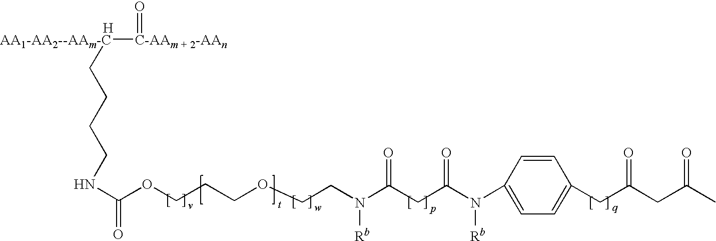

In certain of these embodiments, v is 0, 1, 2, 3, 4, or 5; t is 1, 2, 3, 4, 5, or 6; w is 1, 2, 3, 4, or 5; p is 1, 2, 3, 4, or 5; q is 0, 1, 2, 3, 4, or 5; and R^{b} at each occurrence is independently hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, v is 0; t is 1, 2, 3, 4, 5, or 6; w is 1; p is 3; and q is 0, 1, 2, or 3. In some embodiments, v is 0; t is 1 or 2; w is 1; p is 1 or 2; and q is 1 or 2.

Certain embodiments in accordance with Formula I have the structure:

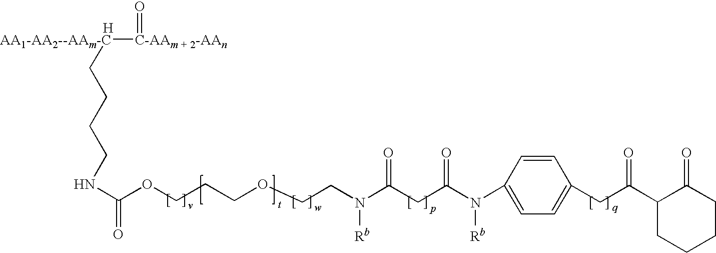

In certain of these embodiments, v is 0, 1, 2, 3, 4, or 5; t is 1, 2, 3, 4, 5, or 6; w is 1, 2, 3, 4, or 5; p is 1, 2, 3, 4, or 5; q is 0, 1, 2, 3, 4, or 5; and R^{b} at each occurrence is independently hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, v is 0; t is 1, 2, 3, 4, 5, or 6; w is 1; p is 3; and q is 0, 1, 2, or 3. In some embodiments, v is 0; t is 1 or 2; w is 1; p is 1 or 2; and q is 1 or 2.

Certain embodiments in accordance with Formula I have the structure:

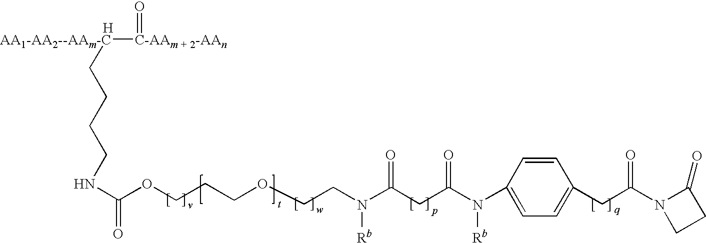

In certain of these embodiments, v is 0, 1, 2, 3, 4, or 5; t is 1, 2, 3, 4, 5, or 6; w is 1, 2, 3, 4, or 5; p is 1, 2, 3, 4, or 5; q is 0, 1, 2, or 3; and R^{b} at each occurrence is independently hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, v is 0; t is 1, 2, 3, 4, 5, or 6; w is 1; p is 3; and q is 0, 1, 2, or 3. In some embodiments, v is 0; t is 1 or 2; w is 1; p is 1 or 2; and q is 2 or 3.

Certain embodiments in accordance with Formula I have the structure:

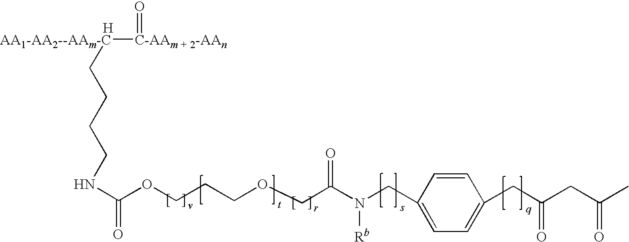

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} is hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, v is 0; t is 1, 2, 3, 4, 5, or 6; r is 1 or 2; s is 3; and q is 0, 1, 2, or 3. In some embodiments, v is 0; t is 1, 2, or 3, r is 1; s is 1 or 2; and q is 1 or 2.

Certain embodiments in accordance with Formula I have the structure:

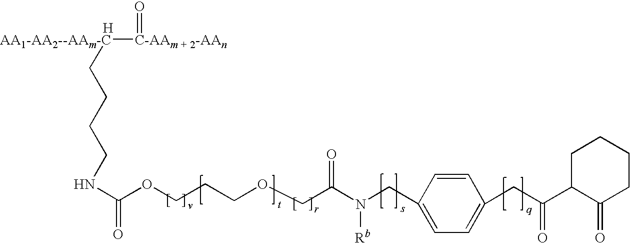

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} is hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, v is 0; t is 1, 2, 3, 4, 5, or 6; r is 1 or 2; s is 3; and q is 0, 1, 2, or 3. In some embodiments, v is 0; t is 1, 2, or 3, r is 1; s is 1 or 2; and q is 1 or 2.

Certain embodiments in accordance with Formula I have the structure:

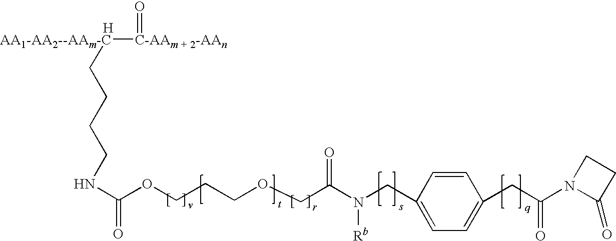

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, or 3; and R^{b} is hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, v is 0; t is 1, 2, 3, 4, 5, or 6; r is 1 or 2; s is 3; and q is 0, 1, 2, or 3. In some embodiments, v is 0; t is 1, 2, or 3, r is 1; s is 1 or 2; and q is 2 or 3.

Certain embodiments in accordance with Formula I have the structure:

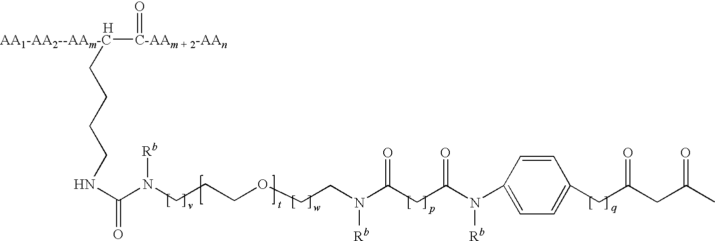

In certain of these embodiments, v is 0, 1, 2, 3, 4, or 5; t is 1, 2, 3, 4, 5, or 6; w is 1, 2, 3, 4, or 5; p is 1, 2, 3, 4, or 5; q is 0, 1, 2, 3, 4, or 5; and R^{b} at each occurrence is independently hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, v is 0; t is 1, 2, 3, 4, 5, or 6; w is 1; p is 3; and q is 0, 1, 2, or 3. In some embodiments, v is 0; t is 1 or 2; w is 1; p is 1 or 2; and q is 1 or 2.

Certain embodiments in accordance with Formula I have the structure:

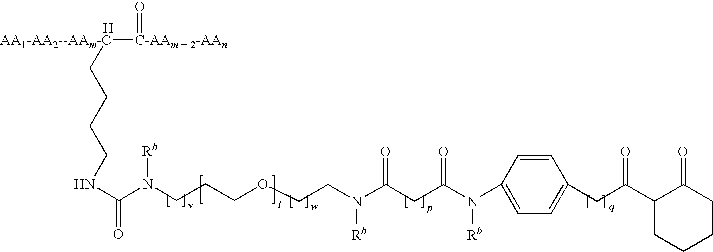

In certain of these embodiments, v is 0, 1, 2, 3, 4, or 5; t is 1, 2, 3, 4, 5, or 6; w is 1, 2, 3, 4, or 5; p is 1, 2, 3, 4, or 5; q is 0, 1, 2, 3, 4, or 5; and R^{b} at each occurrence is independently hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiment v is 0; t is 1, 2, 3, 4, 5, or 6; w is 1; p is 3; and q is 0, 1, 2, or 3. In some embodiments, v is 0; t is 1 or 2; w is 1; p is 1 or 2; and q is 1 or 2.

Certain embodiments in accordance with Formula I have the structure:

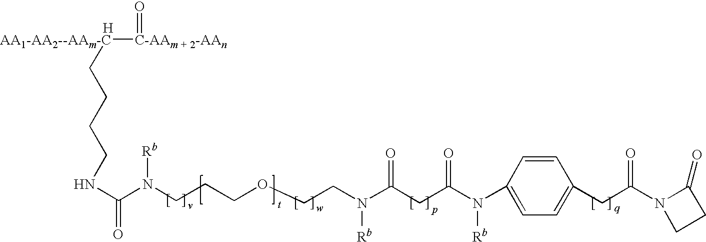

In certain of these embodiments, v is 0, 1, 2, 3, 4, or 5; t is 1, 2, 3, 4, 5, or 6; w is 1, 2, 3, 4, or 5; p is 1, 2, 3, 4, or 5; q is 0, 1, 2, or 3; and R^{b} at each occurrence is independently hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, v is 0; t is 1, 2, 3, 4, 5, or 6; w is 1; p is 3; and q is 0, 1, 2, or 3. In some embodiments, v is 0; t is 1 or 2; w is 1; p is 1 or 2; and q is 2 or 3.

Certain embodiments in accordance with Formula I have the structure:

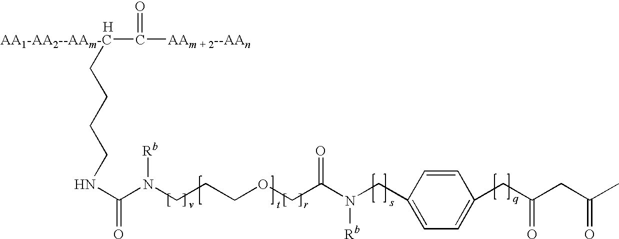

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} at each occurrence is independently hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, v is 0; t is 1, 2, 3, 4, 5, or 6; r is 1 or 2; s is 3; and q is 0, 1, 2, or 3. In some embodiments, v is 0; t is 1, 2, or 3, r is 1; s is 1 or 2; and q is 1 or 2.

Certain embodiments in accordance with Formula I have the structure:

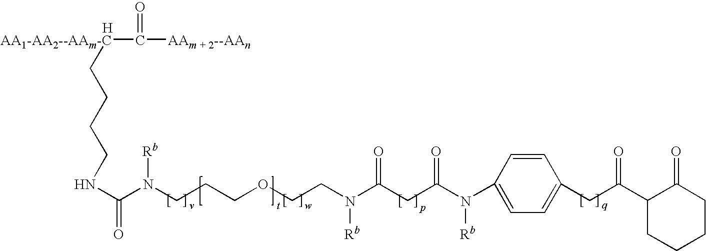

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} at each occurrence is independently hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, v is 0; t is 1, 2, 3, 4, 5, or 6; r is 1 or 2; s is 3; and q is 0, 1, 2, or 3. In some embodiments, v is 0; t is 1, 2, or 3, r is 1; s is 1 or 2; and q is 1 or 2.

Certain embodiments in accordance with Formula I have the structure:

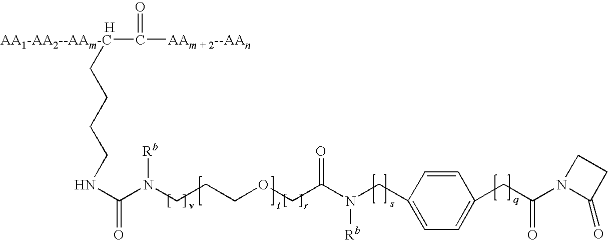

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, or 3; and R^{b} at each occurrence is independently hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, v is 0; t is 1, 2, 3, 4, 5, or 6; r is 1 or 2; s is 3; and q is 0, 1, 2, or 3. In some embodiments, v is 0; t is 1, 2, or 3, r is 1; s is 1 or 2; and q is 2 or 3.

In certain embodiments, linker L in accordance with Formula 1 is:

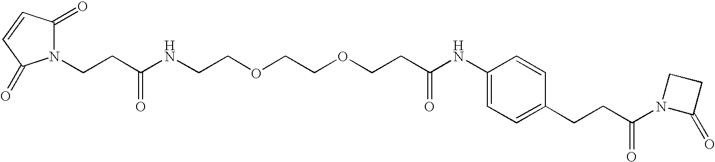

The administration of a GA targeting compound to an immunocompetent individual may result in the production of antibodies against the conjugate. Such antibodies may be directed to the variable region, including the antibody idiotype, as well as to the targeting agent or any linker used to conjugate the targeting agent to the antibody. Reducing the immunogenicity of a GA targeting compound can be addressed by methods well known in the art, such as by attaching long chain polyethylene glycol (PEG)-based spacers and the like to the GA targeting compound. Long chain PEG and other polymers are known for their ability to mask foreign epitopes, resulting in the reduced immunogenicity of therapeutic proteins that display foreign epitopes (N. V. Katre, J. Immunol. 144:209-213 (1990); G. E. Francis et al., Int. J. Hematol. 68:1-18 (1998). Alternatively, or in addition, the individual administered the antibody-GA targeting agent conjugate may be administered an immunosuppressant such as cyclosporin A, anti-CD3 antibody, and the like.

In one embodiment, a GA targeting compound is as shown by Formula II, and includes stereoisomers, tautomers, solvates, prodrugs, and pharmaceutically acceptable salts thereof.

Antibody-L′-[GA targeting agent] (II)

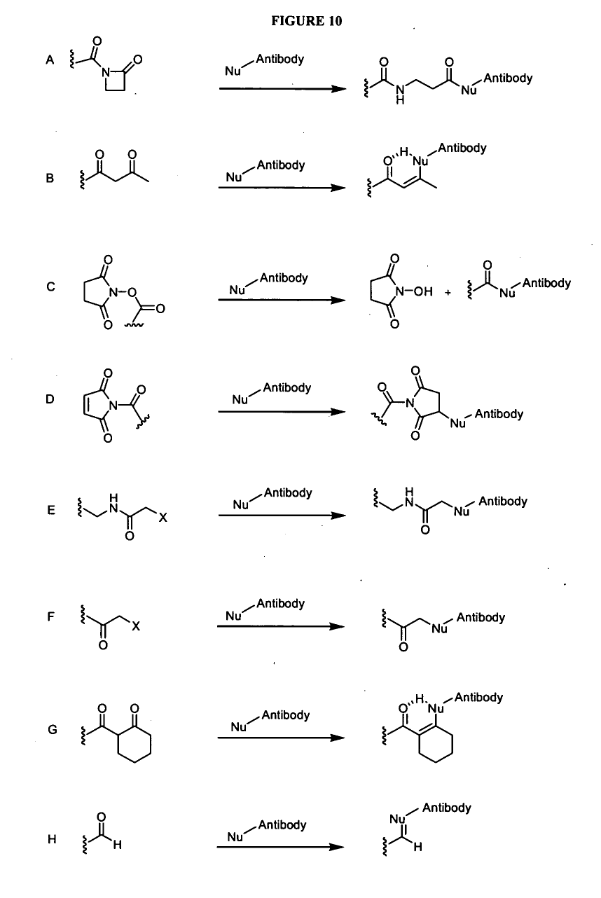

In compounds of Formula II, [GA targeting agent] is defined as in Formula I, and L′ is a linker moiety linking an antibody to the targeting agent and having formula X—Y-Z′-. In compounds of Formula II, X and Y are defined as in Formula I, and Antibody is an antibody as defined herein. FIGS. 9 and 10, respectively, illustrate the addition mechanism of a reactive, nucleophilic sidechain in a combining site of an antibody to the Z moieties illustrated in FIGS. 7 and 8.

In one embodiment, wherein Antibody is an aldolase catalytic antibody, Z′-Antibody has the formula:

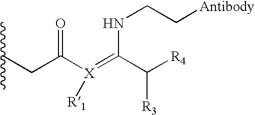

In one embodiment, wherein Antibody is an aldolase catalytic antibody, Z′-Antibody has the formula:

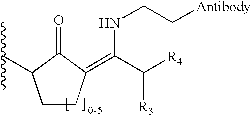

In one embodiment, wherein Antibody is an aldolase catalytic antibody, Z′-Antibody has the formula:

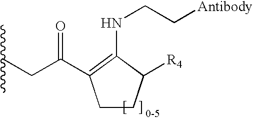

In one embodiment, wherein Antibody is an aldolase catalytic antibody, Z′-Antibody has the formula:

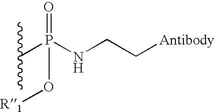

In one embodiment, wherein Antibody is an aldolase catalytic antibody, Z′-Antibody has the formula:

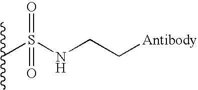

In one embodiment, wherein Antibody is an aldolase catalytic antibody, Z′-Antibody has the formula:

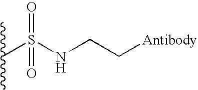

In one embodiment, wherein Antibody is an aldolase catalytic antibody, Z′-Antibody has the formula:

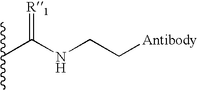

In one embodiment, wherein Antibody is an aldolase catalytic antibody, Z′-Antibody has the formula:

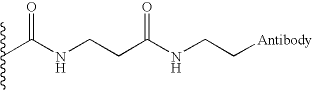

In one embodiment, wherein Antibody is an aldolase catalytic antibody, Z′-Antibody has the formula:

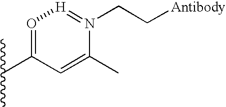

In one embodiment, wherein Antibody is an aldolase catalytic antibody, Z′-Antibody has the formula:

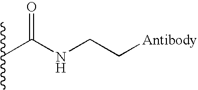

In one embodiment, wherein Antibody is an aldolase catalytic antibody, Z′-Antibody has the formula:

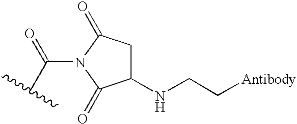

In one embodiment, wherein Antibody is an aldolase catalytic antibody, Z′-Antibody has the formula:

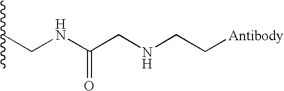

In one embodiment, wherein Antibody is an aldolase catalytic antibody, Z′-Antibody has the formula:

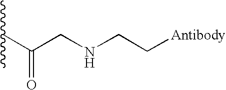

In one embodiment, wherein Antibody is an aldolase catalytic antibody, Z′-Antibody has the formula:

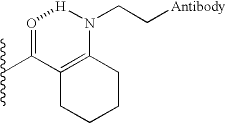

In one embodiment, wherein Antibody is an aldolase catalytic antibody, Z′-Antibody has the formula:

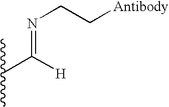

In compounds having Formula II, Z′ is an attachment moiety comprising a covalent bond and 0-20 carbon atoms to which the Antibody is attached. This is shown below for the case where the linker has a diketone moiety as the reactive group (see Z of Formula I) and linkage occurs with the sidechain amino group of a lysine residue in the antibody combining site. The Antibody is shown schematically as bivalent with a reactive amino acid sidechain for each combining site indicated.

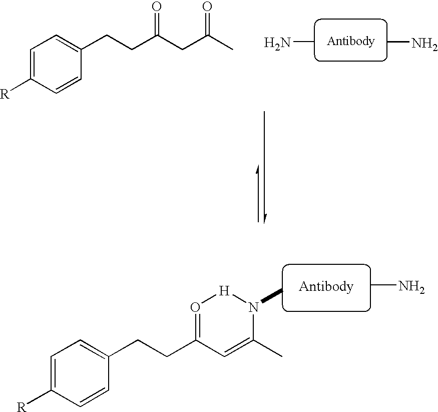

Another embodiment shown below is for the case where the linker has a beta lactam moiety as the reactive group and linkage occurs with the sidechain amino group of a lysine residue in the antibody combining site. The Antibody is shown schematically as bivalent with a reactive amino acid sidechain for each combining site indicated.

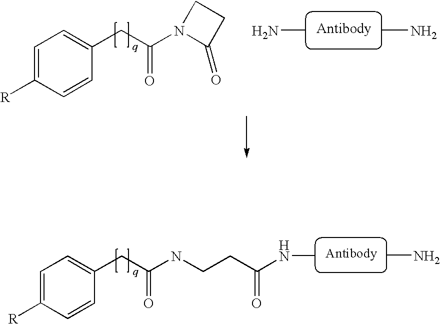

Certain embodiments in accordance with Formula II have the structure:

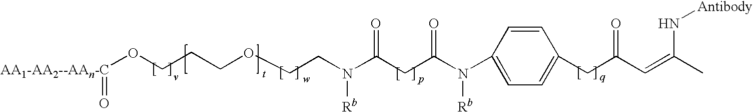

In certain of these embodiments, v is 0, 1, 2, 3, 4, or 5; t is 1, 2, 3, 4, 5, or 6; w is 1, 2, 3, 4, or 5; p is 1, 2, 3, 4, or 5; q is 0, 1, 2, 3, 4, or 5; and R^{b} at each occurrence is independently hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, v is 0; t is 1, 2, 3, 4, 5, or 6; w is 1; p is 3; and q is 0, 1, 2, or 3. In some embodiments, v is 0; t is 1 or 2; w is 1; p is 1 or 2; and q is 1 or 2.

Certain embodiments in accordance with Formula II have the structure:

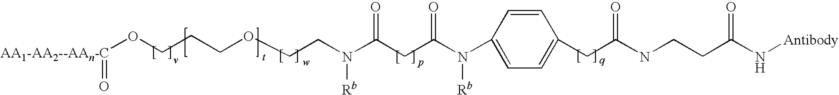

In certain of these embodiments, v is 0, 1, 2, 3, 4, or 5; t is 1, 2, 3, 4, 5, or 6; w is 1, 2, 3, 4, or 5; p is 1, 2, 3, 4, or 5; q is 0, 1, 2, or 3; and R^{b} at each occurrence is independently hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, v is 0; t is 1, 2, 3, 4, 5, or 6; w is 1; p is 3; and q is 0, 1, 2, or 3. In some embodiments, v is 0; t is 1 or 2; w is 1; p is 1 or 2; and q is 2 or 3.

Certain embodiments in accordance with Formula II have the structure:

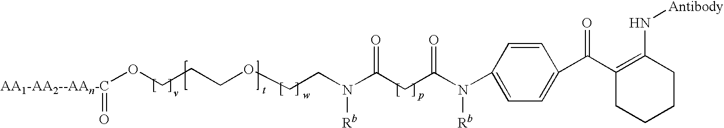

In certain of these embodiments, v is 0, 1, 2, 3, 4, or 5; t is 1, 2, 3, 4, 5, or 6; w is 1, 2, 3, 4, or 5; p is 1, 2, 3, 4, or 5; and R^{b} at each occurrence is independently hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, v is 0; t is 1, 2, 3, 4, 5, or 6; w is 1; and p is 3. In some embodiments, v is 0; t is 1 or 2; w is 1; and p is 1 or 2.

Certain embodiments in accordance with Formula II have the structure:

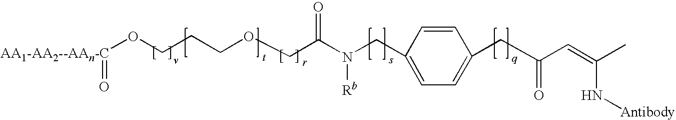

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} is hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, v is 0; t is 1, 2, 3, 4, 5, or 6; r is 1 or 2; s is 3 and q is 0, 1, 2, or 3. In some embodiments, v is 0; t is 1, 2, or 3, r is 1; s is 1 or 2; and q is 1 or 2.

Certain embodiments in accordance with Formula II have the structure:

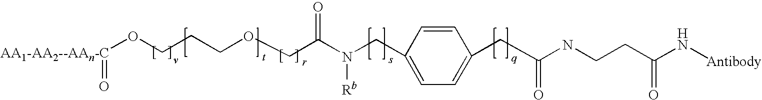

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, or 3; and R^{b} is hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, v is 0; t is 1, 2, 3, 4, 5, or 6; r is 1 or 2; s is 3 and q is 0, 1, 2, or 3. In some embodiments, v is 0; t is 1, 2, or 3, r is 1; s is 1 or 2;and q is 2 or 3.

Certain embodiments in accordance with Formula II have the structure:

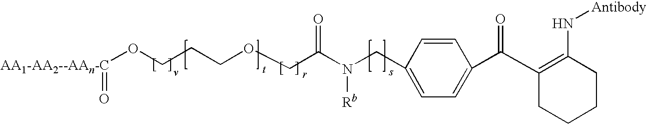

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; and R^{b} is hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, v is 0; t is 1, 2, 3, 4, 5, or 6; r is 1 or 2; and s is 3. In some embodiments, v is 0; t is 1, 2, or 3, r is 1; and s is 1 or 2.

Certain embodiments in accordance with Formula II have the structure:

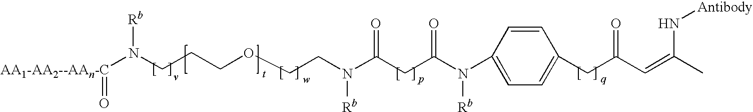

In certain of these embodiments, v is 0, 1, 2, 3, 4, or 5; t is 1, 2, 3, 4, 5, or 6; w is 1, 2, 3, 4, or 5; p is 1, 2, 3, 4, or 5; q is 0, 1, 2, 3, 4, or 5; and R^{b} at each occurrence is independently hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, v is 0; t is 1, 2, 3, 4, 5, or 6; w is 1; p is 3; and q is 0, 1, 2, or 3. In some embodiments, v is 0; t is 1 or 2; w is 1; p is 1 or 2; and q is 1 or 2.

Certain embodiments in accordance with Formula II have the structure:

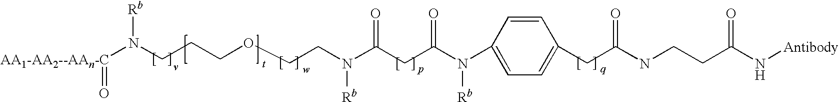

In certain of these embodiments, v is 0, 1, 2, 3, 4, or 5; t is 1, 2, 3, 4, 5, or 6; w is 1, 2, 3, 4, or 5; p is 1, 2, 3, 4, or 5; q is 0, 1, 2, or 3; and R^{b} at each occurrence is independently hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, v is 0; t is 1, 2, 3, 4, 5, or 6; w is 1; p is 3; and q is 0, 1, 2, or 3. In some embodiments, v is 0; t is 1 or 2; w is 1; p is 1 or 2; and q is 2 or 3.

Certain embodiments in accordance with Formula II have the structure:

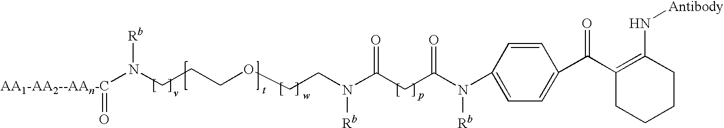

In certain of these embodiments, v is 0, 1, 2, 3, 4, or 5; t is 1, 2, 3, 4, 5, or 6; w is 1, 2, 3, 4, or 5; p is 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; w is 1; and p is 3. In some embodiments, v is 0; t is 1 or 2; w is 1; and p is 1 or 2.

Certain embodiments in accordance with Formula II have the structure:

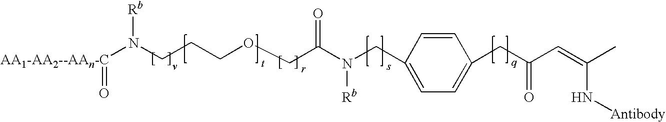

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; r is 1 or 2; s is 3 and q is 0, 1, 2, or 3. In some embodiments, v is 0; t is 1, 2, or 3, r is 1; s is 1 or 2; and q is 1 or 2.

Certain embodiments in accordance with Formula II have the structure:

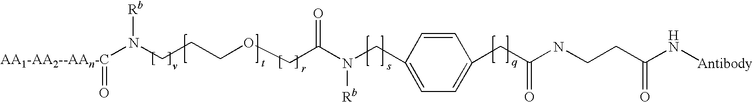

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, or 3; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; r is 1 or 2; s is 3 and q is 0, 1, 2, or 3. In some embodiments, v is 0; t is 1, 2, or 3, r is 1; s is 1 or 2; and q is 2 or 3.

Certain embodiments in accordance with Formula II have the structure:

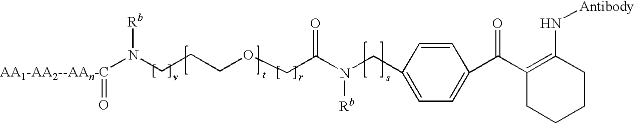

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; r is 1 or 2; and s is 3. In some embodiments, v is 0; t is 1, 2, or 3, r is 1; and s is 1 or 2.

Certain embodiments in accordance with Formula II have the structure:

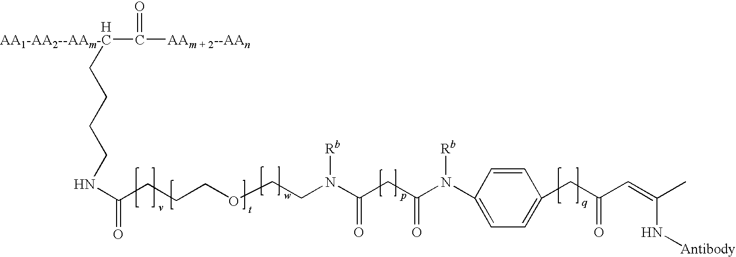

In certain of these embodiments, v is 0, 1, 2, 3, 4, or 5; t is 1, 2, 3, 4, 5, or 6; w is 1, 2, 3, 4, or 5; p is 1, 2, 3, 4, or 5; q is 0, 1, 2, 3, 4, or 5; and R^{b} at each occurrence is independently hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, v is 0; t is 1, 2, 3, 4, 5, or 6; w is 1; p is 3; and q is 0, 1, 2, or 3. In some embodiments, v is 0; t is 1 or 2; w is 1; p is 1 or 2; and q is 1 or 2.

Certain embodiments in accordance with Formula II have the structure:

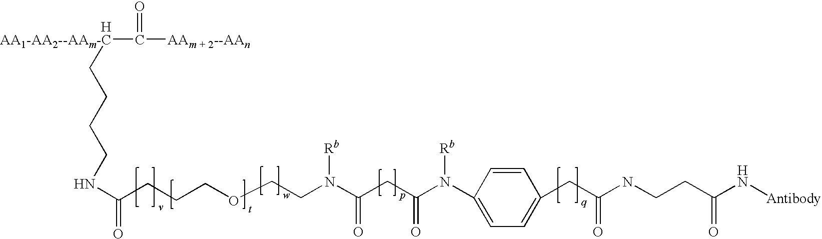

In certain of these embodiments, v is 0, 1, 2, 3, 4, or 5; t is 1, 2, 3, 4, 5, or 6; w is 1, 2, 3, 4, or 5; p is 1, 2, 3, 4, or 5; q is 0, 1, 2, or 3; and R^{b} at each occurrence is independently hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, v is 0; t is 1, 2, 3, 4, 5, or 6; w is 1; p is 3; and q is 0, 1, 2, or 3. In some embodiments, v is 0; t is 1 or 2; w is 1; p is 1 or 2; and q is 2 or 3.

Certain embodiments in accordance with Formula II have the structure:

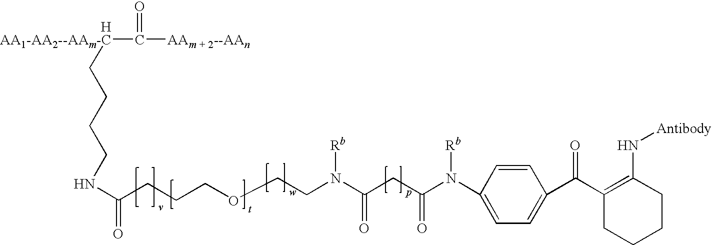

In certain of these embodiments, v is 0, 1, 2, 3, 4, or 5; t is 1, 2, 3, 4, 5, or 6; w is 1, 2, 3, 4, or 5; p is 1, 2, 3, 4, or 5; and R^{b} at each occurrence is independently hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, v is 0; t is 1, 2, 3, 4, 5, or 6; w is 1; and p is 3. In some embodiments, v is 0; t is 1 or 2; w is 1; and p is 1 or 2.

Certain embodiments in accordance with Formula II have the structure:

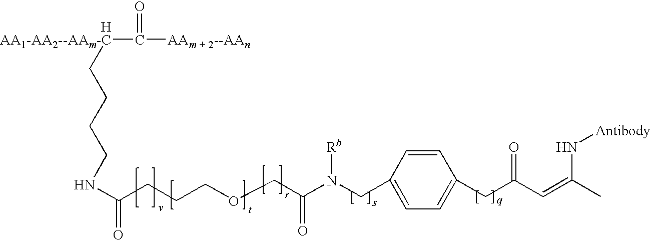

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} is hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl In certain embodiments, v is 0; t is 1, 2, 3, 4, 5, or 6; r is 1 or 2; s is 3; and q is 0, 1, 2, or 3. In some embodiments, v is 0; t is 1, 2, or 3, r is 1; s is 1 or 2; and q is 1 or 2.

Certain embodiments in accordance with Formula II have the structure:

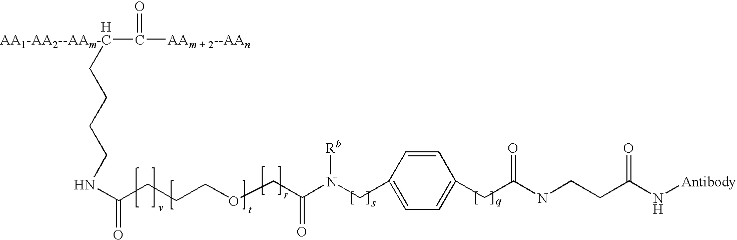

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, or 3; and R^{b} is hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, v is 0; t is 1, 2, 3, 4, 5, or 6; r is 1 or 2; s is 3; and q is 0, 1, 2, or 3. In some embodiments, v is 0; t is 1, 2, or 3, r is 1; s is 1 or 2; and q is 2 or 3.

Certain embodiments in accordance with Formula II have the structure:

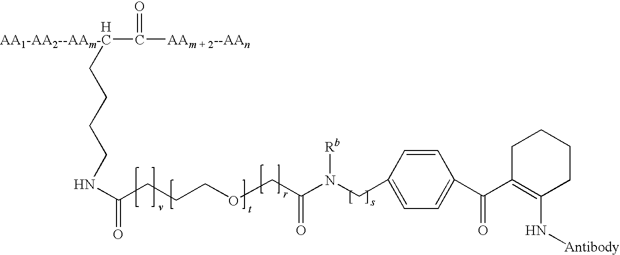

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; and R^{b} is hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, v is 0; t is 1, 2, 3, 4, 5, or 6; r is 1 or 2; and s is 3. In some embodiments, v is 0; t is 1, 2, or 3, r is 1; and s is 1 or 2.

Certain embodiments in accordance with Formula II have the structure:

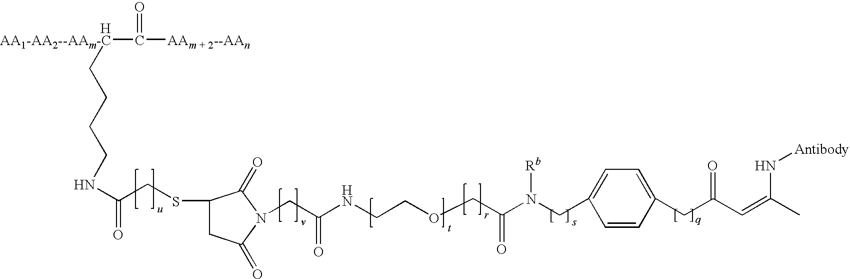

In certain of these embodiments, u is 1, 2, 3, 4, or 5; v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} is hydrogen, substituted or unsubstituted C₁₋₁₀ alkyl substituted or unsubstituted C₃₋₇ cycloalkyl-C₀₋₆ alkyl or substituted or unsubstituted aryl-C₀₋₆ alkyl. In certain embodiments, u is 1, 2, 3, 4, or 5; v is 0; t is 1, 2, 3, 4, 5, or 6; r is 1 or 2; s is 0; and q is 0, 1, 2, or 3. In some embodiments, u is 1, 2 or 3; v is 0; t is 1, 2, or 3, r is 1 or 2; s is 0; and q is 1 or 2.

Certain embodiments in accordance with Formula I have the structure:

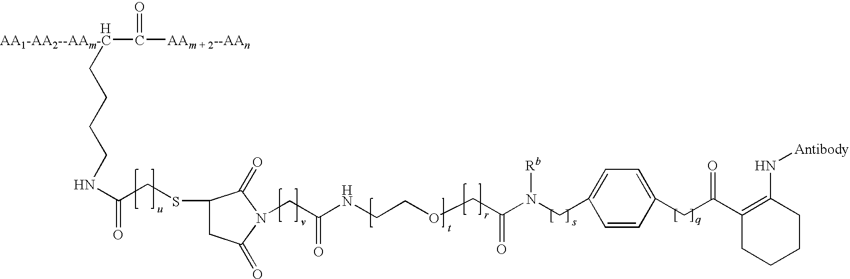

In certain of these embodiments, u is 1, 2, 3, 4, or 5; v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 1, 2, 3, 4, or 5; v is 0; t is 1, 2, 3, 4, 5, or 6; r is 1 or 2; s is 0; and q is 0, 1, 2, or 3. In some embodiments, u is 1, 2 or 3; v is 0; t is 1, 2, or 3, r is 1 or 2; s is 0; and q is 1 or 2.

Certain embodiments in accordance with Formula I have the structure:

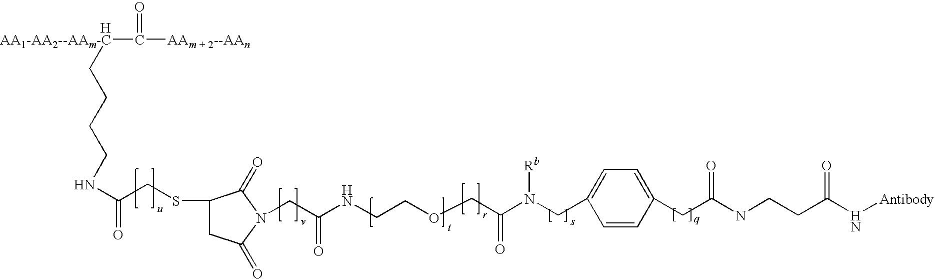

In certain of these embodiments, u is 1, 2, 3, 4, or 5; v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, u is 1, 2, 3, 4, or 5; v is 0; t is 1, 2, 3, 4, 5, or 6; r is 1 or 2; s is 0; and q is 0, 1, 2, or 3. In some embodiments, u is 1, 2 or 3; v is 0; t is 1, 2, or 3, r is 1 or 2; s is 0; and q is 1 or 2.

Certain embodiments in accordance with Formula II have the structure:

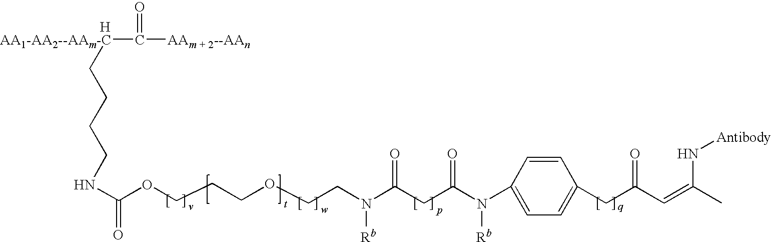

In certain of these embodiments, v is 0, 1, 2, 3, 4, or 5; t is 1, 2, 3, 4, 5, or 6; w is 1, 2, 3, 4, or 5; p is 1, 2, 3, 4, or 5; q is 0, 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; w is 1; p is 3; and q is 0, 1, 2, or 3. In some embodiments, v is 0; t is 1 or 2; w is 1; p is 1 or 2; and q is 1 or 2.

Certain embodiments in accordance with Formula II have the structure:

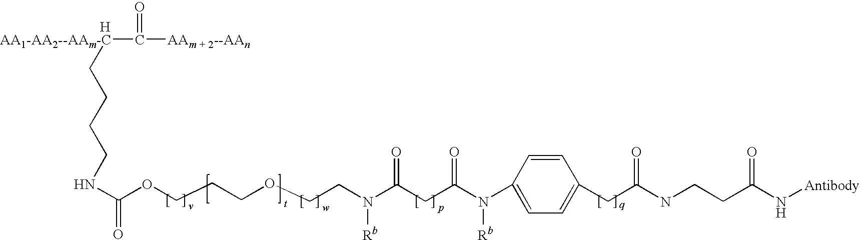

In certain of these embodiments, v is 0, 1, 2, 3, 4, or 5; t is 1, 2, 3, 4, 5, or 6; w is 1, 2, 3, 4, or 5; p is 1, 2, 3, 4, or 5; q is 0, 1, 2, or 3; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; w is 1; p is 3; and q is 0, 1, 2, or 3. In some embodiments, v is 0; t is 1 or 2; w is 1; p is 1 or 2; and q is 2 or 3.

Certain embodiments in accordance with Formula II have the structure:

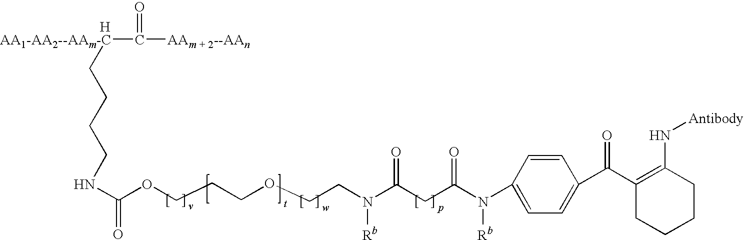

In certain of these embodiments, v is 0, 1, 2, 3, 4, or 5; t is 1, 2, 3, 4, 5, or 6; w is 1, 2, 3, 4, or 5; p is 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; w is 1; and p is 3. In some embodiments, v is 0; t is 1 or 2; w is 1; and p is 1 or 2.

Certain embodiments in accordance with Formula II have the structure:

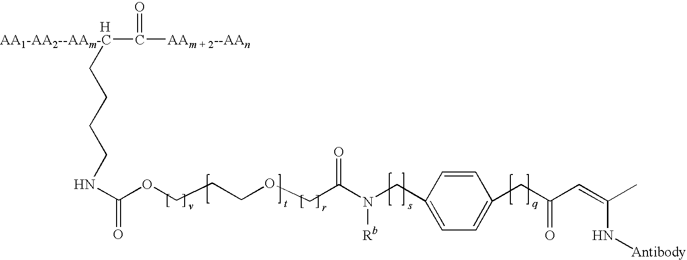

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; r is 1 or 2; s is 3; and q is 0, 1, 2, or 3. In some embodiments, v is 0; t is 1, 2, or 3, r is 1; s is 1 or 2; and q is 1 or 2.

Certain embodiments in accordance with Formula II have the structure:

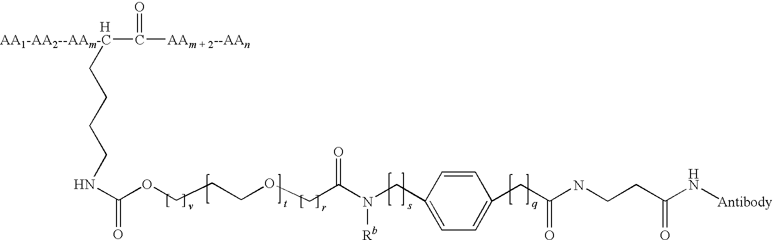

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, or 3; and R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; r is 1 or 2; s is 3; and q is 0, 1, 2, or 3. In some embodiments, v is 0; t is 1, 2, or 3, r is 1; s is 1 or 2; and q is 2 or 3.

Certain embodiments in accordance with Formula II have the structure:

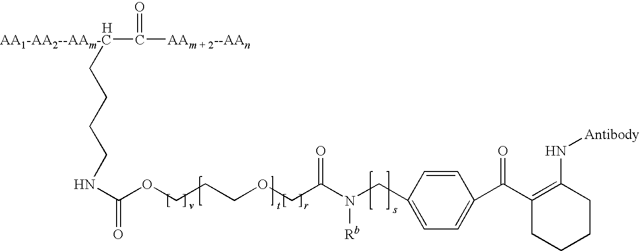

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; and R^{b} is hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; r is 1 or 2; and s is 3. In some embodiments, v is 0; t is 1, 2, or 3, r is 1; and s is 1 or 2.

Certain embodiments in accordance with Formula II have the structure:

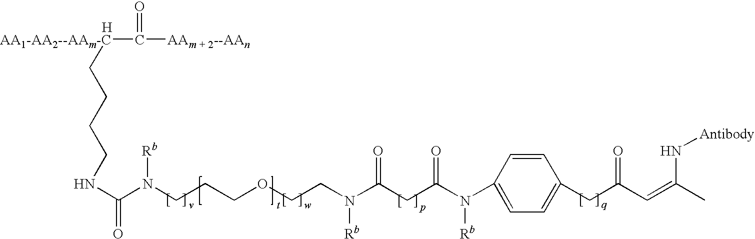

In certain of these embodiments, v is 0, 1, 2, 3, 4, or 5; t is 1, 2, 3, 4, 5, or 6; w is 1, 2, 3, 4, or 5; p is 1, 2, 3, 4, or 5; q is 0, 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; w is 1; p is 3; and q is 0, 1, 2, or 3. In some embodiments, v is 0; t is 1 or 2; w is 1; p is 1 or 2; and q is 1 or 2.

Certain embodiments in accordance with Formula II have the structure:

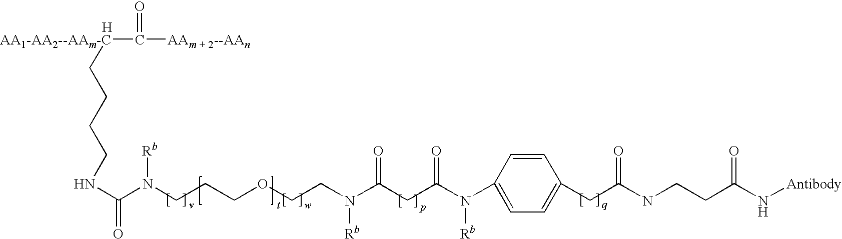

In certain of these embodiments, v is 0, 1, 2, 3, 4, or 5; t is 1, 2, 3, 4, 5, or 6; w is 1, 2, 3, 4, or 5; p is 1, 2, 3, 4, or 5; q is 0, 1, 2, or 3; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; w is 1; p is 3; and q is 0, 1, 2, or 3. In some embodiments, v is 0; t is 1 or 2; w is 1; p is 1 or 2; and q is 2 or 3.

Certain embodiments in accordance with Formula II have the structure:

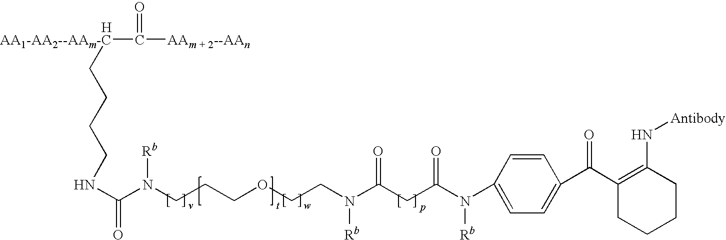

In certain of these embodiments, v is 0, 1, 2, 3, 4, or 5; t is 1, 2, 3, 4, 5, or 6; w is 1, 2, 3, 4, or 5; p is 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; w is 1; and p is 3. In some embodiments, v is 0; t is 1 or 2; w is 1; and p is 1 or 2.

Certain embodiments in accordance with Formula II have the structure:

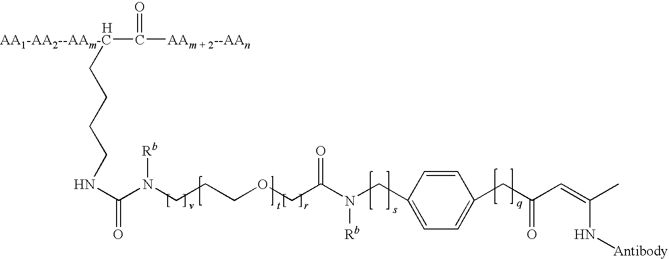

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; r is 1 or 2; s is 3; and q is 0, 1, 2, or 3. In some embodiments, v is 0; t is 1, 2, or 3, r is 1; s is 1 or 2; and q is 1 or 2.

Certain embodiments in accordance with Formula II have the structure:

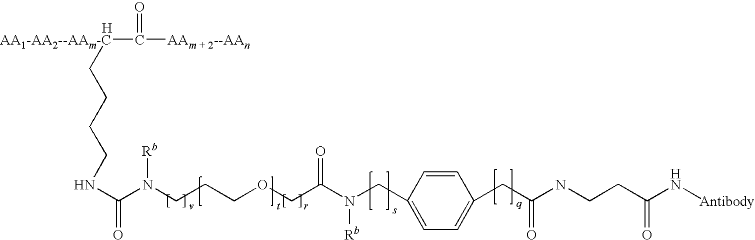

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; q is 0, 1, 2, or 3; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; r is 1 or 2; s is 3; and q is 0, 1, 2, or 3. In some embodiments, v is 0; t is 1, 2, or 3, r is 1; s is 1 or 2; and q is 2 or 3.

Certain embodiments in accordance with Formula II have the structure:

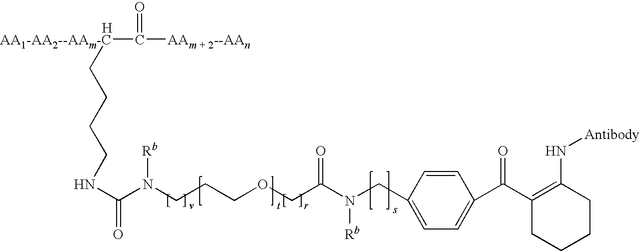

In certain of these embodiments, v is 0, 1, 2, 3, 4, or 5; t is 1, 2, 3, 4, 5, or 6; r is 1, 2, 3, 4, or 5; s is 0, 1, 2, 3, 4, or 5; and R^{b} at each occurrence is independently hydrogen, substituted or unsubstituted C₁₋₁₀ alkyl, substituted or unsubstituted C₃₋₇ cycloalkyl-C₀₋₆ alkyl, or substituted or unsubstituted aryl-C₀₋₆ alkyl. In certain embodiments, v is 0; t is 1, 2, 3, 4, 5, or 6; r is 1 or 2; and s is 3. In some embodiments, v is 0; t is 1, 2, or 3,r is 1;and s is 1 or 2.

Alternatively, the linker may have an amine or hydrazide as the reactive group and the Antibody may be engineered to have a diketone moiety. An unnatural diketone-containing amino acid may be readily incorporated into an antibody combining site using techniques well known in the art; proteins containing unnatural amino acids have been produced in yeast and bacteria. See, e.g., J. W. Chin et al., Science 301:964-966 (2003); L. Wang et al., Science 292:498-500 (2001); J. W. Chin et al., J. Am. Chem. Soc. 124:9026-9027 (2002); L. Wang, et al., J. Am. Chem. Soc. 124:1836-1837 (2002); J. W. Chin and P. G. Schultz, Chembiochem. 3:1135-1137 (2002); J. W. Chin et al., Proc. Natl. Acad. Sci. U.S.A. 99:11020-11024 (2002); L. Wang and P. G. Schultz, Chem. Commun. (1): 1-11 (2002); Z. Zhang et al., Angew. Chem. Int. Ed. Engl. 41:2840-2842 (2002); L. Wang, Proc. Natl. Acad. Sci. U.S.A. 100:56-61 (2003). Thus, for example, to insert an unnatural amino acid containing a diketone moiety into the yeast Saccharomyces cerevisiae requires the addition of new components to the protein biosynthetic machinery including a unique codon, tRNA, and aminoacyl-tRNA synthetase (aa RS). For example, the amber suppressor tyrosyl-tRNA synthetase (TyrRS)-tRNA_{CUA} pair from E. coli may be used as reported for eukaryotes in J. W. Chin et al., Science 301:964-966 (2003). The amber codon is used to code for the unnatural amino acid of interest. Libraries of mutant TyrRS and tRNA_{CUA} may then be produced and selected for those aaRS-tRNA_{CUA} pairs in which the TyrRS charges the tRNA_{CUA} with the unnatural amino acid of interest, e.g., the diketone-containing amino acid. Subsequently, antibodies incorporating the diketone-containing amino acid may be produced by cloning and expressing a gene containing the amber codon at one or more antibody combining sites.

In some embodiments of compounds of Formula II the Antibody is a full length antibody. In other embodiments, the Antibody is Fab, Fab′ F(ab′)₂, Fv, V_{H}, V_{L}, or scFv. In other embodiments, Antibody is a human antibody, humanized antibody or chimeric human antibody. In still other embodiments, the Antibody is a catalytic antibody. In one embodiment, Antibody is a humanized version of a murine 38c2 comprising a constant region from a human IgG, IgA, IgM, IgD, or IgE antibody. In another embodiment, Antibody is a chimeric antibody comprising the variable region from murine 38c2 and a constant region from a human IgG, IgA, IgM, IgD, or IgE antibody.

In some cases, two or more GA targeting agents may be linked to a single full length bivalent

Antibody. This is shown below as Formula III:

Antibody[L′-[GA targeting agent]]₂ (III)

Also provided are stereoisomers, tautomers, solvates, prodrugs, and pharmaceutically acceptable salts thereof.

In compounds of Formula III, [GA targeting agent], L′ and Antibody are each defined as in Formula II.

Targeting compounds such as those of Formula II may also be readily synthesized by covalently linking a targeting agent-linker to a combining site of a multivalent antibody. For example, a GA targeting-agent linker conjugate, where the linker includes a diketone reactive moiety, can be incubated with 0.5 equivalents of an aldolase antibody such as h38C2 IgG1 to produce a GA targeting compound. Alternatively, a GA targeting compound such as those of Formula III may be produced by covalently linking a GA targeting agent-linker compound as described herein to each combining site of a bivalent antibody.

### Pharmaceutical Compositions of GA Targeting Compounds

Another aspect of the invention provides pharmaceutical compositions of the GA targeting compounds. GA targeting compounds may be administered using techniques well known to those in the art. Preferably, agents are formulated and administered systemically. Techniques for formulation and administration may be found in Remington's Pharmaceutical Sciences, 18^{th} Ed., 1990, Mack Publishing Co., Easton, Pa. For injection, GA targeting compounds may be formulated in aqueous solutions, emulsions or suspensions. GA targeting compounds are preferably formulated in aqueous solutions containing physiologically compatible buffers such as citrate, acetate, histidine or phosphate. Where necessary, such formulations may also contain various tonicity adjusting agents, solubilizing agents and/or stabilizing agents (e.g., salts such as sodium chloride, sugars such as sucrose, mannitol, and trehalose, proteins such as albumin, amino acids such as glycine and histidine, surfactants such as polysorbates (Tweens), or cosolvents such as ethanol, polyethylene glycol and propylene glycol).

### Methods of Use for GA Targeting Compounds

One aspect of the invention is a method for treating diabetes or a diabetes-related condition comprising administering a therapeutically effective amount of a GA targeting compound to a subject suffering from diabetes or a diabetes-related condition. For therapeutic use in humans, a human, humanized, or human chimeric antibody is a preferred antibody form of the targeting compound.

Another aspect of the invention is a method for increasing insulin secretion in a subject comprising administering to the subject a therapeutically effective amount of a GA targeting compound or a pharmaceutical derivative thereof.

Yet another aspect of the invention is a method for decreasing blood glucose levels in a subject comprising administering to the subject a therapeutically effective amount of a GA targeting compound or a pharmaceutical derivative thereof.

### Methods of Administration and Dosages

Administration routes of GA targeting compounds may include parenteral delivery, including intramuscular, subcutaneous, or intramedullary injections, as well as intrathecal, direct intraventricular, intravenous, and intraperitoneal delivery. In one embodiment, administration is intravenous. The GA targeting compounds may be administered through any of the parenteral routes either by direct injection of the formulation or by infusion of a mixture of the targeting GA compound formulation with an infusion matrix such as normal saline, D5W, lactated Ringers solution or other commonly used infusion media.

In treating mammals, including humans, having diabetes or a diabetes-related condition, a therapeutically effective amount of a GA targeting compound or a pharmaceutically acceptable derivative is administered. For example, a GA targeting compound may be administered as a daily intravenous infusion from about 0.1 mg/kg body weight to about 15 mg/kg body weight. Accordingly, one embodiment provides a dose of about 0.5 mg/kg body weight. Another embodiment provides a dose of about 0.75 mg/kg body weight. Another embodiment provides a dose of about 1.0 mg/kg body weight. Another embodiment provides a dose of about 2.5 mg/kg body weight. Another embodiment provides a dose of about 5 mg/kg body weight. Another embodiment provides a dose of about 10.0 mg/kg body weight. Another embodiment provides a dose of about 15.0 mg/kg body weight. Doses of a GA targeting compound or a pharmaceutically acceptable derivative should be administered in intervals of from about once per day to 2 times per week, or alternatively, from about once every week to once per month. In one embodiment, a dose is administered to achieve peak plasma concentrations of a GA targeting compound according to the invention or a pharmaceutically acceptable derivative thereof from about 0.002 mg/ml to 30 mg/ml. This may be achieved by the sterile injection of a solution of the administered ingredients in an appropriate formulation (any suitable formulation solutions known to those skilled in the art of chemistry may be used). Desirable blood levels may be maintained by a continuous infusion of a GA targeting compound according to the invention as ascertained by plasma levels measured by a validated analytical methodology.

One method for administering a GA targeting compound to an individual comprises administering a GA targeting agent linker conjugate to the individual and allowing it to form a covalent compound with a combining site of an appropriate antibody in vivo. The antibody portion of a GA targeting compound that forms in vivo may be administered to the individual before, at the same time, or after administration of the targeting agent linker conjugate. As already discussed, a GA targeting agent may include a linker/reactive moiety, or the antibody combining site may be suitably modified to covalently link to the targeting agent. Alternatively, or in addition, an antibody may be present in the circulation of the individual following immunization with an appropriate immunogen. For example, catalytic antibodies may be generated by immunizing with a reactive intermediate of the substrate conjugated to a carrier protein. See R. A. Lerner and C. F. Barbas 3^{rd}, Acta Chem. Scand. 50:672-678 (1996). In particular, aldolase catalytic antibodies may be generated by administering with keyhole limpet hemocyanin linked to a diketone moiety as described by P. Wirsching et al., Science 270:1775-1782 (1995) (commenting on J. Wagner et al., Science 270:1797-1800 (1995)).

### Combination Therapies

In another aspect of the invention, a GA targeting compound may be used in combination with other therapeutic agents used to treat diabetes or diabetes-related conditions, or to increase insulin secretion or decrease blood glucose levels. In one embodiment, a GA targeting compound may be administered in combination with insulin, such as for example synthetic human insulin, including rapid acting, short-acting, intermediate-acting, or long-lasting insulin. In other embodiments, GA targeting compounds may be administered in combination with compounds belonging to the α-glucosidase inhibitor, sulfonylurea, meglitinide, biguanide, or thiazolidinedione (TZD) families. GA targeting compounds may also be administered in combination with metabolism-modifying proteins or peptides such as glucokinase (GK), glucokinase regulatory protein (GKRP), uncoupling proteins 2 and 3 (UCP2 and UCP3), peroxisome proliferator-activated receptor α (PPARα), leptin receptor (OB-R^{b}), DPP-IV inhibitors, sulfonylureas, or other incretin peptides. One of ordinary skill in the art would know of a wide variety of agents that are currently used in the treatment of diabetes or diabetes-related conditions.

In order to evaluate potential therapeutic efficacy of a GA targeting compound or a pharmaceutically acceptable derivative thereof in combination with other therapeutic agents used to treat diabetes or diabetes-related conditions, increase insulin secretion, or decrease blood glucose levels, these combinations may be tested using methods known in the art. For example, the ability of a combination of a GA targeting compound(s) according to the invention and another therapeutic agent to increase insulin secretion may be measured using an in vitro glucose-stimulated insulin secretion assay. In such an assay, pancreatic β cells are treated with various concentrations of glucose for a set period of time, and insulin levels are measured using methods known in the art, such as for example a radioimmunoassay. The effect of GA targeting compound(s) according to the invention and other therapeutic agents on insulin secretion may also be measured in vivo, by administering the agents directly to a subject and measuring insulin levels in bodily fluid samples at various timepoints. Methods for administering known therapeutic agents to a subject for use in combination therapies will be well known to clinical health care providers.

Effective dosages of GA targeting compounds to be administered may be determined through procedures well known to those in the art which address such parameters as biological half-life, bioavailability, and toxicity. Effective amounts of therapeutic agents to be used in combination with GA targeting compounds or pharmaceutically acceptable derivatives thereof are based on the recommended doses known to those skilled in the art for these agents. These recommended or known levels will preferably be lowered by 10% to 50% of the cited dosage after testing the effectiveness of these dosages in combination with a GA targeting compound according to the invention or a pharmaceutically acceptable derivative. It should be noted that the attending physician would know how to and when to terminate, interrupt, or adjust therapy to lower dosage due to toxicity, bone marrow, liver or kidney dysfunctions or adverse drug-drug interaction. Conversely, the attending physician would also know to adjust treatment to higher levels if the clinical response is not adequate (precluding toxicity).

A therapeutically effective dose refers to that amount of the compound sufficient to result in amelioration of symptoms or a prolongation of survival in a patient. The effective in vitro concentration of a GA targeting agent may be determined by measuring the EC50. Toxicity and therapeutic efficacy of such agents in vivo can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. The data obtained from these cell culture assays and animal studies can be used in formulating a range of dosage for use in humans. The dosage of such compounds lies preferably within a range of circulating concentrations that include the ED50 with little or not toxicity. The dosage may vary within this range depending upon the dosage form employed and the route of administration utilized. For any compound, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of RT production from infected cells compared to untreated control as determined in cell culture). Such information can be used to more accurately determine useful doses in humans. Levels in plasma may be measured, for example, by high performance liquid chromatography (HPLC).

In those embodiments wherein GA targeting compounds are administered in combination with other therapeutic agents, the combined effect of the agents can be calculated by the multiple drug analysis method of Chou and Talalay (T. C. Chou and P. Talalay, Adv. Enzyme Regul. 22:27-55 (1984)) using the equation:

$$C I = \frac{{D}_{1}}{{( Dx )}_{1}} + \frac{{D}_{2}}{{( Dx )}_{2}} + \frac{α {D}_{1} {D}_{2}}{{( Dx )}_{1} {( Dx )}_{2}}$$

Where CI is the combination index, (Dx)₁ is the dose of drug 1 required to produce x percent effect alone, D₁ is the dose of drug 1 required to produce the same x percent effect in combination with D₂. The values of (Dx)₂ and (D)₂ are similarly derived from drug 2. The value of α is determined from the plot of the dose effect curve using the median effect equation:

$$\frac{f a}{f u} = {( \frac{D}{Dm} )}^{m}$$

where fa is the fraction affected by dose D, fu is the uninfected fraction, Dm is the dose required for 50% effect and m is the slope of the dose-effect curve. For mutually exclusive drugs (i.e., similar modes of action), both drugs alone and their parallel lines in the median effect plot. Mutually nonexclusive drugs (i.e., independent mode of action) will give parallel lines in the median effect plot, but in mixture will give a concave upward curve. If the agents are mutually exclusive a is 0. and if they are mutually non-exclusive, α is 1. Values obtained assuming mutual nonexclusiveness will always be slightly greater than mutually exclusive drugs. CI values of <1 indicate synergy, values>1 indicate antagonism and values equal to 1 indicate additive effects.

The combined drug effects may also be calculated using the CalcuSyn software package from Biosoft (Cambridge, UK).

### EXAMPLES

### Example 1

### Synthesis of HAEGTFTSDVSSYLEGQAAKEFIAWLVKGR (SEQ ID NO:1)

Solid phase peptide synthesis of the modified peptide on a 100 μmol scale is performed on a Symphony Peptide Synthesizer using Fmoc chemistry employing Fmoc protected PL-Rink resin (0.68 mmol/g, Polymer Laboratories). The following N′-Fmoc protected amino acids are utilized in the synthesis: Fmoc-Arg(Pbf)-OH, Fmoc-Gly-OH, Fmoc-Lys(Boc)-OH, Fmoc-Val-OH, Fmoc-Leu-OH, Fmoc-Trp(Boc)-OH, Fmoc-Ala-OH, Fmoc-Ile-OH, Fmoc-Phe-OH, Fmoc-Glu(tBut)-OH, Fmoc-Gln-OH, Fmoc-Gly-OH, Fmoc-Leu-OH, Fmoc-Tyr(tBut)-OH, Fmoc-Ser(tBut)-OH, Fmoc-Asp(tBut)-OH, Fmoc-Thr(tBut)-OH and Fmoc-His(Trt)-OH. Briefly, the coupling reactions are carried out in N-Methylpyrrolidinone (NMP) using 10 equivalents of amino acids and 10 equivalents of activating agents O-benzotriazol-1-yl-N,N,N¹,N¹-tetramethyl-uronium hexafluorophosphate (HBTU) and N-Hydroxybenzotriazole (HOBT) in the presence of 30 equivalents of N-Methylmorpholine (NMM) with each coupling carried out for 2 hr. Removal of the N′-Fmoc protecting group is achieved using a solution of 25% (V/V) piperidine in NMP four times for five minutes each. Between every coupling, the resin is washed six times with NMP. The peptide is cleaved from the resin using 85% TFA/5% TIS/5% thioanisole and 5% phenol, followed by precipitation by dry-ice cold Et₂O. The crude peptide is centrifuged and lyophilized, and the product is purified by a reverse phase HPLC using a C₁₈ column employing 0.1% TFA in acetonitrile and 0.1% TFA in water as a mobile phase to afford the pure compound as a white solid.

Amino acids and N-Hydroxybenzotriazole (HOBT) are dissolved in NMP and, according to the sequence, activated using HBTU or O-(7-Azabenzotriazole-1-yl)-N,N,N,N′,N′-tetramethyluronium hexafluorophosphate (HATU). For HBTU activation, the amino acid, HBTU added at 10 equivalents relative to resin loading, and NMM is added at 30 equivalents. HBTU activation for each amino acid is performed twice for two hours each time. For HATU activation, the amino acid and HATU are added at 10 equivalents relative to resin loading, and diisopropylethylamine (DIEA) is added at 20 equivalents. HATU activation for each amino acid is carried out for three hours. Removal of the Fmoc protecting group is achieved using a solution of 25% (V/V) piperidine in NMP four times for five minutes each. Between every coupling, the resin is washed six times with NMP. The peptide is cleaved from the resin using 85% TFA/5% TIS/5% thioanisole and 5% phenol, followed by precipitation by dry-ice cold Et₂O. The crude peptide is centrifuged and lyophilized, and the product is purified by a reverse phase HPLC using a Cl₈ column employing 0.1% TFA in acetonitrile and 0.1% TFA in water as a mobile phase to afford the pure compound as a white solid.

### Example 2

### Synthesis of HGEGTFTSDLSKQMEEEAVRLFIEWLKNGGPSSGAPPPS (SEQ ID NO:2)

Solid phase peptide synthesis of the modified peptide on a 100 μmol scale is performed on a Symphony Peptide Synthesizer using Fmoc chemistry employing Fmoc protected PL-Rink resin (0.68 mmol/g, Polymer Laboratories). The following N^{α}-Fmoc protected amino acids are used in the synthesis: Fmoc-Ser(tBu)-OH, Fmoc-Pro-OH, Fmoc-Ala-OH, Fmoc-Gly-OH, Fmoc, Fmoc-Asn(Trt)-OH, Fmoc-Lys(Boc)-OH, Fmoc-Leu-OH, Fmoc-Trp(Boc)-OH, Fmoc-Glu(tBu)-OH, Fmoc-Ile-OH, Fmoc-Phe-OH, Fmoc-Arg(Pbf)-OH, Fmoc-Val-OH, Fmoc-Met-OH, Fmoc-Gln(Trt)-OH, Fmoc-Asp(tBu)-OH, Fmoc-Thr(tBut)-OH, and Fmoc-His(Trt)-OH. Briefly the coupling reactions are carried out in N-Methylpyrrolidinone (NMP) using 10 equivalents of amino acids and 10 equivalents of activating agents O-benzotriazol-1-yl-N,N,N¹,N¹-tetramethyl-uronium hexafluorophosphate (HBTU) or O-(7-Azabenzotriazole-1-yl)-N,N,N,N′,N′-tetramethyluronium hexafluorophosphate (HATU) along with N-Hydroxybenzotriazole (HOBT). For HBTU activation, the amino acid, HBTU added at 10 equivalents relative to resin loading, and NMM is added at 30 equivalents. HBTU activation for each amino acid is performed twice for two hours each time. For HATU activation, the amino acid and HATU are added at 10 equivalents relative to resin loading, and diisopropylethylamine (DIEA) is added at 20 equivalents. HATU activation for each amino acid is carried out for three hours. Removal of the N′-Fmoc protecting group is achieved using a solution of 25% (V/V) piperidine in NMP four times for five minutes each. Between every coupling, the resin is washed six times with NMP. The peptide is cleaved from the resin using 85% TFA/5% TIS/5% thioanisole and 5% phenol, followed by precipitation by dry-ice cold Et₂O. The crude peptide is centrifuged and lyophilized, and the product is purified by a reverse phase HPLC using a Cl₈ column employing 0.1% TFA in acetonitrile and 0.1% TFA in water as a mobile phase to afford the pure compound as a white solid.

### Example 3

### Synthesis of

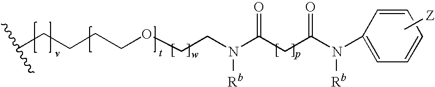

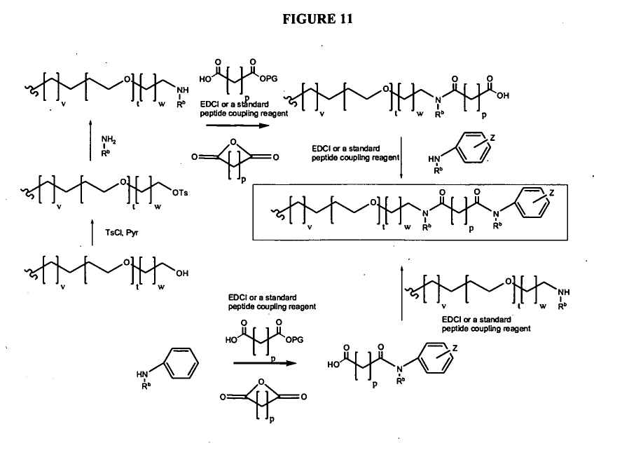

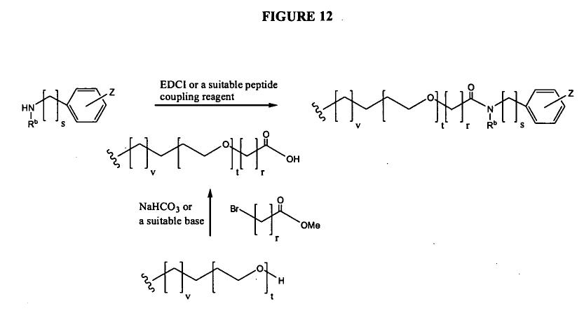

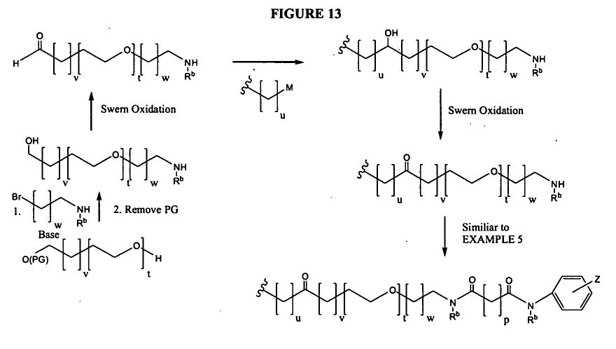

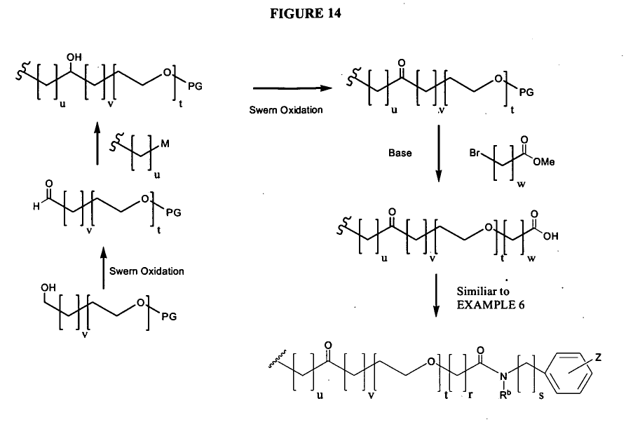

is provided in FIG. 11.

### Example 4

### Synthesis of

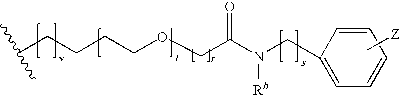

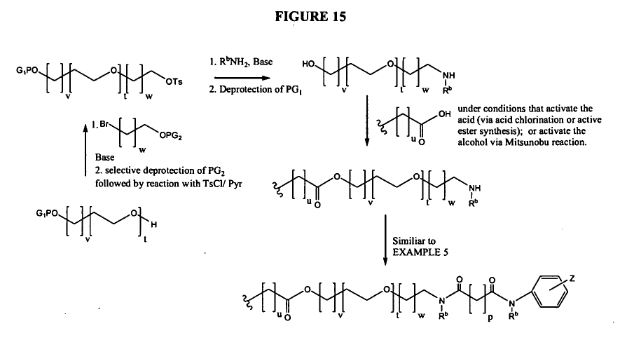

is provided in FIG. 12.

### Example 5

### Synthesis of

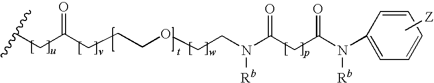

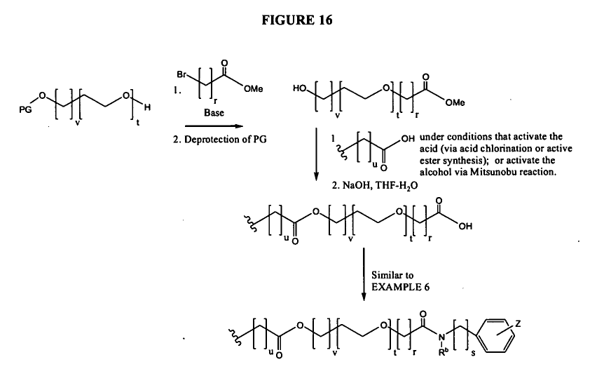

is provided in FIG. 13.

### Example 6

### Synthesis of

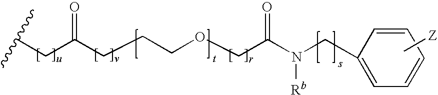

is provided in FIG. 14.

### Example 7

### Synthesis of

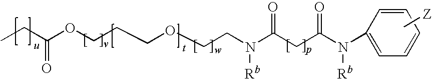

is provided in FIG. 15.

### Example 8

### Synthesis of

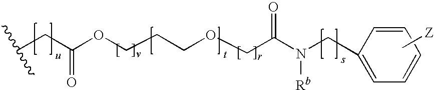

is provided in FIG. 16.

### Example 9

### Synthesis of

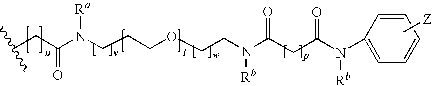

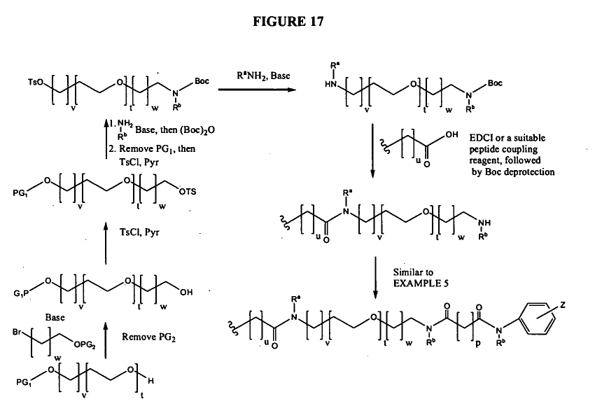

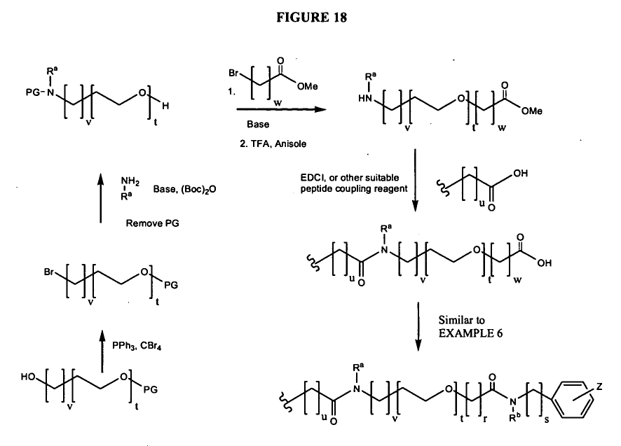

is provided in FIG. 17.

### Example 10

### Synthesis of

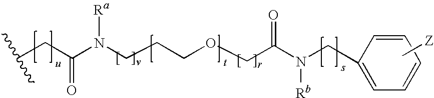

is provided in FIG. 18.

### Example 11

### Synthesis of

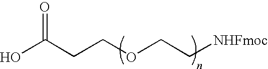

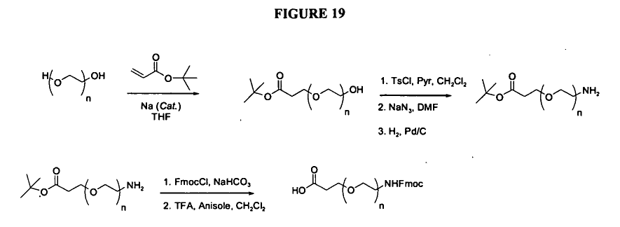

is provided in FIG. 19.

### Example 12

### Synthesis of

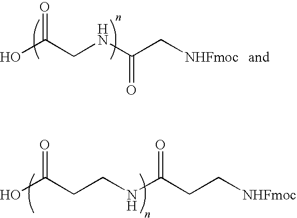

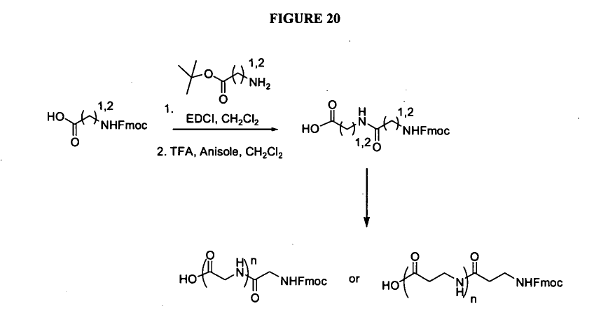

is provided in FIG. 20.

### Example 13

### Synthesis of

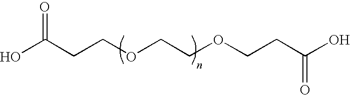

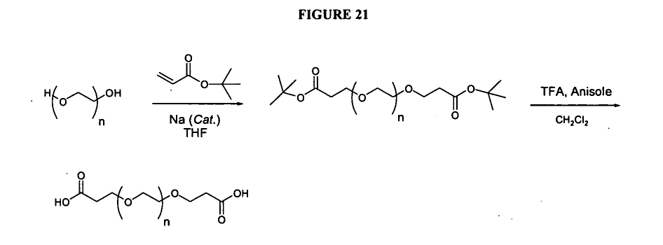

is provided in FIG. 21.

### Example 14

### Synthesis of

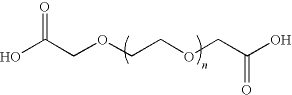

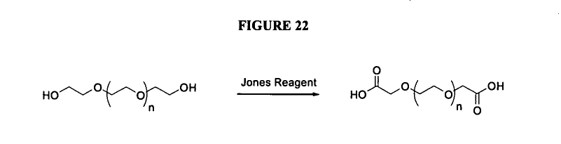

is provided in FIG. 22.

### Example 15

### Synthesis of

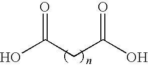

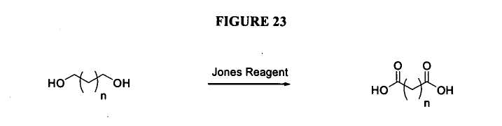

is provided in FIG. 23.

### Example 16

### Synthesis of

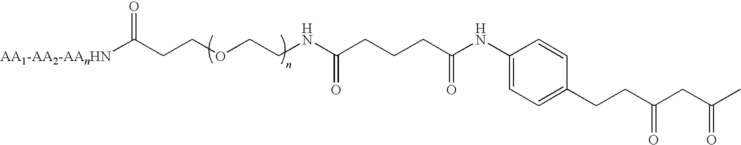

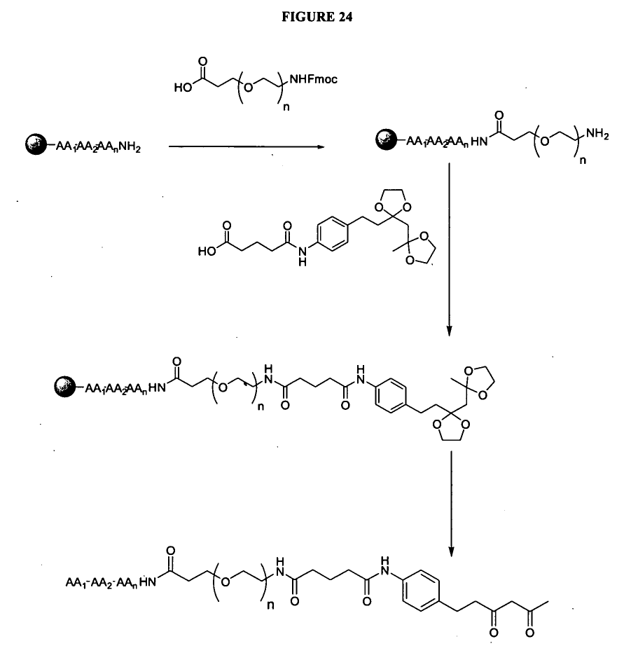

is provided in FIG. 24. While this EXAMPLE uses the compound of EXAMPLE 11, another synthesis could also sufficiently employ the compounds of EXAMPLE 12 in the first step. Further, while this EXAMPLE shows linking to the N-terminus, the free acid on the left side of the compounds of EXAMPLES 11 and 12 may also be linked to any nucleophilic sidechain on a peptide, such as the C, K, S, T or Y sidechains. As is also shown in this EXAMPLE, the Fmoc protected amino group on the right side of the compounds of EXAMPLES 11 and 12 is used to link to the antibody recognition group, G, via an amide bond.

### Example 17

### Synthesis of

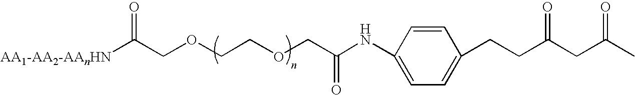

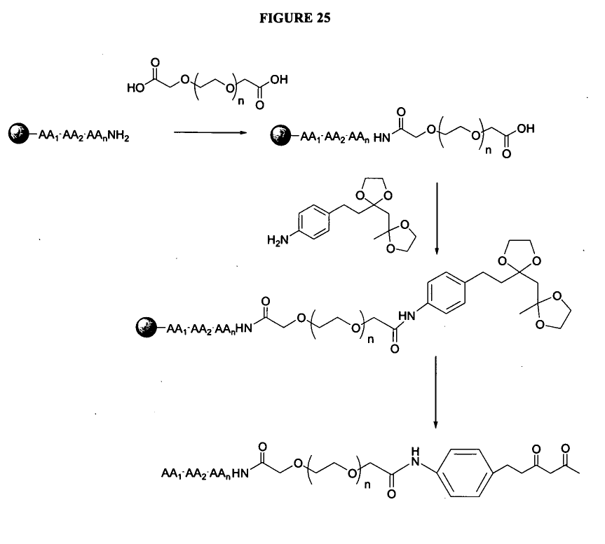

is provided in FIG. 25. While this EXAMPLE uses the compound of EXAMPLE 14, another synthesis could also sufficiently employ the compounds of EXAMPLES 13 or 15 in the first step. Further, while this EXAMPLE shows linking to the N-terminus, the free acid on the left side of the compounds of EXAMPLES 13-15 may also be linked to any nucleophilic sidechain on a peptide, such as the C, K, S, T or Y sidechains. As is also shown in this EXAMPLE, the free acid on the right side of the compounds of EXAMPLES 13-15 is used to link to the antibody recognition group, G, via an amide bond.

### Example 18

### Synthesis of

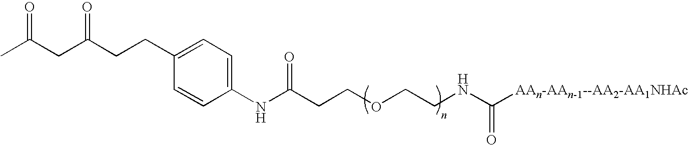

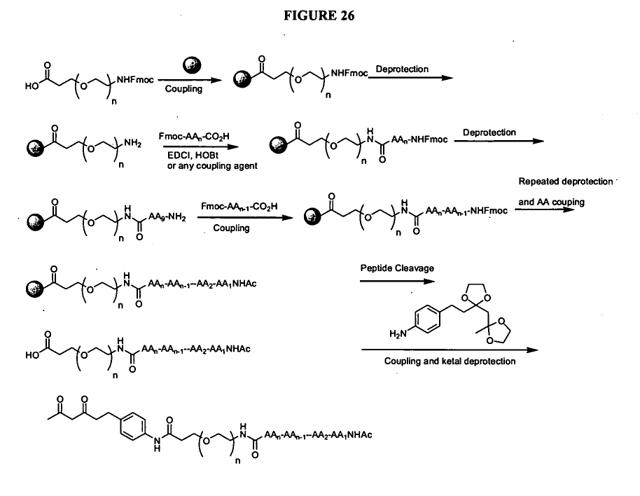

is provided in FIG. 26. While this EXAMPLE uses the compound of EXAMPLE 11, another synthesis could also sufficiently employ the compounds of EXAMPLE 12 in the first step.

### Example 19

### Synthesis of 3-{2-[2-(2-{2-[2-(2-tert-Butoxycarbonyl-ethoxy)-ethoxy]-ethoxy}-ethoxy)-ethoxy]ethoxy}-propionic acid tert-butyl ester

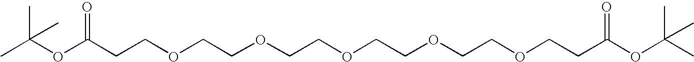

The title compound was prepared using a reported method (O, Seitz and H. Kunz, J. Org. Chem. 62:813-826 (1997)). A small piece of sodium metal was added to a solution of tetra(ethylene glycol) (47.5 g, 244 mmol) in THF (200 ml) and stirred until the sodium was dissolved completely. ^{t}Butyl acrylate (94 g, 730 mmol) was then added and stirring continued for 2 days at RT. Another batch of ^{t}Butyl acrylate (94 g, 730 mmol) was added and stirring continued for another 2 days. The reaction mixture was neutralized with a few drops of 1N HCl and concentrated under reduced pressure. The residue was suspended in water and extracted with ethyl acetate (3×150 ml). Combined organic layers were washed with brine and dried over sodium sulfate. Evaporation of volatiles over reduced pressure provided the crude product as colorless liquid which was purified using a silica gel column (42 g, 51%).

### Example 20

### Synthesis of 3-{2-[2-(2-{2-[2-(2-Carboxy-ethoxy)-ethoxy]-ethoxy}-ethoxy)-ethoxy]-ethoxy}-propionic acid

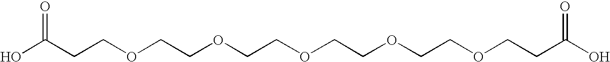

A solution of 3-{2-[2-(2-{2-[2-(2-tert-Butoxycarbonyl-ethoxy)-ethoxy]-ethoxy}-ethoxy)-ethoxy]-ethoxy}-propionic acid tert-butyl ester (6 g, 18.6 mmol) in anisole (20 ml) was cooled in an ice bath and trifluoroacetic acid (65 g) was added. After 3 hrs at RT volatiles were removed under reduced pressure and the residue was partitioned between ethyl acetate (50 ml) and 5% sodium bicarbonate solution. The aqueous layer was acidified with 1 N HCl, saturated with NaCl and then extracted with ethyl acetate (3×50 ml). Combined organic layers were washed with brine and dried over sodium sulfate. Removal of volatiles under the reduced pressure provided the product as colorless liquid which solidified upon refrigeration (3.8 g, 82%).

### Example 21

### Synthesis of 3-(2-{2-[2-(2-{2-[2-(4-{2-[2-(2-Methyl-[1,3]dioxolan-2-ylmethyl)-[1,3]dioxolan-2-yl]-ethyl}-phenylcarbamoyl)-ethoxy]-ethoxy}-ethoxy)-ethoxy]-ethoxy}-ethoxy)-propionic acid

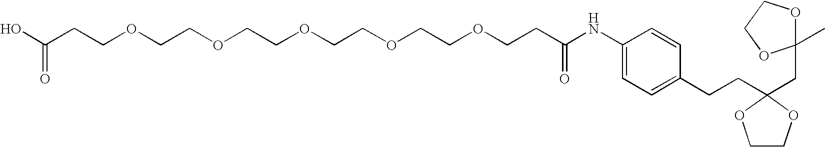

Compound from EXAMPLE 20 (0.6 g, 1.8 mmol) was dissolved in dichloromethane (10 ml) and 4-{2-[2-(2-Methyl-[1,3]dioxolan-2-ylmethyl)-[1,3]dioxolan-2-yl]-ethyl}-phenylamine (0.3 g, 1.4 mmol) followed by EDCI (0.28 g, 1.8 mmol) was added at RT. After 1 hr at RT the RM was washed with water and dried over sodium sulfate. Evaporation of volatiles and purification over silica gel column with 1 to 15% methanol in dichloromethane provided title compound as gum (0.47 g, 32%).

### Example 22

### Synthesis of 4-{2-[2-(2-Methyl-[1,3]dioxolan-2-ylmethyl)-[1,3]dioxolan-2-yl]-ethyl}-phenylamine

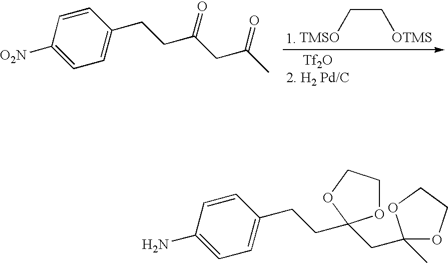

A clean oven dried flask was charged with the 6-(4-nitro-phenyl)-hexane-2,4-dione (3.7 g, 15.72 mmol), dry CH₂Cl₂ (20 ml) followed by bisTMS ethylene glycol (38.5 ml, 157.3 ml) were added to the flask and the resulting solution was cooled to −5° C. with stirring under argon. TMSOTf (300 μl) was added to the reaction mixture and the solution was stirred at −5° C. for 6 h. Reaction was quenched with pyridine (10 ml) and poured into sat. NaHCO₃. The mixture was extracted with EtOAc and the organic layer was washed with water, brine, dried (Na₂SO₄) and concentrated to give a yellow solid. The solid was triturated with hexanes to give a free flowing pale yellow solid (3.5 g, 72%) which was dissolved in EtOAc (50 ml) and hydrogenated on a Parr shaker starting with 50 psi of hydrogen pressure. After two hours the reaction was filtered through a pad of celite, the celite was washed thoroughly with CH₂Cl₂/MeOH and combined organics were concentrated to give title compound (1.46 g, 100%) as an oil that solidifies upon standing.

### Example 23

### Synthesis of 4-[4-(3,5-Dioxo-hexyl)-phenylcarbamoyl]-butyric acid 2,5-dioxo pyrrolidin-1-yl ester (10)

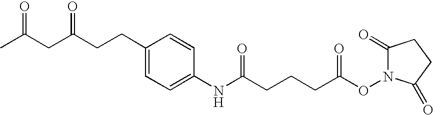

### Step 1: 6-(4-Nitro-phenyl)-hexane-2,4-dione (11)

To a reaction vessel (heat and vacuum dried and equipped with a magnetic spin bar) was added tetrahydrofuran and lithium diisopropylamide (2M heptane/ethylbenzene/tetrahydrofuran; 69.4 mL, 138.9 mmol). The solution cooled to −78° C. Pentane-2,4-dione (7.13 mL, 69.4 mmol) was added dropwise and the solution stirred 30 minutes at −78° C. 4-nitrobenzyl bromide (15.0 g, 69.4 mmol) was added in one portion. The solution was removed from the dry-ice/acetone bath, allowed to warm to room temperature and stirred 16 hours. The solution was cooled to approximately 0° C. and the reaction quenched with 1M HCl. Tetrahydrofuran was removed under reduced pressure. The crude material was taken up into dichloromethane and washed with 1M HCl and brine. The aqueous layers were again washed with dichloromethane. The combined dichloromethane layers were dried (Na₂SO₄) and removed under reduced pressure. Gradient flash column chromatography (FCC) was performed using 5% to 15% ethyl acetate/hexanes to afford title compound (8.5 g, 52%; yellow solid). ¹H NMR (CDCl₃): δ 8.14 (d, J=9.0 Hz, 2H), δ 7.43 (d, J=8.4 Hz, 2H), δ 5.45 (s, 1H), δ 3.06 (t, J=7.5 Hz, 2H), δ 2.64 (t, J=7.8 Hz, 2H), δ 2.04 (s, 3H).

### Step 2: 4-[4-(3,5-Dioxo-hexyl)-phenylcarbamoyl]-butyric acid (12)

200 mL tetrahydrofuran, 6-(4-nitro-phenyl)-hexane-2,4-dione (8.0 g, 34.0 mmol) and dihydro-pyran-2,6-dione (3.88 g, 34.0 mmol) were added to a reaction vessel. The reaction vessel was purged three times with argon. Approximately 200 mg palladium (10 wt % on activated carbon) was added. The reaction vessel was purged again with argon and excess hydrogen introduced via a balloon. Solution stirred 16 hours at room temperature. Hydrogen removed under reduced pressure and catalyst removed by filtration through celite. Tetrahydrofuran removed under reduced pressure to afford title compound (10.5 g, 97%, yellow solid).

### Step 3: 4-[4-(3,5-Dioxo-hexyl)-phenylcarbamoyl]-butyric acid 2,5-dioxo pyrrolidin-1-yl ester (10)

To a reaction vessel (heat and vacuum dried and equipped with a magnetic spin bar) was added 4-[4-(3,5-dioxo-hexyl)-phenylcarbamoyl]-butyric acid (10.53 g, 33.0 mmol), N-hydroxysuccinimide (3.8 g, 33.0 mmol) and 1-[3-(dimethylamino) propyl]-3-ethylcarbodiimide hydrochloride (6.3 g, 33.0 mmol) and dichloromethane (250 mL). The solution was stirred under nitrogen at room temperature for 16 hours then washed with 10% citric acid, brine and dried (Na₂SO₄). Dichloromethane was removed under reduced pressure. FCC with 70% ethyl acetate/hexanes gave title compound (7.4 g, yellow solid, 54%).

¹H NMR (CDCl₃): δ 7.87 (s, 1H), δ 7.43 (d, J=8.4 Hz, 2H), δ 7.12 (d, J=8.4 Hz, 2H), δ 5.46 (s, 1H), δ 2.89 (t(& m), J=8.1 Hz (for the t), 7H), δ 2.73 (t, J=6.0 Hz, 2H), δ 2.56 (t, J=7.2 Hz, 2H), δ 2.47 (t, J=6.9 Hz, 2H), δ 2.21 (p, J=6.6 Hz, 2H), δ 2.04 (s, 3H).

### Example 23

### Synthesis of 3-{2-[2-(2-{4-[4-(3,5-Dioxo-hexyl)-phenylcarbamoyl]-butyrylamino}-ethoxy)-ethoxy]-ethoxy}-propionic acid 2,5-dioxo-pyrrolidin-1-yl ester, (20)

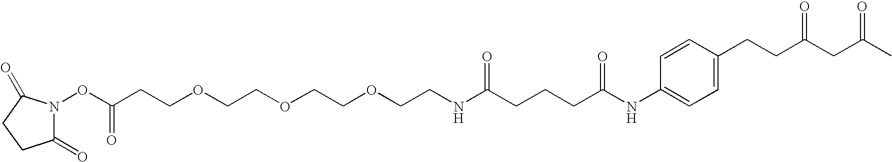

### Step 1: 3-{2-[2-(2-Hydroxy-ethoxy)-ethoxy]-ethoxy}-propionic acid tert-butyl ester

Na metal (catalytic) was added to a stirring solution of acrylic acid tert-butyl ester (6.7 mL, 46 mmol), and 2-[2-(2-hydroxy-ethoxy)-ethoxy]-ethanol (20.7 g, 138 mmol) in THF (100 mL) at 0° C. and the mixture was stirred overnight. Solvent was removed and the remaining oil dissolved in EtOAc (100 mL). The organic layer was washed with water (3×50 mL), and dried over Na₂SO₄ and the solvent removed in vacuo to give an oil which corresponds to the title compound that would be used as is for the next step. (M+1)=279.

### Step 2: 3-{2-[2-(2-Tosylsulfonyloxy-ethoxy)-ethoxy]-ethoxy}-propionic acid tert-butyl ester

Tosyl chloride (22.3 g, 117 mmol) was added in portions to a stirring solution of 3-{2-[2-(2-hydroxy-ethoxy)-ethoxy]-ethoxy}-propionic acid tert-butyl ester (16.3 g, 58.6 mmol) and pyridine 60 mL in (240 mL) and the mixture was stirred overnight. The reaction was quenched with water (300 mL) and the organic layer was separated. The aqueous layer was extracted with CH₂Cl₂ (2×100 mL). The combined organic layer was washed with HCl (1N, 100 mL), water (100 mL), and dried over Na₂SO₄ and the solvent was removed in vacuo to give an oil which corresponds to the title compound that would be used as is for the next step. (M+1)=433.

### Step 3: 3-{2-[2-(2-Amino-ethoxy)-ethoxy]-ethoxy}-propionic acid tert-butyl ester

NaN₃ (35 g, 538 mmol) was added to a stirring solution of 3-{2-[2-(2-tosylsulfonyloxy-ethoxy)-ethoxy]-ethoxy}-propionic acid tert-butyl ester (20 g, 46 mmol) in DMF (150 mL) and the reaction was stirred overnight. Reaction was diluted with water (200 mL) and extracted with EtOAc (4×100 mL). The organic layer was washed with water (100 mL) and brine (100 mL) and dried over Na₂SO₄. The solvent was removed in vacuo to give an oil. Column chromatography EtOAc/Hex (1:4) gave an oil which corresponds to the 3-{2-[2-(2-azido-ethoxy)-ethoxy]-ethoxy}-propionic acid tert-butyl ester, (M+1)=304. This oil was hydrogenated using Pd (5% on carbon) in EtOAc under hydrogen (1 atm.) over 3 days. The catalyst was removed by filtration and solvent removed in vacuo to give an oil corresponding to the title compound, (M+1)=278.

### Step 4: 3-{2-[2-(2-{4-[4-(3,5-Dioxo-hexyl)-phenylcarbamoyl]-butyrylamino}-ethoxy)-ethoxy]-ethoxy}-propionic acid tert-butyl ester

A solution of 4-[4-(3,5-dioxo-hexyl)-phenylcarbamoyl]-butyric acid 2,5-dioxo-pyrrolidin-1-yl ester (1.5 g, 3.6 mmol), 3-{2-[2-(2-amino-ethoxy)-ethoxy]-ethoxy}-propionic acid tert-butyl ester (1.0 g, 3.6 mmol) and DIEA (1.3 μL, 7.2 mmol) in CH₂Cl₂ (10 mL) was stirred at room temperature overnight. The solvent was removed in vacuo and the residual oil purified using column chromatography EtOAc/MeOH (95:5) to give the title compound as a transparent oil, (M+1)=579.

### Step 5: 3-{2-[2-(2-{4-[4-(3,5-Dioxo-hexyl)-phenylcarbamoyl]-butyrylamino}-ethoxy)-ethoxy]-ethoxy}-propionic acid 2,5-dioxo-pyrrolidin-1-yl ester

3-{2-[2-(2-{4-[4-(3,5-Dioxo-hexyl)-phenylcarbamoyl]-butyrylamino}-ethoxy)-ethoxy]-ethoxy}-propionic acid tert-butyl ester (400 mg, 0.692 mmol) was dissolved in TFA/CH₂Cl₂ (1:1, 3 mL) and the mixture stirred overnight. The solvent was removed to give an oil as the acid intermediate. This oil was dissolved in CH₂Cl₂ (4 mL) containing DIEA (569 μL, 3.09 mmol), N-hydroxysuccinimide (119 mg, 1.03 mmol) and EDC (197 mg, 1.0 mmol) and the mixture stirred over the night. The solvent was removed and the residual oil was purified using column chromatography EtOAc/MeOH (95:5) to give an oil as the title compound, (M+1)=620.

### Example 24

### Synthesis of an h38c2 Based GA Targeting Compound

Compounds of EXAMPLES 16 and 17 can be linked to h38c2 by the following procedure: One mL antibody h38c2 in phosphate buffered saline (10 mg/mL) is added to 12 μL of a 10 mg/mL stock solution of targeting compound and the resulting mixture maintained at room temperature for 2 hours prior to use.

### Example 25

C. Rader, et al., J. Mol. Biol. 332:889-899 (2003) details one method of making h38c2. The following details the results, materials and methods in this reference.

Results

Humanization Human V_{κ}, gene DPK-9 and human J_{κ} gene JK4 were used as frameworks for the humanization of the kappa light chain variable domain, and human V_{H} gene DP-47 and human J_{H} gene J_{H}4 are used as frameworks for the humanization of the heavy chain variable domain of m38C2. All complementarity determining region (CDR) residues as defined by Kabat et al., as well as defined framework residues in both light chain and heavy chain variable domain, were grafted from m38C2 onto the human framework. The selection of grafted framework residues may be based on the crystal structure of mouse mAb 33F12 Fab (PDB 1AXT). mAb 33F12 Fab shares a 92% sequence homology with m38c2 in the variable domains and identical CDR lengths. Furthermore, both 33F12 and m38C2 have similar catalytic activity. Grafted framework residues consisted of five residues in the light chain and seven residues in the heavy chain and encompass the residues that are likely to participate directly or indirectly in the catalytic activity of m38C2. These include the reactive lysine of m38C2, LysH⁹³, which is positioned in framework region 3 (FR3) of the heavy chain. Six residues, Ser^{H35}, Val^{H37}, Trp^{H47}, Trp^{H103}, and Phe^{L98}, which are conserved between mouse mAbs 33F12 and 38C2, are within a 5-A radius of the r amino group of LyS^{H93}. These residues were also conserved in the humanization. Lys 93 lies at the bottom of a highly hydrophobic substrate binding sites of mouse mAbs 33F12 and 38C2. In addition to CDR residues, a number of framework residues line this pocket. Among these, Leu^{L37}, Gln^{L42}, Ser^{L43}, Val^{L85}, Phe^{L87}, Val^{H5}, Ser^{H40}, Glu^{H42}, Gly^{H88}, Ile^{H89}, and Thr^{H94} were grafted onto the human framework.

Expression By fusing the humanized variable domains to human constant domains C_{κ} and C_{γ1}1, h38C2 was initially generated as Fab expressed in E. coli. Next, h38c2 IgG was formed from h38c2 Fab using the PIGG vector engineered for human IgG1 expression in mammalian cells. Supernatants from transiently transfected human 293T cells were subjected to affinity chromatography on recombinant protein A, yielding approximately 1 mg/L h38C2 IgG1. Purity was established by SDS-PAGE followed by Coomassie blue staining.

β-Diketone Compounds—

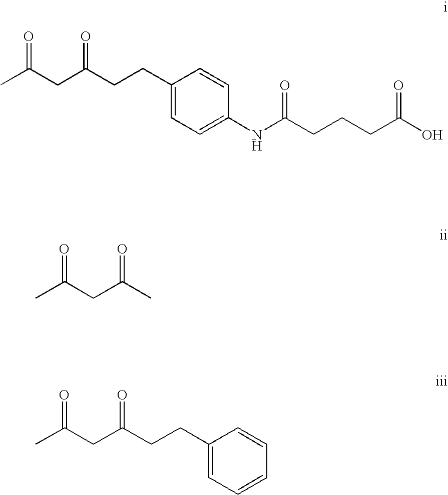

β-Diketone Compounds—The enaminone formed by the covalent addition of a β-diketone with m38c2 has a characteristic UV absorbance at λₘₐₓ=318 nm. Like m38C2 IgG, h38C2 IgG showed the characteristic enaminone absorbance after incubation with β-diketone. As a negative control, recombinant human anti-HIV-1 gp120 mAb b12 with the same IgG1 isotype as h38C2 but without reactive lysine, did not reveal enaminone absorbance after incubation with β-diketone 2. For a quantitative comparison of the binding of β-diketones to m38C2 and h38C2, the authors used a competition ELISA. The antibodies were incubated with increasing concentrations of β-diketones 2 and 3 and assayed against immobilized BSA-conjugated β-diketone 1. The apparent equilibrium dissociation constants were 38 μM (m38C2) and 7.6 μM (h38C2) for β-diketone 2 and 0.43 μM (m38C2) and 1.0 μM (h38C2) for β-diketone 3, revealing similar β-diketone binding properties for mouse and humanized antibody.

Materials and Methods

Molecular modeling A molecular model of h38C2 Fab was constructed by homology modeling using the crystal structure of a related aldolase antibody, mouse 33F12 Fab (Protein Data Bank ID: 1AXT), as a template. The crystal structure of mouse 33F12 Fab was previously determined at a resolution of 2.15 Å.⁴ Alignment of mouse 33F12 and 38C2 amino acid sequences using the HOMOLOGY module within INSIGHT II software (Accelrys) confirmed that both sequences are highly homologous. They differ from each other by 19 out of 226 amino acids in the two variable domains, and their CDRs share the same lengths. In addition to the high sequence homology, both structures exhibit considerable structural similarity, as observed by a low-resolution crystal structure of 38C2. Residues in the model were mutated to conform to the h38C2 amino acid sequence and sidechains were placed based on standard rotamers. This model was then minimized with the DISCOVER module in INSIGHT II using 100 steps each of steepest descent minimization followed by conjugate gradient minimization.

Construction of h38C2 Fab—The sequences of the variable light and heavy chain domains of m38C2 as well as the sequences of human germline sequences DPK-9, JK4, DP-47, and JH4 (V BASE; see the world wide website, mrc-cpe.cam.ac.uk/vbase) were used to design overlapping oligonucleotides for the synthetic assembly of humanized V_{κ}, and V_{H}, respectively. N-glycosylation sites with the sequence NXS/T as well as internal restriction sites HindIII, XbaI, SacI, ApaI, and SfiI were avoided. PCR was carried out by using the Expand High Fidelity PCR System (Roche Molecular Systems). The humanized V_{κ}, oligonucleotides were: L flank sense (C. Rader et al., J. Biol. Chem. 275:13668-13676 (2000)); h38C2L1 (sense; 5′-GAGCTCCAGATGACCCAGTCTCCATCCTCCCTGTCTGCATCTGTAGGTGACCGCGTCACCATC ACTTG-3′) (SEQ. ID. NO:85); h38C2L2 (antisense; 5′-ATTCAGATATGGGCTGCCATAAGTGTGCAGGAGGCTCTGACTGGAGCGGCAAGTGATGGTGA CGCGGTC-3′) (SEQ. ID. NO:86); h38C2L3 (sense; 5′-TATGGCAGCCCATATCTGAATTGGTATCTCCAGAAACCAGGCCAGTCTCCTAAGCTCCTGATC TAT-3′) (SEQ. ID. NO:87); h38C2L4 (antisense; 5′-CTGAAACGTGATGGGACACCACTGAAACGATTGGACACTTTATAGATCAGGAGCTTAGGAGA CTG-3′) (SEQ. ID. NO:88); h38C2L5 (sense; 5′-AGTGGTGTCCCATCACGTTTCAGTGGCAGTGGTTCTGGCACAGATTTCACTCTCACCATCAGC AGTCTGCAACCTGAAGATTTTGCAGTG-3′) (SEQ. ID. NO:89); h38C2L6 (antisense; 5′-GATCTCCACCTTGGTCCCTCCGCCGAAAGTATAAGGGAGGTGGGTGCCCTGACTACAGAAGTA CACTGCAAAATCTTCAGGTTGCAG-3′) (SEQ. ID. NO:90); and L antisense flank (C. Rader et al., J. Biol. Chem. 275:13668-13676 (2000)). The humanized V_{H} oligonucleotides were: H flank sense (C. Rader et al., J. Biol. Chem. 275:13668-13676 (2000)); h38C2H1 (sense; 5′-GAGGTGCAGCTGGTGGAGTCTGGCGGTGGCTTGGTACAGCCTGGCGGTTCCCTGCGCCTCTCC TGTGCAGCCTCTGGCT-3′) (SEQ. ID. NO:91); h38C₂H₂ (antisense; 5′-CTCCAGGCCCTTCTCTGGAGACTGGCGGACCCAGCTCATCCAATAGTTGCTAAAGGTGAAGCC AGAGGCTGCACAGGAGAG-3′) (SEQ. ID. NO:92); h38C₂H₃ (sense; 5′-TCTCCAGAGAAGGGCCTGGAGTGGGTCTCAGAGATTCGTCTGCGCAGTGACAACTACGCCAC GCACTATGCAGAGTCTGTC-3′) (SEQ. ID. NO:93); h38C₂H₄ (antisense; 5′-CAGATACAGCGTGTTCTTGGAATTGTCACGGGAGATGGTGAAGCGGCCCTTGACAGACTCTGC ATAGTGCGTG-3′) (SEQ. ID. NO:94); h38C₂H₅ (sense; 5′-CAATTCCAAGAACACGCTGTATCTGCAAATGAACAGCCTGCGCGCCGAGGACACGGGCATTT ATTACTGTAAAACG-3′) (SEQ. ID. NO:95); h38C₂H₆ (antisense; 5′-TGAGGAGACGGTGACCAGGGTGCCCTGGCCCCAGTAGCTGAAACTGTAGAAGTACGTTTTAC AGTAATAAATGCCCGTG-3′) (SEQ. ID. NO:96); H flank antisense (C. Rader et al., J. Biol. Chem. 275:13668-13676 (2000)). Following assembly, humanized V_{κ}, and V_{H} were fused to human C_{κ}, and C_{γ1}1, respectively, and the resulting light chain and heavy chain fragment were fused and SfiI-cloned into phagemid vector pComb3X as described (C. Rader et al, J. Biol. Chem. 275:13668-13676 (2000); C. F. Barbas 3^{rd} et al., Phage Display. A laboratory manual, Cold Spring Harbor Laboratory, Cold Spring Harbor N.Y. (2001)). To enrich for clones with the correct h38C2 sequence, Fab were displayed on phage and selected by one round of panning against the immobilized β-diketone i (JW) conjugated to BSA. Soluble Fab were produced from single clones and tested for binding to immobilized JW-BSA by ELISA using donkey anti-human F(ab′)₂ polyclonal antibodies conjugated to horseradish peroxidase (Jackson ImmunoResearch Laboratories) as secondary antibody. Light chain and heavy chain encoding sequences of positive clones were analyzed by DNA sequencing using the primers OMPSEQ and PELSEQ (C. F. Barbas 3^{rd} et al., Phage Display. A laboratory manual, Cold Spring Harbor Laboratory, Cold Spring Harbor N.Y., (2001)), respectively, to confirm the assembled V_{κ}, and V_{H} sequences of h38C2.

Construction, production, and purification of h38C2 IgG1 The recently described vector PIGG (C. Rader et al, FASEB J., 16:2000-2002 (2002)) was used for mammalian expression of h38C2 IgG1. The mammalian expression vector PIGG-h38c2 is illustrated in FIG. 23. The 9 kb vector comprises heavy chain γ1 and light chain κ expression cassettes driven by a bidirectional CM promoter construct. Using primers PIGG-h38C2H (sense; 5′-GAGGAGGAGGAGGAGGAGCTCACTCCGAGGTGCAGCTGGTGGAGTCTG-3′) (SEQ. ID. NO:97) and GBACK (C. F. Barbas 3rd et al, Phage Display. A laboratory manual, Cold Spring Harbor Laboratory, Cold Spring Harbor N.Y. (2001)), the V_{H} coding sequence from h38C2 Fab in phagemid vector pComb3X was amplified, digested with SacI and ApaI, and cloned into the appropriately digested vector PIGG. Using primers PIGG-h38C2L (sense: 5′-GAGGAGGAGGAGGAGAAGCTTGTTGCTCTGGATCTCTGGTGCCTACGGGGAGCTCCAGATGA CCCAGTCTCC-3′) (SEQ. ID. NO:98) and LEADB (C. F. Barbas 3^{rd} et al, Phage Display. A laboratory manual, Cold Spring Harbor Laboratory, Cold Spring Harbor N.Y. (2001)) the V_{L} coding sequence from h38C2 Fab in phagemid vector pComb3X was amplified, digested with HindIII and XbaI, and cloned into the appropriately digested vector PIGG that already contained the h38C2 heavy chain. Intermediate and final PIGG vector constructs were amplified in E. coli strain SURE (Stratagene) and prepared with the QIAGEN Plasmid Maxi Kit. h38C2 IgG1 were produced from the prepared final PIGG vector construct by transient transfection of human 293T cells using Lipofectamine 2000 (Invitrogen). Transfected cells were maintained in GIBCO 10% ultra-low IgG (<0.1%) FCS (Invitrogen) in RPMI 1640 (Hyclone) for 2 weeks. During this time, the medium was collected and replaced three times. The collected medium was subjected to affinity chromatography on a recombinant Protein A HiTrap column (Amersham Biosciences). This purification step yielded 2.45 mg h38C2 IgG1 from 2,300 mL collected medium as determined by measuring the optical density at 280 nm using an Eppendorf BioPhotometer. Following dialysis against PBS in a Slide-A-Lyzer 10K dialysis cassette (Pierce), the antibody was concentrated to 760 μg/mL using an Ultrafree-15 Centrifugal Filter Device (UFV2BTK40; Millipore), and sterile filtered through a 0.2-μm Acrodisc 13mM S-200 Syringe Filter (Pall). The final yield was 2.13 mg (87%). Purified h38C2 IgG1 was confirmed by nonreducing SDS-PAGE followed by Coomassie Blue staining.

Enaminone formation Antibody (h38C2 IgG1 or b12 IgG1) was added to β-diketone (ii) to a final concentration of 25 μM antibody binding site and 125 μM β-diketone. This mixture was incubated at room temperature for 10 minutes before a UV spectrum was acquired on a SpectraMax Plus 384 UV plate reader (Molecular Devices) using SOFTmax Pro software (version 3.1.2).

Binding assays Unless noted otherwise, all solutions were phosphate buffered saline (pH 7.4). A 2× solution of either β-diketone (ii) or (iii) (50 μL) was added to 50 μL of the antibody (either h38C2 or m38C2) and allowed to incubate at 37° C. for 1 hr. Solutions were mixed by pipetting. Final concentrations of antibody were 0.4 to 8 nM antibody binding site, and final concentrations of β-diketones (ii) and (iii) were 10⁻⁹ to 10⁻² M and 10⁻¹⁰ to 10⁻⁴ M, respectively. Each well of a Costar 3690 96-well plate (Corning) was coated with 100 ng of the BSA conjugate of β-diketone (i) in TBS. Wells were then blocked with 3% (w/v) BSA in TBS. Then, 50 μL of the antibody/β-diketone mixture was added, followed by 50 μL of a 1:1,000 dilution of either goat anti-human Fc IgG polyclonal antibodies (Pierce) or rabbit anti-mouse Fc IgG polyclonal antibodies (Jackson ImmunoResearch Laboratories) conjugated to horseradish peroxidase. This was followed by 50 μL ABTS substrate solution. Between each addition, the plate was covered, incubated at 37° C. for 1 hr, and then washed five times with deionized H₂O. The absorbance at 405 nm was monitored as described above until the reaction with no β-diketone reached an appropriate value (0.5<A₄₀₅<1.0). For each well, the fractional inhibition of ELISA signal (vᵢ) was calculated using equation (a)

vᵢ=(Aₒ−Aᵢ)/(Aₒ) (a)

where Aₒ is the ELISA absorbance obtained in the absence of β-diketone and Aᵢ is the absorbance obtained in the presence of β-diketone. For monovalent binding proteins, the fraction of antibody bound to soluble β-diketone (fᵢ) is equal to vᵢ. However, the IgG antibody is bivalent, and the ELISA signal is inhibited only by the presence of doubly liganded antibody and not by monovalent binding. Therefore, the Stevens correction for a bivalent antibody was used,

fᵢ=(vᵢ)^{1/2} (b)

The following relationship was used to determine the apparent equilibrium dissociation constant

fᵢ=fₘᵢₙ+(fₘₐₓ−fₘᵢₙ)(1+K_{D}/a₀)⁻¹ (c)

where a₀ corresponds to the total β-diketone concentration, K_{D} is the equilibrium dissociation constant, and fₘᵢₙ and fₘₐₓ represent the experimentally determined values when the antibody binding sites are unoccupied or saturated, respectively. Because this equation is only valid when the K_{D} values are at least 10× higher than the antibody concentration, it was verified that the K_{D} values determined from equation iii met this criterion. Data were fit using a nonlinear least-squares fitting procedure of KaleidaGraph (version 3.0.5, Abelbeck software) with K_{D}, fₘₐₓ, and fₘᵢₙ as the adjustable parameters and normalized using equation (d)

fₙₒᵣₘ=(fᵢ−fₘᵢₙ)/(fₘₐₓ−fₘᵢₙ) (d)

### Example 26

### Synthesis of the 20-Atom AZD Maleimide Linker

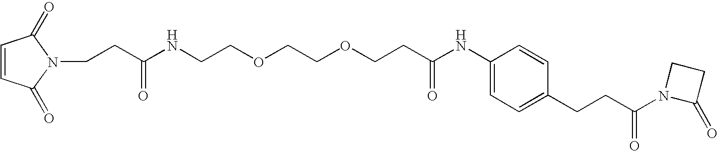

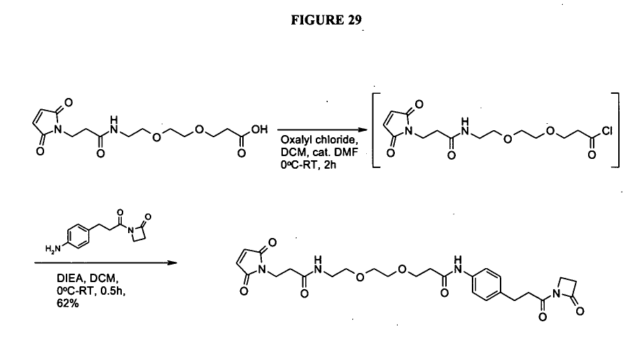

is provided in FIG. 29.

### Example 27

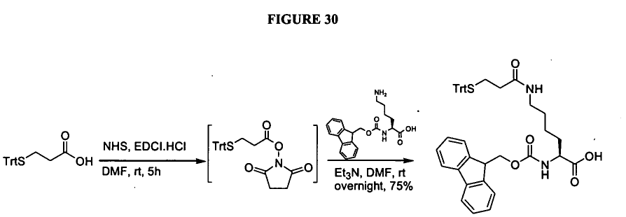

The synthesis of a sidechain modified Lys which may be used with the linker shown in FIG. 29 is provided in FIG. 30.

### Example 28

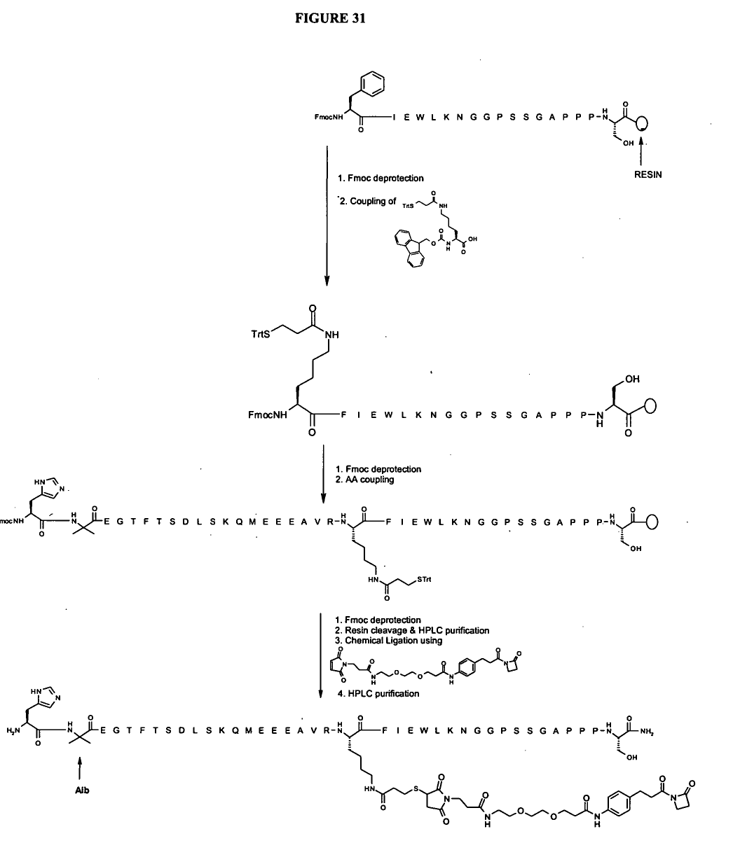

The synthesis of a GA targeting agent-linker conjugate comprising the GA targeting peptide of SEQ ID NO:22 linked to the 20-atom AZD maleimide linker set forth in FIG. 29 via a side-chain modified Lys residue in the peptide is provided in FIG. 31.

### Example 29

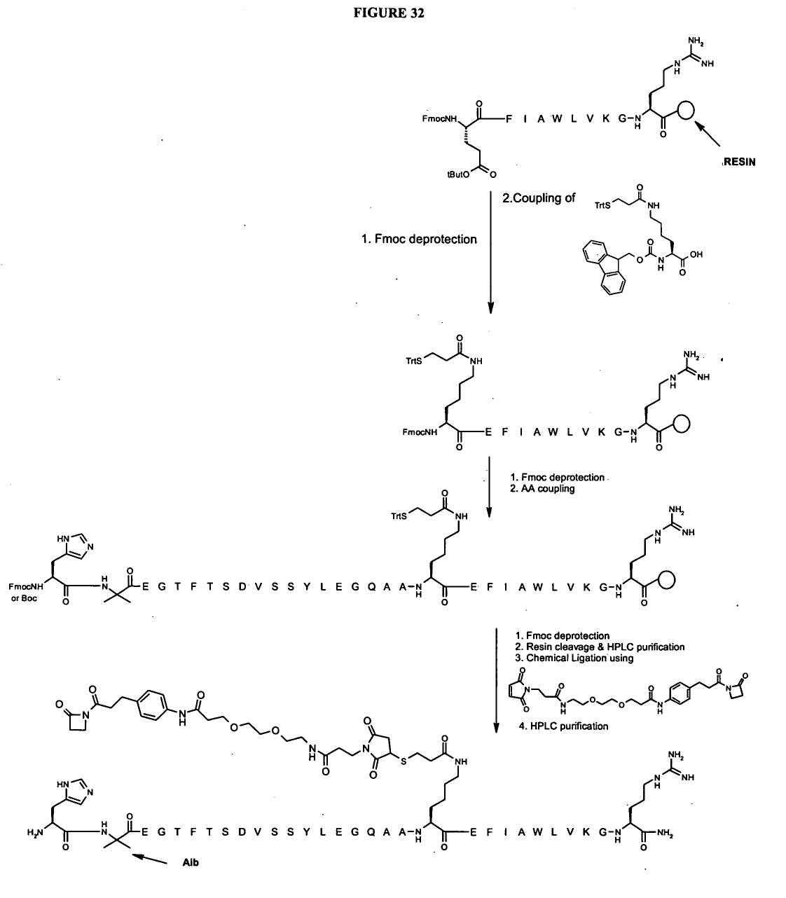

The synthesis of a GA targeting agent-linker conjugate comprising the GA targeting peptide of SEQ ID NO:32 linked to the 20-atom AZD maleimide linker set forth in FIG. 29 via a side-chain modified Lys residue in the peptide is provided in FIG. 32.

### Example 30

### Characterization of GA Targeting Peptide-Mediated Insulin Secretion In Vitro

GA targeting peptides analogs having the amino acid sequences set forth in SEQ ID NOs: 1-76 (see Table I, above) were generated using the same general methods set forth in Example 1 and 2 for the GA targeting peptides of SEQ ID NO:1 and SEQ ID NO:2.

The ability of the GA targeting peptides of SEQ ID NOs:1-13, 32, 35, and 40-47, 49-51, 53-55, and 57-63 to stimulate insulin secretion from pancreatic β cells in vitro was tested using a glucose-stimulated insulin secretion (GSIS) assay. Briefly, glucose and GA targeting peptide was added at various concentrations to pancreatic β cell cultures, and insulin secretion was detected by measuring insulin levels over time. EC50 was calculated for each peptide. The results of this assay are set forth in Table III, below.

TABLE III

| GA targeting peptide | EC50 (μM) |
|---|---|
| SEQ ID NO: 1 (GLP-1 7-36) | <10 |
| SEQ ID NO: 2 (exendin-4) | <10 |
| SEQ ID NO: 3 | <10 |
| SEQ ID NO: 4 | <10 |
| SEQ ID NO: 5 | <10 |
| SEQ ID NO: 6 | <10 |
| SEQ ID NO: 7 | <10 |
| SEQ ID NO: 8 | <10 |
| SEQ ID NO: 9 | <10 |
| SEQ ID NO: 10 | <10 |
| SEQ ID NO: 11 | <10 |
| SEQ ID NO: 12 | <10 |
| SEQ ID NO: 13 | <10 |
| SEQ ID NO: 32 | <10 |
| SEQ ID NO: 35 | <10 |
| SEQ ID NO: 40 | <10 |
| SEQ ID NO: 41 | <10 |
| SEQ ID NO: 42 | <10 |
| SEQ ID NO: 43 | <10 |
| SEQ ID NO: 44 | <10 |
| SEQ ID NO: 45 | <10 |
| SEQ ID NO: 46 | <10 |
| SEQ ID NO: 47 | <10 |
| SEQ ID NO: 49 | <10 |
| SEQ ID NO: 50 | <10 |
| SEQ ID NO: 51 | <10 |
| SEQ ID NO: 53 | <10 |
| SEQ ID NO: 54 | <10 |
| SEQ ID NO: 55 | <10 |
| SEQ ID NO: 57 | <10 |
| SEQ ID NO: 58 | <10 |
| SEQ ID NO: 59 | <10 |
| SEQ ID NO: 60 | <10 |
| SEQ ID NO: 61 | <10 |
| SEQ ID NO: 62 | <10 |
| SEQ ID NO: 63 | <10 |

### Example 28

### Characterization of GA Targeting Peptide-Linker Conjugate-Mediated Insulin Secretion In Vitro

The GA targeting peptides of SEQ ID NOs:1-76 were linked to various linkers to generate GA targeting peptide-linker conjugates. The GA targeting peptides of SEQ ID NOs:3-5, 14-33, 35-37, 57, and 63-72 were linked to the 20-atom AZD maleimide linker (“20-atom AZD”) synthesized in Example 26, which has the following structure:

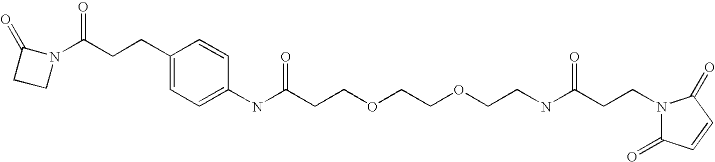

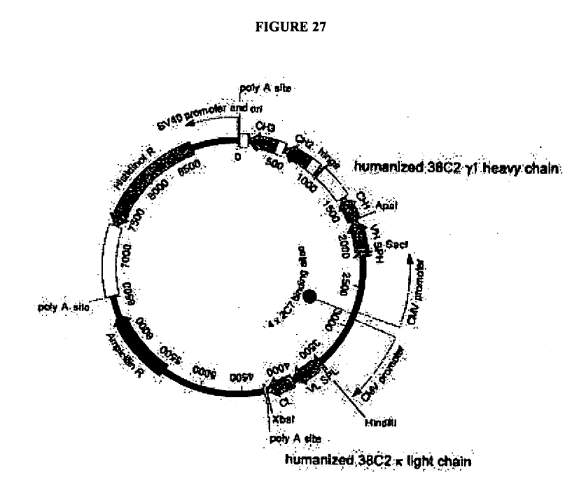

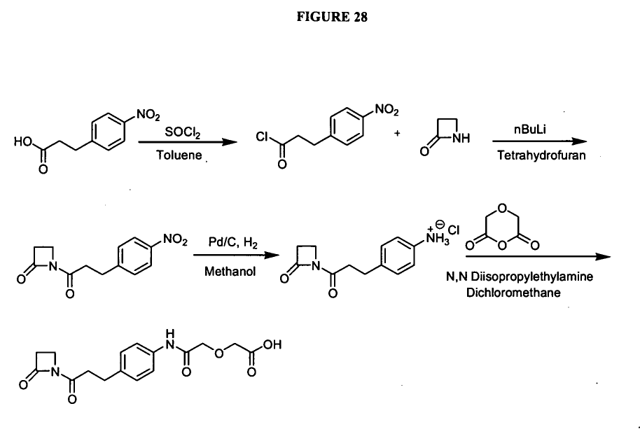

The linkage reaction of peptide to 20-atom AZD is illustrated in FIGS. 26 and 27 for the GA targeting peptides of SEQ ID NO:22 and SEQ ID NO:32, respectively.

The GA targeting peptides of SEQ ID NOs:32 and 37 were linked to the 10-atom AZD maleimide linker “10-atom AZD,” which has the following structure:

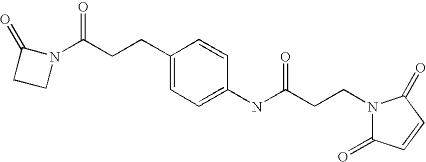

The GA targeting peptide of SEQ ID NO:37 was linked to the 13-atom AZD maleimide linker “13-atom AZD,” which has the following structure:

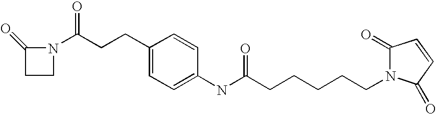

The GA targeting peptides of SEQ ID NOs:35 and 37 were linked to the 16-atom AZD maleimide linker “16-atom AZD,” which has the following structure:

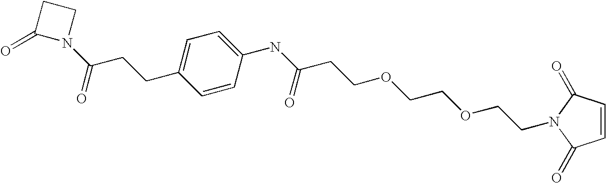

The GA targeting peptide of SEQ ID NO:35 was linked to the 26-atom AZD maleimide linker “26-atom AZD,” which has the following structure:

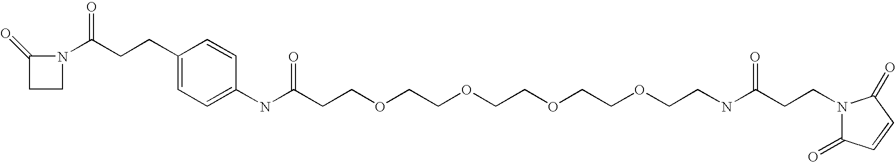

The GA targeting peptides of SEQ ID NOs:33 and 37 were linked to the linker “Gly-AZK,” which has the following structure:

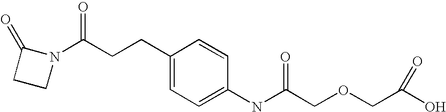

The GA targeting peptides of SEQ ID NOs:1, 33, 34, and 36-37 were linked to the linker “PEG4-Glu-DK linker,” which has the following structure:

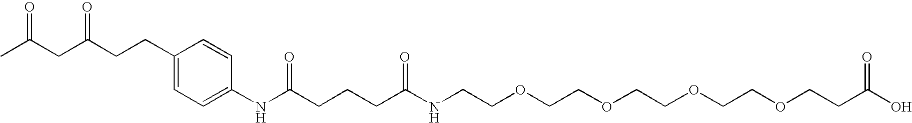

The ability of these GA targeting agent-linker conjugates to stimulate glucose secretion in vitro was measured using the GSIS assay described in Example 27. Those conjugates consisting of the GA targeting peptides of SEQ ID NOs: 4-5 and 14-31 linked to 20-atom AZD were used for a tethered walk experiment to determine the optimal position for linking of GA targeting peptide to linker. Each of these peptides contained a side-chain modified Lys residue according to the scheme illustrated in FIG. 30 at a different position. The results of this experiment are set forth in Table IV, below.

TABLE IV

| GA targeting agent-linker conjugate | EC50 (μM) |
|---|---|
| SEQ ID NO: 4 - 20 atom AZD | <10 |
| SEQ ID NO: 5 - 20 atom AZD | <10 |
| SEQ ID NO: 19 - 20 atom AZD | <10 |
| SEQ ID NO: 20 - 20 atom AZD | <10 |
| SEQ ID NO: 21 - 20 atom AZD | <10 |
| SEQ ID NO: 22 - 20 atom AZD | <10 |
| SEQ ID NO: 23 - 20 atom AZD | <10 |
| SEQ ID NO: 24 - 20 atom AZD | <10 |
| SEQ ID NO: 25 - 20 atom AZD | <10 |
| SEQ ID NO: 26 - 20 atom AZD | <10 |
| SEQ ID NO: 27 - 20 atom AZD | <10 |
| SEQ ID NO: 28 - 20 atom AZD | <10 |
| SEQ ID NO: 32 - 20 atom AZD | <10 |
| SEQ ID NO: 37 - 20 atom AZD | <10 |
| SEQ ID NO: 32 - 10 atom AZD | <10 |
| SEQ ID NO: 37 - 10 atom AZD | <10 |
| SEQ ID NO: 35 - 26 atom AZD | <10 |
| SEQ ID NO: 33 - Gly-AZD | <10 |
| SEQ ID NO: 1 - PEG4-Glu-DK | <10 |
| SEQ ID NO: 33 - PEG4-Glu-DK | <10 |
| SEQ ID NO: 34 - PEG4-Glu-DK (linked via K20) | <10 |
| SEQ ID NO: 34 - PEG4-Glu-DK (linked at N- | <10 |
| terminal) | |
| SEQ ID NO: 36 - PEG4-Glu-DK | <10 |
| SEQ ID NO: 37 - PEG4-Glu-DK | <10 |
| SEQ ID NO: 74 (linked to benzoyl cap via K28) | <10 |
| SEQ ID NO: 75 (linked to trans 3-hexanoyl cap at | <10 |
| N-terminal) | |
| SEQ ID NO: 76 (linked to 3-aminophenylacetyl cap | <10 |
| at N-terminal) | |

### Example 29

### Glucose Tolerance Test (GTT), Body Weight Change and Food Intake

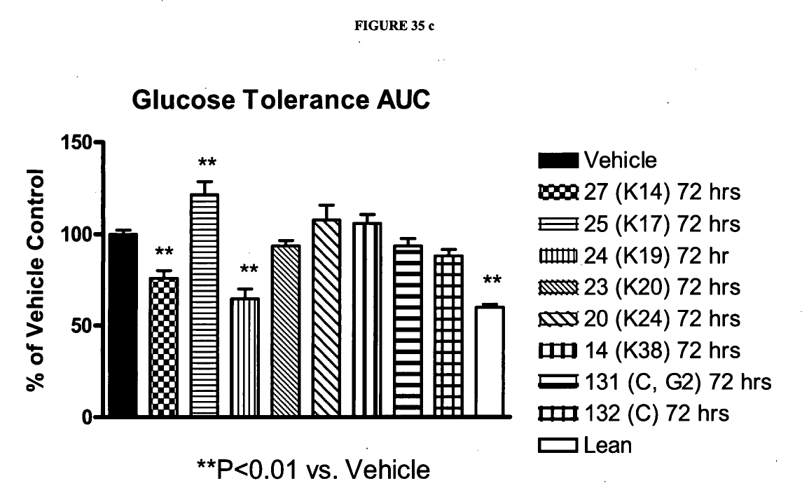

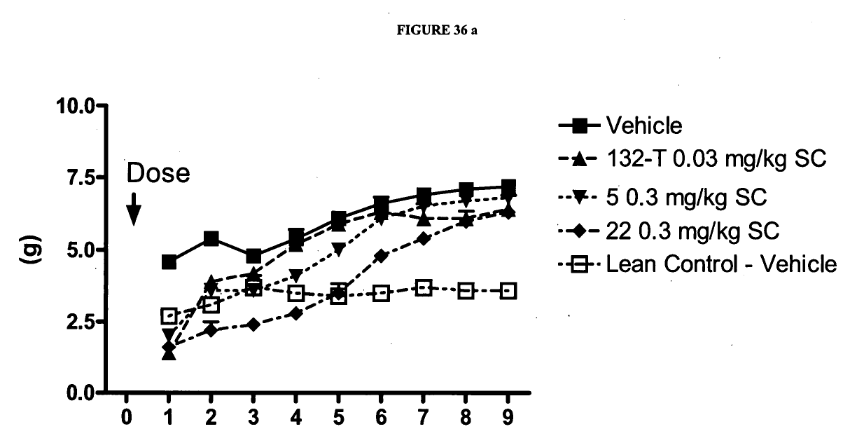

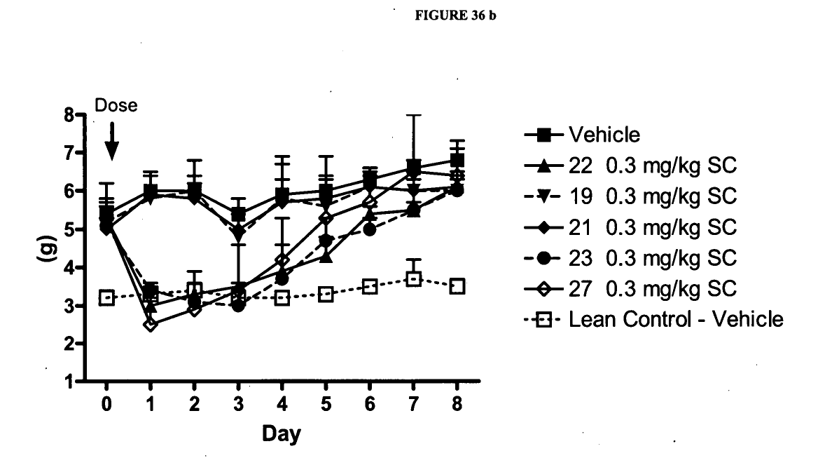

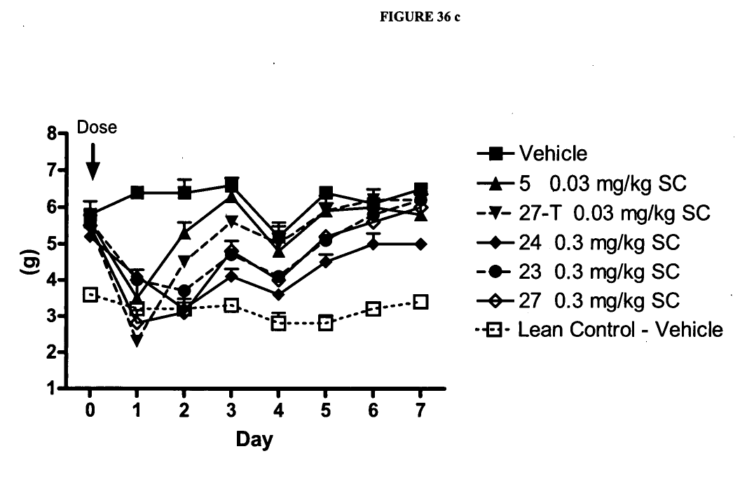

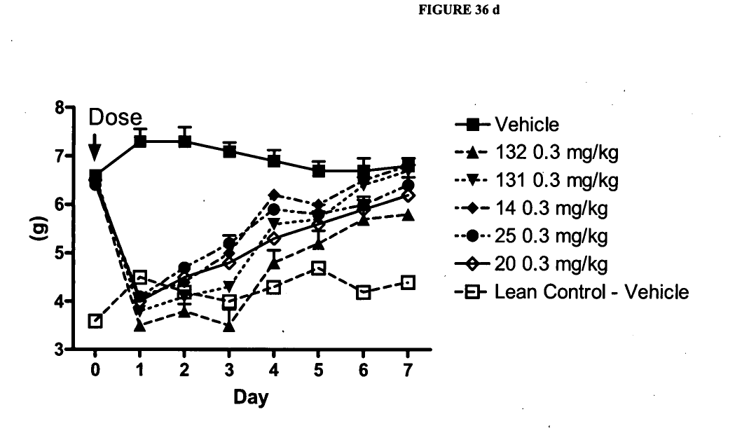

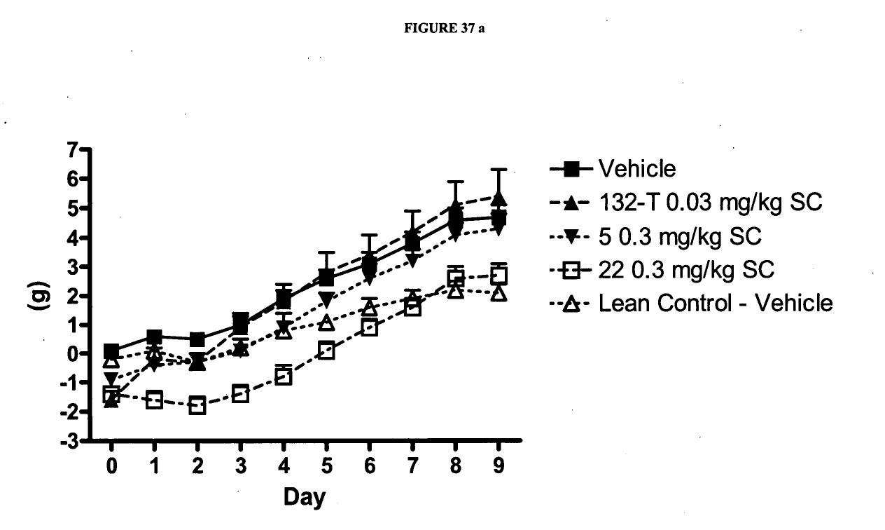

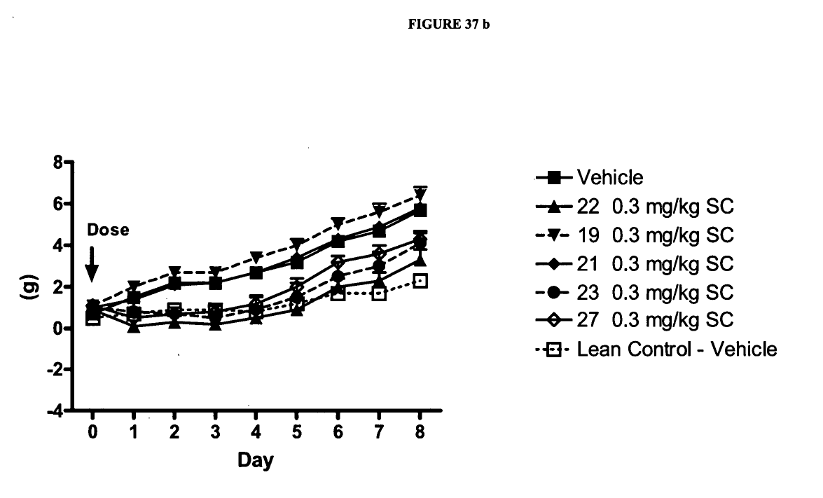

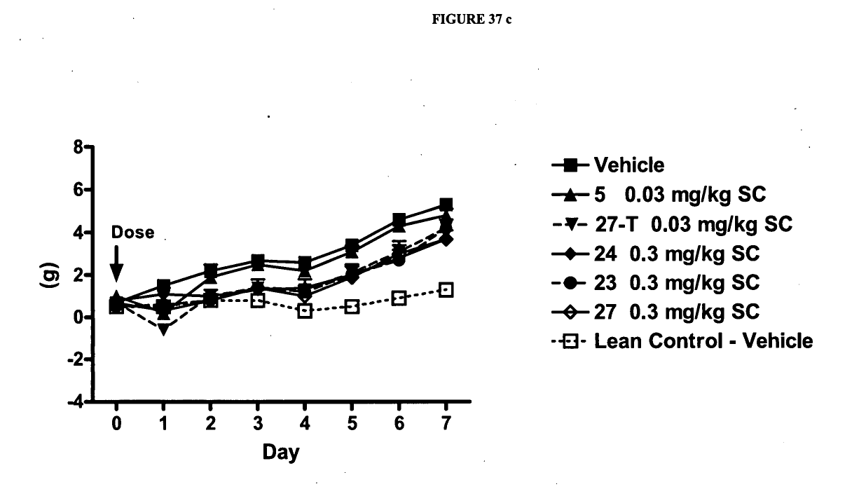

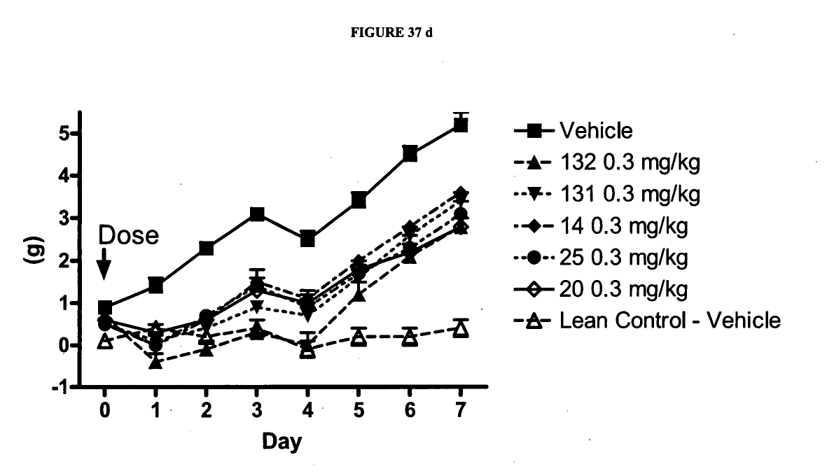

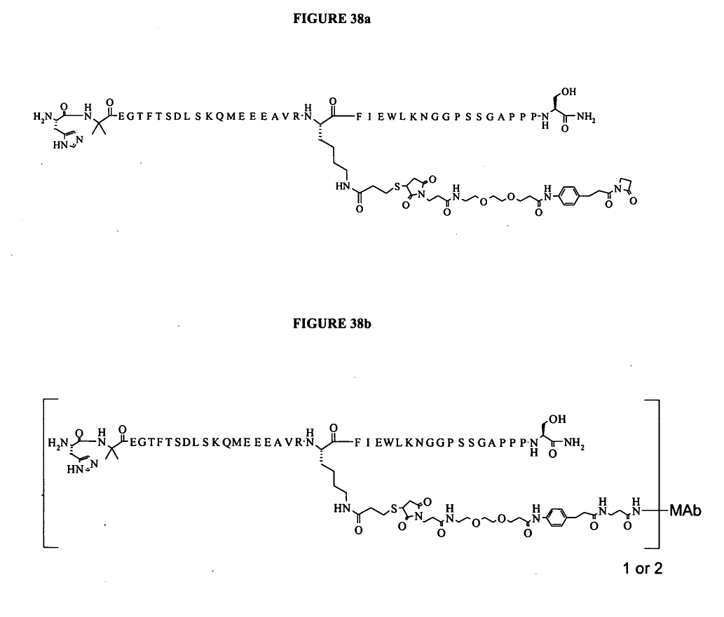

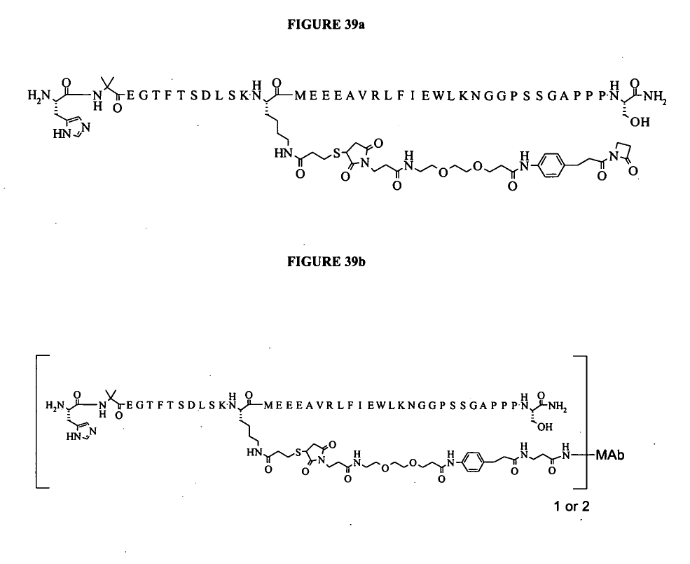

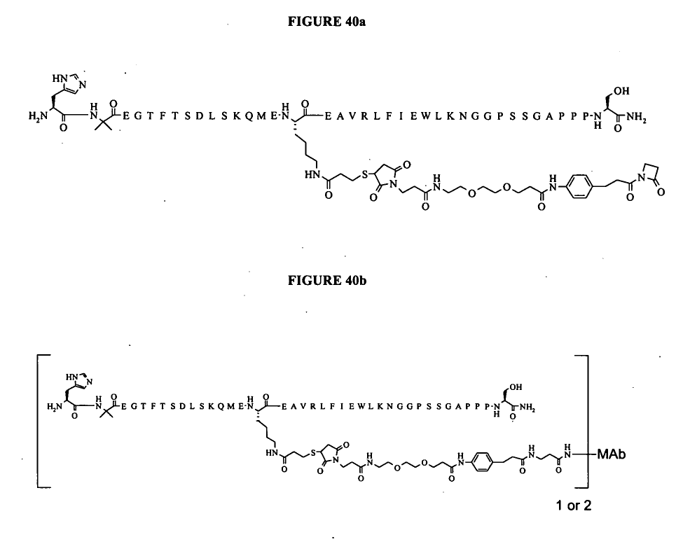

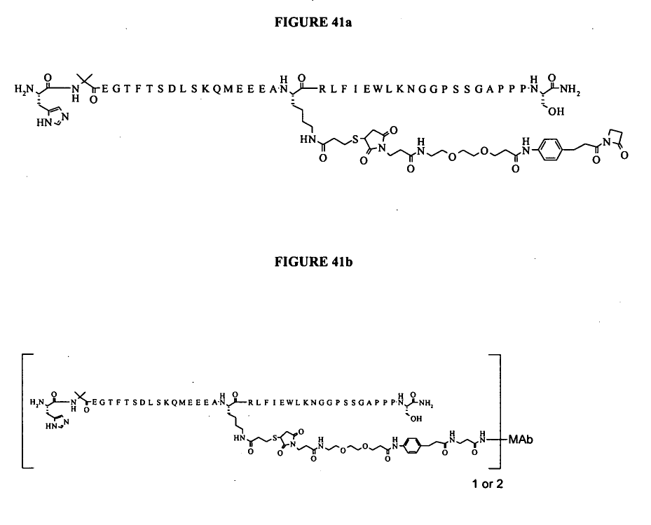

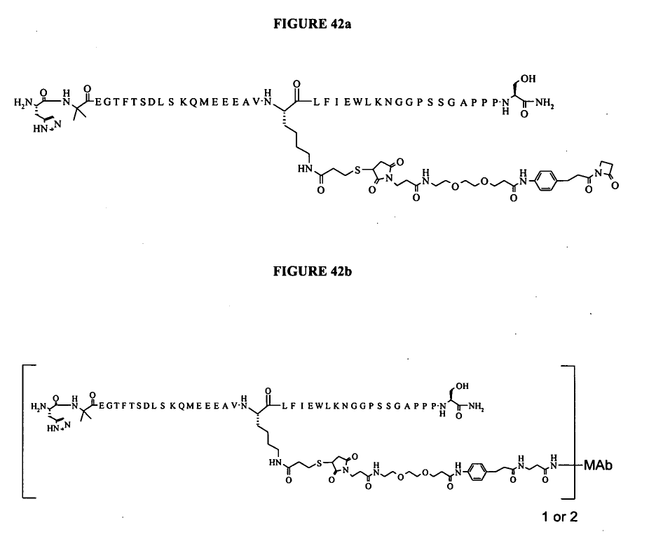

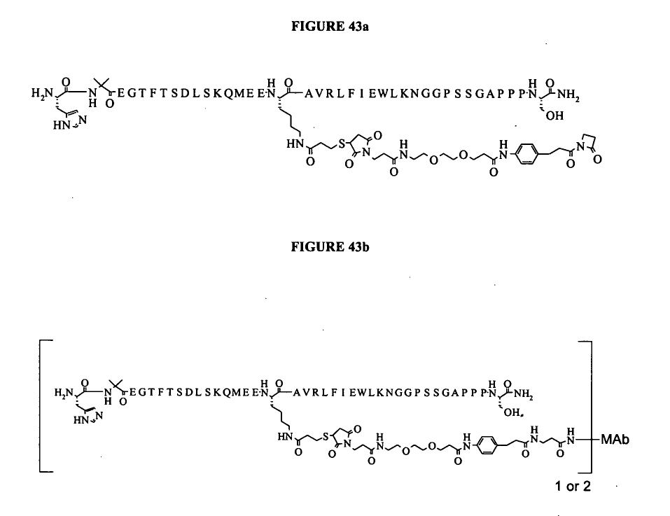

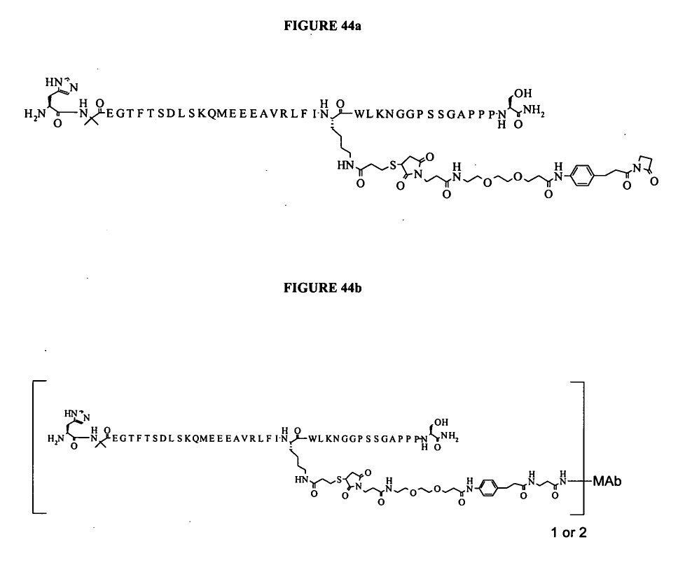

In vivo efficacy of exemplary GA targeting compounds and agents of the invention were assessed using single- or repeat-dose glucose tolerance testing paradigm (FIG. 35). Young adult male ob/ob mice (Jackson Laboratories, Bar Harbor, Me.) were dosed with compounds of the invention subcutaneously (SC) in the mid-scapular region, using brief manual restraint., with injection volumes of 0.2-0.3 ml. Lean littermate control mice (n=8/group, Jackson Laboratories, Bar Harbor, Me.) were similarly dosed with Vehicle. Food intake (FIG. 36) and cumulative body weight change (FIG. 37) were monitored daily in the morning (08:00-09:00 H; lights on at 06:00 H and off at 18:00 H).

Mice underwent oral glucose tolerance testing (OGTT) following a standard protocol. Briefly, mice were fasted for 4-5 hrs at the beginning of the lights-on phase in the colony. At the end of this period (early afternoon), mice were tail-bled immediately prior to and at regular intervals from 15 to 120 minutes after an oral glucose challenge (1.5 g/kg). Food was returned to the cages following collection of the 120 minute time point. Glucose levels were determined using self-test blood glucose meters, and the area-under-the-curve (AUC) for glucose as a function of time after oral glucose challenge was calculated using a linear trapezoidal equation (FIG. 35).

Linking at position 23 (SEQ ID NO:21) did not decrease body weight or feeding and did not improve glucose tolerance @48 hrs. Linking at positions 17, 24, 38 and at the C-terminus (SEQ ID NOs: 25, 20, 14, 131, 132) decreased body weight and feed but did not improve glucose tolerance at 72 hrs. Linking at position 26 (SEQ ID NO:19) did not decreased body weight or feeding but did improve glucose tolerance at 48 hrs. All examples used K or K(SH) residues as the linking residue. In some aspects of the invention, compounds that perform well under some conditions may be suitable for certain applications. In other aspects of the invention, compounds that provide advantages under multiple test conditions may be advantageous.

Data are depicted as the mean±standard error and were analyzed by one-way ANOVA (GraphPad Prism 4.0, GraphPad Software Inc., San Diego, Calif.) with Dunnett's post-hoc test for between group differences.

The invention thus has been disclosed broadly and illustrated in reference to representative embodiments described above. Those skilled in the art will recognize that various modifications can be made to the present invention without departing from the spirit and scope thereof. All publications, patent applications, and issued patents, are herein incorporated by reference to the same extent as if each individual publication, patent application or issued patent were specifically and individually indicated to be incorporated by reference in its entirety. Definitions that are contained in text incorporated by reference are excluded to the extent that they contradict definitions in this disclosure.

The words “comprises/comprising” and the words “having/including” when used herein with reference to the present invention are used to specify the presence of stated features, integers, steps or components but does not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

From the foregoing, it will be apparent that numerous modifications and variations can be effected without departing from the true spirit and scope of the novel concept of the present invention. It will be appreciated that the present disclosure is intended to set forth the exemplifications of the invention which are not intended to limit the invention to the specific embodiments illustrated. The disclosure is intended to cover by the appended claims all such modifications as fall within the scope of the claims. All references cited herein are incorporated by reference as if fully set forth herein.

Where technical features mentioned in any claim are followed by reference signs, these reference signs have been included for the sole purpose of increasing the intelligibility of the claims and accordingly, such reference signs do not have any limiting effect on the scope of each element identified by way of example by such reference signs.

1. A peptide agonist of the GLP-1 receptor (GA targeting agent), comprising a peptide comprising a sequence:
R¹—H¹x²E³G⁴T⁵F⁶T⁷S⁸D⁹x¹⁰S¹¹x¹²x¹³x¹⁴E¹⁵x¹⁶x¹⁷A¹⁸x¹⁹x²⁰x²¹F²²x²³x²⁴x²⁵x²⁶x²⁷x²⁸x²⁹x³⁰x³¹x³²x³³x³⁴x³⁵x³⁶x³⁷x³⁸x³⁹-R²,
wherein
R¹ is absent, CH₃, C(O)CH3, C(O)CH₂CH₃, C(O)CH₂CH₂CH₃, or C(O)CH(CH₃)CH₃;
R² is absent, OH, NH₂, NH(CH₃), NHCH₂CH₃, NHCH₂CH₂CH₃, NHCH(CH₃)CH₃, NHCH₂CH₂CH₂CH₃, NHCH(CH₃)CH₂CH₃, NHC₆H₅, NHCH₂CH₂OCH₃, NHOCH₃, NHOCH₂CH₃, a carboxy protecting group, a lipid fatty acid group, or a carbohydrate,
x² is blocking group such as Aib, A, S, T, V, L, I, or D-Ala;
x¹⁰ is V, L, I, or A;
x¹² is S or K;
x¹³ is Q or Y;
x¹⁴ is G, C, F, Y, W, M, or L;
x¹⁶ is K, D, E, or G;
x¹⁷ is E or Q;
x¹⁹ is L, I, V, or A;
x²⁰ is Orn, K(SH), R, or K;
x²¹ is L or E;
x²³ is I or L;
x²⁴ is A or E;
x²⁵ is W or F;
x²⁶ is L or I;
x²⁷ is 1, K, or V;
x²⁸ is R, Orn, N, or K;
x²⁹ is Aib or G;
x³⁰ is any amino acid, preferably G or R;
x³¹ is P or absent;
x³² is S or absent;
x³³ is S or absent;
x³⁴ is G or absent;
x³⁵ is A or absent;
x³⁶ is P or absent;
x³⁷ is P or absent;
x³⁸ is P or absent;
x³⁹ is S or absent;
x⁴⁰ is a linking residue or absent; and
wherein one of x¹⁰, x¹¹, x¹², x¹³, x¹⁴, x¹⁶, x¹⁷, x¹⁹, x²⁰, x²¹, x²⁴, x²⁶, x²⁷, x²⁸, x³², x³³, x³⁴, x³⁵, x³⁶, x³⁷, x³⁸, x³⁹, or x⁴⁰ is substituted with a linking residue (-[LR]-) comprising a nucleophilic sidechain wherein the linking residue is K(SH).

2. The GA targeting agent as claimed in claim 1, wherein x² is Aib.

3. The GA targeting agent as claimed in claim 1, comprising a peptide comprising a sequence substantially homologous to:
R¹—H¹Aib²E³G⁴T⁵F⁶T⁷S⁸D⁹V¹⁰S¹¹S¹²Y¹³x¹⁴E¹⁵x¹⁶Q¹⁷A¹⁸x¹⁹x²⁰E²¹F²²I²³A²⁴x²⁵L²⁶x²⁷x²⁸x²⁹R³⁰—R²
x¹⁴ is G, C, F, Y, W, or L,
x¹⁶ is K, D, E, or G,
x¹⁹ is L, I, V, or A,
x²⁰ is Orn, R, or K,
x²⁵ is W or F,
x²⁷ is I or V,
x²⁸ is R or K, and
x²⁹ is Aib or G.

4. The GA targeting agent as claimed in claim 1, comprising a peptide comprising a sequence substantially homologous to:
R¹—H¹Aib²E³G⁴T⁵F⁶T⁷S⁸D⁹V¹⁰S¹¹K¹²Q¹³M¹⁴E¹⁵E¹⁶E¹⁷A¹⁸V¹⁹R²⁰L²¹F²²I²³E²⁴W²⁵L²⁶K²⁷N²⁸G²⁹G³⁰P³¹S³²S³³G³⁴A³⁵P³⁶P³⁷P³⁸S³⁹—R².

5. The GA targeting agent as claimed in claim 1, wherein the linking residue is selected from the group consisting of x¹¹, x¹², x¹³, x¹⁴, x¹⁶, x¹⁷, x¹⁹, x²⁰, x²¹, x²⁴, x²⁷, x²⁸, x³², x³⁴, x³⁸, and C-terminus.

6-8. (canceled)

9. The GA targeting agent as claimed in claim 1, wherein x¹⁴ is the linking residue.

10. The GA targeting agent as claimed in claim 1, wherein R¹ is C(O)CH₃.

11. The GA targeting agent as claimed in claim 1, wherein R² is NH₂.

12. The GA targeting agent as claimed in claim 1, comprising a peptide comprising a sequence at least 90% homologous to one or more compounds selected from the group consisting of:
(SEQ ID NO:172)
R¹-HGEGTFTSDLSKQMEEEAVRLFIEWLKNGGPSSGAPPPSK(SH)-
R²,
(SEQ ID NO:173)
R¹-HAibEGTFTSDLSKQMEEEAVRLFIEWLKNGGPSSGAPPPSK(SH)-
R²,
(SEQ ID NO:99)
R¹-HAibEGTFTSDLSKQMEEEAVRLFIEWLKNGGPSSGAPPK(SH)S-
R²,
(SEQ ID NO:100)
R¹-HAibEGTFTSDLSKQMEEEAVRLFIEWLKNGGPSSGAK(SH)PPS-
R²,
(SEQ ID NO:101)
R¹-HAibEGTFTSDLSKQMEEEAVRLFIEWLKNGGPSSK(SH)APPPS-
R²,
(SEQ ID NO:168)
R¹-HAibEGTFTSDLSKQMEEEAVRLFIEWLKNGGPK(SH)SGAPPPS-
R²,
(SEQ ID NO:102)
R¹-HAibEGTFTSDLSKQMEEEAVRLFIEWLKK(SH)GGPSSGAPPPS-
R²,
(SEQ ID NO:170)
R¹-HAibEGTFTSDLSKQMEEEAVRLFIEWLK(SH)NGGPSSGAPPPS-
R²,
(SEQ ID NO:103)
R¹-HAibEGTFTSDLSKQMEEEAVRLFIEWK(SH)KNGGPSSGAPPPS-
R²,
(SEQ ID NO:104)
R¹-HAibEGTFTSDLSKQMEEEAVRLFIK(SH)WLKNGGPSSGAPPPS-
R²,
(SEQ ID NO:105)
R¹-HAibEGTFTSDLSKQMEEEAVRLFK(SH)EWLKNGGPSSGAPPPS-
R²,
(SEQ ID NO:106)
R¹-HAibEGTFTSDLSKQMEEEAVRK(SH)FIEWLKNGGPSSGAPPPS-
R²,
(SEQ ID NO:107)
R¹-HAibEGTFTSDLSKQMEEEAVK(SH)LFIEWLKNGGPSSGAPPPS-
R²,
(SEQ ID NO:108)
R¹-HAibEGTFTSDLSKQMEEEAK(SH)RLFIEWLKNGGPSSGAPPPS-
R²,
(SEQ ID NO:109)
R¹-HAibEGTFTSDLSKQMEEK(SH)AVRLFIEWLKNGGPSSGAPPPS-
R²,
(SEQ ID NO:110)
R¹-HAibEGTFTSDLSKQMEK(SH)EAVRLFIEWLKNGGPSSGAPPPS-
R²,
(SEQ ID NO:111)
R¹-HAibEGTFTSDLSKQK(SH)EEEAVRLFIEWLKNGGPSSGAPPPS-
R²,
(SEQ ID NO:112)
R¹-HAibEGTFTSDLSKK(SH)MEEEAVRLFIEWLKNGGPSSGAPPPS-
R²,
(SEQ ID NO:169)
R¹-HAibEGTFTSDLSK(SH)QMEEEAVRLFIEWLKNGGPSSGAPPPS-
R²,
(SEQ ID NO:113)
R¹-HAibEGTFTSDLK(SH)KQMEEEAVRLFIEWLKNGGPSSGAPPPS-
R²,
(SEQ ID NO:114)
R¹-HAibEGTFTSDLSKQMEEEAVRLFIEWLKNGGK(SH)-R,
(SEQ ID NO:115)
R¹-HAibEGTFTSDLSKQMEEEAVRKFIEWLK(SH)NGGPSS-R²,
(SEQ ID NO:38)
R¹-HAibEGTFTSDVSSYLEGQAAKEFIAWLVKGRK(SH)-R²,
(SEQ ID NO:39)
R¹-HAibEGTFTSDVSSYLEGQAAK(SH)EFIAWLVKGR-R²,
(SEQ ID NO:171)
R¹-HAibEGTFTSDVSSYLEEQAVK(SH)EFIAWLIKAibRPSSGAPPP
S-R²,
(SEQ ID NO:116)
R¹-HAibEGTFTSDK(SH)SSYLEEQAVKEFIAWLIKAibR-R²,
(SEQ ID NO:117)
R¹-HAibEGTFTSDVSK(SH)YLEEQAVKEFIAWLIKAibR-R²,
(SEQ ID NO:118)
R¹-HAibEGTFTSDVSSYK(SH)EEQAVKEFIAWLIKAibR-R²,
(SEQ ID NO:119)
R¹-HAibEGTFTSDVSSYLEK(SH)QAVKEFIAWLIKAibR-R²,
(SEQ ID NO:120)
R¹-HAibEGTFTSDVSSYLEEQK(SH)VKEFIAWLIKAibR-R²,
(SEQ ID NO:121)
R¹-HAibEGTFTSDVSSYLEEQAVK(SH)EKIAWLIKAibR-R²,
(SEQ ID NO:122)
R¹-HAibEGTFTSDVSSYLEEQAVKEFIK(SH)WLIKAibR-R²,
(SEQ ID NO:123)
R¹-HAibEGTFTSDVSSYLEEQAVKEFIAWK(SH)IKAibR-R²,
and
(SEQ ID NO:124)
R¹-HAibEGTFTSDVSSYLEEQAVKEFIAWLIKAibRPSSGAPPPSK
(SH)-R².

13-15. (canceled)

16. The GA targeting agent claimed in claim 1, comprising a peptide comprising a sequence selected from the group consisting of:
(SEQ ID NO:99)
R¹-HAibEGTFTSDLSKQMEEEAVRLFIEWLKNGGPSSGAPPK(SH)S-
R²,
(SEQ ID NO:100)
R¹-HAibEGTFTSDLSKQMEEEAVRLFIEWLKNGGPSSGAK(SH)PPS-
R²,
(SEQ ID NO:101)
R¹-HAibEGTFTSDLSKQMEEEAVRLFIEWLKNGGPSSK(SH)APPPS-
R²,
(SEQ ID NO:168)
R¹-HAibEGTFTSDLSKQMEEEAVRLFIEWLKNGGPK(SH)SGAPPPS-
R²,
(SEQ ID NO:102)
R¹-HAibEGTFTSDLSKQMEEEAVRLFIEWLKK(SH)GGPSSGAPPPS-
R²,
(SEQ ID NO:170)
R¹-HAibEGTFTSDLSKQMEEEAVRLFIEWLK(SH)NGGPSSGAPPPS-
R²,
(SEQ ID NO:106)
R¹-HAibEGTFTSDLSKQMEEEAVRK(SH)FIEWLKNGGPSSGAPPPS-
R²,
(SEQ ID NO:107)
R¹-HAibEGTFTSDLSKQMEEEAVK(SH)LFIEWLKNGGPSSGAPPPS-
R²,
(SEQ ID NO:108)
R¹-HAibEGTFTSDLSKQMEEEAK(SH)RLFIEWLKNGGPSSGAPPPS-
R²,
(SEQ ID NO:110)
R¹-HAibEGTFTSDLSKQMEK(SH)EAVRLFIEWLKNGGPSSGAPPPS-
R²,
(SEQ ID NO:111)
R¹-HAibEGTFTSDLSKQK(SH)EEEAVRLFIEWLKNGGPSSGAPPPS-
R²,
(SEQ ID NO:112)
R¹-HAibEGTFTSDLSKK(SH)MEEEAVRLFIEWLKNGGPSSGAPPPS-
R²,
(SEQ ID NO:169)
R¹-HAibEGTFTSDLSK(SH)QMEEEAVRLFIEWLKNGGPSSGAPPPS-
R²,
(SEQ ID NO:113)
R¹-HAibEGTFTSDLK(SH)KQMEEEAVRLFIEWLKNGGPSSGAPPPS-
R²,
and
(SEQ ID NO:115)
R¹-HAibEGTFTSDLSKQMEEEAVRKFIEWLK(SH)NGGPSS-R².

17. The GA targeting agent claimed in claim 1, comprising a peptide comprising a sequence selected from the group consisting of:
(SEQ ID NO:106)
R¹-HAibEGTFTSDLSKQMEEEAVRK(SH)FIEWLKNGGPSSGAPPPS-
R²,
(SEQ ID NO:107)
R¹-HAibEGTFTSDLSKQMEEEAVK(SH)LFIEWLKNGGPSSGAPPPS-
R²,
(SEQ ID NO:108)
R¹-HAibEGTFTSDLSKQMEEEAK(SH)RLFIEWLKNGGPSSGAPPPS-
R²,
(SEQ ID NO:110)
R¹-HAibEGTFTSDLSKQMEK(SH)EAVRLFIEWLKNGGPSSGAPPPS-
R²,
(SEQ ID NO:111)
R¹-HAibEGTFTSDLSKQK(SH)EEEAVRLFIEWLKNGGPSSGAPPPS-
R²,
and
(SEQ ID NO:112)
R¹-HAibEGTFTSDLSKK(SH)MEEEAVRLFIEWLKNGGPSSGAPPPS-
R².

18. A compound as claimed in claim 17 comprising the structure:

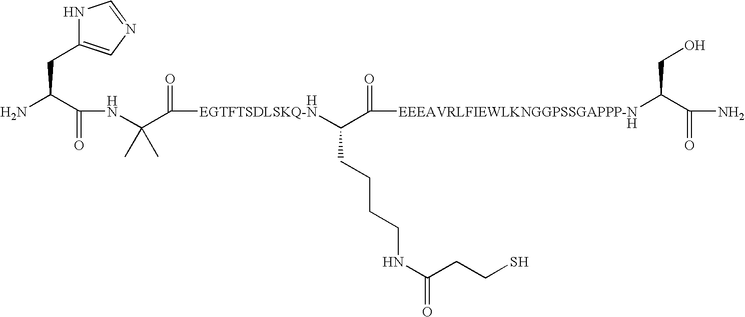

19-22. (canceled)

23. A compound having the formula:
L-[GA targeting agent]
wherein:
[GA targeting agent] is a GA targeting agent as claimed in claim 1 and
L is a linker moiety having the formula —X—Y-Z-, wherein:
X is optionally present and when present is attached to the K(SH) sidechain of the GA targeting agent, is substituted or unsubstituted, and is selected from —R²²—[CH₂—CH₂—O]ₜ—R²³—, —R²²-cycloalkyl-R²³—, —R²²-aryl-R²³—, or —R²²-heterocyclyl-R²³, wherein:
t=1-10; and
the size of R²² and R²³ are such that the backbone length of X remains about 200 atoms or less;
Y is an optionally present recognition group comprising at least a ring structure; and
Z is a reactive group that is capable of forming a covalent bond with an amino acid sidechain in a combining site of an antibody.

24-29. (canceled)

30. The compound according to claim 23, wherein Y is present and has the optionally substituted structure: 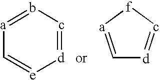
wherein a, b, c, d, and e are independently carbon or nitrogen; f is carbon, nitrogen, oxygen, or sulfur; Y is attached to X and Z independently at any two ring positions of sufficient valence; and no more than four of a, b, c, d, e, or f are simultaneously nitrogen.

31. The compound according to claim 30, wherein a, b, c, d, and e in the ring structure are each carbon.

32-35. (canceled)

36. The compound according to claim 30, wherein Z has the structure: 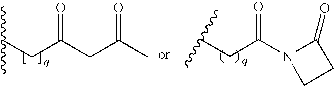
wherein q=0-5.

37-44. (canceled)

45. A compound as claimed in claim 23, selected from the group consisting of:

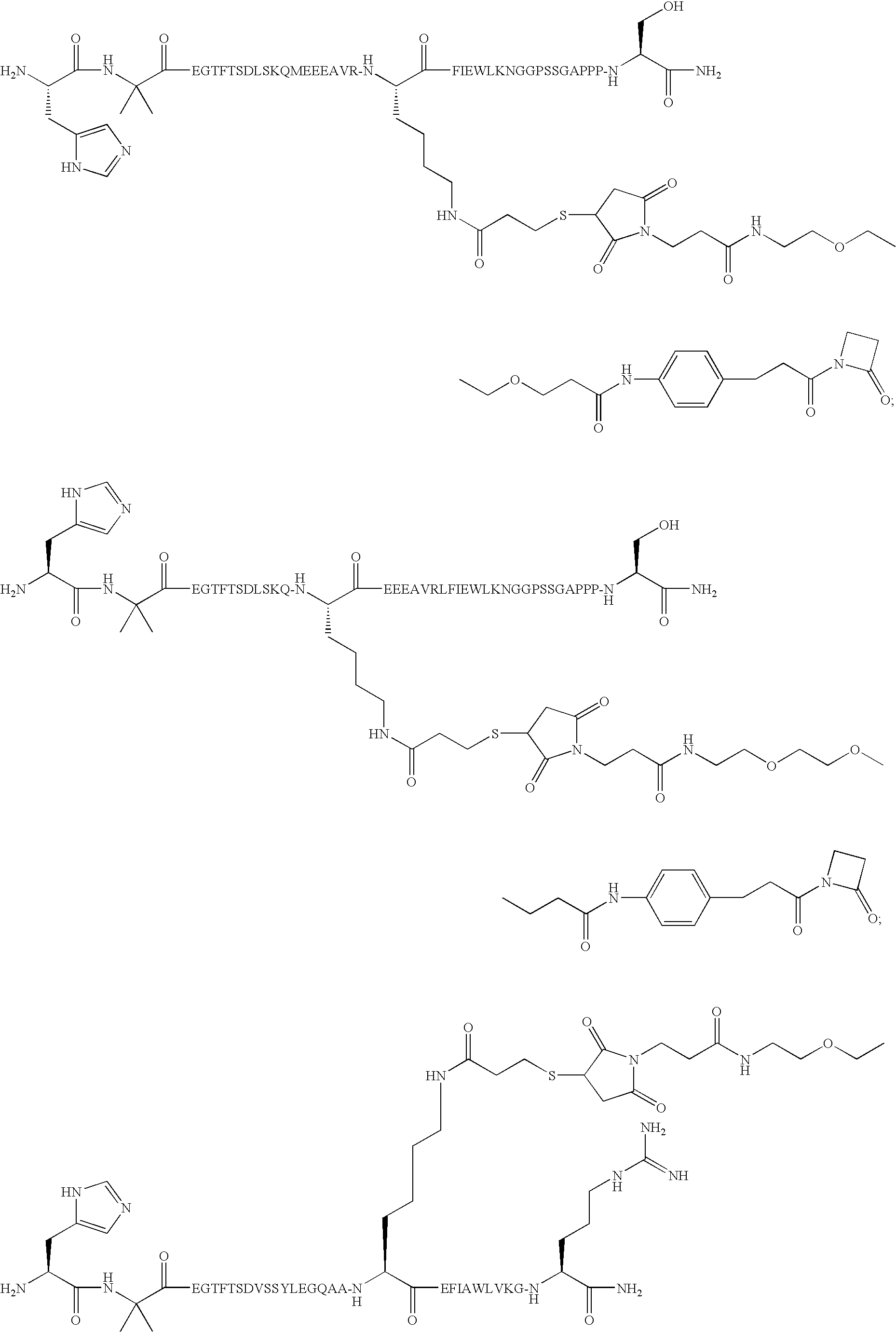

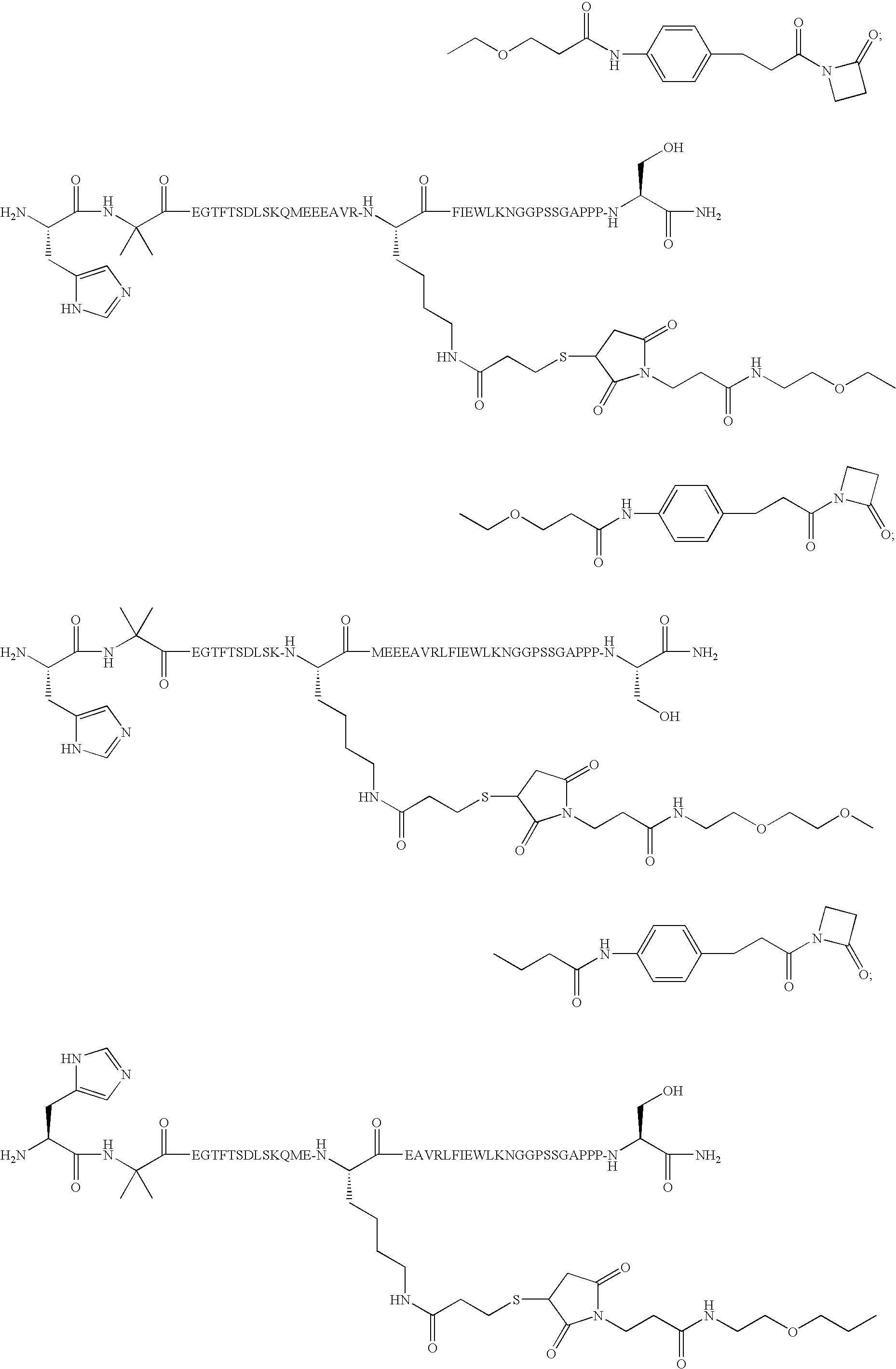

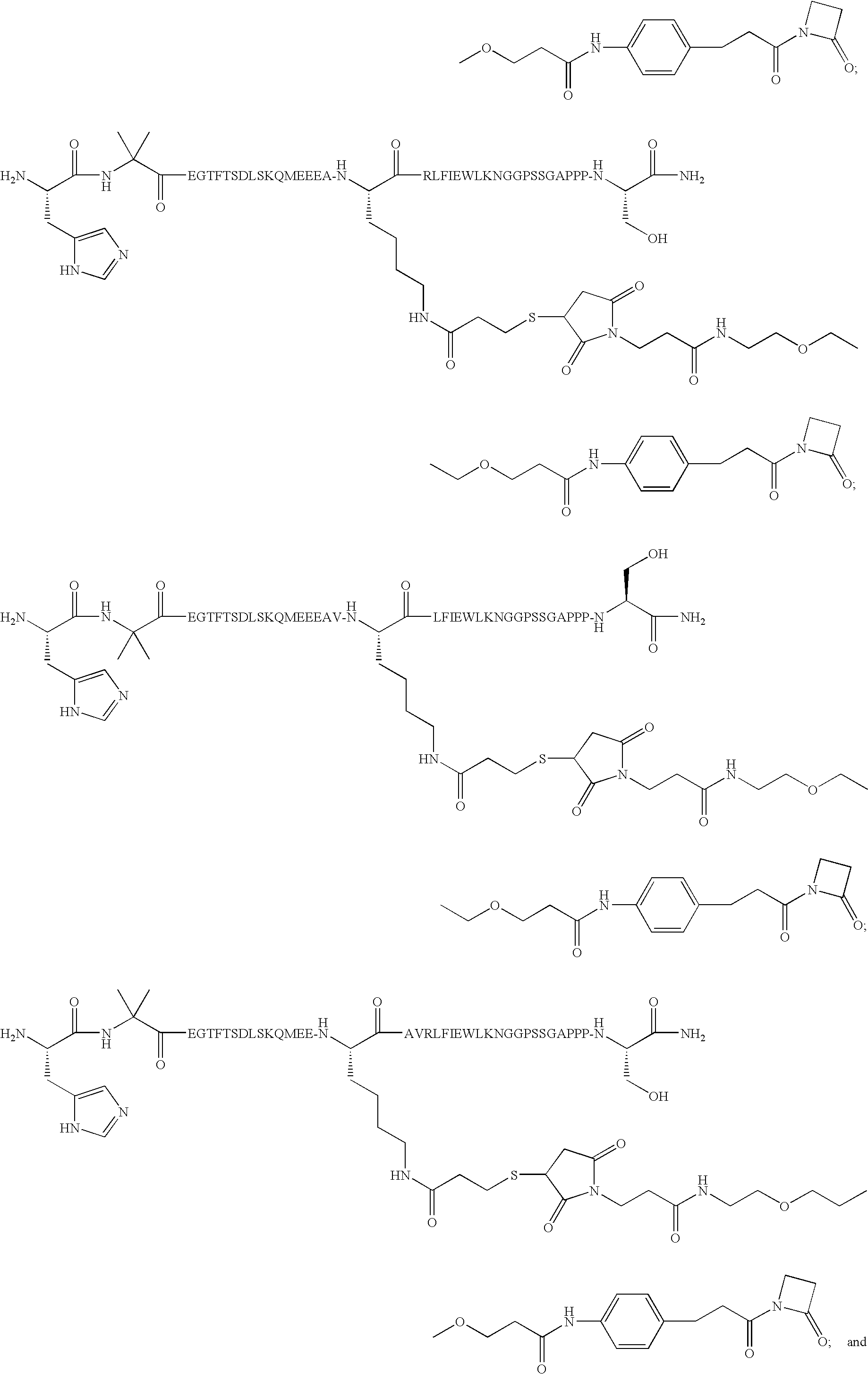

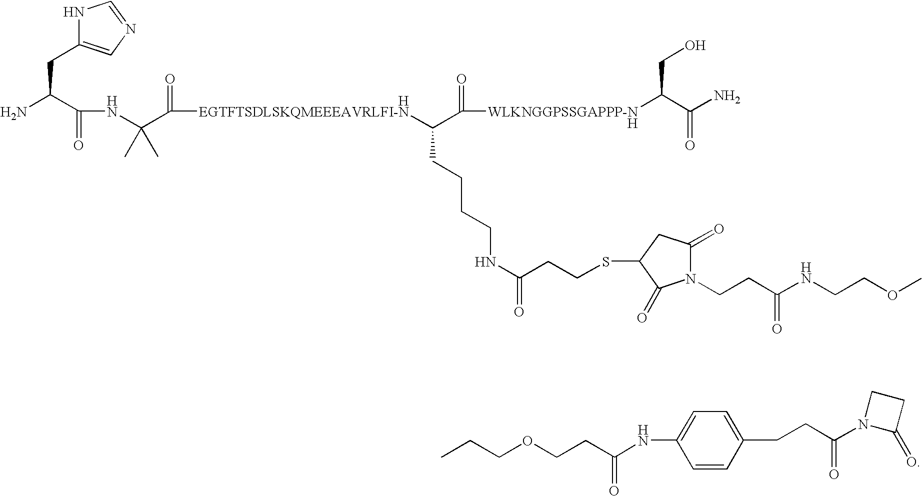

46-59. (canceled)

60. A peptide agonist of the GLP-1 receptor (GA targeting agent), comprising a peptide sequence:
R¹—H¹x²E³G⁴T⁵F⁶T⁷S⁸D⁹x¹⁰S¹¹x¹²x¹³x¹⁴E¹⁵x¹⁶x¹⁷A¹⁸x¹⁹x²⁰x²¹F²²x²³x²⁴x²⁵x²⁶x²⁷x²⁸x²⁹x³⁰x³¹x³²x³³x³⁴x³⁵x³⁶x³⁷x³⁸x³⁹-R²,
wherein
R¹ is absent, CH₃, C(O)CH3, C(O)CH₂CH₃, C(O)CH₂CH₂CH₃, or C(O)CH(CH₃)CH₃;
R² is absent, OH, NH₂, NH(CH₃), NHCH₂CH₃, NHCH₂CH₂CH₃, NHCH(CH₃)CH₃, NHCH₂CH₂CH₂CH₃, NHCH(CH₃)CH₂CH₃, NHC₆H₅, NHCH₂CH₂OCH₃, NHOCH₃, NHOCH₂CH₃, a carboxy protecting group, a lipid fatty acid group or a carbohydrate, and
wherein x² is blocking group such as Aib, A, S, T, V, L, I, D-Ala, and
x¹⁰ is V, L, I, or A,
x¹² is S or K,
x¹³ is Q or Y,
x¹⁴ is G, C, F, Y, W, M, or L,
x¹⁶ is K, D, E, or G,
x¹⁷ is E or Q,
x¹⁹ is L, I, V, or A,
x²⁰ is Orn, K(SH), R, or K,
x²¹ is L or E,
x²³ is I or L,
x²⁴ is A or E,
x²⁵ is W or F,
x²⁶ is L or I,
x²⁷ is I, K, or V,
x²⁸ is R, Orn, N, or K,
x²⁹ is Aib or G,
x³⁰ is any amino acid, preferably G or R,
x³¹ is P or absent,
x³² is S or absent,
x³³ is S or absent,
x³⁴ is G or absent,
x³⁵ is A or absent,
x³⁶ is P or absent,
x³⁷ is P or absent,
x³⁸ is P or absent,
x³⁹ is S or absent,
x⁴⁰ is a linking residue or absent,
and wherein the peptide is covalently linked to the combining site of an antibody via an intermediate linker (L′), and L′ is covalently linked to either the C-terminus or a nucleophilic sidechain of a Linking Residue (-[LR]-),
such that -[LR]- is selected from the group comprising K, R, Y, C, T, S, K(SH)), homocysteine, and homoserine, and when present, substitutes one of x¹⁰, x¹¹, x¹², x¹³, x¹⁴, x¹⁶, x¹⁷, x¹⁹, x²⁰, x²¹, x²⁴, x²⁶, x²⁷, x²⁸, x³², x³⁴, x³⁵, x³⁶, x³⁷, x³⁸, x³⁹, or x⁴⁰.

61. The GA targeting agent as claimed in claim 60, wherein x² is Aib.

62. The GA targeting agent as claimed in claim 60, comprising a peptide sequence:
R¹—H¹Aib²E³G⁴T⁵F⁶T⁷S⁸D⁹V¹⁰S¹¹S¹²Y¹³x¹⁴E¹⁵x¹⁶Q¹⁷A¹⁸x¹⁹x²⁰E²¹F²²I²³A²⁴x²⁵L²⁶x²⁷x²⁸x²⁹R³⁰—R²,
wherein x¹⁴ is G, C, F, Y, W, or L,
x¹⁶ is K, D, E, or G,
x¹⁹ is L, I, V, or A,
x²⁰ is Orn, R, or K,
x²⁵ is W or F,
x²⁷ is I or V,
x²⁸ is R or K, and
x²⁹ is Aib or G.

63. (canceled)

64. The GA targeting agent as claimed in claim 60, wherein the linking residue is selected from the group consisting of K, Y, T, and K(SH).

65-68. (canceled)

69. The compound as claimed in claim 60,
wherein:
L′ has the formula —X—Y-Z′, wherein
X is optionally present and is a biologically compatible connecting chain including any atom selected from the group consisting of C, H, N, O, P, S, F, CL, Br, and I, and may comprise a polymer or block co-polymer
Y is an optionally present recognition group comprising at least a ring structure; and
Z′ is an attachment moiety comprising a covalent link to an amino acid side in a combining site of an antibody.

70-75. (canceled)

76. The compound as claimed in claim 69, wherein X is: 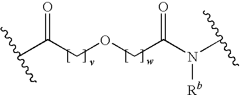
wherein v and w are selected such that the backbone length of X is 6-12 atoms;

77. The compound as claimed in claim 76, wherein X—Y is: 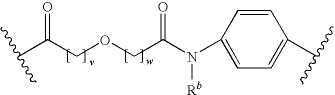
v=1 or 2; w=1 or 2; and R^{b} is hydrogen.

78. The compound according to claim 69, wherein the ring structure Y is present and has the optionally substituted structure: 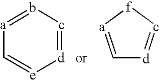
wherein a, b, c, d, and e are independently carbon or nitrogen; f is carbon, nitrogen, oxygen, or sulfur; Y is attached to X and Z or Z′ independently at any two ring positions of sufficient valence; and no more than four of a, b, c, d, e, or f are simultaneously nitrogen and preferably a, b, c, d, and e in the ring structure are each carbon.

79. The compound according to claim 78, wherein the ring structure Y is phenyl.

80. The compound according to claim 69, wherein Z′ is substituted alkyl, substituted cycloalkyl, substituted aryl, substituted arylalkyl, substituted heterocyclyl, or substituted heterocyclylalkyl, wherein at least one substituent is a 1,3-diketone moiety, an acyl beta-lactam, an active ester, an alpha-haloketone, an aldehyde, a maleimide, a lactone, an anhydride, an alpha-haloacetamide, an amine, a hydrazide, or an epoxide or the product formed by the addition of an amino group on the sidechain of an amino acid in the combining site of an antibody with an 1,3-diketone moiety, an acyl beta-lactam, an active ester, an alpha-haloketone, an aldehyde, a maleimide, a lactone, an anhydride, an alpha-haloacetamide, an amine, a hydrazide, or an epoxide.

81-82. (canceled)

83. The compound according to claim 69, wherein Z′ has the structure: 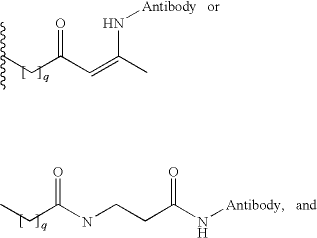
wherein q=0-5 and Antibody if present is a covalent bond to a sidechain in a combining site of an antibody.

84. A GA targeting agent as claimed in claim 60, comprising a peptide sequence selected from the group consisting of:
(SEQ ID NO:3)
R¹-HGEGTFTSDLSKQMEEEAVRLFIEWLKNGGPSSGAPPPSK-R²,
(SEQ ID NO:4)
R¹-HAibEGTFTSDLSKQMEEEAVRLFIEWLKNGGPSSGAPPPSK-R²,
(SEQ ID NO:5)
R¹-HAibEGTFTSDLSKQMEEEAVRLFIEWLKNGGPSSGAPPPS-R²,
(SEQ ID NO:6)
R¹-HAibEGTFTSDLSKQMEEEAVRLFIEWLKNGG-R²,
(SEQ ID NO:7)
R¹-HAibEGTFTSDLSKQMEEEAVRLFIEWLKN-R²,
(SEQ ID NO:8)
R¹-HAibEGTFTSDLSKQLEEEAVRLFIEFLKN-R²,
(SEQ ID NO:9)
R¹-HAibEGTFTSDLSKQLEEEAVRLAIEFLKN-R²,
(SEQ ID NO:10)
R¹-HAibEGTFTSDLSKQLEEEAVRLAIEFLKNGGPSSGAPPPS-R²,
(SEQ ID NO:11)
R¹-HAibEGTFTSDLSKQLEEEAVRLFIEFLKNGGPSSGAPPPS-R²,
(SEQ ID NO:12)
R¹-HAibEGTFTSDLSK(Ac)QMEEEAVRLFIEWLK(Ac)NGGPSSGAPP
PS-R²,
(SEQ ID NO:13)
HAibEGTFTSDLSK(benzoyl)QMEEEAVRLFIEWLK(benzoyl)NGG
PSSGAPPPS-R²,
(SEQ ID NO:14)
R¹-HAibEGTFTSDLSKQMEEEAVRLFIEWLKNGGPSSGAPPKS-R²,
(SEQ ID NO:99)
R¹-HAibEGTFTSDLSKQMEEEAVRLFIEWLKNGGPSSGAPPK(SH)S-
R²,
(SEQ ID NO:15)
R¹-HAibEGTFTSDLSKQMEEEAVRLFIEWLKNGGPSSGAKPPS-R²,
(SEQ ID NO:100)
R¹-HAibEGTFTSDLSKQMEEEAVRLFIEWLKNGGPSSGAK(SH)PPS-
R²,
(SEQ ID NO:16)
R¹-HAibEGTFTSDLSKQMEEEAVRLFIEWLKNGGPSSKAPPPS-R²,
(SEQ ID NO:101)
R¹-HAibEGTFTSDLSKQMEEEAVRLFIEWLKNGGPSSK(SH)APPPS-
R²,
(SEQ ID NO:17)
R¹-HAibEGTFTSDLSKQMEEEAVRLFIEWLKNGGPKSGAPPPS-R²,
(SEQ ID NO:18)
R¹-HAibEGTFTSDLSKQMEEEAVRLFIEWLKKGGPSSGAPPPS-R²,
(SEQ ID NO:102)
R¹-HAibEGTFTSDLSKQMEEEAVRLFIEWLKK(SH)GGPSSGAPPPS-
R²,
(SEQ ID NO:19)
R¹-HAibEGTFTSDLSKQMEEEAVRLFIEWKKNGGPSSGAPPPS-R²,
(SEQ ID NO:103)
R¹-HAibEGTFTSDLSKQMEEEAVRLFIEWK(SH)KNGGPSSGAPPPS-
R²,
(SEQ ID NO:20)
R¹-HAibEGTFTSDLSKQMEEEAVRLFIKWLKNGGPSSGAPPPS-R²
(SEQ ID NO:104)
R¹-HAibEGTFTSDLSKQMEEEAVRLFIK(SH)WLKNGGPSSGAPPPS-
R²,
(SEQ ID NO:21)
R¹-HAibEGTFTSDLSKQMEEEAVRLFKEWLKNGGPSSGAPPPS-R²,
(SEQ ID NO:105)
R¹-HAibEGTFTSDLSKQMEEEAVRLFK(SH)EWLKNGGPSSGAPPPS-
R²,
(SEQ ID NO:22)
R¹-HAibEGTFTSDLSKQMEEEAVRKFIEWLKNGGPSSGAPPPS-R²,
(SEQ ID NO:106)
R¹-HAibEGTFTSDLSKQMEEEAVRK(SH)FIEWLKNGGPSSGAPPPS-
R²,
(SEQ ID NO:23)
R¹-HAibEGTFTSDLSKQMEEEAVKLFIEWLKNGGPSSGAPPPS-R²,
(SEQ ID NO:107)
R¹-HAibEGTFTSDLSKQMEEEAVK(SH)LFIEWLKNGGPSSGAPPPS-
R²,
(SEQ ID NO:24)
R¹-HAibEGTFTSDLSKQMEEEAKRLFIEWLKNGGPSSGAPPPS-R²,
(SEQ ID NO:108)
R¹-HAibEGTFTSDLSKQMEEEAK(SH)RLFIEWLKNGGPSSGAPPPS-
R²,
(SEQ ID NO:25)
R¹-HAibEGTFTSDLSKQMEEKAVRLFIEWLKNGGPSSGAPPPS-R²,
(SEQ ID NO:109)
R¹-HAibEGTFTSDLSKQMEEK(SH)AVRLFIEWLKNGGPSSGAPPPS-
R²,
(SEQ ID NO:26)
R¹-HAibEGTFTSDLSKQMEKEAVRLFIEWLKNGGPSSGAPPPS-R²,
(SEQ ID NO:110)
R¹-HAibEGTFTSDLSKQMEK(SH)EAVRLFIEWLKNGGPSSGAPPPS-
R²,
(SEQ ID NO:27)
R¹-HAibEGTFTSDLSKQKEEEAVRLFIEWLKNGGPSSGAPPPS-R²,
(SEQ ID NO:111)
R¹-HAibEGTFTSDLSKQK(SH)EEEAVRLFIEWLKNGGPSSGAPPPS-
R²,
(SEQ ID NO:28)
R¹-HAibEGTFTSDLSKKMEEEAVRLFIEWLKNGGPSSGAPPPS-R²,
(SEQ ID NO:112)
R¹-HAibEGTFTSDLSKK(SH)MEEEAVRLFIEWLKNGGPSSGAPPPS-
R²,
(SEQ ID NO:29)
R¹-HAibEGTFTSDLKKQMEEEAVRLFIEWLKNGGPSSGAPPPS-R²,
(SEQ ID NO:113)
R¹-HAibEGTFTSDLK(SH)KQMEEEAVRLFIEWLKNGGPSSGAPPPS-
R²,
(SEQ ID NO:30)
R¹-HAibEGTFTSDLSKQMEEEAVRLFIEWLKNGGK-R²,
(SEQ ID NO:114)
R¹-HAibEGTFTSDLSKQMEEEAVRLFIEWLKNGGK(SH)-R²,
(SEQ ID NO:31)
R¹-HAibEGTFTSDLSKQMEEEAVRKFIEWLKNGGPSS-R²,
(SEQ ID NO:115)
R¹-HAibEGTFTSDLSKQMEEEAVRKFIEWLK(SH)NGGPSS-R²,
(SEQ ID NO:32)
R¹-HAibEGTFTSDVSSYLEGQAAKEFIAWLVKGR-R²,
(SEQ ID NO:33)
R¹-HAEGTFTSDVSSYLEGQAAKEFIAWLVKGRK-R²,
(SEQ ID NO:34)
R¹-HGEGTFTSDVSSYLEGQAAKEFIAWLVKGRK-R²,
(SEQ ID NO:35)
R¹-HAibEGTFTSDVSSYLEGQAAKEFIAWLVKAibR-R²,
(SEQ ID NO:36)
R¹-HAibEGTFTSDVSSYLEGQAAKEFIAWLVKAibRK-R²,
(SEQ ID NO:37)
R¹-HAibEGTFTSDVSSYLEGQAAKEFIAWLVKGRK-R²,
(SEQ ID NO:38)
R¹-HAibEGTFTSDVSSYLEGQAAKEFIAWLVKGRK(SH)-R²,
(SEQ ID NO:39)
R¹-HAibEGTFTSDVSSYLEGQMK(SH)EFIAWLVKGR-R²,
(SEQ ID NO:40)
R¹-HAibEGTFTSDVSSYGEGQAAKEFIAWLVKAibR-R²,
(SEQ ID NO:41)
R¹-HAibEGTFTSDVSSYCEGQAAKEFIAWLVKAibR-R²,
(SEQ ID NO:42)
R¹-HAibEGTFTSDVSSYFEGQAAKEFIAWLVKAibR-R²,
(SEQ ID NO:43)
R¹-HAibEGTFTSDVSSYYEGQAAKEFIAWLVKAibR-R²,
(SEQ ID NO:44)
R¹-HAibEGTFTSDVSSYWEGQAAKEFIAWLVKAibR-R²,
(SEQ ID NO:45)
R¹-HAibEGTFTSDVSSYLEEQAAKEFIAWLVKAibR-R²,
(SEQ ID NO:46)
R¹-HAibEGTFTSDVSSYLEDQAAKEFIAWLVKAibR-R²,
(SEQ ID NO:47)
R¹-HAibEGTFTSDVSSYLEKQAAKEFIAWLVKAibR-R²,
(SEQ ID NO:48)
R¹-HAibEGTFTSDVSSYLEGQAVKEFIAWLVKAibR-R²,
(SEQ ID NO:49)
R¹-HAibEGTFTSDVSSYLEGQAIKEFIAWLVKAibR-R²,
(SEQ ID NO:50)
R¹-HAibEGTFTSDVSSYLEGQALKEFIAWLVKAibR-R²,
(SEQ ID NO:51)
R¹-HAibEGTFTSDVSSYLEGQAAREFIAWLVKAibR-R²,
(SEQ ID NO:52)
R¹-HAibEGTFTSDVSSYLEGQAAOrnEFIAWLVKAibR-R²,
(SEQ ID NO:53)
R¹-HAIbEGTFTSDVSSYLEGQAAKEFIAFLVKAibR-R²,
(SEQ ID NO:54)
R¹-HAibEGTFTSDVSSYLEGQAAKEFIAWLIKAibR-R²,
(SEQ ID NO:55)
R¹-HAibEGTFTSDVSSYLEGQAAKEFIAWLVRAibR-R²,
(SEQ ID NO:56)
R¹-HAibEGTFTSDVSSYLEGQAAKEFIAWLVOrnAibR-R²,
(SEQ ID NO:57)
R¹-HAibEGTFTSDVSSYLEEQAVKEFIAWLIKAibR-R²,
(SEQ ID NO:58)
R¹-HAibEGTFTSDVSSYFEEQAVKEFIAWLIKAibR-R²,
(SEQ ID NO:59)
R¹-HAibEGTFTSDVSSYYEEQAVKEFIAWLIKAibR-R²,
(SEQ ID NO:60)
R¹-HAibEGTFTSDVSSYWEEQAVKEFIAWLIKAibR-R²,
(SEQ ID NO:61)
R¹-HAibEGTFTSDVSSYLEEQAVKEFIAWLIRAibR-R²,
(SEQ ID NO:62)
R¹-HAibEGTFTSDVSSYLEEQAVREFIAWLIRAibR-R²,
(SEQ ID NO:63)
R¹-HAibEGTFTSDVSSYLEEQAVKEFIAWLIKAibRPSSGAPPPS-R²,
(SEQ ID NO:64)
R¹-HAibEGTFTSDKSSYLEEQAVKEFIAWLIKAibR-R²,
(SEQ ID NO:116)
R¹-HAibEGTFTSDK(SH)SSYLEEQAVKEFIAWLIKAibR-R²,
(SEQ ID NO:65)
R¹-HAibEGTFTSDVSKYLEEQAVKEFIAWLIKAibR-R²,
(SEQ ID NO:117)
R¹-HAibEGTFTSDVSK(SH)YLEEQAVKEFIAWLIKAibR-R²,
(SEQ ID NO:66)
R¹-HAibEGTFTSDVSSYKEEQAVKEFIAWLIKAibR-R²,
(SEQ ID NO:118)
R¹-HAibEGTFTSDVSSYK(SH)EEQAVKEFIAWLIKAibR-R²,
(SEQ ID NO:67)
R¹-HAibEGTFTSDVSSYLEKQAVKEFIAWLIKAibR-R²,
(SEQ ID NO:119)
R¹-HAibEGTFTSDVSSYLEK(SH)QAVKEFIAWLIKAibR-R²,
(SEQ ID NO:68)
R¹-HAibEGTFTSDVSSYLEEQKVKEFIAWLIKAibR-R²,
(SEQ ID NO:120)
R¹-HAibEGTFTSDVSSYLEEQK(SH)VKEFIAWLIKAibR-R2
(SEQ ID NO:69)
R¹-HAibEGTFTSDVSSYLEEQAVKEKIAWLIKAibR-R,
(SEQ ID NO:121)
R¹-HAibEGTFTSDVSSYLEEQAVK(SH)EKIAWLIKAibR-R²,
(SEQ ID NO:70)
R¹-HAibEGTFTSDVSSYLEEQAVKEFIKWLIKAibR-R²,
(SEQ ID NO:122)
R¹-HAibEGTFTSDVSSYLEEQAVKEFIK(SH)WLIKAibR-R²,
(SEQ ID NO:71)
R¹-HAibEGTFTSDVSSYLEEQAVKEFIAWKIKAibR-R²,
(SEQ ID NO:123)
R¹-HAibEGTFTSDVSSYLEEQAVKEFIAWK(SH)IKAibR-R²,
(SEQ ID NO:72)
R¹-HAibEGTFTSDVSSYLEEQAVKEFIAWLIKAIbRPSSGAPPPSK-R²,
(SEQ ID NO:124)
R¹-HAibEGTFTSDVSSYLEEQAVKEFIAWLIKAibRPSSGAPPPSK
(SH)-R²,
(SEQ ID NO:73)
R¹-HAibEGTFTSDVSSYLEEQAVKEFIAWLIK(Ac)AibR-R²,
(SEQ ID NO:74)
R¹-HAibEGTFTSDVSSYLEEQAVKEFIAWLIK(benzoyl)AibR-R²,
(SEQ ID NO:75)
R¹-H(Trans 3-hexanoyl)AibEGTFTSDVSSYLEEQAVKEFIAWLI
KAibR-R²,
(SEQ ID NO:76)
R¹-H(3-Aminophenylacetyl)AibEGTFTSDVSSYLEEQAVKEFIA
WLIKAibR-R²,
(SEQ ID NO:168)
R¹-HAibEGTFTSDLSKQMEEEAVRLFIEWLKNGGPK(SH)SGAPPPS-
R²,
(SEQ ID NO:169)
R¹-HAibEGTFTSDLSK(SH)QMEEEAVRLFIEWLKNGGPSSGAPPPS-
R²,
(SEQ ID NO:170)
R¹-HAibEGTFTSDLSKQMEEEAVRLFIEWLK(SH)NGGPSSGAPPPS-
R²,
(SEQ ID NO:171)
R¹-HAibEGTFTSDVSSYLEEQAVK(SH)EFIAWLIKAibRPSSGAPPP
S-R²,
(SEQ ID NO:172)
R¹-HGEGTFTSDLSKQMEEEAVRLFIEWLKNGGPSSGAPPPSK(SH)-
R²,
and
(SEQ ID NO:173)
R¹-HAibEGTFTSDLSKQMEEEAVRLFIEWLKNGGPSSGAPPPSK(SH)-
R².

85. The GA targeting agent as claimed in claim 60, comprising a peptide comprising a sequence at least 90% homologous to one or more compounds selected from the group consisting of:
(SEQ ID NO:33)
R¹-HAEGTFTSDVSSYLEGQAAKEFIAWLVKGRK-R²,
(SEQ ID NO:34)
R¹-HGEGTFTSDVSSYLEGQAAKEFIAWLVKGRK-R²,
(SEQ ID NO:35)
R¹-HAibEGTFTSDVSSYLEGQAAKEFIAWLVKAibR-R²,
(SEQ ID NO:36)
R¹-HAibEGTFTSDVSSYLEGQAAKEFIAWLVKAibRK-R²,
(SEQ ID NO:37)
R¹-HAibEGTFTSDVSSYLEGQAAKEFIAWLVKGRK-R²,
(SEQ ID NO:38)
R¹-HAibEGTFTSDVSSYLEGQAAKEFIAWLVKGRK(SH)-R²,
(SEQ ID NO:39)
R¹-HAibEGTFTSDVSSYLEGQAAK(SH)EFIAWLVKGR-R²,
(SEQ ID NO:40)
R¹-HAibEGTFTSDVSSYGEGQAAKEFIAWLVKAibR-R²,
(SEQ ID NO:41)
R¹-HAibEGTFTSDVSSYCEGQAAKEFIAWLVKAibR-R²,
(SEQ ID NO:42)
R¹-HAibEGTFTSDVSSYFEGQAAKEFIAWLVKAibR-R²,
(SEQ ID NO:43)
R¹-HAibEGTFTSDVSSYYEGQAAKEFIAWLVKAibR-R²,
(SEQ ID NO:44)
R¹-HAibEGTFTSDVSSYWEGQAAKEFIAWLVKAibR-R²,
(SEQ ID NO:45)
R¹-HAibEGTFTSDVSSYLEEQAAKEFIAWLVKAibR-R²,
(SEQ ID NO:46)
R¹-HAibEGTFTSDVSSYLEDQAAKEFIAWLVKAibR-R²,
(SEQ ID NO:47)
R¹-HAibEGTFTSDVSSYLEKQAAKEFIAWLVKAibR-R²,
(SEQ ID NO:48)
R¹-HAibEGTFTSDVSSYLEGQAVKEFIAWLVKAibR-R²,
(SEQ ID NO:49)
R¹-HAibEGTFTSDVSSYLEGQAIKEFIAWLVKAibR-R²,
(SEQ ID NO:50)
R¹-HAibEGTFTSDVSSYLEGQALKEFIAWLVKAibR-R²,
(SEQ ID NO:51)
R¹-HAibEGTFTSDVSSYLEGQAAREFIAWLVKAibR-R²,
(SEQ ID NO:52)
R¹-HAibEGTFTSDVSSYLEGQAAOrnEFIAWLVKAibR-R²,
(SEQ ID NO:53)
R¹-HAibEGTFTSDVSSYLEGQAAKEFIAFLVKAibR-R²,
(SEQ ID NO:54)
R¹-HAibEGTFTSDVSSYLEGQAAKEFIAWLIKAibR-R²,
(SEQ ID NO:55)
R¹-HAibEGTFTSDVSSYLEGQMKEFIAWLVRAibR-R²,
(SEQ ID NO:56)
R¹-HAibEGTFTSDVSSYLEGQMKEFIAWLVOrnAibR-R²,
(SEQ ID NO:57)
R¹-HAibEGTFTSDVSSYLEEQAVKEFIAWLIKAibR-R²,
(SEQ ID NO:58)
R¹-HAibEGTFTSDVSSYFEEQAVKEFIAWLIKAibR-R²,
(SEQ ID NO:59)
R¹-HAibEGTFTSDVSSYYEEQAVKEFIAWLIKAibR-R²,
(SEQ ID NO:60)
R¹-HAibEGTFTSDVSSYWEEQAVKEFIAWLIKAibR-R²,
(SEQ ID NO:61)
R¹-HAibEGTFTSDVSSYLEEQAVKEFIAWLIRAibR-R²,
(SEQ ID NO:62)
R¹-HAibEGTFTSDVSSYLEEQAVREFIAWLIRAibR-R²,
(SEQ ID NO:63)
R¹-HAibEGTFTSDVSSYLEEQAVKEFIAWLIKAibRPSSGAPPPS-R²,
(SEQ ID NO:64)
R¹-HAibEGTFTSDKSSYLEEQAVKEFIAWLIKAibR-R²,
(SEQ ID NO:116)
R¹-HAibEGTFTSDK(SH)SSYLEEQAVKEFIAWLIKAibR-R²,
(SEQ ID NO:65)
R¹-HAibEGTFTSDVSKYLEEQAVKEFIAWLIKAibR-R²,
(SEQ ID NO:117)
R¹-HAibEGTFTSDVSK(SH)YLEEQAVKEFIAWLIKAibR-R²,
(SEQ ID NO:66)
R¹-HAibEGTFTSDVSSYKEEQAVKEFIAWLIKAibR-R²,
(SEQ ID NO:118)
R¹-HAibEGTFTSDVSSYK(SH)EEQAVKEFIAWLIKAibR-R²,
(SEQ ID NO:67)
R¹-HAibEGTFTSDVSSYLEKQAVKEFIAWLIKAibR-R²,
(SEQ ID NO:119)
R¹-HAibEGTFTSDVSSYLEK(SH)QAVKEFIAWLIKAibR-R²,
(SEQ ID NO:68)
R¹-HAibEGTFTSDVSSYLEEQKVKEFIAWLIKAibR-R²,
(SEQ ID NO:120)
R¹-HAibEGTFTSDVSSYLEEQK(SH)VKEFIAWLIKAibR-R²,
(SEQ ID NO:69)
R¹-HAibEGTFTSDVSSYLEEQAVKEKIAWLIKAibR-R²,
(SEQ ID NO:121)
R¹-HAibEGTFTSDVSSYLEEQAVK(SH)EKIAWLIKAibR-R²,
(SEQ ID NO:70)
R¹-HAibEGTFTSDVSSYLEEQAVKEFIKWLIKAibR-R²,
(SEQ ID NO:122)
R¹-HAibEGTFTSDVSSYLEEQAVKEFIK(SH)WLIKAibR-R²,
(SEQ ID NO:71)
R¹-HAibEGTFTSDVSSYLEEQAVKEFIAWKIKAibR-R²,
(SEQ ID NO:123)
R¹-HAibEGTFTSDVSSYLEEQAVKEFIAWK(SH)IKAibR-R²,
(SEQ ID NO:72)
R¹-HAibEGTFTSDVSSYLEEQAVKEFIAWLIKAibRPSSGAPPPSK-
R²,
(SEQ ID NO:124)
R¹-HAibEGTFTSDVSSYLEEQAVKEFIAWLIKAibRPSSGAPPPSK
(SH)-R²,
(SEQ ID NO:73)
R¹-HAibEGTFTSDVSSYLEEQAVKEFIAWLIK(Ac)AibR-R²,
(SEQ ID NO:74)
R¹-HAibEGTFTSDVSSYLEEQAVKEFIAWLIK(benzoyl)AibR-R²,
(SEQ ID NO:1)
R¹-HAEGTFTSDVSSYLEGQAAKEFIAWLVKGR-R²,
(SEQ ID NO:125)
R¹-HAEGTFTSDVSSYLEGQAAKEFIAWLVKGRK-R²,
(SEQ ID NO:126)
R¹-HAibEGTFTSDVSSYLEGQMKEFIAWLVKGRK-R²,
(SEQ ID NO:127)
R¹-HAibEGTFTSDVSSYLEGQAAKEFIAWLVKGR-R²,
(SEQ ID NO:128)
R¹-HAibEGTFTSDVSSYLEEQAVKEFIAWLIKAibR-R²,
and
(SEQ ID NO:129)
R¹-HAibEGTFTSDVSSYLEEQAVKEFIAWLIKAibRPSSGAPPPS-R².

86-87. (canceled)

88. The GA targeting agent as claimed in claim 60, comprising a peptide comprising a sequence selected from the group consisting of:
(SEQ ID NO:29)
R¹-HAibEGTFTSDLKKQMEEEAVRLFIEWLKNGGPSSGAPPPS-R²,
(SEQ ID NO:113)
R¹-HAibEGTFTSDLK(SH)KQMEEEAVRLFIEWLKNGGPSSGAPPPS-
R²,
(SEQ ID NO:5)
R¹-HAibEGTFTSDLSKQMEEEAVRLFIEWLKNGGPSSGAPPPS-R²,
(SEQ ID NO:28)
R¹-HAibEGTFTSDLSKKMEEEAVRLFIEWLKNGGPSSGAPPPS-R²,
(SEQ ID NO:112)
R¹-HAibEGTFTSDLSKK(SH)MEEEAVRLFIEWLKNGGPSSGAPPPS-
R²,
(SEQ ID NO:113)
R¹-HAibEGTFTSDLSKQMEEEAVRLFIEWLKNGGPSSGAPPPS-R²,
(SEQ ID NO:115)
R¹-HAibEGTFTSDLSKQMEEEAVRLFIEWLK(SH)NGGPSSGAPPPS-
R2
(SEQ ID NO:156)
R¹-HAibEGTFTSDLSKQMEEEAVRLFIEWLK(L′)NGGPSSGAPPPS-
R²,
(SEQ ID NO:18)
R¹-HAibEGTFTSDLSKQMEEEAVRLFIEWLKKGGPSSGAPPPS-R²,
(SEQ ID NO:102)
R¹-HAibEGTFTSDLSKQMEEEAVRLFIEWLKK(SH)GGPSSGAPPPS-
R²,
(SEQ ID NO:17)
R¹-HAibEGTFTSDLSKQMEEEAVRLFIEWLKNGGPKSGAPPPS-R²,
(SEQ ID NO:168)
R¹-HAibEGTFTSDLSKQMEEEAVRLFIEWLKNGGPK(SH)SGAPPPS-
R²,
(SEQ ID NO:16)
R¹-HAibEGTFTSDLSKQMEEEAVRLFIEWLKNGGPSSKAPPPS-R²,
(SEQ ID NO:101)
R¹-HAibEGTFTSDLSKQMEEEAVRLFIEWLKNGGPSSK(SH)APPPS-
R²,
(SEQ ID NO:15)
R¹-HAibEGTFTSDLSKQMEEEAVRLFIEWLKNGGPSSGAKPPS-R²,
(SEQ ID NO:100)
R¹-HAibEGTFTSDLSKQMEEEAVRLFIEWLKNGGPSSGAK(SH)PPS-
R²,
(SEQ ID NO:14)
R¹-HAibEGTFTSDLSKQMEEEAVRLFIEWLKNGGPSSGAPPKS-R²,
(SEQ ID NO:99)
R¹-HAibEGTFTSDLSKQMEEEAVRLFIEWLKNGGPSSGAPPK(SH)S-
R²,
(SEQ ID NO:22)
R¹-HAibEGTFTSDLSKQMEEEAVRK(L)FIEWLKNGGPSSGAPPPS-
R²,
(SEQ ID NO:23)
R¹-HAibEGTFTSDLSKQMEEEAVK(L)LFIEWLKNGGPSSGAPPPS-
R²,
(SEQ ID NO:24)
R¹-HAibEGTFTSDLSKQMEEEAK(L)RLFIEWLKNGGPSSGAPPPS-
R²,
(SEQ ID NO:26)
R¹-HAibEGTFTSDLSKQMEK(L)EAVRLFIEWLKNGGPSSGAPPPS-
R²,
(SEQ ID NO:27)
R¹-HAibEGTFTSDLSKQK(L)EEEAVRLFIEWLKNGGPSSGAPPPS-
R²,
(SEQ ID NO:106)
R¹-HAibEGTFTSDLSKQMEEEAVRK(L)FIEWLKNGGPSSGAPPPS-
R²,
(SEQ ID NO:107)
R¹-HAibEGTFTSDLSKQMEEEAVK(L)LFIEWLKNGGPSSGAPPPS-
R²,
(SEQ ID NO:108)
R¹-HAibEGTFTSDLSKQMEEEAK(L)RLFIEWLKNGGPSSGAPPPS-
R²,
(SEQ ID NO:110)
R¹-HAibEGTFTSDLSKQMEK(L)EAVRLFIEWLKNGGPSSGAPPPS-
R²,
(SEQ ID NO:111)
R¹-HAibEGTFTSDLSKQK(L)EEEAVRLFIEWLKNGGPSSGAPPPS-
R².
(SEQ ID NO:157)
R¹-HAibEGTFTSDLSKQMEEEAVRK(L)FIEWLKNGGPSSGAPPPS-
R²,
(SEQ ID NO:158)
R¹-HAibEGTFTSDLSKQMEEEAVK(L)LFIEWLKNGGPSSGAPPPS-
R²,
(SEQ ID NO:159)
R¹-HAibEGTFTSDLSKQMEEEAK(L)RLFIEWLKNGGPSSGAPPPS-
R²,
(SEQ ID NO:165)
R¹-HAibEGTFTSDLSKQMEK(L)EAVRLFIEWLKNGGPSSGAPPPS-
R²,
and
(SEQ ID NO:160)
R¹-HAibEGTFTSDLSKQK(L)EEEAVRLFIEWLKNGGPSSGAPPPS-
R².

89-114. (canceled)

115. A compound or pharmaceutically acceptable salt thereof selected from the group consisting of: 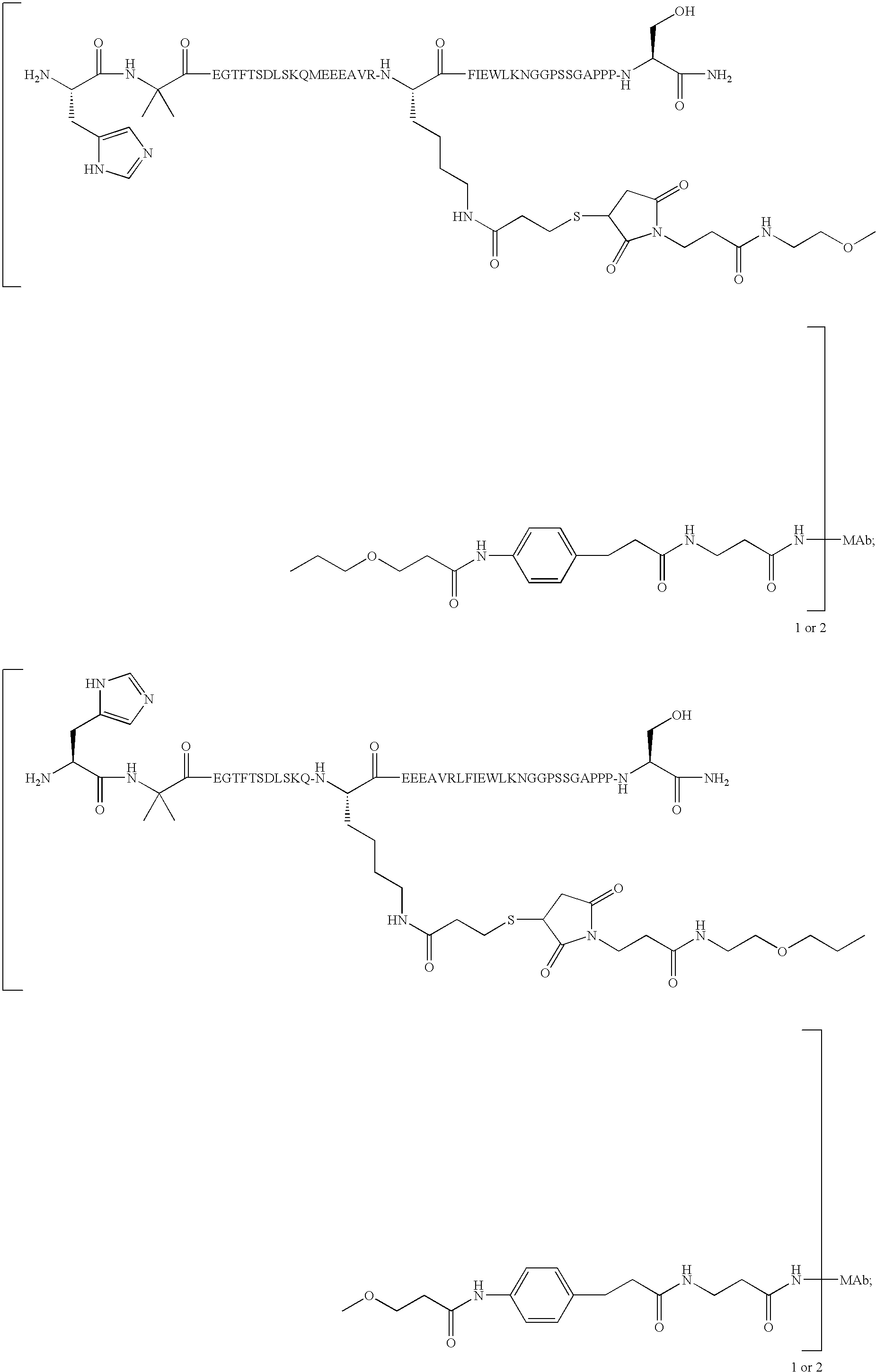 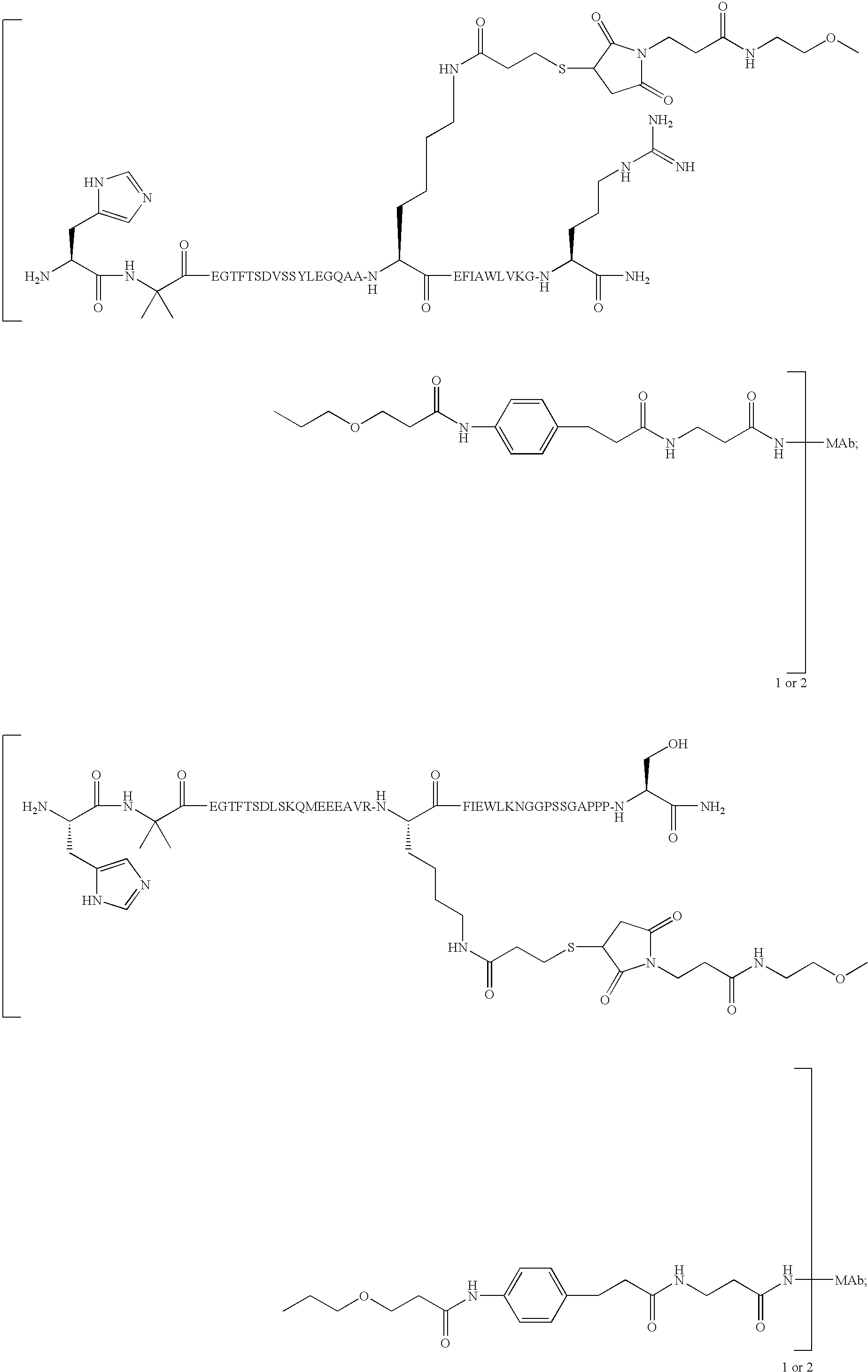 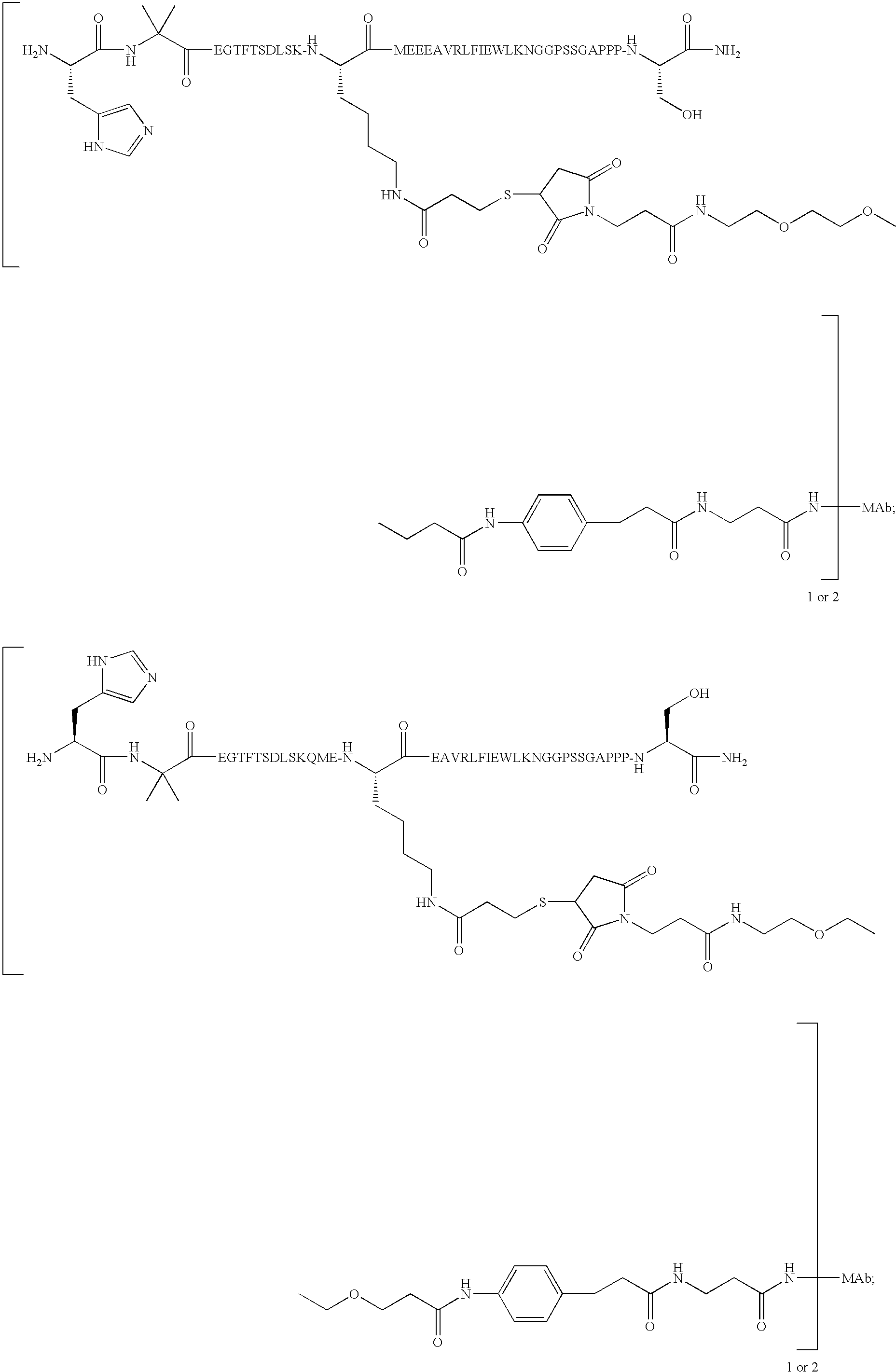 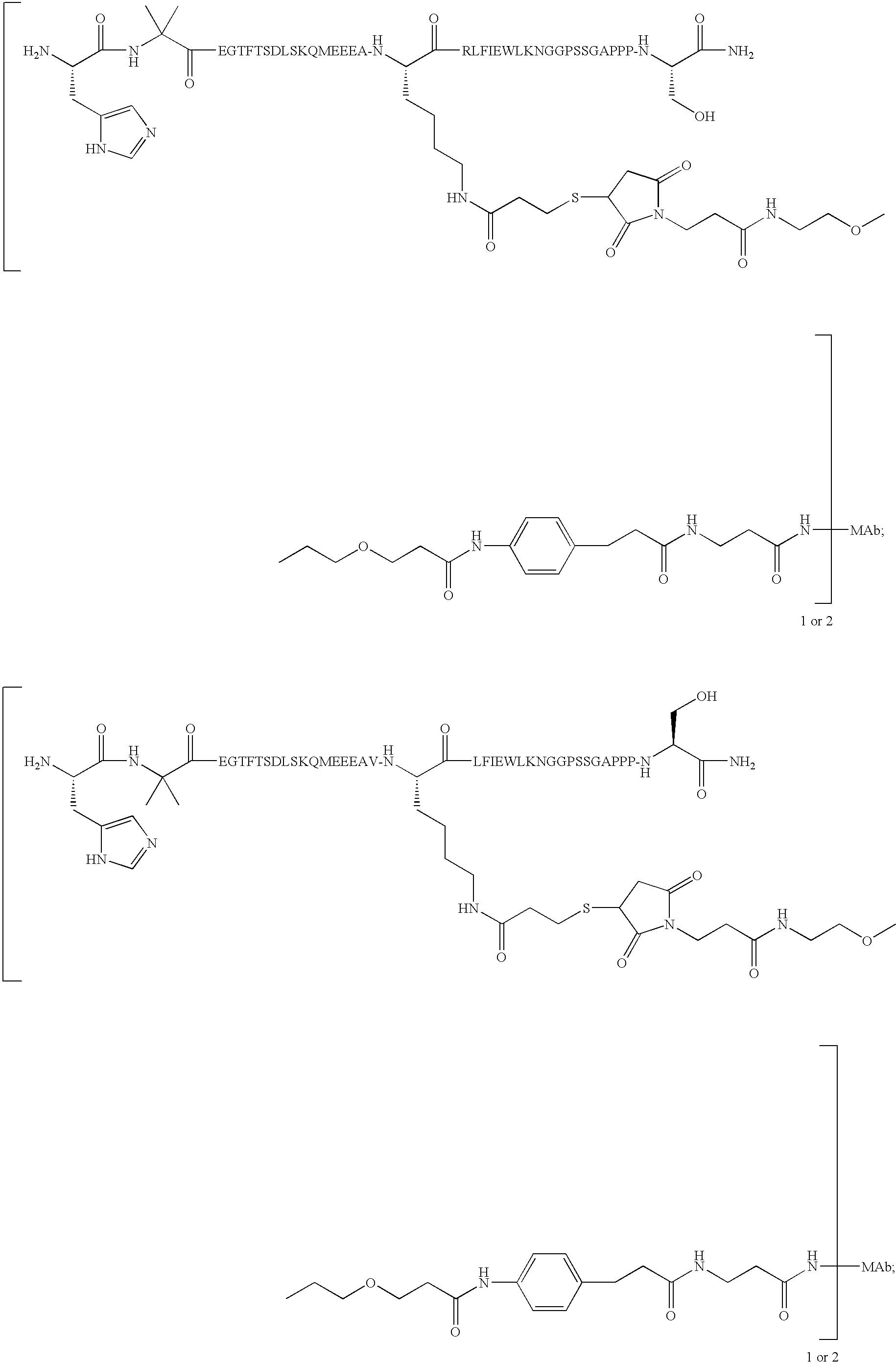 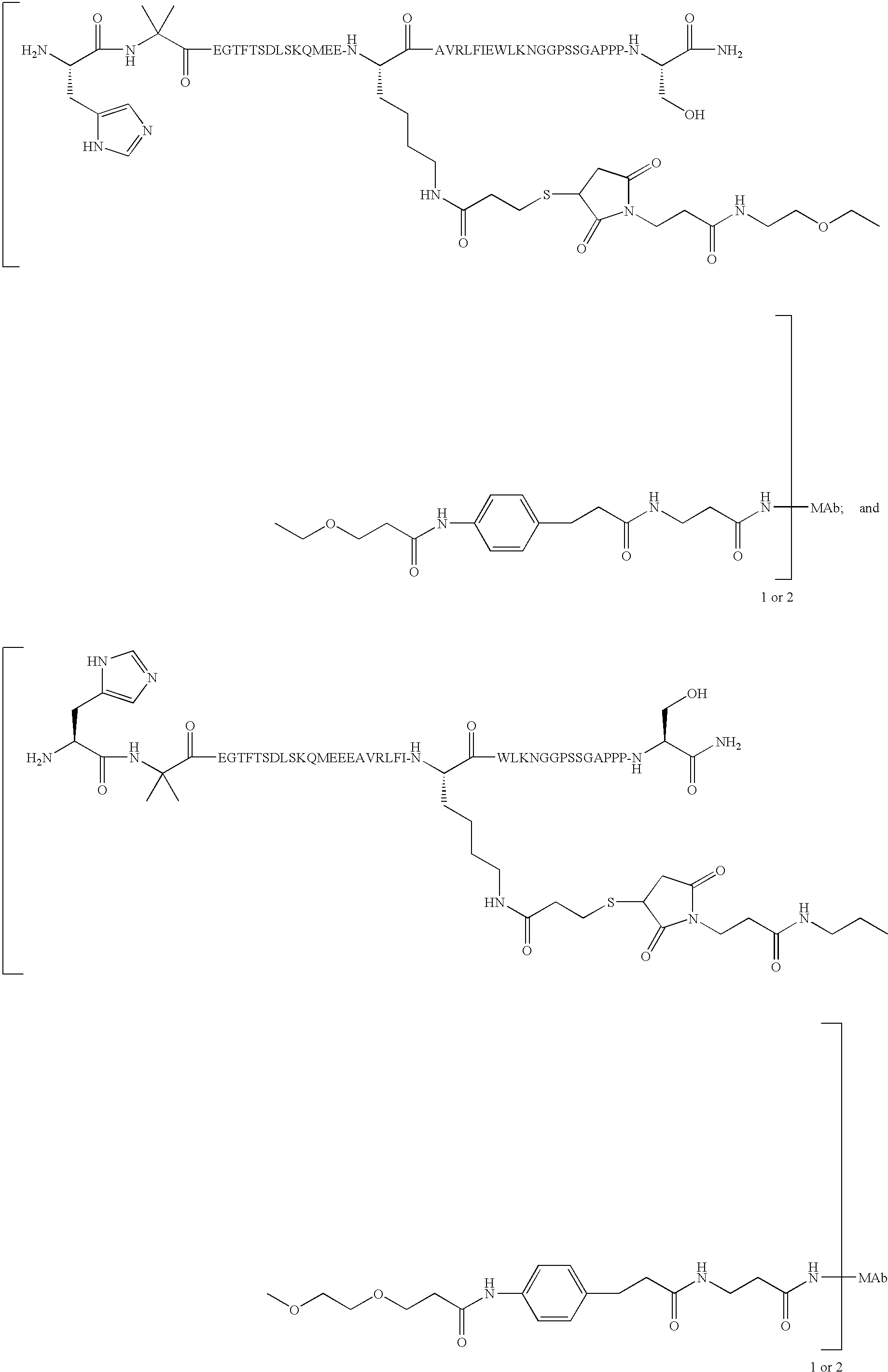
wherein MAb is an antibody comprising the V_{H} and V_{L} domains from h38c2.

116-133. (canceled)

134. The compound of claim 115, wherein MAb is h38c2 IgG1.

135. A pharmaceutical composition comprising a therapeutically effective amount of a compound of claim 134.

136. The pharmaceutical composition of claim 135, further comprising a therapeutically effective amount of a compound selected from the group consisting of sulfonylureas, biguanides, thiazolidinediones, alpha glucosidase inhibitors, and meglinitides.

137. Use of the compound of claim 134 in a method of increasing the blood glucose level of an individual.

138. A method of increasing the blood glucose level of an individual comprising treating the individual with a therapeutically effective amount of the compound of claim 134.

139. A method of producing a GA targeting compound, comprising covalently linking a compound of claim 45 with at least one of the combining sites of h38c2 IgG1.

140. A compound as claimed in claim 23, wherein X is: 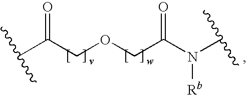
wherein v and w are selected such that the backbone length of X is 6-12 atoms.

141. A compound according to claim 69, wherein the antibody is a catalytic antibody.

142. A compound according to claim 141, wherein the antibody is an aldolase antibody.
